SEQUENCE LISTING (134 sequences)

SEQ ID NO: 1 - DNA, 989 bp, Artificial Sequence
  3′ arm of target sequence
caaaggcttacctatcactagcgcgacacgttcctaagtgaaaagcttaggcactgtcgaactcaacagttcagcagtgaactgtcattctaagaagtcaaatgaaggagtaacgtctggaagggcttcccttaatcctccgacatgcaggaaagtaggcaagtactgaactgtgtgaagctcggtgaagtcggttgaaggttaccgtaaattagtatctctaatacgaaagctatccagcggtggatggtgtaactgatagaccggaggtctataaaacactcaaggttaggatgcgcgatgaactagaggcgatcgctagtaagcgcagacgaatccctgatggtacgggtctatatcgggagggaatcgaaaggttctctgacacaaataagtgtcgctactgtgggtgagtaaaactctcctttatgaaagcccatatatcgttacaggcgttattaaggtagcaggctcataggggaaacctaaaagtgtatgtacagataagaatgacggaacgtggtaagctgccgacatggagggcttgttctctttgaagtgttgccaaggaaagtcacaatgagattagttgtcgatataacttggtttaacggcagtgaaagtggtggcacagtaccgatgaaacgtgtaatgaacgtggagggatagccactagtcgattgaagattgaaggttactattggttaacatggtttcgagtaagactaagagatgtaatgctccaaagtaataaggaggttacagcccatgttaaagaaaaccaagctaagacataacgaatattatgatacacaaaaaaagtgtatgacaatttatactcgaacagtcttaacggtaacaatttctttcaattggaaacgatggaacgccgtatgcccggaaacgggcgcgtacggtgtggagtgggggaaaagctggagataatctcaaaggcttacctatcactatcgcgacacgttcctaagtga

SEQ ID NO: 2 - DNA, 1028 bp, Clostridium tetani
gccatacaataaaagtgcgaaacgttatcctataagtaagaaagttttaaaattttcttacgaaaaggatagaacttaaaagttctaactgttctactaaagtaataagtgaaaatcttatttaaagcaaacaaccaagtagctttaagtctaagtcccctacacaagttttatactactatgcaaaacttgtgaagctaggtaaggtcgtaatccgtgaaagtcggatgcggggctccttaaaagattactatggtaaacataagctaatccattaagatgcgatttatatgtattttatactgttaaatatttttgtgcttgtggcttggtataaaacagttaagatgaagtacttaactggttttggaataattggttgttaaactaaaacattataaatcgttagtggatacctaaggtaatcaaaaatagggataggtagaatggaacgtttgatgctgtatatgaagaggtttagtagaacctaggacacatatacgggctcagcaggttcatagtagctatgatactcagccggaagtcaattaattttgaaatacttctatggtaacataggagaaggataaaactgagtgagccaaggaacctagtcggtaatagaaaagtggaagttaaaacaaatataagattttagaattaatttaattaatgaacggaattaatttaatgatatttaaagttagacggttataaattaaacatttcaaaattaaaccatatccaaattcataaatatagctagatcatatcactagtttaaaaataaataaatcatttcaaattactattaagtaaggtattaataccttacttaatagtaatctcattacataagagaattactagattagcagacagattcataaaaactatatcaactaggacaatagaaaatatatttatacacttcctattatcgagcgaacgccttatgcgatgaaagtcgcacgtagggtgtagaccaagcgaaatcctatgcatttaggatagtgaggtatagcaaa

SEQ ID NO: 3 - DNA, 1028 bp, Artificial Sequence
  Group II intron Cte_mut
gcaagacaataaaagtttgacacgtgatcctataagtaagaaagttttaaaattttcttacgaaaaggatagaacttaaaagttctaactgttctactaaagtaataagtgaaaatcttatttaaagcaaacaaccaagtagctttaagtctaagtcccctacacaagttttatactactatgcaaaacttgtgaagctaggtaaggtcgtaatccgtgaaagtcggatgcggggctccttaaaagattactatggtaaacataagctaatccattaagatgcgatttatatgtattttatactgttaaatatttttgtgcttgtggcttggtataaaacagttaagatgaagtacttaactggttttggaataattggttgttaaactaaaacattataaatcgttagtggatacctaaggtaatcaaaaatagggataggtagaatggaacgtttgatgctgtatatgaagaggtttagtagaacctaggacacatatacgggctcagcaggttcatagtagctatgatactcagccggaagtcaattaattttgaaatacttctatggtaacataggagaaggataaaactgagtgagccaaggaacctagtcggtaatagaaaagtggaagttaaaacaaatataagattttagaattaatttaattaatgaacggaattaatttaatgatatttaaagttagacggttataaattaaacatttcaaaattaaaccatatccaaattcataaatatagctagatcatatcactagtttaaaaataaataaatcatttcaaattactattaagtaaggtattaataccttacttaatagtaatctcattacataagagaattactagattagcagacagattcataaaaactatatcaactaggacaatagaaaatatatttatacacttcctattatcgagcgaacgccttatgcgatgaaagtcgcacgtagggtgtagaccaagcgaaatcctatgcatttaggatagtgaggtatagcaaa

SEQ ID NO: 4 - DNA, 60 bp, Artificial Sequence
  Spacer sequence1
gcaatagccgaaaaacaaaaaacaaaaaaaacaaaaaaaaaaccaaaaaaacaaaacaca

SEQ ID NO: 5 - DNA, 20 bp, Artificial Sequence
  Spacer sequence2
aaattataataattataata

SEQ ID NO: 6 - DNA, 224 bp, Artificial Sequence
  Spacer sequence3
atgaaaccggctcggattccgcccgcgtgcgccatcccctcagctagcaggtgtgagcggctttctgcccgcagtctctacacagctcagcatcctgacgcctcctccccttgcaggggcgtgaagctacttcagactctgctgtgacgacttggccgccaggcaccgatcctccccggtgagaaggtccacgaatcttactgcagacagatttgctcagcgcg

SEQ ID NO: 7 - DNA, 24 bp, Artificial Sequence
  Probe1
ctttcactactcctacgagcacca

SEQ ID NO: 8 - DNA, 25 bp, Artificial Sequence
  Probe2
gaccatgctcccaagcaagatcatg

SEQ ID NO: 9 - DNA, 555 bp, Artificial Sequence
  Gluc
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgac

SEQ ID NO: 10 - DNA, 936 bp, Artificial Sequence
  Rluc1
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 11 - DNA, 1160 bp, Artificial Sequence
  Rluc2
atgaaaccggctcggattccgcccgcgtgcgccatcccctcagctagcaggtgtgagcggctttctgcccgcagtctctacacagctcagcatcctgacgcctcctccccttgcaggggcgtgaagctacttcagactctgctgtgacgacttggccgccaggcaccgatcctccccggtgagaaggtccacgaatcttactgcagacagatttgctcagcgcgatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 12 - DNA, 1061 bp, Artificial Sequence
  IRES-EgFP
agcaaagaccccaacgagaagcgcgatcacatggtcctgctggagttcgtgaccgccgccgggatcactctcggcatggacgagctgtacaagtaacaaacaaacaaaacaaaaacactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctg

SEQ ID NO: 13 - DNA, 899 bp, Artificial Sequence
  IRES-gluc
agcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacaaacaaacaaaacaaaaacactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatgg

SEQ ID NO: 14 - DNA, 32 bp, Artificial Sequence
  arm1
aataccttacttaatagtaacaatagaaaatc

SEQ ID NO: 15 - DNA, 33 bp, Artificial Sequence
  arm2
aagctagatcatattactattaagtaaggtatt

SEQ ID NO: 16 - DNA, 1719 bp, Artificial Sequence
  cRNAzyme (Cte-Rluc)
aataccttacttaatagtaacaatagaaaatcctattatcgagcgaacgccttatgcgatgaaagtcgcacgtagggtgtagaccaagcgaaatcctatgcatttaggatagtgaggtatagcaaaatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaagccatacaataaaagtgcgaaacgttatcctataagtaagaaagttttaaaattttcttacgaaaaggatagaacttaaaagttctaactgttctactaaagtaataagtgaaaatcttatttaaagcaaacaaccaagtagctttaagtctaagtcccctacacaagttttatactactatgcaaaacttgtgaagctaggtaaggtcgtaatccgtgaaagtcggatgcggggctccttaaaagattactatggtaaacataagctaatccattaagatgcgatttatatgtattttatactgttaaatatttttgtgcttgtggcttggtataaaacagttaagatgaagtacttaactggttttggaataattggttgttaaactaaaacattataaatcgttagtggatacctaaggtaatcaaaaatagggataggtagaatggaacgtttgatgctgtatatgaagaggtttagtagaacctaggacacatatacgggctcagcaggttcatagtagctatgatactcagccggaagtcaattaattttgaaatacttctatggtaacataggagaaggataaaactgagtgagccaaggaacctagtcggtaatagaagctagatcatattactattaagtaaggtatt

SEQ ID NO: 17 - RNA, 989 nt, Bacillus thuringiensis
caaaggcuuaccuaucacuagcgcgacacguuccuaagugaaaagcuuaggcacugucgaacucaacaguucagcagugaacugucauucuaagaagucaaaugaaggaguaacgucuggaagggcuucccuuaauccuccgacaugcaggaaaguaggcaaguacugaacugugugaagcucggugaagucgguugaagguuaccguaaauuaguaucucuaauacgaaagcuauccagcgguggaugguguaacugauagaccggaggucuauaaaacacucaagguuaggaugcgcgaugaacuagaggcgaucgcuaguaagcgcagacgaaucccugaugguacgggucuauaucgggagggaaucgaaagguucucugacacaaauaagugucgcuacugugggugaguaaaacucuccuuuaugaaagcccauauaucguuacaggcguuauuaagguagcaggcucauaggggaaaccuaaaaguguauguacagauaagaaugacggaacgugguaagcugccgacauggagggcuuguucucuuugaaguguugccaaggaaagucacaaugagauuaguugucgauauaacuugguuuaacggcagugaaagugguggcacaguaccgaugaaacguguaaugaacguggagggauagccacuagucgauugaagauugaagguuacuauugguuaacaugguuucgaguaagacuaagagauguaaugcuccaaaguaauaaggagguuacagcccauguuaaagaaaaccaagcuaagacauaacgaauauuaugauacacaaaaaaaguguaugacaauuuauacucgaacagucuuaacgguaacaauuucuuucaauuggaaacgauggaacgccguaugcccggaaacgggcgcguacgguguggagugggggaaaagcuggagauaaucucaaaggcuuaccuaucacuaucgcgacacguuccuaaguga

SEQ ID NO: 18 - RNA, 1028 nt, Clostridium tetani
gccauacaauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauagaaaaguggaaguuaaaacaaauauaagauuuuagaauuaauuuaauuaaugaacggaauuaauuuaaugauauuuaaaguuagacgguuauaaauuaaacauuucaaaauuaaaccauauccaaauucauaaauauagcuagaucauaucacuaguuuaaaaauaaauaaaucauuucaaauuacuauuaaguaagguauuaauaccuuacuuaauaguaaucucauuacauaagagaauuacuagauuagcagacagauucauaaaaacuauaucaacuaggacaauagaaaauauauuuauacacuuccuauuaucgagcgaacgccuuaugcgaugaaagucgcacguaggguguagaccaagcgaaauccuaugcauuuaggauagugagguauagcaaa

SEQ ID NO: 19 - RNA, 1028 nt, Artificial Sequence
  Group II intron Cte_mut
gcaagacaauaaaaguuugacacgugauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauagaaaaguggaaguuaaaacaaauauaagauuuuagaauuaauuuaauuaaugaacggaauuaauuuaaugauauuuaaaguuagacgguuauaaauuaaacauuucaaaauuaaaccauauccaaauucauaaauauagcuagaucauaucacuaguuuaaaaauaaauaaaucauuucaaauuacuauuaaguaagguauuaauaccuuacuuaauaguaaucucauuacauaagagaauuacuagauuagcagacagauucauaaaaacuauaucaacuaggacaauagaaaauauauuuauacacuuccuauuaucgagcgaacgccuuaugcgaugaaagucgcacguaggguguagaccaagcgaaauccuaugcauuuaggauagugagguauagcaaa

SEQ ID NO: 20 - RNA, 60 nt, Artificial Sequence
  Spacer sequence 1
gcaauagccgaaaaacaaaaaacaaaaaaaacaaaaaaaaaaccaaaaaaacaaaacaca

SEQ ID NO: 21 - RNA, 20 nt, Artificial Sequence
  Spacer sequence2
aaauuauaauaauuauaaua

SEQ ID NO: 22 - RNA, 224 nt, Artificial Sequence
  Spacer sequence 3
augaaaccggcucggauuccgcccgcgugcgccauccccucagcuagcaggugugagcggcuuucugcccgcagucucuacacagcucagcauccugacgccuccuccccuugcaggggcgugaagcuacuucagacucugcugugacgacuuggccgccaggcaccgauccuccccggugagaagguccacgaaucuuacugcagacagauuugcucagcgcg

SEQ ID NO: 23 - RNA, 24 nt, Artificial Sequence
  Probe1
cuuucacuacuccuacgagcacca

SEQ ID NO: 24 - RNA, 25 nt, Artificial Sequence
  Probe2
gaccaugcucccaagcaagaucaug

SEQ ID NO: 25 - RNA, 555 nt, Artificial Sequence
  Gluc
augggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagagauggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccauggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugac

SEQ ID NO: 26 - RNA, 936 nt, Artificial Sequence
  Rluc1
auggcuuccaagguguacgaccccgagcaacgcaaacgcaugaucacugggccucaguggugggcucgcugcaagcaaaugaacgugcuggacuccuucaucaacuacuaugauuccgagaagcacgccgagaacgccgugauuuuucugcaugguaacgcugccuccagcuaccuguggaggcacgucgugccucacaucgagcccguggcuagaugcaucaucccugaucugaucggaauggguaaguccggcaagagcgggaauggcucauaucgccuccuggaucacuacaaguaccucaccgcuugguucgagcugcugaaccuuccaaagaaaaucaucuuugugggccacgacuggggggcuugucuggccuuucacuacuccuacgagcaccaagacaagaucaaggccaucguccaugcugagagugucguggacgugaucgaguccugggacgaguggccugacaucgaggaggauaucgcccugaucaagagcgaagagggcgagaaaauggugcuugagaauaacuucuucgucgagaccaugcucccaagcaagaucaugcggaaacuggagccugaggaguucgcugccuaccuggagccauucaaggagaagggcgagguuagacggccuacccucuccuggccucgcgagaucccucucguuaagggaggcaagcccgacgucguccagauuguccgcaacuacaacgccuaccuucgggccagcgacgaucugccuaagauguucaucgaguccgacccuggguucuuuuccaacgcuauugucgagggagcuaagaaguucccuaacaccgaguucgugaaggugaagggccuccacuucagccaggaggacgcuccagaugaaauggguaaguacaucaagagcuucguggagcgcgugcugaagaacgagcaguaa

SEQ ID NO: 27 - RNA, 1160 nt, Artificial Sequence
  Rluc2
augaaaccggcucggauuccgcccgcgugcgccauccccucagcuagcaggugugagcggcuuucugcccgcagucucuacacagcucagcauccugacgccuccuccccuugcaggggcgugaagcuacuucagacucugcugugacgacuuggccgccaggcaccgauccuccccggugagaagguccacgaaucuuacugcagacagauuugcucagcgcgauggcuuccaagguguacgaccccgagcaacgcaaacgcaugaucacugggccucaguggugggcucgcugcaagcaaaugaacgugcuggacuccuucaucaacuacuaugauuccgagaagcacgccgagaacgccgugauuuuucugcaugguaacgcugccuccagcuaccuguggaggcacgucgugccucacaucgagcccguggcuagaugcaucaucccugaucugaucggaauggguaaguccggcaagagcgggaauggcucauaucgccuccuggaucacuacaaguaccucaccgcuugguucgagcugcugaaccuuccaaagaaaaucaucuuugugggccacgacuggggggcuugucuggccuuucacuacuccuacgagcaccaagacaagaucaaggccaucguccaugcugagagugucguggacgugaucgaguccugggacgaguggccugacaucgaggaggauaucgcccugaucaagagcgaagagggcgagaaaauggugcuugagaauaacuucuucgucgagaccaugcucccaagcaagaucaugcggaaacuggagccugaggaguucgcugccuaccuggagccauucaaggagaagggcgagguuagacggccuacccucuccuggccucgcgagaucccucucguuaagggaggcaagcccgacgucguccagauuguccgcaacuacaacgccuaccuucgggccagcgacgaucugccuaagauguucaucgaguccgacccuggguucuuuuccaacgcuauugucgagggagcuaagaaguucccuaacaccgaguucgugaaggugaagggccuccacuucagccaggaggacgcuccagaugaaauggguaaguacaucaagagcuucguggagcgcgugcugaagaacgagcaguaa

SEQ ID NO: 28 - RNA, 1061 nt, Artificial Sequence
  IRES-EgFP
agcaaagaccccaacgagaagcgcgaucacaugguccugcuggaguucgugaccgccgccgggaucacucucggcauggacgagcuguacaaguaacaaacaaacaaaacaaaaacacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaauggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccug

SEQ ID NO: 29 - RNA, 899 nt, Artificial Sequence
  IRES-gluc
agcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaacaaacaaacaaaacaaaaacacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucaaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaaugggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagagauggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccaugg

SEQ ID NO: 30 - RNA, 32 nt, Artificial Sequence
  arm1
aauaccuuacuuaauaguaacaauagaaaauc

SEQ ID NO: 31 - RNA, 33 nt, Artificial Sequence
  arm2
aagcuagaucauauuacuauuaaguaagguauu

SEQ ID NO: 32 - RNA, 1719 nt, Artificial Sequence
  cRNAzyme (Cte-Rluc)
aauaccuuacuuaauaguaacaauagaaaauccuauuaucgagcgaacgccuuaugcgaugaaagucgcacguaggguguagaccaagcgaaauccuaugcauuuaggauagugagguauagcaaaauggcuuccaagguguacgaccccgagcaacgcaaacgcaugaucacugggccucaguggugggcucgcugcaagcaaaugaacgugcuggacuccuucaucaacuacuaugauuccgagaagcacgccgagaacgccgugauuuuucugcaugguaacgcugccuccagcuaccuguggaggcacgucgugccucacaucgagcccguggcuagaugcaucaucccugaucugaucggaauggguaaguccggcaagagcgggaauggcucauaucgccuccuggaucacuacaaguaccucaccgcuugguucgagcugcugaaccuuccaaagaaaaucaucuuugugggccacgacuggggggcuugucuggccuuucacuacuccuacgagcaccaagacaagaucaaggccaucguccaugcugagagugucguggacgugaucgaguccugggacgaguggccugacaucgaggaggauaucgcccugaucaagagcgaagagggcgagaaaauggugcuugagaauaacuucuucgucgagaccaugcucccaagcaagaucaugcggaaacuggagccugaggaguucgcugccuaccuggagccauucaaggagaagggcgagguuagacggccuacccucuccuggccucgcgagaucccucucguuaagggaggcaagcccgacgucguccagauuguccgcaacuacaacgccuaccuucgggccagcgacgaucugccuaagauguucaucgaguccgacccuggguucuuuuccaacgcuauugucgagggagcuaagaaguucccuaacaccgaguucgugaaggugaagggccuccacuucagccaggaggacgcuccagaugaaauggguaaguacaucaagagcuucguggagcgcgugcugaagaacgagcaguaagccauacaauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauagaagcuagaucauauuacuauuaaguaagguauu

SEQ ID NO: 33 - RNA, 426 nt, Artificial Sequence
  Group II Intron
cucucuaaauagcaauauuuaccuuuggagggaaaaguuaucaggcaugcaccugguagcuagucuuuaaaccaauagauugcaucgguuuaaaaggcaagaccgucaaauugcgggaaaggggucaacagccguucaguaccaagucucaggggaaacuuugagauggccuugcaaaggguaugguaauaagcugacggacaugguccuaaccacgcagccaaguccuaagucaacagaucuucuguugauauggaugcaguucacagacuaaaugucggucggggaagauguauucuucucauaagauauagucggaccucuccuuaaugggagcuagcggaugaagugaugcaacacuggagccgcugggaacuaauuuguaugcgaaaguauauugauuaguuuuggaguacucguaaggua

SEQ ID NO: 34 - RNA, 787 nt, Artificial Sequence
  Group II Intron
ugcaugugaugcaggucguggugcgaaucguuccuaagugaaaagcuuaggcaucuuagacaagggaaggccccauuaagauagaacuaaugauucgagagacagaaugauagaguaacgucuugaaauguuuccccuaagucuccgacaugcauugaaagguaggauguuuaaaugugugaagcucggugaagacggcugacagauaccguagugaaaaauguguuuuaaccgaaaguccuuuuaguaagaggauguaucccauaggccggggguccguaaaguaucuauggugagaauguuauaugaaauaacugacgaacuuucgaauuaacgagucuauaaccauacaaucagaaaugauaguaaaccauauuuuguguugugugugagguuaaguaaauucgcggcuaugaacaaccuuacaguauuauagguacugucuagcacgcaggcucauagaaggcaccuaaggguaccauauaguggauagaaucauuggaacgaugaaagcucaggacgcagagaaccuacauucugugaagcggugguaaggaaaaggaggaauccuauaacuuaucuuuauuugagugauagcgaugucauaguagcguggaauuuauggaaacauaaaggagcgaagggcauuagucaauauugugaggaaaaagcaauauugauggcaagccguaugaagggaaacuuucauguacgguuuagugugggggaaaaagcagagauuauaucaaaguuuuaccuaucacaauaaggagagaauauuuuuaau

SEQ ID NO: 35 - RNA, 989 nt, artificial sequence; identical to SEQ ID NO: 17
  Group II Intron

SEQ ID NO: 36 - RNA, 867 nt, artificial sequence
  Group II Intron
uuauaacaaaguaguuauuugugcgacacguuucuuuauaagugugcaaacacgaaguaggaggguuaucaauuugaugacaacaacggauugacugcaaguaggaaugaaagccgucagguugagcugaaacuacuuaucugauacuccuauaugcaaggcgugauaguagucacaaauuguaugaagcuaggugaagucggcugaacaaaaccuaagugagaaaucauaugguaauggauaggucgggaugcuacaaaacaucuauggugagaauguccuaacggacuggcgaauguacagguuuaaaggauuaauucauuagaaauguguauauugucaacgacgacgcuaucuaccgaaaaguaagaguaaauaauaugaaauucggaagaucuaacgaugaggauguaaagauaacaggcuuacagcaagcaccuaaagauauauguauagcuaagucauucagaacgugguaagcaagagacugucacaaaugccuacuaacagacaaggugcauauaagguucuaacgaaccaaaauugcuuuauucuugugaagguggggacacaguaccgacgaagcauguaacaaauguggagggauagucccuagucuuguucguugaaaacuaaaucaacuggauauaacucacaggaucgaguaagaugaugugacuuuucguaagaaaagggaaugaauacguuggugaguacaacaugguuuguaaucuaguuguuuuaguauaaaaggauaagauggggcgcuguaugcgaugaaagucgcacguacagugucaagcgggggaaaagauggagauaacuuuaaagucuuaccuaucgcaacggauaaaaagaaucccugcu

SEQ ID NO: 37 - RNA, 1046 nt, artificial sequence
  Group II Intron
gaauauacuauagccauacaauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauagaaaaguggaaguuaaaacaaauauaagauuuuagaauuaauuuaauuaaugaacggaauuaauuuaaugauauuuaaaguuagacgguuauaaauuaaacauuucaaaauuaaaccauauccaaauucauaaauauagcuagaucauaucacuaguuuaaaaauaaauaaaucauuucaaauuacuauuaaguaagguauuaauaccuuacuuaauaguaaucucauuacauaagagaauuacuagauuagcagacagauucauaaaaacuauaucaacuaggacaauagaaaauauauuuauacacuuccuauuaucgagcgaacgccuuaugcgaugaaagucgcacguaggguguagaccaagcgaaauccuaugcauuuaggauagugagguauagcaaaggagaa

SEQ ID NO: 38 - RNA, 626 nt, artificial sequence
  Group II Intron
acucuagguagacgagagugcgacuaguaaagugcuuaauaacaauguggugaaagcccaccagauaacccauuaucugagcucagacgguaugcauuagaaguaauucuuuuguguaaagcccguuuaauuugguaaacagaccaaccaaccuauauauaaggauggugagcuauauuacuauggauaaaauuuuuaugaauucacguucgaagcguauuagagugaguuuuauuuagaaggaaaaaaguaaauaaaauucuaauuaauuguauaaacaauuuuucguuuguuuauuaaugggucuuauauuaauguacguauagugaaauccuaagguaguaaauaagguguuauuaaguaaacuagguaagcccaauaauaucuucauaugauaguaugaagaaguucaaguguaaauuugaauauauauuaguggguaaaggauauuuuaaaaagcgaaugucucauauuaauagugagaauagguuuaugacuaauucgaaagaaugcugacuuaaaauuaauauuaauauuucgauauuaauauuugagccguaugcgaugaaagucgcacguacgguucuuagagggggaaaguccaagagggccuaccuaucucaaca

SEQ ID NO: 39 - RNA, 639 nt, artificial sequence
  Group II Intron
cucuagauagacgagagugcgacuagauaaguacuuaauagcaauguggugaaaguccaccugauaacccauagucugagcucagacgguaugcauuagaaguaauucuuuuguguaaagcccguuuaaucuggucuaaaggaccuuccacccuaaauauauagggggagagcuauaauacuaaggauaaaauuuuuuugaauucauguuuaaagcguauuagaggaaauuuauuucuaugaaaaagaagaaauaaauuuuuaauuaacuguauaaacaaguuuucguuuguuuauuagugggucuuauguuaauguacguauaguggaauccuaagguauuaauuaagguguuauuaaguaaacuagguaagcccaauagugucuucauauuauaauaugaagaaguucaauugugaaauugaauaugcauuaguggguaaaggauauuuuaaaaagcgaaugucucauauuaauagugagaauaggucuaugacuaauucgaaagaaugcugacuuaaaauugguauuaauauaugcguuucgauguauauauuaauauuugagccguaugcgaugaaagucgcacguacgguucuuagagggggaaaucuuaguaauaagacgaccuaucucgaca

SEQ ID NO: 40 - RNA, 573 nt, artificial sequence
  Group II Intron
ggguagcaauaaggaugugcgacuuguuaaguuuuaacaaaaauuguauaacguuuauuaaugauuauacauuguauuucaucuuacaauagccuaauuagauaugcauuuaggguaacuuuuuuguauaaagcucuaauuauaaguguaaauacacuuuuaggcuucuucuaugguuagagaaaucgaaccaauguaauuaaaacuuugauguauuaggcauuuaacguguccuugguuaaaugaagaugaacauaaguauacaaaguaaaauuggaaccuaaggaagaauuguuuuuguuaagaaacaagguaauaccuauaacuggcuaauauaaauuugcaagguuuauuguaaaauaaacuauagguuagagguaaaaggauaauguaaaaagcgaaugcaauucuguaauggaauugauaggguauauaccuaacuugaaagagugcugacuuacauauagauguuauuuacguuucgacguaaauaauguuugagccguaugcuaugaaaguagcacguacgguucuaagagggggaaaguccgagaggaccuaccuaucucaacu

SEQ ID NO: 41 - RNA, 659 nt, artificial sequence
  Group II Intron
acucuagguagacgagagugcgacuaguaaaguguuuaauaauaauguggugaaaacccaccagauaacccauuaucugagcucauacggugugcauuaaaaguaauuuuuuuguauaaagcccguuuaaucagguaaauaauccuuccauccuaauuauaucuauagauauaaaaggauggugagcuauauuacuauggauaagauuuuuuugaauucacguuugaagcguauuagaguaaguuuuauuuaaaaggaaaaaaaaaaauuaaauaaaauuaaauuaauuguauaaauaaguuuucguuuauuuauuaaugggucuuauauuaauguacguauagugaaauccuaauguaguaauuaagguguuauuaaauaaacuagguaagcccaauaaugucuucauaauaugaagaaguucaaguguaaauuugaauauacauuaguggguaaaggauauuuuaaaaagcgaaugucucauauuaauagugagaauaggucuaugacuaauucgaaagaaugcugacuuaaaauuaauauuaauauuaauauauauauauuaauauuugagccguaugcgaugaaaaucgcacguacgguucuuagagggggaaagcucgagagggccuaccuaucuccacagacccuaugcagcu

SEQ ID NO: 42 - RNA, 261 nt, Artificial Sequence
  3′ intron fragment
cuuugagauggccuugcaaaggguaugguaauaagcugacggacaugguccuaaccacgcagccaaguccuaagucaacagaucuucuguugauauggaugcaguucacagacuaaaugucggucggggaagauguauucuucucauaagauauagucggaccucuccuuaaugggagcuagcggaugaagugaugcaacacuggagccgcugggaacuaauuuguaugcgaaaguauauugauuaguuuuggaguacucg

SEQ ID NO: 43 - RNA, 97 nt, artificial sequence
  3′ intron fragment
gcaauauugauggcaagccguaugaagggaaacuuucauguacgguuuagugugggggaaaaagcagagauuauaucaaaguuuuaccuaucacaau

SEQ ID NO: 44 - RNA, 98 nt, artificial sequence
  3′ intron fragment
auuggaaacgauggaacgccguaugcccggaaacgggcgcguacgguguggagugggggaaaagcuggagauaaucucaaaggcuuaccuaucacuau

SEQ ID NO: 45 - RNA, 95 nt, artificial sequence
  3′ intron fragment
ggauaagauggggcgcuguaugcgaugaaagucgcacguacagugucaagcgggggaaaagauggagauaacuuuaaagucuuaccuaucgcaac

SEQ ID NO: 46 - RNA, 88 nt, artificial sequence
  3′ intron fragment
cuauuaucgagcgaacgccuuaugcgaugaaagucgcacguaggguguagaccaagcgaaauccuaugcauuuaggauagugagguau

SEQ ID NO: 47 - RNA, 85 nt, artificial sequence
  3′ intron fragment
auauuaauauuugagccguaugcgaugaaagucgcacguacgguucuuagagggggaaaguccaagagggccuaccuaucucaac

SEQ ID NO: 48 - RNA, 93 nt, artificial sequence
  3′ intron fragment
auguauauauuaauauuugagccguaugcgaugaaagucgcacguacgguucuuagagggggaaaucuuaguaauaagacgaccuaucucgac

SEQ ID NO: 49 - RNA, 88 nt, artificial sequence
  3′ intron fragment
acguaaauaauguuugagccguaugcuaugaaaguagcacguacgguucuaagagggggaaaguccgagaggaccuaccuaucucaac

SEQ ID NO: 50 - RNA, 85 nt, artificial sequence
  3′ intron fragment
auauuaauauuugagccguaugcgaugaaaaucgcacguacgguucuuagagggggaaagcucgagagggccuaccuaucuccac

SEQ ID NO: 51 - RNA, 408 nt, artificial sequence
  3′ intron fragment
gaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauagaagcuagaucauauuacuauuaaguaagguauuaauaccuuacuuaauaguaacaauagaaaauccuauuaucgagcgaacgccuuaugcgaugaaagucgcacguaggguguagaccaagcgaaauccuaugcauuuaggauagugagguau

SEQ ID NO: 52 - RNA, 217 nt, artificial sequence
  3′ intron fragment
uacuucuaugguaacauaggagaaggauaaaacugagugagccaaggaaccuagucgguaauagaagcuagaucauauuacuauuaaguaagguauuaauaccuuacuuaauaguaacaauagaaaauccuauuaucgagcgaacgccuuaugcgaugaaagucgcacguaggguguagaccaagcgaaauccuaugcauuuaggauagugagguau

SEQ ID NO: 53 - RNA, 7 nt, Artificial Sequence
  E2
uaaggua

SEQ ID NO: 54 - RNA, 6 nt, artificial sequence
  E2
aaggag

SEQ ID NO: 55 - RNA, 6 nt, artificial sequence
  E2
aaguga

SEQ ID NO: 56 - RNA, 6 nt, artificial sequence
  E2
ccugcu

SEQ ID NO: 57 - RNA, 6 nt, artificial sequence
  E2
agcagu

SEQ ID NO: 58 - RNA, 6 nt, artificial sequence
  E2
agcaaa

SEQ ID NO: 59 - RNA, 6 nt, artificial sequence
  E2
agagaa

SEQ ID NO: 60 - RNA, 6 nt, artificial sequence
  E2
agcaaa

SEQ ID NO: 61 - RNA, 1 nt, artificial sequence
  E2
a

SEQ ID NO: 62 - RNA, 1 nt, artificial sequence
  E2
u

SEQ ID NO: 63 - RNA, 6 nt, artificial sequence
  E2
agaccc

SEQ ID NO: 64 - RNA, 6 nt, artificial sequence
  E1
cucucu

SEQ ID NO: 65 - RNA, 6 nt, artificial sequence
  E1
gucgug

SEQ ID NO: 66 - RNA, 6 nt, Artificial Sequence
  E1
caaagg

SEQ ID NO: 67 - RNA, 6 nt, artificial sequence
  E1
uuauuu

SEQ ID NO: 68 - RNA, 6 nt, artificial sequence
  E1
ccaugg

SEQ ID NO: 69 - RNA, 6 nt, artificial sequence
  E1
gcccug

SEQ ID NO: 70 - RNA, 6 nt, artificial sequence
  E1
cguuga

SEQ ID NO: 71 - RNA, 6 nt, artificial sequence
  E1
gccaua

SEQ ID NO: 72 - RNA, 6 nt, artificial sequence
  E1
acgaga

SEQ ID NO: 73 - RNA, 6 nt, artificial sequence
  E1
aaggau

SEQ ID NO: 74 - RNA, 8 nt, artificial sequence
  E1
agacgaga

SEQ ID NO: 75 - RNA, 148 nt, artificial sequence
  5′ intron fragment
aaauagcaauauuuaccuuuggagggaaaaguuaucaggcaugcaccugguagcuagucuuuaaaccaauagauugcaucgguuuaaaaggcaagaccgucaaauugcgggaaaggggucaacagccguucaguaccaagucucaggg

SEQ ID NO: 76 - RNA, 641 nt, artificial sequence
  5′ intron fragment
gugcgaaucguuccuaagugaaaagcuuaggcaucuuagacaagggaaggccccauuaagauagaacuaaugauucgagagacagaaugauagaguaacgucuugaaauguuuccccuaagucuccgacaugcauugaaagguaggauguuuaaaugugugaagcucggugaagacggcugacagauaccguagugaaaaauguguuuuaaccgaaaguccuuuuaguaagaggauguaucccauaggccggggguccguaaaguaucuauggugagaauguuauaugaaauaacugacgaacuuucgaauuaacgagucuauaaccauacaaucagaaaugauaguaaaccauauuuuguguugugugugagguuaaguaaauucgcggcuaugaacaaccuuacaguauuauagguacugucuagcacgcaggcucauagaaggcaccuaaggguaccauauaguggauagaaucauuggaacgaugaaagcucaggacgcagagaaccuacauucugugaagcggugguaaggaaaaggaggaauccuauaacuuaucuuuauuugagugauagcgaugucauaguagcguggaauuuauggaaacauaaaggagcgaagggcauuagucaauauugu

SEQ ID NO: 77 - RNA, 666 nt, artificial sequence
  5′ intron fragment
gcgcgacacguuccuaagugaaaagcuuaggcacugucgaacucaacaguucagcagugaacugucauucuaagaagucaaaugaaggaguaacgucuggaagggcuucccuuaauccuccgacaugcaggaaaguaggcaaguacugaacugugugaagcucggugaagucgguugaagguuaccguaaauuaguaucucuaauacgaaagcuauccagcgguggaugguguaacugauagaccggaggucuauaaaacacucaagguuaggaugcgcgaugaacuagaggcgaucgcuaguaagcgcagacgaaucccugaugguacgggucuauaucgggagggaaucgaaagguucucugacacaaauaagugucgcuacugugggugaguaaaacucuccuuuaugaaagcccauauaucguuacaggcguuauuaagguagcaggcucauaggggaaaccuaaaaguguauguacagauaagaaugacggaacgugguaagcugccgacauggagggcuuguucucuuugaaguguugccaaggaaagucacaaugagauuaguugucgauauaacuugguuuaacggcagugaaagugguggcacaguaccgaugaaacguguaaugaacguggagggauagccacuagucgauugaag

SEQ ID NO: 78 - RNA, 604 nt, artificial sequence
  5′ intron fragment
gugcgacacguuucuuuauaagugugcaaacacgaaguaggaggguuaucaauuugaugacaacaacggauugacugcaaguaggaaugaaagccgucagguugagcugaaacuacuuaucugauacuccuauaugcaaggcgugauaguagucacaaauuguaugaagcuaggugaagucggcugaacaaaaccuaagugagaaaucauaugguaauggauaggucgggaugcuacaaaacaucuauggugagaauguccuaacggacuggcgaauguacagguuuaaaggauuaauucauuagaaauguguauauugucaacgacgacgcuaucuaccgaaaaguaagaguaaauaauaugaaauucggaagaucuaacgaugaggauguaaagauaacaggcuuacagcaagcaccuaaagauauauguauagcuaagucauucagaacgugguaagcaagagacugucacaaaugccuacuaacagacaaggugcauauaagguucuaacgaaccaaaauugcuuuauucuugugaagguggggacacaguaccgacgaagcauguaacaaauguggagggauagucccuagucuuguuc

SEQ ID NO: 79 - RNA, 618 nt, artificial sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuaaugcuuuaugguugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauag

SEQ ID NO: 80 - RNA, 618 nt, artificial sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuagggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauag

SEQ ID NO: 81 - RNA, 618 nt, artificial sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugcucuucaacguugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauag

SEQ ID NO: 82 - RNA, 618 nt, artificial sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaauggaacguuugaugcuguauaugaagagguuuaguagaaccuaggacacauauacgggcucagcagguucauaguagcuaugauacucagccggaagucaauuaauuuugaaauacuucuaugguaacauaggagaaggauaaaacugagugagccaaggaaccuagucgguaauag

SEQ ID NO: 83 - RNA, 519 nt, artificial sequence
  5′ intron fragment
gugcgacuaguaaagugcuuaauaacaauguggugaaagcccaccagauaacccauuaucugagcucagacgguaugcauuagaaguaauucuuuuguguaaagcccguuuaauuugguaaacagaccaaccaaccuauauauaaggauggugagcuauauuacuauggauaaaauuuuuaugaauucacguucgaagcguauuagagugaguuuuauuuagaaggaaaaaaguaaauaaaauucuaauuaauuguauaaacaauuuuucguuuguuuauuaaugggucuuauauuaauguacguauagugaaauccuaagguaguaaauaagguguuauuaaguaaacuagguaagcccaauaauaucuucauaugauaguaugaagaaguucaaguguaaauuugaauauauauuaguggguaaaggauauuuuaaaaagcgaaugucucauauuaauagugagaauagguuuaugacuaauucgaaagaaugcugacuuaaaauuaauauuaauau

SEQ ID NO: 84 - RNA, 525 nt, artificial sequence
  5′ intron fragment
gugcgacuagauaaguacuuaauagcaauguggugaaaguccaccugauaacccauagucugagcucagacgguaugcauuagaaguaauucuuuuguguaaagcccguuuaaucuggucuaaaggaccuuccacccuaaauauauagggggagagcuauaauacuaaggauaaaauuuuuuugaauucauguuuaaagcguauuagaggaaauuuauuucuaugaaaaagaagaaauaaauuuuuaauuaacuguauaaacaaguuuucguuuguuuauuagugggucuuauguuaauguacguauaguggaauccuaagguauuaauuaagguguuauuaaguaaacuagguaagcccaauagugucuucauauuauaauaugaagaaguucaauugugaaauugaauaugcauuaguggguaaaggauauuuuaaaaagcgaaugucucauauuaauagugagaauaggucuaugacuaauucgaaagaaugcugacuuaaaauugguauuaauauaugcgu

SEQ ID NO: 85 - RNA, 464 nt, artificial sequence
  5′ intron fragment
gugcgacuuguuaaguuuuaacaaaaauuguauaacguuuauuaaugauuauacauuguauuucaucuuacaauagccuaauuagauaugcauuuaggguaacuuuuuuguauaaagcucuaauuauaaguguaaauacacuuuuaggcuucuucuaugguuagagaaaucgaaccaauguaauuaaaacuuugauguauuaggcauuuaacguguccuugguuaaaugaagaugaacauaaguauacaaaguaaaauuggaaccuaaggaagaauuguuuuuguuaagaaacaagguaauaccuauaacuggcuaauauaaauuugcaagguuuauuguaaaauaaacuauagguuagagguaaaaggauaauguaaaaagcgaaugcaauucuguaauggaauugauaggguauauaccuaacuugaaagagugcugacuuacauauagauguuauuuacgu

SEQ ID NO: 86 - RNA, 538 nt, artificial sequence
  5′ intron fragment
gugcgacuaguaaaguguuuaauaauaauguggugaaaacccaccagauaacccauuaucugagcucauacggugugcauuaaaaguaauuuuuuuguauaaagcccguuuaaucagguaaauaauccuuccauccuaauuauaucuauagauauaaaaggauggugagcuauauuacuauggauaagauuuuuuugaauucacguuugaagcguauuagaguaaguuuuauuuaaaaggaaaaaaaaaaauuaaauaaaauuaaauuaauuguauaaauaaguuuucguuuauuuauuaaugggucuuauauuaauguacguauagugaaauccuaauguaguaauuaagguguuauuaaauaaacuagguaagcccaauaaugucuucauaauaugaagaaguucaaguguaaauuugaauauacauuaguggguaaaggauauuuuaaaaagcgaaugucucauauuaauagugagaauaggucuaugacuaauucgaaagaaugcugacuuaaaauuaauauuaauauuaauau

SEQ ID NO: 87 - RNA, 363 nt, artificial sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuug

SEQ ID NO: 88 - RNA, 441 nt, Artificial Sequence
  5′ intron fragment
caauaaaagugcgaaacguuauccuauaaguaagaaaguuuuaaaauuuucuuacgaaaaggauagaacuuaaaaguucuaacuguucuacuaaaguaauaagugaaaaucuuauuuaaagcaaacaaccaaguagcuuuaagucuaaguccccuacacaaguuuuauacuacuaugcaaaacuugugaagcuagguaaggucguaauccgugaaagucggaugcggggcuccuuaaaagauuacuaugguaaacauaagcuaauccauuaagaugcgauuuauauguauuuuauacuguuaaauauuuuugugcuuguggcuugguauaaaacaguuaagaugaaguacuuaacugguuuuggaauaauugguuguuaaacuaaaacauuauaaaucguuaguggauaccuaagguaaucaaaaauagggauagguagaa

SEQ ID NO: 89 - RNA, 60 nt, Artificial Sequence; identical to SEQ ID NO: 20
  5′ arm of target sequence

SEQ ID NO: 90 - RNA, 58 nt, artificial sequence
  5′ arm of target sequence
cgaaaggaggaggaggaggaaaaaaaggaggaggaggaaaccaaaaaaacaaaacaca

SEQ ID NO: 91 - RNA, 93 nt, artificial sequence
  5′ arm of target sequence
gcaauggaggaggaggaggaaaaaaaggaggaggaggaagccgaaaaacaaaaaacaaaaaaaacaaaaaaaaaaccaaaaaaacaaaacaca

SEQ ID NO: 92 - RNA, 93 nt, artificial sequence
  5′ arm of target sequence
gcaauccuccuccuccuaaaaaaccuccuccuccuccuagccgaaaaacaaaaaacaaaaaaaacaaaaaaaaaaccaaaaaaacaaaacaca

SEQ ID NO: 93 - RNA, 39 nt, artificial sequence
  5′ arm of target sequence
cgaaaggaggaggaggaggaaccaaaaaaacaaaacaca

SEQ ID NO: 94 - RNA, 40 nt, artificial sequence
  5′ arm of target sequence
cgaaaccuccuccuccuccuaaccaaaaaaacaaaacaca

SEQ ID NO: 95 - RNA, 58 nt, artificial sequence
  5′ arm of target sequence
cgaaaccuccuccuccuaaaaaaccuccuccuccuccuaaccaaaaaaacaaaacaca

SEQ ID NO: 96 - RNA, 58 nt, artificial sequence; identical to SEQ ID NO: 95
  5′ arm of target sequence

SEQ ID NO: 97 - RNA, 19 nt, artificial sequence
  3′ arm of target sequence
aaaaaacaaaaaacaaaac

SEQ ID NO: 98 - RNA, 52 nt, artificial sequence
  3′ arm of target sequence
aaaaaacaaaaaacaccuccuccuccuaaaaaaccuccuccuccuccuaaaa

SEQ ID NO: 99 - RNA, 52 nt, artificial sequence
  3′ arm of target sequence
aaaaaacaaaaaacaccuccuccuccuaaaaaaccuccuccuccuccuaaac

SEQ ID NO: 100 - RNA, 52 nt, artificial sequence
  3′ arm of target sequence
aaaaaacaaaaaacaggaggaggaggaggaaaaaaaggaggaggaggaaaac

SEQ ID NO: 101 - RNA, 34 nt, Artificial Sequence
  3′ arm of target sequence
aaaaaacaaaaaacaccuccuccuccuccuaaaa

SEQ ID NO: 102 - RNA, 33 nt, Artificial Sequence
  3′ arm of target sequence
aaaaaacaaaaaacaggaggaggaggaggaaaa

SEQ ID NO: 103 - RNA, 51 nt, artificial sequence
  3′ arm of target sequence
aaaaaacaaaaaacaggaggaggaggaggaaaaaaaggaggaggaggaaaa

SEQ ID NO: 104 - RNA, 100 nt, artificial sequence
  3′ arm of target sequence
auuagagacaauuugaaauaauuuagauuggcuuaacccuacugugcuaaccgaaccagauaacgguacaguagggguaaauucuccgcauucggugcgg

SEQ ID NO: 105 - RNA, 20 nt, Artificial Sequence
  5′ homology arm sequence
aauaccuuacuuaauaguaa

SEQ ID NO: 106 - RNA, 20 nt, Artificial Sequence
  3′ homology arm sequence
uuacuauuaaguaagguauu

SEQ ID NO: 107 - RNA, 558 nt, Artificial Sequence
  target sequence
augggagucaaaguucuguuugcccugaucugcaucgcuguggccgaggccaagcccaccgagaacaacgaagacuucaacaucguggccguggccagcaacuucgcgaccacggaucucgaugcugaccgcgggaaguugcccggcaagaagcugccgcuggaggugcucaaagagauggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccauggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuag

SEQ ID NO: 108 - RNA, 720 nt, artificial sequence
  target sequence
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaaguag

SEQ ID NO: 109 - RNA, 936 nt, artificial sequence; identical to SEQ ID NO: 26
  target sequence

SEQ ID NO: 110 - RNA, 1653 nt, artificial sequence
  target sequence
auggccgaugcuaagaacauuaagaagggcccugcucccuucuacccucuggaggauggcaccgcuggcgagcagcugcacaaggccaugaagagguaugcccuggugccuggcaccauugccuucaccgaugcccacauugagguggacaucaccuaugccgaguacuucgagaugucugugcgccuggccgaggccaugaagagguacggccugaacaccaaccaccgcaucguggugugcucugagaacucucugcaguucuucaugccagugcugggcgcccuguucaucggaguggccguggccccugcuaacgacauuuacaacgagcgcgagcugcugaacagcaugggcauuucucagccuaccgugguguucgugucuaagaagggccugcagaagauccugaacgugcagaagaagcugccuaucauccagaagaucaucaucauggacucuaagaccgacuaccagggcuuccagagcauguacacauucgugacaucucaucugccuccuggcuucaacgaguacgacuucgugccagagucuuucgacagggacaaaaccauugcccugaucaugaacagcucugggucuaccggccugccuaagggcguggcccugccucaucgcaccgccugugugcgcuucucucacgcccgcgacccuauuuucggcaaccagaucauccccgacaccgcuauucugagcguggugccauuccaccacggcuucggcauguucaccacccugggcuaccugauuugcggcuuucggguggugcugauguaccgcuucgaggaggagcuguuccugcgcagccugcaagacuacaaaauucagucugcccugcuggugccaacccuguucagcuucuucgcuaagagcacccugaucgacaaguacgaccugucuaaccugcacgagauugccucuggcggcgccccacugucuaaggaggugggcgaagccguggccaagcgcuuucaucugccaggcauccgccagggcuacggccugaccgagacaaccagcgccauucugauuaccccagagggcgacgacaagccuggcgccgugggcaagguggugccauucuucgaggccaaggugguggaccuggacaccggcaagacccugggagugaaccagcgcggcgagcugugugugcgcggcccuaugauuauguccggcuacgugaauaacccugaggccacaaacgcccugaucgacaaggacggcuggcugcacucuggcgacauugccuacugggacgaggacgagcacuucuucaucguggaccgccugaagucucugaucaaguacaagggcuaccagguggccccagccgagcuggagucuauccugcugcagcacccuaacauuuucgacgccggaguggccggccugcccgacgacgaugccggcgagcugccugccgccgucgucgugcuggaacacggcaagaccaugaccgagaaggagaucguggacuauguggccagccaggugacaaccgccaagaagcugcgcggcggagugguguucguggacgaggugcccaagggccugaccggcaagcuggacgcccgcaagauccgcgagauccugaucaaggcuaagaaaggcggcaagaucgccguguaa

SEQ ID NO: 111 - RNA, 816 nt, artificial sequence
  target sequence
auggacgccaugaaacggggacugugcugcgugcugcugcuguguggcgccguguucgugucaccuagccgggugcagccuaccgagagcaucgugcgguucccuaacaucacaaaccuguguccauucggcgagguguucaacgccaccagauucgccagcguguacgcuuggaauagaaaaagaaucucuaauugcguggccgauuacagcgugcuguacaacagcgccuccuucagcaccuucaagugcuacggcgugucccccaccaagcugaacgaccugugcuucacaaaugucuacgccgauagcuucgugauuagaggcgacgaggugaggcagaucgcuccaggccagaccggcaagaucgcugauuacaacuacaagcugccugaugacuucacaggaugugugaucgccuggaacagcaacaaccucgacagcaaggugggaggcaacuacaauuaccuguauagacuguucagaaaguccaaccugaagcccuucgagagagacaucagcaccgaaaucuaccaggccggcuccaccccuugcaacggaguggaaggcuucaacugcuacuucccccugcagagcuacgguuuucagccuaccaacggcgugggcuaccagcccuaccgcgugguugugcugagcuucgagcugcugcacgccccagcuacagugugcggcccuaagaaaucuaccaaccuggugaagaacaagggcuauauccccgaggccccuagagacggccaagccuacgugcggaaggacggcgaauggguccugcucagcacauuccugggcagcuga

SEQ ID NO: 112 - RNA, 3345 nt, artificial sequence
  target sequence
auggccccaaagaagaagcggaaggucgguauccacggagucccagcagccaagcggaacuacauccugggccuggacaucggcaucaccagcgugggcuacggcaucaucgacuacgagacacgggacgugaucgaugccggcgugcggcuguucaaagaggccaacguggaaaacaacgagggcaggcggagcaagagaggcgccagaaggcugaagcggcggaggcggcauagaauccagagagugaagaagcugcuguucgacuacaaccugcugaccgaccacagcgagcugagcggcaucaaccccuacgaggccagagugaagggccugagccagaagcugagcgaggaagaguucucugccgcccugcugcaccuggccaagagaagaggcgugcacaacgugaacgagguggaagaggacaccggcaacgagcuguccaccaaagagcagaucagccggaacagcaaggcccuggaagagaaauacguggccgaacugcagcuggaacggcugaagaaagacggcgaagugcggggcagcaucaacagauucaagaccagcgacuacgugaaagaagccaaacagcugcugaaggugcagaaggccuaccaccagcuggaccagagcuucaucgacaccuacaucgaccugcuggaaacccggcggaccuacuaugagggaccuggcgagggcagccccuucggcuggaaggacaucaaagaaugguacgagaugcugaugggccacugcaccuacuuccccgaggaacugcggagcgugaaguacgccuacaacgccgaccuguacaacgcccugaacgaccugaacaaucucgugaucaccagggacgagaacgagaagcuggaauauuacgagaaguuccagaucaucgagaacguguucaagcagaagaagaagcccacccugaagcagaucgccaaagaaauccucgugaacgaagaggauauuaagggcuacagagugaccagcaccggcaagcccgaguucaccaaccugaagguguaccacgacaucaaggacauuaccgcccggaaagagauuauugagaacgccgagcugcuggaucagauugccaagauccugaccaucuaccagagcagcgaggacauccaggaagaacugaccaaucugaacuccgagcugacccaggaagagaucgagcagaucucuaaucugaagggcuauaccggcacccacaaccugagccugaaggccaucaaccugauccuggacgagcuguggcacaccaacgacaaccagaucgcuaucuucaaccggcugaagcuggugcccaagaagguggaccugucccagcagaaagagauccccaccacccugguggacgacuucauccugagccccgucgugaagagaagcuucauccagagcaucaaagugaucaacgccaucaucaagaaguacggccugcccaacgacaucauuaucgagcuggcccgcgagaagaacuccaaggacgcccagaaaaugaucaacgagaugcagaagcggaaccggcagaccaacgagcggaucgaggaaaucauccggaccaccggcaaagagaacgccaaguaccugaucgagaagaucaagcugcacgacaugcaggaaggcaagugccuguacagccuggaagccaucccucuggaagaucugcugaacaaccccuucaacuaugagguggaccacaucauccccagaagcguguccuucgacaacagcuucaacaacaaggugcucgugaagcaggaagaaaacagcaagaagggcaaccggaccccauuccaguaccugagcagcagcgacagcaagaucagcuacgaaaccuucaagaagcacauccugaaucuggccaagggcaagggcagaaucagcaagaccaagaaagaguaucugcuggaagaacgggacaucaacagguucuccgugcagaaagacuucaucaaccggaaccugguggauaccagauacgccaccagaggccugaugaaccugcugcggagcuacuucagagugaacaaccuggacgugaaagugaaguccaucaauggcggcuucaccagcuuucugcggcggaaguggaaguuuaagaaagagcggaacaagggguacaagcaccacgccgaggacgcccugaucauugccaacgccgauuucaucuucaaagaguggaagaaacuggacaaggccaaaaaagugauggaaaaccagauguucgaggaaaagcaggccgagagcaugcccgagaucgaaaccgagcaggaguacaaagagaucuucaucaccccccaccagaucaagcacauuaaggacuucaaggacuacaaguacagccaccggguggacaagaagccuaauagagagcugauuaacgacacccuguacuccacccggaaggacgacaagggcaacacccugaucgugaacaaucugaacggccuguacgacaaggacaaugacaagcugaaaaagcugaucaacaagagccccgaaaagcugcugauguaccaccacgacccccagaccuaccagaaacugaagcugauuauggaacaguacggcgacgagaagaauccccuguacaaguacuacgaggaaaccgggaacuaccugaccaaguacuccaaaaaggacaacggccccgugaucaagaagauuaaguauuacggcaacaaacugaacgcccaucuggacaucaccgacgacuaccccaacagcagaaacaaggucgugaagcugucccugaagcccuacagauucgacguguaccuggacaauggcguguacaaguucgugaccgugaagaaucuggaugugaucaaaaaagaaaacuacuacgaagugaauagcaagugcuaugaggaagcuaagaagcugaagaagaucagcaaccaggccgaguuuaucgccuccuucuacaacaacgaucugaucaagaucaacggcgagcuguauagagugaucggcgugaacaacgaccugcugaaccggaucgaagugaacaugaucgacaucaccuaccgcgaguaccuggaaaacaugaacgacaagaggccccccaggaucauuaagacaaucgccuccaagacccagagcauuaagaaguacagcacagacauucugggcaaccuguaugaagugaaaucuaagaagcacccucagaucaucaaaaagggcaaaaggccggcggccacgaaaaaggccggccaggcaaaaaagaaaaagggauccuacccauacgauguuccagauuacgcuuacccauacgauguuccagauuacgcuuacccauacgauguuccagauuacgcuuaa

SEQ ID NO: 113 - protein, 185 aa, artificial sequence
  target sequence
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 114 - protein, 239 aa, artificial sequence
  target sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 115 - protein, 311 aa, artificial sequence
  target sequence
MASKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 116 - protein, 550 aa, artificial sequence
  target sequence
MADAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 117 - protein, 271 aa, artificial sequence
  target sequence
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKGYIPEAPRDGQAYVRKDGEWVLLSTFLGS

SEQ ID NO: 118 - protein, 1114 aa, artificial sequence
  target sequence
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSYPYDVPDYAYPYDVPDYAYPYDVPDYA

SEQ ID NO: 119 - RNA, 6 nt, Artificial Sequence
  EBS1
uagggc

SEQ ID NO: 120 - RNA, 6 nt, artificial sequence
  EBS1
uuaugg

SEQ ID NO: 121 - RNA, 6 nt, artificial sequence
  EBS1
ucaacg

SEQ ID NO: 122 - RNA, 6 nt, artificial sequence
  EBS1
uguggc

SEQ ID NO: 123 - RNA, 6 nt, Artificial Sequence
  IBS1
gcccug

SEQ ID NO: 124 - RNA, 6 nt, Artificial Sequence
  IBS1
ccaugg

SEQ ID NO: 125 - RNA, 6 nt, Artificial Sequence
  IBS1
cguuga

SEQ ID NO: 126 - RNA, 6 nt, Artificial Sequence
  IBS1
gccaua

SEQ ID NO: 127 - RNA, 6 nt, Artificial Sequence
  IBS3 and its upstream sequence
ugugcu

SEQ ID NO: 128 - RNA, 6 nt, Artificial Sequence
  IBS3 and its upstream sequence
aaugcu

SEQ ID NO: 129 - RNA, 6 nt, Artificial Sequence
  IBS3 and its upstream sequence
ugcucu

SEQ ID NO: 130 - RNA, 6 nt, Artificial Sequence
  IBS3 and its upstream sequence
ugugcu

SEQ ID NO: 131 - RNA, 6 nt, Artificial Sequence
  IBS3 and its downstream sequence
agcaaa

SEQ ID NO: 132 - RNA, 6 nt, Artificial Sequence
  IBS3 and its downstream sequence
agcagu

SEQ ID NO: 133 - RNA, 6 nt, Artificial Sequence
  IBS3 and its downstream sequence
agagaa

SEQ ID NO: 134 - RNA, 6 nt, Artificial Sequence
  IBS3 and its downstream sequence
agcaaa

(19) United States
(12) Patent Application Publication
(10) Pub. No.: US 2024/0279676 A1
(43) Pub. Date: Aug. 22, 2024

(54) CONSTRUCTS AND METHODS FOR PREPARING CIRCULAR RNAS AND USE THEREOF

(71) Applicant: Shanghai Circode Biomed Co., Ltd., Shanghai (CN)

(72) Inventors: Yun YANG, Shanghai (CN); Zefeng WANG, Shanghai (CN)

(21) Appl. No.: 18/564,274

(22) PCT Filed: May 27, 2022
(86) PCT No.: PCT/CN2022/095749

§ 371 Date: Nov. 27, 2023

(30) Foreign Application Priority Data
May 28, 2021 (CN) 202110594352.4

(51) Int. Cl. C12N 15/85; C12N 15/64

(52) U.S. Cl. CPC C12N 15/85; C12N 15/64; C12N 2830/42; C12N 2830/50

(57) ABSTRACT

The present invention relates to a construct and method for preparing a circular RNA, and the use of the circular RNA. In particular, the present invention relates to a construct and method for preparing a circular RNA on the basis of a group II intron, and the use of the circular RNA.

This application claims the benefit of Chinese Application No. 202110594352.4, filed on May 28, 2021, which is hereby incorporated by reference in its entirety.

### REFERENCE TO SEQUENCE LISTING SUBMITTED ELECTRONICALLY

This application incorporates by reference a Sequence Listing submitted with this application as text file entitled “S2901CCD33CN_Sequence_Listing.txt” created on May 27, 2022 and having a size of 101,197 bytes.

### 1. TECHNICAL FIELD

The present invention relates to the field of molecular biology, in particular to a construct and method for preparing a circular RNA and application of the circular RNA. The circular RNA may be used to express a protein of interest in a eukaryotic cell or perform corresponding functions in the form of a noncoding RNA.

### 2. BACKGROUND OF THE INVENTION

Circular RNAs (circRNAs) are a category of circular RNA molecules formed by head-to-tail ligation. In recent years, it has been reported that circular RNAs may regulate gene transcription, and neutralize miRNA activity and binding of RNA-binding proteins, and may also be used as templates to be translated into proteins (Yang, Y., et al., “Extensive translation of circular RNAs driven by N(6)-methyladenosine,” Cell Research, 27(5):626-641 (2017); Abe, N., et al., “Rolling Circle Translation of Circular RNA in Living Human Cells”, Scientific Reports, 5:16435 (2015); Gao, X., et al., “Circular RNA-encoded oncogenic E-cadherin variant promotes glioblastoma tumorigenicity through activation of EGFR-STAT3 signalling,” Nature Cell Biology, 23(3):278-291 (2021); Pamudurti, N R., et al., “Translation of CircRNAs,” Molecular Cell, 66(1):9-21 (2017)). Compared with a linear RNA, the circular RNA has stronger stability because its covalently closed circular head-to-tail structure is not easily recognized by the RNA degradation system, and has a potential and prospect of becoming a new generation of RNA drug platform.

At present, there are three main methods for preparing a circular RNA in vitro. One method involves linking the 5′ end and 3′ end of a linear RNA in a head-to-tail manner through an RNA ligation reaction catalyzed by a nucleic acid ligase to obtain a circular RNA. The RNA ligase is a foreign protein, such as T4 RNA ligase. One method is chemical ligation, in which the 5′ end and 3′ end of an RNA are linked by the catalysis of bromine cyanide and a morpholinyl derivative. Another more advanced method involves obtaining a head-to-tail circular RNA through ribozyme-catalyzed RNA splicing. The circular RNA is expressed by this method by designing a ribozyme sequence-containing expression framework with self-splicing function.

Currently, ribozymes capable of RNA self-splicing are generally divided into two major categories, namely group I and group II introns, respectively. It has been reported in the literature that both categories of introns are capable of self-splicing under appropriate reaction conditions, linking two RNA fragments together. Although the splicing products of the two categories of ribozymes are similar, the structures and splicing mechanisms of the ribozymes themselves are quite different.

The group I intron has a 9-helix structure, which requires an external hydroxyl group in guanosine monophosphate (pG-OH) to trigger the reaction during catalytic splicing, and are highly dependent on the sequences of exons located at both ends of the group I intron.

The group II intron relies on its own hydroxyl groups within the nucleic acid sequence to trigger splicing. This splicing mechanism is closer to the splicing reaction mediated by a spliceosome, that is, it may better simulate splicing in higher organisms.

The above-mentioned structural difference determines that self-splicing of the group I intron requires a longer original exon sequence, also known as a scar sequence.

Previous studies have shown that the circular RNA may be prepared in vitro by using these two categories of intron ribozymes respectively, but the efficiency is relatively low (Puttaraju, M. & Been, M D., “Group I permuted intron-exon (PIE) sequences self-splice to produce circular exons,” Nucleic Acids Research, 20(20):5357-64 (1992); Mikheeva, S. et al., “Use of an engineered ribozyme to produce a circular human exon,” Nucleic Acids Research, 25(24):5085-94 (1997)).

The article by Wesselhoeft et al. reported a method for improving the efficiency of RNA circularization by optimizing a construct comprising a group I intron (Wesselhoeft, R A., et al., “Engineering circular RNA for potent and stable translation in eukaryotic cells,” Nature Communications, 9(1):2629 (2018)), and a related patent application (WO 2019/236673 A1) discloses a group I intron containing construct for the formation of a circular coding RNA. Wesselhoeft et al. rearranged a group I intron and the exons at its both ends, and constructed a protein of interest (POI) with an internal ribosome entry site (IRES) into this framework, and then a circular coding RNA from which the POI may be translated is obtained by self-splicing reaction in the presence of GTP. By selecting different group I introns and carrying out design and engineering, the efficiency of RNA circularization is improved. Specifically, in the technique, some deletions were firstly made in the Td gene of T4 phage, retaining the sequence that may be folded correctly to maintain the ribozyme activity, comprising introns and a portion of exons; then the sequence was divided into two portions; a 3′-end intron and an exon fragment 2 (E2) were constructed to the 5′ end of IRES-POI, and an exon fragment 1 (E1) and a 5′-end intron were constructed to the 3′ end of IRES-POI; and a circular RNA was obtained by self-splicing in the presence of GTP and magnesium ions. However, Wesselhoeft et al. found that the 5′-end and 3′-end splice sites can not be efficiently spliced due to the insertion of the target gene. To address this issue, Wesselhoeft et al. inserted complementary paired “homology arms” near the splice site, thereby increasing splicing efficiency. Furthermore, according to an existing literature (Mikheeva, S. et al., (1997), supra), another group I intron, Anabaena, was selected, and it was found that its splicing efficiency was higher than that of the Td intron, and similar design and engineering were carried out on it to further improve the splicing efficiency. The article finally verifies that the POI may be effectively translated from the expression framework.

However, the design of Wesselhoeft et al. has the following disadvantages:
1. When using a group I intron, a longer original exon sequence must be comprised, and thus an original exogenous sequence (scar sequence) will be comprised in the expression product. In the process of preparing the target sequence into a circular RNA, it is usually desirable to remove the sequence that does not belong to the target sequence for the convenience of subsequent applications.
2. The group I intron requires the participation of GTP to provide energy for self-splicing. On the other hand, the splicing efficiency of the group II intron in previous literature is relatively low (about 10%) (Mikheeva, S. et al., (1997), supra). Therefore, there remains a need in the art for improved constructs and methods for preparing circular RNAs.

### 3. SUMMARY OF THE INVENTION

Through screening and design optimization, the inventors of the present application have created a methodology for preparing a circular RNA by self-splicing of a group II intron, which overcomes the above problems.

Accordingly, the present invention provides a polynucleotide construct with self-splicing activity in vitro, comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a polynucleotide construct with self-splicing activity in vitro, comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a polynucleotide construct with self-splicing activity in vitro, comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a polynucleotide construct with self-splicing activity in vitro, comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron into two fragments, and the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

In some embodiments, the polynucleotide construct is an RNA polynucleotide construct.

In some embodiments, the polynucleotide construct is capable of forming a circular RNA of a target sequence in vitro.

In some embodiments, the polynucleotide construct is capable of forming a circular RNA of a target sequence in vivo.

The present invention provides a circular RNA produced by the polynucleotide construct of the present invention. In some embodiments, the circular RNA is at least 500 nucleotides in length, at least 1,000 nucleotides in length, or at least 1,500 nucleotides in length.

The present invention provides a method of making a circular RNA using the polynucleotide construct of the present invention.

The present invention provides a method of making a circular RNA, said method comprising: preparing a vector comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a method of making a circular RNA, said method comprising: preparing a vector comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a method of making a circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention also provides a method of making a circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron into two fragments, and the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

The present invention provides a method for expressing a protein in a cell, comprising transfecting the cell with the circular RNA of the present invention.

The present invention provides a method for expressing a protein in a cell, comprising (a) transfecting the cell with the circular RNA of the present invention, or (b) subjecting the polynucleotide construct of the present invention to a self-splicing circularization reaction to form a circular RNA, and transfecting the cell with the circular RNA; wherein, preferably the cell is a eukaryotic cell.

The present invention provides a method for generating a sequence with self-splicing activity using a group II intron, the method comprising the steps of:
(a) defining the sequence of the group II intron; optionally examining the in vitro self-splicing activity of the group II intron using a splicing assay (linear splicing);
(b) splitting the group II intron into two fragments,
(c) reversing the order of the two intron fragments, and
(d) confirming the in vitro circularization of RNA using a splicing assay.

The construct, method and application of the present invention have at least the following advantages:
1. A circular RNA is produced without a scar sequence, which is more conducive to orderly application;
2. GTP is not required for the self-splicing reaction of the polynucleotide to form a circular RNA (e.g., in some embodiments, only Mg ions and Na ions are needed); and/or
3. The splicing efficiency of group II introns is greatly improved, which may be increased from 10% to about 50%, and even the highest splicing efficiency up to 98% may be achieved.

In a specific embodiment, the E1 and/or the E2 is 0 to 20 nucleotides in length, preferably 0 to 10 nucleotides, such as 0, 1, 2, 3, 4, 5, 6, 7, 8, 9 or 10 nucleotides.

In a specific embodiment, the 5′ intron fragment and the 3′ intron fragment segment a group II intron at an unpaired region into two fragments. In a specific embodiment, the unpaired region is selected from a linear region between two adjacent domains of the group II intron or a loop region of a stem-loop structure of domain 4.

In a specific embodiment, the group II intron comprises a modification of one or more nucleotides relative to its wild-type form, and the modification is selected from one or more of a deletion, a substitution, and an addition.

In a specific embodiment, the 5′ intron fragment and the 3′ intron fragment respectively comprise one or more pairs of paired sequences that are complementary to each other. In a preferred embodiment, the complementary paired sequence is greater than 20 nucleotides in length.

In a specific embodiment, the 5′ intron fragment and/or the 3′ intron fragment comprises one or more affinity tag sequences selected from one or more of a group of: a probe binding sequence, an MS2 binding site, a PP7 binding site, and a streptavidin binding site.

In a specific embodiment, the E1 and the E2 are 0, and the modification comprises a modification of one or more EBS sequences of the group II intron so that the EBS sequences are complementarily paired with one or more regions of a corresponding length in a target sequence on at least 60% of the nucleotide positions respectively. The EBS sequence is selected from one or more of EBS1, EBS2 and EBS3, preferably any two of them, more preferably EBS1 and EBS3. In a preferred embodiment, the modification is a modification of the two EBS sequences of the group II intron, preferably EBS1 and EBS3, so that the EBS sequences are complementarily paired with two regions of a corresponding length in a target sequence on at least 60% of the nucleotide positions respectively. In a preferred embodiment, the modification is a modification of the two EBS sequences of the group II intron, preferably EBS1′ and EBS3′, so that the EBS sequences are complementarily paired with two regions of a corresponding length in a target sequence on at least 60% of the nucleotide positions respectively. In a preferred embodiment, the modification is a modification of the two EBS sequences of the group II intron, preferably EBS1″ and EBS3″, so that the EBS sequences are complementarily paired with two regions of a corresponding length in a target sequence on at least 60% of the nucleotide positions respectively. In another preferred embodiment, the modification is a modification of the δ or δ″ sequence of the group II intron, wherein the δ or δ″ sequence is complementarily paired with a region of a corresponding length in a target sequence on at least 60% of the nucleotide positions respectively; preferably, the region is located at one end of the target sequence.

In a preferred embodiment, the two regions of a corresponding length in a target sequence are located at both ends of the target sequence, respectively.

In a specific embodiment, the modification is a deletion of part or all of domain 4, such as a deletion of an IEP sequence in domain 4, preferably a deletion of all of domain 4.

In a specific embodiment, the group II intron is a group II intron derived from a microorganism. Preferably, the group II intron has in vitro self-splicing activity. In a specific embodiment, the group II intron is a group II intron from Clostridium, such as Clostridium tetani, or Bacillus, such as Bacillus thuringiensis. In a specific embodiment, the group II intron is the group II intron contained in the nucleotide sequence of SEQ ID NO: 1 or 2.

In a specific embodiment, the protein noncoding sequence is selected from one or more of a group of: a spacer sequence such as any of SEQ ID NOs: 4-6, an A- and/or T-rich sequence, a polyA sequence, a polyA-C sequence, a polyC sequence, a poly-U sequence, an IRES, a ribosome binding site, an aptamer sequence, an RNA scaffold, a riboswitch, a ribozyme other than a self-splicing ribozyme, a small RNA, a translational regulatory sequence, and a protein binding site.

In a specific embodiment, the polynucleotide construct is capable of forming a circular RNA of a target sequence in vitro.

In a specific embodiment, the polynucleotide construct is capable of forming a circular RNA of a target sequence in vivo.

In a second aspect, the present invention provides a circular RNA produced by the construct of the first aspect. Preferably, the circular RNA does not comprise any other sequences that do not belong to the target sequence, such as not comprising an E2 sequence and an E1 sequence.

In a specific embodiment, in the technical solution in which the target sequence is a protein coding sequence, the circular RNA is at least 500 nucleotides in length, preferably at least 1,000 nucleotides, and preferably at least 1,500 nucleotides. In the technical solution in which the target sequence is a noncoding RNA, the target sequence may be shorter.

In a third aspect, the present invention provides a method for expressing a protein in a cell, comprising transfecting the cell with the circular RNA of the second aspect.

In a fourth aspect, the present invention provides a method for expressing a protein in a cell, comprising subjecting the construct of the first aspect to a self-splicing circularization reaction to form a circular RNA, and transfecting the cell with the circular RNA.

In specific embodiments of the third and fourth aspects, the cell is a eukaryotic cell.

The construct, method and application of the present invention have at least the following advantages:
1. In a preferred technical solution, a circular RNA without a scar sequence may be produced, which is more conducive to orderly application;
2. GTP is not required to participate in the self-splicing reaction to form a circular RNA, only Mg ions and Na ions need to be provided; and
3. The splicing efficiency of group II introns is greatly improved, which may be increased from 10% to about 50%, and even the highest splicing efficiency up to 98% may be achieved.

### 4. ILLUSTRATIVE EMBODIMENTS

### Set 1

1. A polynucleotide construct with self-splicing activity in vitro, comprising the following operably linked elements from 5′ to 3′:
   (1) a 3′ intron fragment;
   (2) an exon fragment 2 (E2); and
   (3) a target sequence;
   (4) an exon fragment 1 (E1); and
   (5) a 5′ intron fragment,
   wherein the 5′ intron fragment and the 3′ intron fragment are obtained by segmenting a group II intron into two fragments, and the 5′ intron fragment is located on the 5′ side of the 3′ intron fragment in the group II intron,
   the E1 is a 5′ adjacent exon fragment of the group II intron, which is ≥0 nucleotides in length,
   the E2 is a 3′ adjacent exon fragment of the group II intron, which is ≥0 nucleotides in length, and
   the target sequence is empty, or is a protein coding sequence and/or a noncoding sequence.
2. The polynucleotide construct of paragraph 1, wherein the E1 and/or the E2 is 0 to 20 nucleotides in length, preferably 0 to 10 nucleotides, such as 0, 1, 2, 3, 4, 5, 6, 7, 8, 9 or 10 nucleotides.
3. The polynucleotide construct of paragraph 1, wherein the 5′ intron fragment and the 3′ intron fragment are obtained by segmenting a group II intron at an unpaired region into two fragments, and the unpaired region is preferably selected from a linear region between two adjacent domains of the group II intron or a loop region of a stem-loop structure of domain 4.
4. The polynucleotide construct of paragraph 1, wherein the group II intron comprises a modification of one or more nucleotides relative to its wild-type form, and the modification is selected from one or more of a deletion, a substitution, and an addition.
5. The polynucleotide construct of paragraph 4, wherein the E1 and the E2 are 0, and the modification comprises a modification of one or more EBS sequences of the group II intron so that the EBS sequences are complementarily paired with one or more regions of a corresponding length in a target sequence on at least 60% of the nucleotide positions respectively.
6. The polynucleotide construct of paragraph 5, wherein the modification is a modification of the two EBS sequences of the group II intron, such as EBS1 and EBS3, so that the EBS sequences are complementarily paired with two regions of a corresponding length in a target sequence on at least 60% of the nucleotide positions respectively; preferably, the two regions are located at both ends of the target sequence, respectively.
7. The polynucleotide construct of paragraph 4, wherein the modification comprises a deletion of part or all of domain 4, such as a deletion of an IEP sequence in domain 4, preferably a deletion of all of domain 4.
8. The polynucleotide construct of paragraph 1, wherein the group II intron is a group II intron derived from a microorganism.
9. The polynucleotide construct of paragraph 1, wherein the noncoding sequence is selected from sequences of a group of: any of the spacer sequences of SEQ ID NOs: 4-6, a polyA sequence, a polyA-C sequence, a polyC sequence, a poly-U sequence, an IRES, a ribosome binding site, an aptamer sequence, an RNA scaffold, a riboswitch, a ribozyme other than a self-splicing ribozyme, a small RNA binding site, a translational regulatory sequence, and a protein binding site.
10. A circular RNA produced by the polynucleotide construct of any of paragraphs 1 to 9.
11. The circular RNA of paragraph 10, not comprising any other sequences that do not belong to the target sequence, such as not comprising all or part of an E2 sequence and an E1 sequence.
12. A method for expressing a protein in a cell, comprising (a) transfecting of the cell with the circular RNA of paragraph 10 or 11, or (b) subjecting the construct of any of paragraphs 1 to 9 to a self-splicing circularization reaction to form a circular RNA, and transfecting the cell with the circular RNA;
   wherein, preferably the cell is a eukaryotic cell.

### Set 2

### Embodiment 1

A polynucleotide construct with self-splicing activity, comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 2

A polynucleotide construct with self-splicing activity, comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 3

A polynucleotide construct with self-splicing activity, comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 4

A polynucleotide construct with self-splicing activity, comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron into two fragments, and the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 5

The polynucleotide construct of any one of Embodiments 1-4, wherein the polynucleotide construct has self-splicing activity in vitro.

### Embodiment 6

The polynucleotide construct of any one of Embodiments 1-5, wherein the E1 and/or the E2 is 0 to 20 nucleotides in length, preferably 0 to 10 nucleotides in length, such as 0, 1, 2, 3, 4, 5, 6, 7, 8, 9 or 10 nucleotides in length.

### Embodiment 7

The polynucleotide construct of any one of Embodiments 1-6, wherein the 5′ intron fragment and the 3′ intron fragment are obtained by segmenting a group II intron at an unpaired region into two fragments, for example, an unpaired region which is a linear region between two adjacent domains of the group II intron.

### Embodiment 8

The polynucleotide construct of any one of Embodiments 1-6, wherein the 5′ intron fragment and the 3′ intron fragment are obtained by segmenting a group II intron at a loop region of a stem-loop structure of domain 1.

### Embodiment 9

The polynucleotide construct of any one of Embodiments 1-6, wherein the 5′ intron fragment and the 3′ intron fragment are obtained by segmenting a group II intron at a loop region of a stem-loop structure of domain 2.

### Embodiment 10

The polynucleotide construct of any one of Embodiments 1-6, wherein the 5′ intron fragment and the 3′ intron fragment are obtained by segmenting a group II intron at a loop region of a stem-loop structure of domain 3.

### Embodiment 11

The polynucleotide construct of any one of Embodiments 1-6, wherein the 5′ intron fragment and the 3′ intron fragment are obtained by segmenting a group II intron at a loop region of a stem-loop structure of domain 4.

### Embodiment 12

The polynucleotide construct of any one of Embodiments 1-6, wherein the 5′ intron fragment and the 3′ intron fragment are obtained by segmenting a group II intron at a loop region of a stem-loop structure of domain 5.

### Embodiment 13

The polynucleotide construct of any one of Embodiments 1-6, wherein the 5′ intron fragment and the 3′ intron fragment are obtained by segmenting a group II intron at a loop region of a stem-loop structure of domain 6.

### Embodiment 14

The polynucleotide construct of any one of Embodiments 1-6, wherein the 5′ intron fragment and the 3′ intron fragment are obtained by segmenting a group II intron at a linear region between domain 1 and domain 2.

### Embodiment 15

The polynucleotide construct of any one of Embodiments 1-6, wherein the 5′ intron fragment and the 3′ intron fragment are obtained by segmenting a group II intron at a linear region between domain 2 and domain 3.

### Embodiment 16

The polynucleotide construct of any one of Embodiments 1-6, wherein the 5′ intron fragment and the 3′ intron fragment are obtained by segmenting a group II intron at a linear region between domain 3 and domain 4.

### Embodiment 17

The polynucleotide construct of any one of Embodiments 1-6, wherein the 5′ intron fragment and the 3′ intron fragment are obtained by segmenting a group II intron at a linear region between domain 4 and domain 5.

### Embodiment 18

The polynucleotide construct of any one of Embodiments 1-6, wherein the 5′ intron fragment and the 3′ intron fragment are obtained by segmenting a group II intron at a linear region between domain 5 and domain 6.

### Embodiment 19

The polynucleotide construct of any one of Embodiments 1-18, wherein the group II intron comprises a modification of one or more nucleotides relative to its wild-type form, and the modification is selected from one or more of a deletion, a substitution, and an addition.

### Embodiment 20

The polynucleotide construct of Embodiment 19, wherein the modification comprises a modification of one or more EBS sequences of the group II intron, wherein the EBS sequences are complementarily paired with one or more regions of a corresponding length in a target sequence on at least 60% of the nucleotide positions respectively.

### Embodiment 21

The polynucleotide construct of Embodiment 19, wherein the modification is a modification of the two EBS sequences of the group II intron, such as EBS1 and EBS3, wherein the EBS sequences are complementarily paired with two regions of a corresponding length in a target sequence on at least 60% of the nucleotide positions respectively; preferably, the two regions are located at both ends of the target sequence, respectively.

The polynucleotide construct of Embodiment 19, wherein the modification is a modification of EBS1 and/or δ sequence of the group II intron, or a modification of EBS1′ and/or δ″ sequence, wherein the EBS1 and/or δ sequence is complementarily paired with a region of a corresponding length in a target sequence on at least 60% of the nucleotide, optionally the modification is a modification of EBS1 and/or δ sequence and its upstream sequence, wherein the EBS1 and/or δ sequence and its upstream sequence is complementarily paired with a region of a corresponding length in a target sequence on at least 60% of the nucleotide. In some embodiments, the region of a corresponding length in a target sequence is IBS3, IBS3′, IBS3 with downstream sequence, or IBS3′ with downstream sequence. In some embodiments, the δ sequence and its upstream comprises a nucleic acid sequence selected from the group consisting: (a) wherein the modification is a modification of a δ or δ″ sequence of the group II intron, wherein the δ or δ″ sequence is complementarily paired with a region of a corresponding length in a target sequence on at least 60% of the nucleotide positions respectively; preferably, the region is located at one end of the target sequence. In some embodiments, the δ sequence and its upstream comprises SEQ ID NO: 127, (b) SEQ ID NO:128, (c) SEQ ID NO:129, and (d) SEQ ID NO 130. In some embodiments, the IBS3 and its downstream comprises a nucleic acid sequence selected from the group consisting: (a) SEQ ID NO: 131, (b) SEQ ID NO:132, (c) SEQ ID NO:133, and (d) SEQ ID NO 134.

### Embodiment 22

The polynucleotide construct of Embodiment 19, wherein the modification comprises a deletion of part or all of domain 4, such as a deletion of an intron-encoded protein (IEP) sequence in domain 4, preferably a deletion of all of domain 4.

### Embodiment 23

The polynucleotide construct of Embodiment 19, wherein the modification comprises a deletion of an open reading frame (ORF).

### Embodiment 24

The polynucleotide construct of any one of Embodiments 1-23, wherein the polynucleotide construct is capable of forming a near-scarless circular RNA of the target sequence.

### Embodiment 25

The polynucleotide construct of Embodiment 24, wherein the near-scarless circular RNA has a scar region equal to or less than 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides in length.

### Embodiment 26

The polynucleotide construct of any one of Embodiments 1-23, wherein the polynucleotide construct is capable of forming a scarless circular RNA of the target sequence.

### Embodiment 27

The polynucleotide construct of any one of Embodiments 1-26, wherein E1 and E2 are each 0 nucleotide in length.

### Embodiment 28

The polynucleotide construct of any one of Embodiments 1-26, wherein the E1 is 0 nucleotide in length.

### Embodiment 29

The polynucleotide construct of any one of Embodiments 1-26, wherein the E2 is 0 nucleotide in length.

### Embodiment 30

The polynucleotide construct of any one of Embodiments 1-29, wherein the group II intron is a group II intron derived from a microorganism (such as Clostridium tetani, or Bacillus, such as Bacillus thuringiensis).

### Embodiment 31

The polynucleotide construct of any one of Embodiments 1-30, wherein the noncoding sequence is selected from the group consisting of: a spacer sequence of SEQ ID NOs: 4-6, a polyA sequence, a poly-A-C sequence, a poly-C sequence, a poly-U sequence, an IRES, a ribosome binding site, an aptamer sequence, an RNA scaffold, a riboswitch, a ribozyme other than a self-splicing ribozyme, an antisense oligonucleotide (ASO), a scaffold, a small RNA binding site, a translational regulatory sequence, and a protein binding site.

### Embodiment 32

The polynucleotide construct of any one of Embodiments 1-31, wherein the group II intron comprises a nucleic acid sequence selected from the group consisting of:
(a) SEQ ID NO: 33;
(b) SEQ ID NO: 34;
(c) SEQ ID NO: 35;
(d) SEQ ID NO: 36;
(e) SEQ ID NO: 37;
(f) SEQ ID NO: 38;
(g) SEQ ID NO: 39;
(h) SEQ ID NO: 40; and
(i) SEQ ID NO: 41.

### Embodiment 32-1

The polynucleotide construct of Embodiment 32, wherein the group II intron consists essentially of a nucleic acid sequence selected from the group consisting of SEQ ID NO: 33-SEQ ID NO: 41.

### Embodiment 32-2

The polynucleotide construct of Embodiment 32, wherein the group II intron consists of a nucleic acid sequence selected from the group consisting of SEQ ID NO: 33-SEQ ID NO: 41.

### Embodiment 33

The polynucleotide construct of any one of Embodiments 1-32, wherein the polynucleotide construct is an RNA polynucleotide construct.

### Embodiment 34

The polynucleotide construct of Embodiment 33, wherein the 3′ intron fragment comprises a nucleic acid sequence selected from the group consisting of:
(a) a nucleic acid sequence 95% identical to SEQ ID NO: 42;
(b) a nucleic acid sequence 98% identical to SEQ ID NO: 42;
(c) a nucleic acid sequence 99% identical to SEQ ID NO: 42;
(d) SEQ ID NO: 42;
(e) a nucleic acid sequence 95% identical to SEQ ID NO: 43;
(f) a nucleic acid sequence 98% identical to SEQ ID NO: 43;
(g) a nucleic acid sequence 99% identical to SEQ ID NO: 43;
(h) SEQ ID NO: 43;
(i) a nucleic acid sequence 95% identical to SEQ ID NO: 44;
(j) a nucleic acid sequence 98% identical to SEQ ID NO: 44;
(k) a nucleic acid sequence 99% identical to SEQ ID NO: 44;
(l) SEQ ID NO: 44;
(m) a nucleic acid sequence 95% identical to SEQ ID NO: 45;
(n) a nucleic acid sequence 98% identical to SEQ ID NO: 45;
(o) a nucleic acid sequence 99% identical to SEQ ID NO: 45;
(p) SEQ ID NO: 45;
(q) a nucleic acid sequence 95% identical to SEQ ID NO: 46;
(r) a nucleic acid sequence 98% identical to SEQ ID NO: 46;
(s) a nucleic acid sequence 99% identical to SEQ ID NO: 46;
(t) SEQ ID NO: 46;
(u) a nucleic acid sequence 95% identical to SEQ ID NO: 47;
(v) a nucleic acid sequence 98% identical to SEQ ID NO: 47;
(w) a nucleic acid sequence 99% identical to SEQ ID NO: 47;
(x) SEQ ID NO: 47;
(y) a nucleic acid sequence 95% identical to SEQ ID NO: 48;
(z) a nucleic acid sequence 98% identical to SEQ ID NO: 48;
(aa) a nucleic acid sequence 99% identical to SEQ ID NO: 48;
(bb) a nucleic acid sequence SEQ ID NO: 48;
(cc) a nucleic acid sequence 95% identical to SEQ ID NO: 49;
(dd) a nucleic acid sequence 98% identical to SEQ ID NO: 49;
(ee) a nucleic acid sequence 99% identical to SEQ ID NO: 49;
(ff) SEQ ID NO: 49;
(gg) a nucleic acid sequence 95% identical to SEQ ID NO: 50;
(hh) a nucleic acid sequence 98% identical to SEQ ID NO: 50;
(ii) a nucleic acid sequence 99% identical to SEQ ID NO: 50;
(jj) SEQ ID NO: 50;
(kk) a nucleic acid sequence 95% identical to SEQ ID NO: 51;
(ll) a nucleic acid sequence 98% identical to SEQ ID NO: 51;
(mm) a nucleic acid sequence 99% identical to SEQ ID NO: 51;
(nn) SEQ ID NO: 51;
(oo) a nucleic acid sequence 95% identical to SEQ ID NO: 52;
(pp) a nucleic acid sequence 98% identical to SEQ ID NO: 52;
(qq) a nucleic acid sequence 99% identical to SEQ ID NO: 52; and
(rr) SEQ ID NO: 52.

### Embodiment 34-1

The polynucleotide construct of Embodiment 34, wherein the 3′ intron fragment consists essentially of a nucleic acid sequence selected from the group consisting of a nucleic acid sequence 95% identical to any one of SEQ ID NO: 42-SEQ ID NO: 52, a nucleic acid sequence 98% identical to any one of SEQ ID NO: 42-SEQ ID NO: 52, a nucleic acid sequence 99% identical to any one of SEQ ID NO: 42-SEQ ID NO: 52, and any one of SEQ ID NO: 42-SEQ ID NO: 52.

### Embodiment 34-2

The polynucleotide construct of Embodiment 34, wherein the 3′ intron fragment consists of a nucleic acid sequence selected from the group consisting of a nucleic acid sequence 95% identical to any one of SEQ ID NO: 42-SEQ ID NO: 52, a nucleic acid sequence 98% identical to any one of SEQ ID NO: 42-SEQ ID NO: 52, a nucleic acid sequence 99% identical to any one of SEQ ID NO: 42-SEQ ID NO: 52, and any one of SEQ ID NO: 42-SEQ ID NO: 52.

### Embodiment 35

The polynucleotide construct of Embodiment 33 or 34, wherein the E2 comprises a nucleic acid sequence selected from the group consisting of:
(a) SEQ ID NO: 53;
(b) SEQ ID NO: 54;
(c) SEQ ID NO: 55;
(d) SEQ ID NO: 56.
(e) SEQ ID NO: 57;
(f) SEQ ID NO: 58;
(g) SEQ ID NO: 59;
(h) SEQ ID NO: 60.
(i) SEQ ID NO: 61;
(j) SEQ ID NO: 62; and
(k) SEQ ID NO: 63.

### Embodiment 35-1

The polynucleotide construct of Embodiment 35, wherein the E2 consists essentially of a nucleic acid sequence selected from the group consisting of SEQ ID NO: 53-SEQ ID NO: 63.

### Embodiment 35-2

The polynucleotide construct of Embodiment 35, wherein the E2 consists of a nucleic acid sequence selected from the group consisting of SEQ ID NO: 53-SEQ ID NO: 63.

### Embodiment 36

The polynucleotide construct of any one of Embodiments 33-35, wherein the E1 comprises a nucleic acid sequence selected from the group consisting of:
(a) SEQ ID NO: 64;
(b) SEQ ID NO: 65;
(c) SEQ ID NO: 66;
(d) SEQ ID NO: 67.
(e) SEQ ID NO: 68;
(f) SEQ ID NO: 69;
(g) SEQ ID NO: 70;
(h) SEQ ID NO: 71.
(i) SEQ ID NO: 72;
(j) SEQ ID NO: 73; and
(k) SEQ ID NO: 74.

### Embodiment 36-1

The polynucleotide construct of Embodiment 36, wherein the E1 consists essentially of a nucleic acid sequence selected from the group consisting of SEQ ID NO: 64-SEQ ID NO: 74.

### Embodiment 36-2

The polynucleotide construct of Embodiment 36, wherein the E1 consists of a nucleic acid sequence selected from the group consisting of SEQ ID NO: 64-SEQ ID NO: 74.

### Embodiment 37

The polynucleotide construct of any one of Embodiments 33-36, wherein the 5′ intron fragment comprises a nucleic acid sequence selected from the group consisting of:
(a) a nucleic acid sequence 95% identical to SEQ ID NO: 75;
(b) a nucleic acid sequence 98% identical to SEQ ID NO: 75;
(c) a nucleic acid sequence 99% identical to SEQ ID NO: 75;
(d) SEQ ID NO: 75;
(e) a nucleic acid sequence 95% identical to SEQ ID NO: 76;
(f) a nucleic acid sequence 98% identical to SEQ ID NO: 76;
(g) a nucleic acid sequence 99% identical to SEQ ID NO: 76;
(h) SEQ ID NO: 76;
(i) a nucleic acid sequence 95% identical to SEQ ID NO: 77;
(j) a nucleic acid sequence 98% identical to SEQ ID NO: 77;
(k) a nucleic acid sequence 99% identical to SEQ ID NO: 77;
(l) SEQ ID NO: 77;
(m) a nucleic acid sequence 95% identical to SEQ ID NO: 78;
(n) a nucleic acid sequence 98% identical to SEQ ID NO: 78;
(o) a nucleic acid sequence 99% identical to SEQ ID NO: 78;
(p) SEQ ID NO: 78;
(q) a nucleic acid sequence 95% identical to SEQ ID NO: 79;
(r) a nucleic acid sequence 98% identical to SEQ ID NO: 79;
(s) a nucleic acid sequence 99% identical to SEQ ID NO: 79;
(t) SEQ ID NO: 79;
(u) a nucleic acid sequence 95% identical to SEQ ID NO: 80;
(v) a nucleic acid sequence 98% identical to SEQ ID NO: 80;
(w) a nucleic acid sequence 99% identical to SEQ ID NO: 80;
(x) SEQ ID NO: 80;
(y) a nucleic acid sequence 95% identical to SEQ ID NO: 81;
(z) a nucleic acid sequence 98% identical to SEQ ID NO: 81;
(aa) a nucleic acid sequence 99% identical to SEQ ID NO: 81;
(bb) SEQ ID NO: 81;
(cc) a nucleic acid sequence 95% identical to SEQ ID NO: 82;
(dd) a nucleic acid sequence 98% identical to SEQ ID NO: 82;
(ee) a nucleic acid sequence 99% identical to SEQ ID NO: 82;
(ff) SEQ ID NO: 82;
(gg) a nucleic acid sequence 95% identical to SEQ ID NO: 83;
(hh) a nucleic acid sequence 98% identical to SEQ ID NO: 83;
(ii) a nucleic acid sequence 99% identical to SEQ ID NO: 83;
(jj) SEQ ID NO: 83;
(kk) a nucleic acid sequence 95% identical to SEQ ID NO: 84;
(ll) a nucleic acid sequence 98% identical to SEQ ID NO: 84;
(mm) a nucleic acid sequence 99% identical to SEQ ID NO: 84;
(nn) SEQ ID NO: 84;
(oo) a nucleic acid sequence 95% identical to SEQ ID NO: 85;
(pp) a nucleic acid sequence 98% identical to SEQ ID NO: 85;
(qq) a nucleic acid sequence 99% identical to SEQ ID NO: 85;
(rr) SEQ ID NO: 85;
(ss) a nucleic acid sequence 95% identical to SEQ ID NO: 86;
(tt) a nucleic acid sequence 98% identical to SEQ ID NO: 86;
(uu) a nucleic acid sequence 99% identical to SEQ ID NO: 86;
(vv) SEQ ID NO: 86;
(ww) a nucleic acid sequence 95% identical to SEQ ID NO: 87;
(xx) a nucleic acid sequence 98% identical to SEQ ID NO: 87;
(yy) a nucleic acid sequence 99% identical to SEQ ID NO: 87;
(zz) SEQ ID NO: 87;
(aaa) a nucleic acid sequence 95% identical to SEQ ID NO: 88;
(bbb) a nucleic acid sequence 98% identical to SEQ ID NO: 88;
(ccc) a nucleic acid sequence 99% identical to SEQ ID NO: 88; and
(ddd) SEQ ID NO: 88.

### Embodiment 37-1

The polynucleotide construct of Embodiment 37, wherein the 5′ intron fragment consists essentially of a nucleic acid sequence selected from the group consisting of a nucleic acid sequence 95% identical to any one of SEQ ID NO: 75-SEQ ID NO: 88, a nucleic acid sequence 98% identical to any one of SEQ ID NO: 75-SEQ ID NO: 88, a nucleic acid sequence 99% identical to any one of SEQ ID NO: 75-SEQ ID NO: 88, and any one of SEQ ID NO: 75-SEQ ID NO: 88.

### Embodiment 37-2

The polynucleotide construct of Embodiment 37, wherein the 5′ intron fragment consists of a nucleic acid sequence selected from the group consisting of a nucleic acid sequence 95% identical to any one of SEQ ID NO: 75-SEQ ID NO: 88, a nucleic acid sequence 98% identical to any one of SEQ ID NO: 75-SEQ ID NO: 88, a nucleic acid sequence 99% identical to any one of SEQ ID NO: 75-SEQ ID NO: 88, and any one of SEQ ID NO: 75-SEQ ID NO: 88.

### Embodiment 38

The polynucleotide construct of any one of Embodiments 3-37, wherein the 5′ homology arm comprises the nucleic acid sequence of SEQ ID NO: 105.

### Embodiment 38-1

The polynucleotide construct of Embodiment 38, wherein the 5′ homology arm consists essentially of the nucleic acid sequence of SEQ ID NO: 105.

### Embodiment 38-2

The polynucleotide construct of Embodiment 38, wherein the 5′ homology arm consists of the nucleic acid sequence of SEQ ID NO: 105.

### Embodiment 39

The polynucleotide construct of any one of Embodiments 3-38, wherein the 3′ homology arm comprises the nucleic acid sequence of SEQ ID NO: 106.

### Embodiment 39-1

The polynucleotide construct of Embodiment 39, wherein the 3′ homology arm consists essentially of the nucleic acid sequence of SEQ ID NO: 106.

### Embodiment 39-2

The polynucleotide construct of Embodiment 39, wherein the 3′ homology arm consists of the nucleic acid sequence of SEQ ID NO: 106.

### Embodiment 40

The polynucleotide construct of any one of Embodiments 3-39, wherein the 5′ homology arm or 3′ homology arm is 15 to 60 nucleotides in length.

### Embodiment 41

The polynucleotide construct of any one of Embodiments 3-40, wherein the 5′ homology arm or 3′ homology arm sequence has up to 10% base mismatches.

### Embodiment 42

The polynucleotide construct of any one of Embodiments 1-41, wherein the target sequence comprises a 5′ arm sequence selected from the group consisting of:
(a) SEQ ID NO: 89;
(b) SEQ ID NO: 90;
(c) SEQ ID NO: 91;
(d) SEQ ID NO: 92;
(e) SEQ ID NO: 93;
(f) SEQ ID NO: 94;
(g) SEQ ID NO: 95; and
(h) SEQ ID NO: 96.

### Embodiment 43

The polynucleotide construct of any one of Embodiments 1-42, wherein the target sequence comprises a 3′ arm sequence selected from the group consisting of:
(a) SEQ ID NO: 97;
(b) SEQ ID NO: 98;
(c) SEQ ID NO: 99;
(d) SEQ ID NO: 100;
(e) SEQ ID NO: 101;
(f) SEQ ID NO: 102;
(g) SEQ ID NO: 103; and
(h) SEQ ID NO: 104.

### Embodiment 44

The polynucleotide construct of any one of Embodiments 1-43, wherein the target sequence comprises Formula I:

TI-(L)ₙ-Z1 (I)

wherein:
TI is an engineered translation initiation element comprising an internal ribosome entry site (IRES)-like polynucleotide sequence or a natural IRES sequence,
Z1 is an expression sequence encoding a therapeutic product; L is a linker sequence;
A1 and B1 are a pair of sequences capable of circularization of the RNA polynucleotide; and
n is an integer selected from 0 to 2.

### Embodiment 45

The polynucleotide construct of Embodiment 44, wherein Z1 comprises a nucleic acid sequence selected from the group consisting of:
(a) a nucleic acid sequence 95% identical to SEQ ID NO: 107;
(b) a nucleic acid sequence 98% identical to SEQ ID NO: 107;
(c) a nucleic acid sequence 99% identical to SEQ ID NO: 107;
(d) SEQ ID NO: 107;
(e) a nucleic acid sequence 95% identical to SEQ ID NO: 108;
(f) a nucleic acid sequence 98% identical to SEQ ID NO: 108;
(g) a nucleic acid sequence 99% identical to SEQ ID NO: 108;
(h) SEQ ID NO: 108;
(i) a nucleic acid sequence 95% identical to SEQ ID NO: 109;
(j) a nucleic acid sequence 98% identical to SEQ ID NO: 109;
(k) a nucleic acid sequence 99% identical to SEQ ID NO: 109;
(l) SEQ ID NO: 109;
(m) a nucleic acid sequence 95% identical to SEQ ID NO: 110;
(n) a nucleic acid sequence 98% identical to SEQ ID NO: 110;
(o) a nucleic acid sequence 99% identical to SEQ ID NO: 110;
(p) SEQ ID NO: 110;
(q) a nucleic acid sequence 95% identical to SEQ ID NO: 111;
(r) a nucleic acid sequence 98% identical to SEQ ID NO: 111;
(s) a nucleic acid sequence 99% identical to SEQ ID NO: 111;
(t) SEQ ID NO: 111;
(u) a nucleic acid sequence 95% identical to SEQ ID NO: 112;
(v) a nucleic acid sequence 98% identical to SEQ ID NO: 112;
(w) a nucleic acid sequence 99% identical to SEQ ID NO: 112;
(x) SEQ ID NO: 112.

### Embodiment 45-1

The polynucleotide construct of Embodiment 45, wherein Z1 consists essentially of a nucleic acid sequence selected from the group consisting of a nucleic acid sequence 95% identical to any one of SEQ ID NO: 107-SEQ ID NO: 112, a nucleic acid sequence 98% identical to any one of SEQ ID NO: 107-SEQ ID NO: 112, a nucleic acid sequence 99% identical to any one of SEQ ID NO: 107-SEQ ID NO: 112, and any one of SEQ ID NO: 107-SEQ ID NO: 112.

### Embodiment 45-2

The polynucleotide construct of Embodiment 45, wherein Z1 consists of a nucleic acid sequence selected from the group consisting of a nucleic acid sequence 95% identical to any one of SEQ ID NO: 107-SEQ ID NO: 112, a nucleic acid sequence 98% identical to any one of SEQ ID NO: 107-SEQ ID NO: 112, a nucleic acid sequence 99% identical to any one of SEQ ID NO: 107-SEQ ID NO: 112, and any one of SEQ ID NO: 107-SEQ ID NO: 112.

### Embodiment 46

The polynucleotide construct of Embodiment 44, wherein Z1 comprises a nucleic acid sequence encoding the amino acid sequence selected from the group consisting of:
(a) SEQ ID NO: 113;
(b) SEQ ID NO: 114;
(c) SEQ ID NO: 115;
(d) SEQ ID NO: 116;
(e) SEQ ID NO: 117;
(f) SEQ ID NO: 118; and

### Embodiment 46-1

The polynucleotide construct of Embodiment 46, wherein the Z1 consists essentially of a nucleic acid sequence encoding the amino acid sequence selected from the group consisting of SEQ ID NO: 113-SEQ ID NO: 118.

### Embodiment 46-2

The polynucleotide construct of Embodiment 46, wherein the Z1 consists of a nucleic acid sequence encoding the amino acid sequence selected from the group consisting of SEQ ID NO: 113-SEQ ID NO: 118.

### Embodiment 47

The polynucleotide construct of any one of Embodiments 1-46, comprising a modified RNA nucleotide and/or modified nucleoside.

### Embodiment 48

The polynucleotide construct of any one of Embodiments 1-47, comprising 10% to 100% modified RNA nucleotide and/or modified nucleoside.

### Embodiment 49

The polynucleotide construct of any one of Embodiments 47-48, wherein at least one of the modified RNA nucleotide and/or modified nucleoside is m5C (5-methylcytidine).

### Embodiment 50

The polynucleotide construct of any one of Embodiments 47-48, wherein at least one of the modified RNA nucleotide and/or modified nucleoside is m5U (5-methyluridine).

### Embodiment 51

The polynucleotide construct of any one of Embodiments 47-48, wherein at least one of the modified RNA nucleotide and/or modified nucleoside is m6A (N6-methyladenosine).

### Embodiment 52

The polynucleotide construct of any one of Embodiments 47-48, wherein at least one of the modified RNA nucleotide and/or modified nucleoside is Y (pseudouridine).

### Embodiment 53

The polynucleotide construct of any one of Embodiments 47-48, wherein at least one of the modified RNA nucleotide and/or modified nucleoside is m1A (1-methyladenosine).

### Embodiment 54

The polynucleotide construct of any one of Embodiments 47-53, wherein at least one of the modified RNA nucleotide and/or modified nucleoside is introduced at in vitro transcription (IVT).

### Embodiment 55

The polynucleotide construct of any one of Embodiments 47-48, wherein the modified nucleoside is selected from the group consisting of: m5C (5-methylcytidine), m5U (5-methyluridine), m6A (N6-methyladenosine), s2U (2-thiouridine), Y (pseudouridine), Um (2′-O-methyluridine), m1A (1-methyladenosine), m2A (2-methyladenosine), Am (2′-O-methyladenosine), ms2 m6A (2-methylthio-N6-methyladenosine), i6A (N6-isopentenyladenosine), ms2i6A (2-methylthio-N6 isopentenyladenosine), io6A (N6-(cis-hydroxyisopentenyl)adenosine), ms2io6A (2-methylthio-N6-(cis-hydroxyisopentenyl)adenosine), g6A (N6-glycinylcarbamoyladenosine), t6A (N6-threonylcarbamoyladeno sine), ms2t6A (2-methylthio-N6-threonyl carbamoyladenosine), m6t6A (N6-methyl-N6-threonylcarbamoyladenosine), hn6A(N6-hydroxynorvalylcarbamoyladenosine), ms2hn6A (2-methylthio-N6-hydroxynorvalyl carbamoyladenosine), Ar(p) (2′-O-ribosyladenosine (phosphate)), I (inosine), m1I (1-methylinosine), mlhn (1,2′-O-dimethylinosine), m3C (3-methylcytidine), Cm (2′-O-methylcytidine), s2C (2-thiocytidine), ac4C (N4-acetylcytidine), (5-formylcytidine), m5Cm (5,2′-O-dimethylcytidine), ac4Cm (N4-acetyl-2′-O-methylcytidine), k2C (lysidine), m!G (1-methylguanosine), m2G (N2-methylguanosine), m7G (7-methylguanosine), Gm (2′-O-methylguanosine), m2 2G (N2,N2-dimethylguanosine), m2Gm (N2,2′-O-dimethylguanosine), m2 aGm (N2,N2,2′-O-trimethylguanosine), Gr(p) (2′-O-ribosylguanosine(phosphate)), yW (wybutosine), oayW (peroxywybutosine), OHyW (hydroxy wybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQo (7-cyano-7-deazaguanosine), preQi (7-aminomethyl-7-deazaguanosine), G+ (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxy acetic acid), mcmo5U (uridine 5-oxy acetic acid methyl ester), chm5U (5-(carboxyhydroxymethyl)uridine)), mchm5U (5-(carboxyhydroxymethyl)uridine methyl ester), mcm5U (5-methoxycarbonylmethyluridine), mcm5Um (5-methoxycarbonylmethyl-2′-O-methyluridine), mcm5s2U (5-methoxycarbonylmethyl-2-thiouridine), nm5S2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyluridine), ncm5Um (5-carbamoylmethyl-2′-O-methyluridine), cmnm5U (5-carboxymethylaminomethyluridine), cmnm5Um (5-carboxymethylaminomethyl-2′-O-methyluridine), cmnm5s2U (5-carboxymethylaminomethyl-2-thiouridine), m6 2A (N6,N6-dimethyladenosine), Im (2′-O-methylinosine), m4C (N4-methylcytidine), m4Cm (N4,2′-O-dimethylcytidine), hm5C (5-hydraxymethylcytidine), m3U (3-methyluridine), cm5U (5-carboxymethyluridine), m6Am (N6,2′-O-dimethyladenosine), m6 2Am (N6,N6,0-2′-trimethyladenosine), m2,7G (N2,7-dimethylguanosine), m2,2,7G (N2,N2,7-trimethylguanosine), m3Um (3,2′-O-dimethyluridine), m5D (5-methyldihydrouridine), f5Cm (5-formyl-2′-O-methylcytidine), m′Gm (1,2′-O-dimethylguanosine), m′Am (1,2′-O-dimethyladenosine), rm 5U (5-taurinomethyluridine), rm5s2U (5-taurinomethyl-2-thiouridine)), imG-14 (4-demethylwyosine), imG2 (isowyosine), or ac6A (N6-acetyladenosine), pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5-methylcytosine, pseudouridine, and 1-methylpseudouridine.

### Embodiment 56

A circular RNA produced by the polynucleotide construct of any of Embodiments 1-55, for example, the circular RNA is at least 500 nucleotides in length, at least 1,000 nucleotides in length, or at least 1,500 nucleotides in length.

### Embodiment 57

The circular RNA of Embodiment 56, not comprising any other sequences that do not belong to the target sequence, such as not comprising all or part of an E2 sequence and an E1 sequence.

### Embodiment 58

A method of making a circular RNA, said method comprising: preparing a vector comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 59

A method of making circular RNA, said method comprising: preparing a vector comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 60

A method of making circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 61

A method of making circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

### Embodiment 62

A method for expressing a protein in a cell, comprising (a) transfecting the cell with the circular RNA of any one of Embodiments 58-61, or (b) subjecting the polynucleotide construct of any of Embodiments 1-57 to a self-splicing circularization reaction to form a circular RNA, and transfecting the cell with the circular RNA; wherein, preferably the cell is a eukaryotic cell.

### Embodiment 63

A method for expressing a protein in a cell, comprising (a) transfecting the cell with the circular RNA of any one of Embodiments 58-61, or (b) subjecting the construct of any of Embodiments 1-57 to a self-splicing circularization reaction to form a circular RNA, and transfecting the cell with the circular RNA; wherein, preferably the cell is a hepatocyte, epithelial cell, hematopoietic cell, epithelial cell, endothelial cell, lung cell, bone cell, stem cell, mesenchymal cell, neural cell (e.g., meninge, astrocyte, motor neuron, cell of the dorsal root ganglia and anterior horn motor neuron), photoreceptor cell (e.g., rod and cone), retinal pigmented epithelial cell, secretory cell, cardiac cell, adipocyte, vascular smooth muscle cell, cardiomyocyte, skeletal muscle cell, beta cell, pituitary cell, synovial lining cell, ovarian cell, testicular cell, fibroblast, B cell, T cell, dendritic cell, macrophage, reticulocyte, leukocyte, granulocyte, tumor cell, NK cell, liver starlet cell, HEK293, HEK293T, HeLa, MCF7, PC3, A549, NCI-H727, HCT-116, MCF10A, HPReC, FHC, immortalized cell lines, primary cell, yeast cell, Saccharomyces cerevisiae, Pichia pastoris, bacteria cell, Escherichia coli, insect cell, Spodoptera frugiperda sf9, Mimic Sf9, sf21, or Drosophila S2.

### Embodiment 64

A method for generating a sequence with self-splicing activity using a group II intron, the method comprising the steps of:
(a) defining the sequence of the group II intron; optionally examining the in vitro self-splicing activity of the group II intron using a splicing assay (linear splicing);
(b) splitting the group II intron into two fragments,
(c) reversing the order of the two intron fragments, and
(d) confirming the in vitro circularization of RNA using a splicing assay.

The present invention also includes the following embodiments.

### Embodiment 65

The polynucleotide construct, circular RNA, or method of any one of the preceding Embodiments, wherein the 5′ intron fragment and the 3′ intron fragment respectively comprise one or more pairs of paired sequences that are complementary to each other. In a preferred embodiment, the complementary paired sequence is greater than 20 nucleotides in length.

### Embodiment 66

The polynucleotide construct, circular RNA, or method of any one of the preceding Embodiments, wherein the 5′ intron fragment and/or the 3′ intron fragment comprises one or more affinity tag sequences selected from the group consisting of: a probe binding sequence, an MS2 binding site, a PP7 binding site, and a streptavidin binding site.

### Embodiment 67

The polynucleotide construct, circular RNA, or method of any one of the preceding Embodiments, wherein the EBS sequence is selected from one or more of EBS1, EBS2 and EBS3, preferably two of them, more preferably EBS1 and EBS3.

### Embodiment 68

The polynucleotide construct, circular RNA, or method of any one of the preceding Embodiments, wherein one or more EBS sequences of the group II intron, preferably EBS1 and EBS3, are modified, wherein the EBS sequences are complementarily paired with two regions of a corresponding length in a target sequence on at least 60% of the nucleotide positions respectively. In a preferred embodiment, the two regions of a corresponding length in a target sequence are located at both ends of the target sequence, respectively.

### Embodiment 69

The polynucleotide construct, circular RNA, or method of any one of the preceding Embodiments, wherein the polynucleotide construct is capable of forming a circular RNA of a target sequence in vitro.

### Embodiment 70

The polynucleotide construct, circular RNA, or method of any one of the preceding Embodiments, wherein the polynucleotide construct is capable of forming a circular RNA of a target sequence in vivo.

### 5. BRIEF DESCRIPTION OF THE DRAWINGS

Embodiments of the present invention are described with reference to the various drawings.

FIG. 1 is a flow chart introducing the method of the present invention, showing a process of obtaining a circular RNA starting from a natural self-splicing ribozyme, through design, engineering, and final reaction.

FIGS. 2A-B illustrate a screening process for the group II introns in Example 1. (A) A DNA construct comprising a Gluc coding sequence fragment and an E1-group II intron (self-splicing ribozyme)-E2 was prepared, and a linear RNA was prepared by in vitro transcription using this DNA construct as a template and purified. In vitro self-splicing activity is supported if the linear RNA produces two fragments of different sizes (the excised intron, and the remainder of the construct) by the in vitro self-splicing reaction. The group II intron and its flanking E1 and E2 sequences may be used as a cRNAzyme precursor for designing a cRNAzyme construct. (B) In vitro self-splicing reaction conditions for screening cRNAzyme precursors.

FIG. 3 is a gel electrophoretogram of two group II introns confirmed to have self-splicing activity according to the method of Example 1. The names of the group II introns were marked with a 3-letter code on the respective electrophoretograms.

FIGS. 4A-C show the scheme of designing a cRNAzyme construct with Cte as an example, and comparative experimental results among different schemes. (A) cRNAzyme construct design; (B) percent of circularizing determined by gel electrophoresis after segmenting a II Cte intron at different positions and obtaining the construct; and (C) graphs of the results of experiments verifying the successful formation of a circular RNA by different methods.

FIGS. 5A-C show the results obtained under different conditions during the optimization. (A) The percent of circularizing of the cRNAzyme construct has been improved by optimizing reaction conditions and engineering sequences; (B) graphs of the gel electrophoresis results of circularization products under different reaction conditions, with Cte as an exemplary self-splicing ribozyme; the lower histogram shows the quantified percent of circularizing PC % (percent of circularizing (PC %)=circular/(circular+linear)×100%); and (C) a gel electrophoretogram of the circularization products produced by three constructs with Cte as an exemplary ribozyme and Renilla Luciferase (Rluc) as an insert, with the addition of different spacer sequences; and the lower histogram shows the quantified percent of circularizing PC %.

FIGS. 6A-B relate to the improved construct prepared in Example 4 capable of eliminating the scar sequence. (A) A structural diagram of the construct. (B) Gel electrophoretograms and sequencing results of the circularization products of the three target sequences under different magnesium ion concentrations.

FIG. 7 shows the results of gel electrophoresis of circular RNAs generated upon insertion of target sequences of different lengths.

FIGS. 8A-B are the results of intracellular expression of circular RNAs of different target sequences generated using the construct and method of the present invention. (A) A “scarless” construct was used with GFP as the target sequence to form a circular RNA, and the results of GFP expression were detected by Western blotting after transfection of cells; and (B) a “scarless” construct was used with Gluc as the target sequence to form a circular RNA, and the results of Gluc expression were detected by a microplate reader after transfection of cells.

FIG. 9 is a structural diagram of group II introns.

FIG. 10 shows (a.) the branching pathway and (b.) the hydrolytic pathway of group II introns.

FIG. 11 shows the splicing mechanisms of group I and group II introns.

FIG. 12A is a schematic diagram of a near-scarless system which is designed based on the interactions between IBS1 and EBS1; IBS2 and EBS2; IBS3 and EBS3. The autocatalytic self-splicing group II intron is split into two fragments at the D4 domain, and a customized exons containing E1, E2, and a target sequence are inserted between the split intron. Arrows indicate the interactions between IBS1 and EBS1; IBS2 and EBS2; IBS3 and EBS3.

FIG. 12B is a schematic diagram for the design of a near-scarless system which is designed based on the interactions between a δ and IBS3. The autocatalytic self-splicing group II intron is split into two fragments at the D4 domain, and a customized exons containing E1, E2, and a target sequence were inserted between the split intron. Arrows indicate the interactions between IBS1 and EBS1; IBS2 and EBS2; and IBS3 and δ.

FIG. 12C is a schematic diagram of a scarless system which is designed based on the interactions between IBS1′ and EBS1. The autocatalytic self-splicing group II intron is split into two fragments at the D4 domain, and a target sequence is inserted between the split intron. Arrows indicate the interactions between IBS1′ and EBS1, and IBS3′ and EBS3. IBS1′ is a region on the target sequence which has similar function of IBS1. IBS3′ is a region on the target sequence which has similar function of IBS3.

FIG. 12D is a schematic diagram of a scarless system which is designed based on the interactions between a δ and IBS3′. The autocatalytic self-splicing group II intron is split into two fragments at the D4 domain, and a target sequence is inserted between the split intron. Arrows indicate the interactions between IBS1′ and EBS1, and IBS3′ and δ.

FIG. 13 is results of circRNA in vitro synthesized and analyzed with agarose gel. IBS1 was mutated to disable self-splicing. After IVT, circularized RNAs were confirmed by Poly A tailing and RNase R treatment.

FIG. 14 is an updated figure of FIG. 4C which shows the results of experiments verifying the successful formation of a circular RNA by different methods and a diagram of linear and circular RNA construct.

FIG. 15 shows sanger sequencing output of RT-PCR across the splice junction of the CircRNA sample depicted in lane 1 and lane 3 from FIG. 14.

FIG. 16 is an updated figure of FIG. 6B which shows a diagram of the construct, a gel electrophoretograms and sequencing results of the circularization products of the three target sequences under different magnesium ion concentrations.

FIG. 17 shows circularization efficiency using different spacer region before the CVB3 IRES.

FIG. 18 shows the luminescent signal of luciferase protein expression from circular RNAs derived from different cRNAzyme variants.

FIG. 19 shows transfection and translation of circRNA in different doses. The circRNAs containing two different spacers were gel purified and transfected into three cell lines cultured in 24-well plate at different doses, the activity of luciferase were measured 24 hours after transfection.

FIG. 20 shows the circularization efficiency of cRNAzyme variant CV4 containing different genes. The different RNAs were in vitro transcribed and circularized in the vitro transcription reaction. Gene 1: Gluc, Gene2: EGFP, Gene 3: RBD, Gene 4: Rluc, Gene 5: Fluc, Gene 6: saCAS9.

FIG. 21 shows result of a time course experiment for circRNA translation. The circRNAs encoding the Rluc gene were transfected into transfected into 293T cells (500 ng circRNAs were used in each transfection), and the luciferase activity were measured at 6, 12 and 24 hours after transfection.

FIG. 22 shows production from linear mRNA and circRNAs once transfected into cells.

FIG. 23 is a comparison of the protein production from the linear mRNAs with the circRNAs produced using PIE protocol or the new CirCode systems.

FIG. 24 shows HPLC purification of CVB3-Gluc circRNA from spin column purified sample after IVT. The top panel is the HPLC chromatogram indicating the peak of precursor, circular and intron RNA, respectively. The bottom panel demonstrates the agarose gel of input and collected fractions.

FIG. 25 shows the amount of cell death caused by transfection of the unpurified circRNAs and the purified circRNAs compared to mock transfection.

FIG. 26 shows a comparison of the unpurified circRNAs that stimulated innate immune response by inducing RIG-I and IFN-β1 with the purified circRNAs.

FIG. 27 demonstrates a scale up the production of circRNAs.

FIG. 28 demonstrates four batches of CVB3-Gluc circRNA purified from HPLC were analyzed using capillary electrophoresis with Agilent 2100 Bioanalyzer.

FIG. 29 is a schematic illustration of CircRNA-LNP complex and particle size of CircRNAGluc-LNP.

FIG. 30 shows the Gaussia luciferase activity assayed from mice serum 24 hours post-injection of CircRNAGluc-LNP with different formulation.

FIG. 31 shows representative IVIS images of BALB/c mice administrated with 20 ug CircRNAGluc-LNP with two formulation by the intramuscular (i.m.) routes. Relative luminescence plot is shown and the scale of luminescence is indicated.

FIG. 32 shows circRNA-RBD and circRNA-RBD dimer purified from HPLC, which were analyzed using capillary electrophoresis with Agilent 2100 Bioanalyzer (left bottom) and agarose gel electrophoresis.

FIG. 33 shows the particle size and the encapsulate efficiency of CircRNARBD-LNP complexes.

FIG. 34 shows a schematic diagram of the CircRNARBD-LNP vaccination process in BALB/c mice and serum collection schedule.

FIG. 35 shows results of RBD-biding B cells. A flow cytometry antibody panel was designed to identify naïve B cells (CD19+IgD+CD27−), total memory B cells (CD19+CD27+), including an unswitched IgD+population and a switched IgD− population, plasma cells (CD19+IgD-CD38+CD27+), and transitional B cell (CD19+IgDdimCD38+). To determine whether LNP-circRNA-RBP vaccination induced the activation and expansion of antigen-specific B cells, we measured the frequency of RBD-binding B cells using Alexa 647 labeled RBD (RBD-Alexa 647). we found that a large fraction of RBD-specific lymphocytes were detected in the memory B-cells (i.e., CD19+CD27+ B-lymphocytes) including an RBD specific switched B-cell population (CD19+CD27+IgD−RBD+) and an RBD specific unswitched memory B-cell population (CD19+CD27+IgD+RBD+).

FIG. 36 shows that CircRNARBD-LNP vaccination-elicited antibody responses. Sera were collected 2 weeks post-boost and assessed for RBD-specific IgG1, IgG2a, IgG2c by ELISA.

FIG. 37 shows inhibition of RBD binding to the hACE2 overexpressed cell line.

FIG. 38 shows the ratio between IgG2a/IgG1 and IgG2c/IgG1.

FIG. 39 shows that CircRNARBD-LNP vaccination-elicited neutralization antibody responses. Pseudovirus neutralization titers were accessed for the sera collected 2 weeks post-boost.

FIG. 40 shows a schematic diagram of group II intron with IBS1, IBS2, IBS3, EBS1, EBS2, EBS3 showing with bold line.

FIG. 41 is a structural diagram of group II intron with IBS1, IBS2, IBS3, EBS1, EBS2, EBS3 and δ showing in bold.

### 6. DETAILED DESCRIPTION OF EMBODIMENTS

### 6.1. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.1%, preferably below 0.05%, and more preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” or “additional” may mean at least a second or more.

As used herein, the term “about” is used to indicate that a value includes the inherent variation of error for the device, the method being employed to determine the value, or the variation that exists among the study subjects. In some embodiments, “about” means that the variation is ±5%, ±4%, ±3%, ±2%, ±1%, ±0.5%, ±0.2%, or ±0.1% of the value to which “about” refers. In some embodiments, “about” means that the variation is ±1%, ±0.5%, ±0.2%, or ±0.1% of the value to which “about” refers.

The term “cRNAzyme” is used herein to refer a linear ribonucleic acid (RNA) which is capable of producing a circular RNA via a self-catalyzed back-splicing reaction.

The term “cRNAzyme construct” is a linear RNA construct which has cRNAzyme activity.

The term “EBS” is used herein to refer to an exon binding sequence, which interact (e.g. forming a complementarily pair) with the intron binding sequences (IBSs) in exon regions, triggering splicing by virtue of their own hydroxyl groups within the EBS nucleic acid sequences

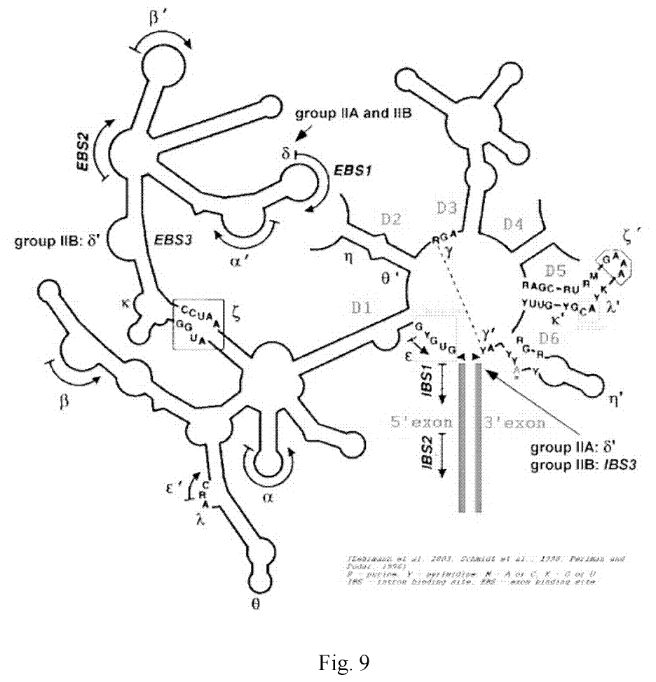

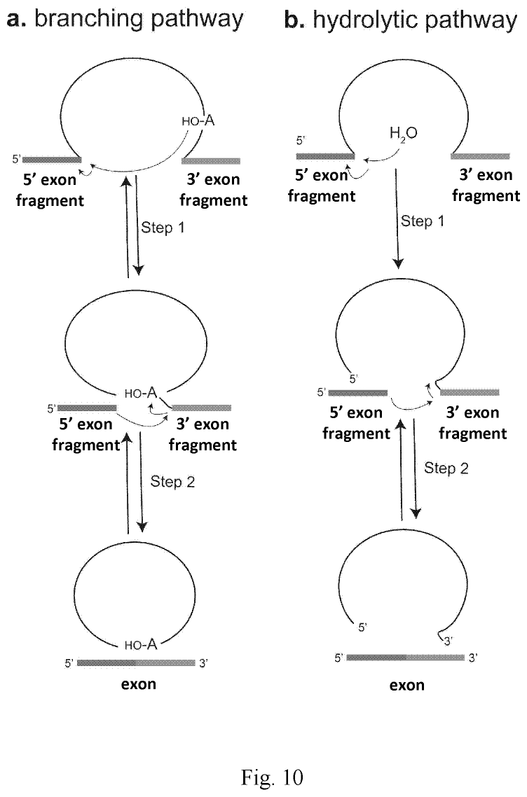

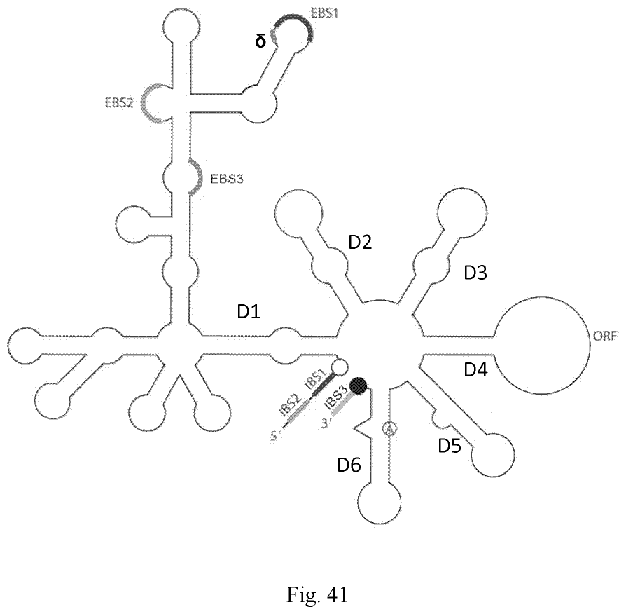

The term “EBS1” is used herein to refer exon binding sequence 1. See FIGS. 9 and 41.

The term “EBS2” is used herein to refer exon binding sequence 2. See FIGS. 9 and 41.

The term “EBS3” is used herein to refer exon binding sequence 3. See FIGS. 9 and 41.

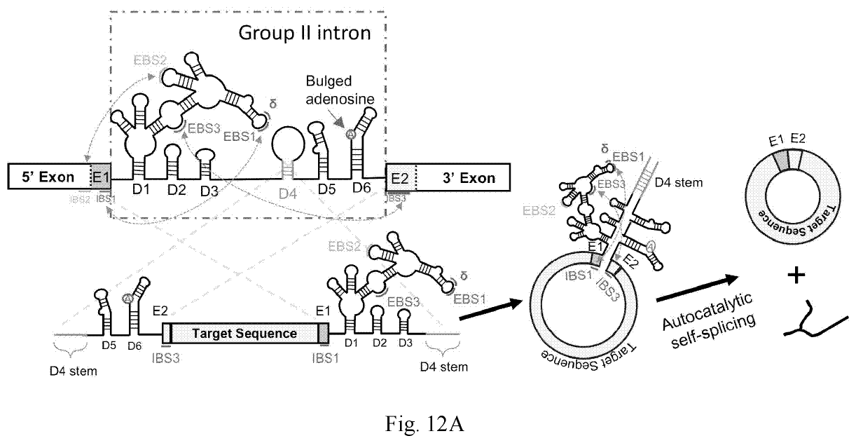

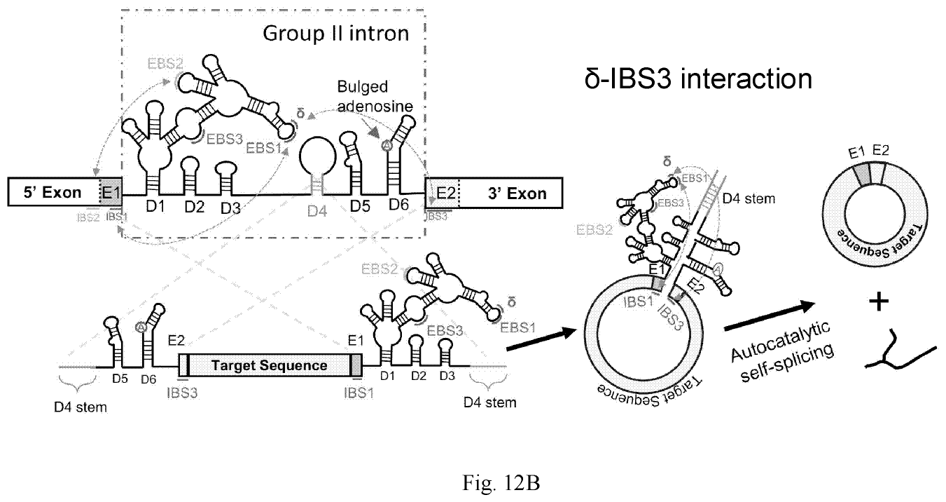

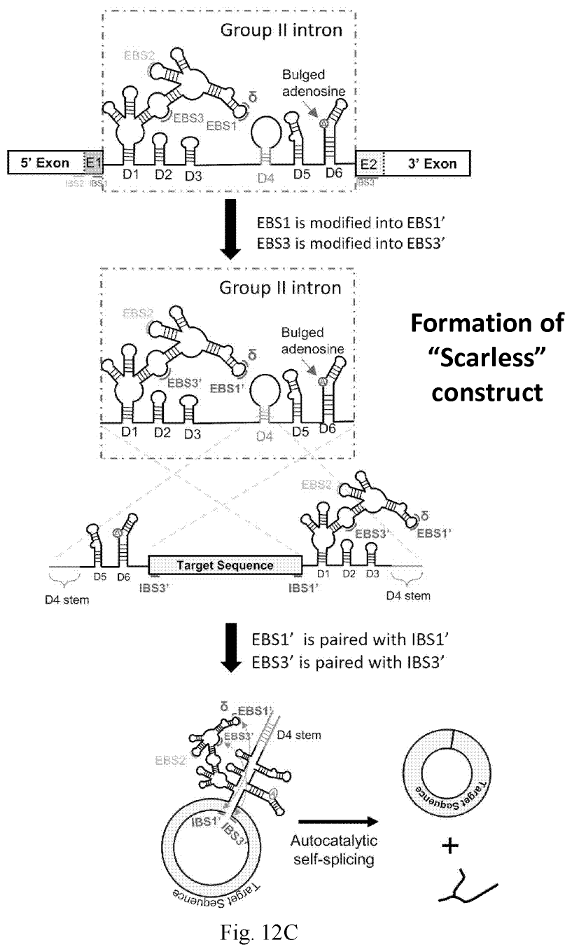

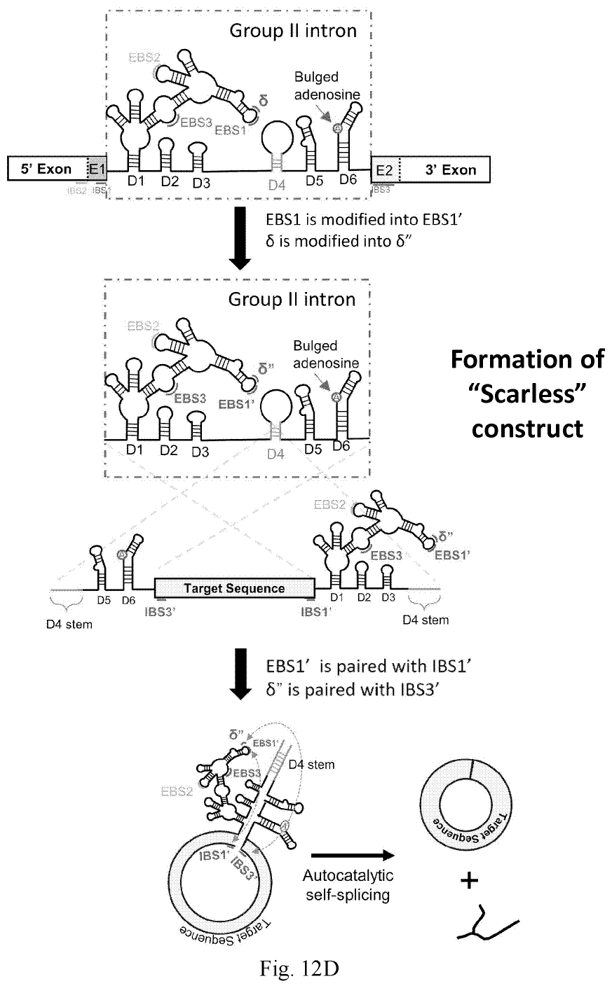

The term “EBS1′” is used herein to refer a modified EBS1 sequence which interacts with IBS1′. The interaction between EBS1′ and IBS1′ is similar as the interaction between EBS1 and IBS1. See FIGS. 12C and 12D.

The term “EBS3′” is used herein to refer a modified EBS3 sequence which interacts with IBS3′. The interaction between EBS3′ and IBS3′ is similar as the interaction between EBS3 and IBS3. See FIG. 12C.

The term “domain 1” or “D1” is used herein to refer to a stem-loop structure of domain 1 of a Group II intron. The term “domain 2” or “D2” is used herein to refer to a stem-loop structure of domain 2 of a Group II intron. The term “domain 3” or “D3” is used herein to refer to a stem-loop structure of domain 3 of a Group II intron. The term “domain 4” or “D4” is used herein to refer to a stem-loop structure of domain 4 of a Group II intron. The term “domain 5” or “D5” is used herein to refer to a stem-loop structure of domain 5 of a Group II intron. The term “domain 6” or “D6” is used herein to refer to a stem-loop structure of domain 6 of a Group II intron. Stem-loop structure is a type of an RNA secondary structure, which can be determined by any suitable polynucleotide folding algorithm. Some programs are based on the calculation of the minimum Gibbs free energy. An example of one such algorithm is mFold and is described by Zuker and Stiegler (Nucleic Acids Res. 9 (1981), 133-148). Another exemplary folding algorithm is the online web server RNAfold developed by the Institute for Theoretical Chemistry at the University of Vienna using a centroid structure prediction algorithm (e.g. AR Gruber et al., 2008, Cell 106). (1): 23-24; and PA Carr and GM Church, 2009, Nature Biotechnology 27 (12): 1151-62). Additional algorithms can be found in US Provisional Patent Application No. 61/836,080 (Attorney Docket No. 44790.11.2022; Broad reference number BI-2013/004A), which is incorporated herein by reference. Group II intron mainly comprises 6 stem-loop structures, called domains 1 to 6 (D1 to D6), and the 6 domains are arranged in sequence, comprising multiple exon binding sequences (EBSs), such as EBS1, EBS2, and EBS3. These EBS sequences interact, such as complementarily pair, with the intron binding sequences (IBSs) in exon regions, triggering splicing by virtue of their own hydroxyl groups within the EBS nucleic acid sequences.

As used herein, the term “group II intron” is used herein to refer to RNA molecules which are encoded by the group II introns, share a similar secondary and tertiary structure. The group II intron RNA molecules typically have six domains. See FIG. 9 and FIG. 41. Domain 4 (also known as domain IV) of the group II intron RNA contains the nucleotide sequence which encodes the “group II intron-encoded protein.”

The term “IBS” is used herein to refer to an intron binding sequence, which interacts with exon binding sequence (EBS) to locate splicing site.

The term “IBS1” is used herein to refer to an intron binding sequence 1, which interacts with exon binding sequence 1 (EBS1) to locate splicing site.

The term “IBS1′” is used herein to refer to a region on a target sequence which has similar function of IBS1.

The term “IBS2” is used herein to refer to an intron binding sequence 2, which interacts with exon binding sequence 2 (EBS2) to locate splicing site.

The term “IBS3” is used herein to refer to an intron binding sequence 3, which interacts with exon binding sequence 3 (EBS3) to locate splicing site.

The term “IBS3′” is used herein to refer to a region on a target sequence which has similar function of IBS3.

The term “δ” (delta) is used herein to refer to a region on domain 1 of a group II intron which is the single nucleotide directly upstream of EBS1. δ pairs with IBS3 and the interaction between δ and IBS3 is called δ-IBS3 pairing. see FIG. 41, 12B.

The term “δ” “(delta”) is used herein to refer to a region on domain 1 of a group II intron which is the single nucleotide directly upstream of EBS1′. δ″ pairs with IBS3′ and the interaction between δ″ and IBS3′ is called δ″-IBS3′ pairing. see FIG. 41, 12D.

The term “IVT” is used herein to refer in vitro transcription which is a versatile method to produce RNA in vitro that uses an RNA polymerase, ribonucleotides, and appropriate buffer conditions to synthesis RNA from a DNA template.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

The terms “nucleic acid sequence” “polynucleotide”, and “oligonucleotide” are used interchangeably herein and refer to a polymer or oligomer of pyrimidine and/or purine bases, such as cytosine, thymine, and uracil, adenine and guanine, respectively (see Albert L. Lehninger, Principles of Biochemistry, at 793-800 (Worth Pub. 1982)), unless specified otherwise or the context indicates to the contrary. The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. A nucleic acid or nucleic acid sequence may comprise other kinds of nucleic acid structures such as, for instance, a DNA/RNA helix, peptide nucleic acid (PNA), morpholino nucleic acid (see, e.g., Braasch and Corey, Biochemistry, 4/(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, Am. Chem. Soc., 122: 8595-8602 (2000)), and/or a ribozyme. The terms “nucleic acid”, “nucleic acid sequence”, “polynucleotide”, and “oligonucleotide” may also encompass a chain comprising non-natural nucleotides, modified nucleotides, and/or non-nucleotide building blocks that can exhibit the same function as natural nucleotides (e.g., “nucleotide analogs”). The term “DNA sequence” is used herein to refer to a nucleic acid comprising a series of DNA bases.

The terms “polypeptide” and “protein” are used interchangeably herein, refer to a polymeric form of amino acids comprising at least two or more contiguous amino acids chemically or biochemically modified or derivatized amino acids. The term “peptide” as used herein refers to a class of short polypeptides. The term peptide may refer to a polymer of amino acid's (natural or non-naturally occurring) having a length of up to about 100 amino acid. For example, peptides may be about 1 to about 10, about 10 to about 2, about 25 to about 50, about 50 to about 75, about 75 to about 100 amino acid residues in length. In some embodiments, the peptides may be about 100, about 200, about 300, about 400, about 500, about 600, about 700, about 800, about 900, about 1000, about 1250, about 1500, about 1750, about 2000, about 2250 about 2500, about 2750, about 3000, about 3250, about 3500, about 3750, about 4000, about 4250, about 4500, about 4750, are about 5000 amino acid residues in length.

Nomenclature for nucleotides, nucleic acids, nucleosides, and amino acid use herein is consistent with International Union of Pure and Applied Chemistry (IUPAC) standards (see, e.g., bioinformatics.org/smsylupac.html).

When referring to a nucleic acid sequence or protein sequence the term “identity” is used to denote similarity between two sequences. Sequence similarity or identity may be determined using standard techniques known in the art, including but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetic-s Software Pack, Genetics Computer Group, 575 Science Drive, Madison, WI), the Best Fit sequence program described by Devereux et al., Nucl. Acid Res. 12, 387-395 (1984), or by inspection. Another algorithm is the BLAST algorithm describe in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

The terms “internal ribosome entry site,” “internal ribosome entry site sequence,” “IRES” and “IRES sequence region” are used interchangeably herein and refer to cis elements of viral or human cellular RNAs (e.g., messenger RNA (mRNA) and/or circRNAs) that bypass the steps of canonical eukaryotic cap-dependent translation initiation. The canonical cap-dependent mechanism used by the vast majority of eukaryotic mRNAs requires an m⁷G cap at the 5′ end of the mRNA, initiator Met-tRNA met, more than a dozen initiation factor proteins, directional scanning, and GTP hydrolysis to place a translationally competent ribosome at the start codon. IRESs typically are comprised of a long and highly structured 5-UTR which mediates the translation initiation complex binding and catalyzes the formation of a functional ribosome.

The term “IRES-like sequence” or “Internal Ribosome Entry Site-like sequence” refer to synthetic nucleotide sequences that display a function of a natural IRES. In some embodiments, the IRES-like sequence can recruit ribosomal components to mediate cap-independent translation.

The terms “coding sequence,” “coding sequence region,” “coding region,” and “CDS” when referring to nucleic acid sequences may be used interchangeably herein to refer to the portion of a DNA or RNA sequence, for example, that is or may be translated to protein. The terms “reading frame,” “open reading frame,” and “ORF,” may be used interchangeably herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The terms “complementary” and “complementarity” refers to the relationship between two nucleic acid sequences or nucleic acid monomers having the capacity to form hydrogen bond(s) with one another by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., about 50%, about 60%, about 70%, about 80%, about 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., at least 65%, at least 70%, at least 75%, at least 80%, at least 85%, at least 90%, at least 95%, at least 97%, at least 98%, at least 99%, or 100%) over a region of at least 8 nucleotides (e.g., at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, or more nucleotides), or if the two nucleic acid sequences hybridize under at least moderate, or, in some embodiments high, stringency conditions. Exemplary moderate stringency conditions include overnight incubation at 37° C. in a solution comprising 20% formamide, 5% SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1*SSC at about 37-50° C., or substantially similar conditions, e.g., the moderately stringent conditions described in Sambrook, J., Molecular Cloning: A Laboratory Manual, Cold Spring Harbor Laboratory Press; 4th edition (Jun. 15, 2012). High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 pg/ml), 0.1% SDS, and 10% dextran sulfate at 42° C., with washes at (i) 42° C. in 0.2*SSC, (ii) 55° C. in 50% formamide, and (iii) 55° C. in 0.1*SSC (optionally in combination with EDTA). Additional details and an explanation of stringency of hybridization reactions are provided in, e.g., Sambrook, supra, and Ausubel et al., eds., Short Protocols in Molecular Biology, 5th ed., John Wiley & Sons, Inc., Hoboken, N.J. (2002).

The term “hybridization” or “hybridized” when referring to nucleic acid sequences is the association formed between and/or among sequences having complementarity.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need to be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to an RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and a functional effector element) are connected in such a way as to permit transcription of the nucleic acid molecule.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Two polynucleotide (e.g., DNA) or two polypeptide sequences are “substantially homologous” to each other when at least about 80%, preferably at least about 90%, and most preferably at least about 95% of the nucleotides, or amino acids, respectively match over a defined length of the molecules, as determined using the methods above.

The terms “scar” refer to the length of the region in a circular product excluding the target sequence. A scarless cirRNA contains 0 nucleotide scar sequence. A near-scarless cirRNA contains a scar sequence that is equal to or less than 20 nucleotide in length.

“Treating” or “treatment of a disease or condition” refers to executing a protocol or treatment plan, which may include administering one or more drugs or active agents to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. In addition, “treating” or “treatment” does not require complete alleviation of signs or symptoms, and does not require a cure.

The term “therapeutic benefit” or “therapeutically effective” as used throughout this application refers to anything that promotes or enhances the well-being of the subject with respect to the medical treatment of this condition. This includes, but is not limited to, a reduction in the frequency, severity, or rate of progression of the signs or symptoms of a disease. For example, treatment of cancer may involve, for example, a reduction in the size of a tumor, a reduction in the invasiveness of a tumor, reduction in the growth rate of the cancer, or a reduction in the rate of metastasis or recurrence. Treatment of cancer may also refer to prolonging survival of a subject with cancer.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous biocompatible solvents (e.g., saline solutions, phosphate buffered saline, parenteral vehicles, such as sodium chloride, Ringer's dextrose, etc.), antioxidants, preservatives (e.g., antibacterial or antifungal agents, anti-oxidants, chelating agents, and inert gases), isotonic agents, such like materials and combinations thereof, as would be known to one of ordinary skill in the art. The pH and exact concentration of the various components in a pharmaceutical composition are adjusted according to well-known parameters.

As used herein and unless otherwise specified, the term “about” means within plus or minus 10% of a given value or range. In certain embodiments, the term “about” encompasses the exact number recited.

### 6.2. Ribozymes and Group II Introns

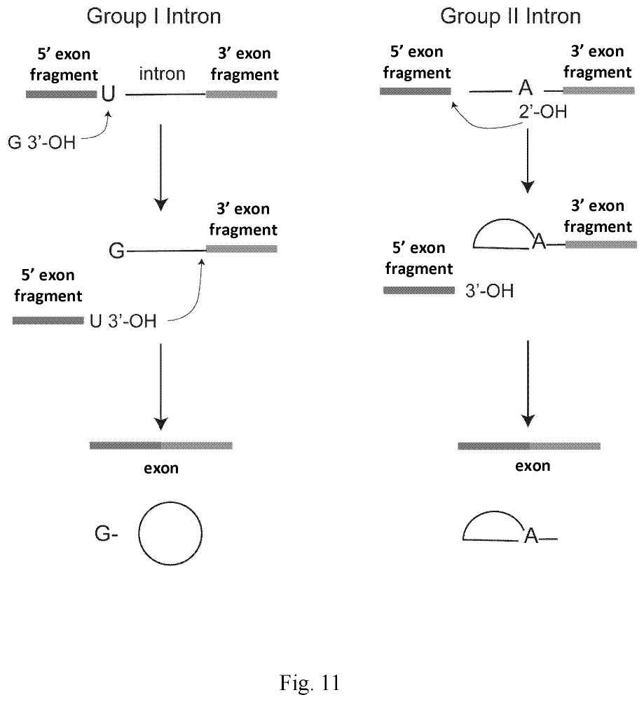

The ribozyme itself is a stretch of RNA nucleic acid molecule. Since such nucleic acid sequences have enzymatic activity, they are called ribozymes. For example, some intron sequences from some mitochondrion or bacteria may directly catalyze the occurrence of splicing independent of the spliceosome, and are referred to as “ribozymes with self-splicing activity”, “self-splicing ribozymes” or “self-splicing introns”. Self-splicing introns that may perform splicing without any protein comprise both group I and group II introns. As mentioned above, the two introns are significantly different in structure and in the mechanism of the self-splicing reaction. See FIG. 10. The present invention specifically relates to group II self-splicing introns, also simply referred to as “group II introns”.

The method for preparing a circular RNA using a self-splicing ribozyme has the following advantages:
1) Reduction of the use of biological and chemical reagents. The use of ribozymes may effectively reduce the contamination of exogenous biological products (such as ligase) and the contamination of other chemical reagents during the preparation. When using ribozymes for catalytic self-splicing, only a few reagents, such as Tris-HCl buffer, Mg ions, sodium ions, and GTP, are required in the reaction system. In the case of the present invention, GTP may also be omitted due to the use of group II introns. In contrast, when using a ligase for the ligation reaction to prepare a circular RNA, in addition to the ligase itself, on the one hand, corresponding chemical reagents, such as Tris-HCl buffer, KCl, DTT, EDTA and glycerol need to be used for the preservation of the enzyme; on the other hand, chemical reagents, such as Mg ions, DTT and ATP are also required to be added to the reaction system. Reducing the variety of reagents may save costs and simplify operations.
2) Ease of operation. As mentioned above, due to the small variety of reagents required for the reaction, the circularization reaction may be completed in one step on a PCR instrument by only adding a buffer containing GTP (for group I self-splicing introns only) and ions to the RNA. In contrast, when using an RNA ligase for the ligation reaction, at least an additional ligase needs to be added.
3) Simple design. For circular RNAs with larger molecular weights, such as circular RNAs containing coding sequences, the efficiency of direct ligation is very low, and exogenous DNA splint usually needs to be introduced, which requires precise pairing of RNA and DNA, thereby increasing the complexity of design and operation.

FIG. 9 shows a schematic diagram of the secondary structures of group II introns. As shown in FIG. 9, the group II intron mainly comprises 6 stem-loop structures, called domains 1 to 6 (D1 to D6), and the 6 domains are arranged in sequence, comprising multiple exon binding sequences (EBSs), such as EBS1, EBS2, and EBS3. These EBS sequences interact, such as complementarily pair, with the intron binding sequences (IBSs) in exon regions, triggering splicing by virtue of their own hydroxyl groups within the EBS nucleic acid sequences. This splicing mechanism is closer to the splicing reaction mediated by the spliceosome, and more similar to splicing in higher organisms.

In a preferred embodiment, the group II intron is derived from the microorganism kingdom (bacteria domain). In a specific embodiment, the group II intron is derived from Clostridium, such as Clostridium tetani, or Bacillus, such as Bacillus thuringiensis. It is understood by those skilled in the art that the key to the present invention lies in the design of a construct and a method, that is applicable to various group II introns. The implementation of the present invention is not limited to a specific group II intron type, as long as the group II intron has self-splicing circularization activity in vitro, which can be confirmed by those skilled in the art by conventional means.

In some embodiments of the present invention, the group II intron may be a wild-type group II intron or a modified group II intron. The modified group II intron comprises a substitution, a deletion and/or an addition of one or more nucleotides. Preferably, the modification does not affect the self-splicing activity of the group II intron, especially the in vitro self-splicing activity.

### 6.3. Constructs of the Present Invention

In the context of the present invention, natural self-splicing ribozymes may be referred to as self-splicing ribozymes or cRNAzyme precursors, and rearranged and engineered self-splicing ribozymes may be referred to as cRNAzymes. Further, a cRNAzyme linked to a target sequence, such as a protein coding sequence or a protein noncoding sequence, is referred to as a cRNAzyme construct, i.e., the polynucleotide construct of the present invention.

Specifically, by bisecting a stretch of sequence (E1-intron-E2) consisting of a natural group II intron and its two flanking exon fragments (E1 and E2), two fragments are formed, i.e., a first fragment having the structure E1-5′ intron fragment, and a second fragment having the structure 3′ intron fragment-E2. The 5′ intron fragment was originally located at the 5′ end of the 3′ intron fragment, and was immediately adjacent to each other. When constructing the cRNAzyme, the first and second fragments are swapped in position and religated. The rearranged sequence structure is “3′ intron fragment-E2-E1-5′ intron fragment”. Sequences with this structure and self-splicing activity are called cRNAzymes. The self-splicing activity is preferably an activity that causes self-splicing and causes the POI sequence inserted therein to form a circular RNA. The self-splicing activity is preferably an activity by which self-splicing occurs in vitro.

When the cRNAzyme is used to catalyze the POI to form a circular RNA, the POI sequence, comprising the POI coding sequence and/or noncoding sequence, is constructed into the position between E2 and E1 of the cRNAzyme, thereby forming a cRNAzyme construct. The cRNAzyme construct may be transcribed into an RNA, and then subjected to self-splicing through the cRNAzyme structural elements contained therein, so that the POI sequence contained therein forms a circular RNA.

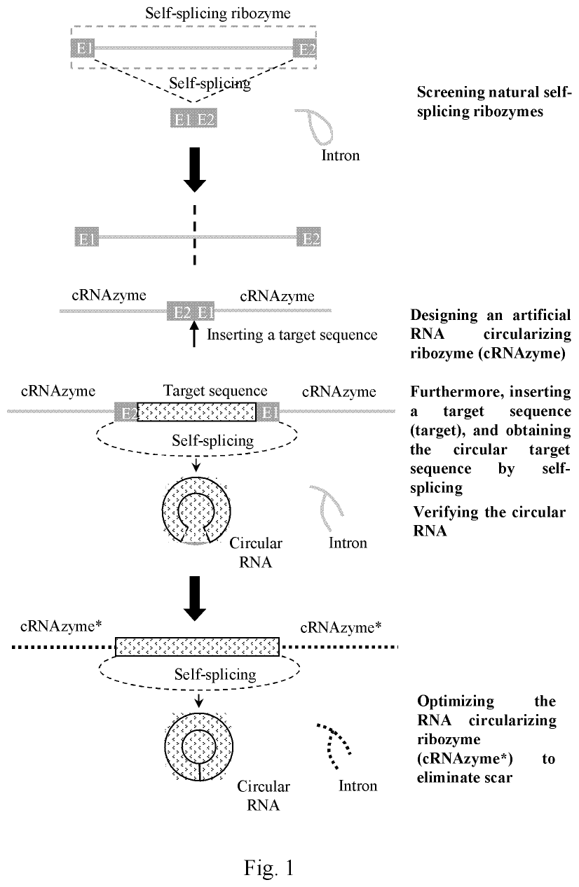

In general, the principle of designing a cRNAzyme construct on the basis of a group II intron (cRNAzyme precursor) is to preserve maximum percent of circularizing while keeping the overall length as short as possible. After the self-splicing circularization reaction occurs, the intron portion is excised, as shown in FIG. 1. The obtained circular RNA product no longer comprises the intron portion. Therefore, the circular RNA product has fewer total nucleotides than the linear cRNAzyme construct structure without splicing reaction. Based on this, the circular RNA product and the cRNAzyme construct may be distinguished by agarose gel electrophoresis. In the context of the present invention, the percent of circularizing (PC) is defined as the percentage of circular RNAs relative to the sum of linear RNAs and circular RNAs. The specific quantitative method adopted a semi-quantitative method commonly used in the art, and the amount was determined according to the intensity of the bands in the gel electrophoretogram.

Based on the above principle, the E1 and/or the E2 is preferably no more than 20 nucleotides in length, such as no more than 10 nucleotides, such as 1, 2, 3, 4, 5, 6, 7, 8, 9 or 10 nucleotides. In a particular embodiment, the E1 and the E2 may be 0.

Also based on the above principle, in a cRNAzyme construct, an intron sequence, such as a 5′ intron fragment and/or a 3′ intron fragment, and/or an exon sequence, such as E1 and/or E2, may comprise a modification of one or more nucleotides, such as an addition, a deletion, and a substitution of one or more nucleotides, relative to their naturally occurring wild-type sequences.

Stem-loop structure is a type of an RNA secondary structure, which can be determined by any suitable polynucleotide folding algorithm. Some programs are based on the calculation of the minimum Gibbs free energy. An example of one such algorithm is mFold and is described by Zuker and Stiegler (Nucleic Acids Res. 9 (1981), 133-148). Another exemplary folding algorithm is the online web server RNAfold developed by the Institute for Theoretical Chemistry at the University of Vienna using a centroid structure prediction algorithm (e.g. AR Gruber et al., 2008, Cell 106). (1): 23-24; and PA Carr and GM Church, 2009, Nature Biotechnology 27 (12): 1151-62). Additional algorithms can be found in US Provisional Patent Application No. 61/836,080 (Attorney Docket No. 44790.11.2022; Broad reference number BI-2013/004A), which is incorporated herein by reference.

In one embodiment, in order to shorten the sequence, a portion of the sequence or nucleotides may be deleted without affecting activity. For example, an intron encoded protein (IEP) sequence in group II intron domain 4 may be deleted. The IEP sequence or similar structures in domain 4 are present in all group II introns, and encode proteins with reverse transcriptase activity which may catalyze the intron to act as a reverse transcription factor and move in its genome via an RNA intermediate. This function is required for retrotransposition of natural group II introns in the genome, but is not required for in vitro transcription. Therefore, part or all of this stretch of sequence in domain 4 may be deleted in the construct of the present invention.

E1 and E2 typically need to comprise an IBS sequence to interact with the EBS sequence contained in the intron for self-splicing. In one embodiment of the present invention, the E1 and E2 sequences may be 0, which has the advantage that the final circular RNA does not comprise any sequence other than the target sequence. In this case, in order to ensure that there can still be an “IBS” sequence paired with the EBS in the intron, the EBS sequence of the intron needs to be modified so that it is complementarily paired with a stretch of sequence in the target sequence, thereby allowing interaction. In other words, a stretch of sequence in the target sequence is regarded as an “IBS” that interacts with the modified EBS sequence in the intron to ensure completion of self-splicing.

Therefore, in one embodiment of the present invention, the group II intron is a modified group II intron, in particular a group II intron having a modified EBS region. The modification may be a substitution of one or more nucleotides, specifically a substitution of one or more nucleotides in the EBS region, so that the modified EBS region is complementarily paired with a region of a corresponding length in a target sequence. The expression “complementarily paired” means that two sequences can be complementarily paired after being transcribed into an RNA, and the pairing covers the pairing manner of G and U in an RNA. The modified EBS may be 3 to 20 nucleotides in length, preferably 5 to 15 nucleotides, more preferably 6 to 10 nucleotides, such as 6, 7, 8, 9 or 10 nucleotides.

The region of the target sequence that is complementary paired with the modified EBS may exist anywhere in the target sequence, as long as the pairing with the EBS can be achieved, thereby forming a secondary structure that is capable of facilitating self-splicing. In general, sequences at both ends of the target sequence may be used as the basis for the design of modified EBS, as the sequences at both ends are located in the construct where E1 and E2 were originally located, and the positions of E1 and E2 are also the positions of the IBS sequences that originally interacted with EBS. Therefore, in a specific embodiment, the modified EBS regions are modified EBS1 and EBS3 regions. In a specific embodiment, the region in the target sequence that is complementarily paired with the modified EBS region is located at the 3′ and/or 5′ end of the target sequence.

Since the purpose of this complementary pairing is to ensure the interaction between the EBS and the target sequence fragment that acts as the IBS, a certain degree of mismatch may be tolerated as long as the interaction exists. In some embodiments, the modified EBS region is complementarily paired with a region of a corresponding length in the target sequence on at least 60%, such as at least 70%, at least 80%, at least 90%, at least 95%, or 100% of the nucleotide positions, or is at least 60% identical, such as at least 70%, at least 80%, at least 90%, at least 95%, or 100% identical to a complementary paired sequence of a region of a corresponding length in the target sequence.

In another embodiment, the 5′ intron fragment and the 3′ intron fragment may comprise one or more pairs of paired sequences that are complementary to each other. Such paired sequences shorten the spatial distance between the 5′ intron fragment and the 3′ intron fragment, thereby facilitating the circularization reaction. In a preferred embodiment, the complementary paired sequence is at least about 20 nucleotides in length.

The target sequence in the construct may comprise any sequence desired to be prepared into a circular RNA. The target sequence may be a protein coding sequence, or a protein noncoding sequence, or a combination thereof. In other words, various elements may be comprised in the target sequence. The protein coding sequence may encode any protein, e.g., selected from a functional protein, an antigenic protein, a signal peptide, a tag protein, and the like.

For example, the protein noncoding sequence comprised in the target sequence may be a spacer sequence, such as an AT-rich sequence, which may modulate the flexibility of the sequence. Such spacer sequences may be located anywhere in the target sequence, e.g., at one end of the target sequence, immediately adjacent to E1 and/or E2.

For example, the protein noncoding sequence comprised in the target sequence may be a translational regulatory sequence, such as an internal ribosome entry site (IRES). IRESs available for the present invention may come from any source.

The cRNAzyme and cRNAzyme construct of the present invention are prepared intact in the form of DNA, which are then transcribed and self-spliced to form the desired circular RNA.

### 6.4. Self-Splicing Reaction System

Self-splicing of group II introns needs to be accomplished under high-salinity conditions, and does not require the introduction of GTP as compared with group I introns.

In a specific embodiment of the present invention, the self-splicing buffer used in the self-splicing reaction comprises 10 mM to 100 mM, such as 10 mM, 20 mM, 30 mM, 40 mM, 50 mM, 60 mM, 70 mM, 80 mM, 90 mM, and 100 mM divalent magnesium ions, such as MgCl2. The self-splicing buffer may comprise 10 mM to 100 mM, such as 10 mM, 20 mM, 30 mM, 40 mM, 50 mM, 60 mM, 70 mM, 80 mM, 90 mM, and 100 mM NaCl.

In a preferred embodiment, the self-splicing reaction of the present invention is performed in vitro for about 5 min to about 1 h, such as about 5 min, about 10 min, about 15 min, about 20 min, about 25 min, about 30 min, about 35 min, about 40 min, about 45 min, about 50 min, about 55 min, and about 1 h.

In a preferred embodiment, the construct of the present invention is capable of achieving a circularization rate of at least 30%, such as a circularization rate of at least 50%, at least 60%, at least 70%, at least 80%, at least 90%, and at least 95%.

### 6.5. Target Sequence

In some embodiments, the target sequence is empty. In some embodiments, the target sequence is a protein coding sequence. In some embodiments, the target sequence is a noncoding sequence.

In some embodiments, the target sequence encodes a therapeutic product.

In a specific embodiment, the therapeutic product is a polypeptide, a protein, an enzyme or an antibody. In a specific embodiment, the therapeutic product comprises one or more polypeptide, protein, enzyme, antibody, or a combination thereof.

In a specific embodiment, the protein or enzyme is associated with diseases with pathological manifestation which can be traced to genetic alterations, and/or protein dysregulations.

In a specific embodiment, the polypeptide or protein resembles a weakened or dead form of disease-causing agent, which could be a microorganism, such as bacteria, virus, fungi, parasites, or one or more toxins and/or one or more proteins, for example, surface proteins, (i.e., antigens) of such a microorganism. In a specific embodiment, the therapeutic product is an antigen or agent which can stimulate the body's immune system to recognize the agent as a foreign invader, generate antibodies against it, destroy it and develop a memory of it. In a specific embodiment, the therapeutic product is an antigen or agent which can induce vaccine-induced memory and/or enable the immune system to act quickly to protect the body from any of these agents in later encounters.

In some embodiments, the therapeutic product is derived from an infectious agent. In some embodiments, the infectious agent is selected from a member of the group consisting of strains of viruses and strains of bacteria.

In any of the embodiments provided herein, the infectious agent is a strain of virus selected from the group consisting of adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabii virus; Crimean-Congo hemorrhagic fever virus; Ebola virus; Marburg virus; Measles virus; Mumps virus; Parainfluenza virus; Respiratory syncytial virus (RSV); Human metapneumovirus; Hendra virus; Nipah virus; Rabies virus; Hepatitis D; Rotavirus; Orbivirus; Coltivirus; Banna virus; Human Enterovirus; Hanta virus; West Nile virus; Corona virus, Severe acute respiratory syndrome (SARS)-associated coronavirus (SARS-CoV), SARS-CoV-2 virus (COVID-19 associated); Middle East Respiratory Syndrome Corona Virus; Japanese encephalitis virus; Vesicular exanthernavirus; Eastern equine encephalitis; and Influenza virus. In some embodiments, the infectious agent is a strain of bacteria selected from Tuberculosis (Mycobacterium tuberculosis), clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA).

In some embodiments, the infectious agent is associated with birds, pigs, horses, dogs, humans or non-human primates.

In some embodiments, the antibodies include, but not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)2S, and scFvs (single-chain variable fragments).

In a specific embodiment, nucleic acids (e.g, polynucleotides) and nucleic acid sequences disclosed herein may be codon-optimized, for example, via any codon-optimization technique known to one of skill in the art (see, e.g., review by Quax el al., 2015, Mol Cell 59: 149-161).

In some embodiments, the target sequence encodes an aptamer sequence. In some embodiments, the target sequence encodes a single-stranded DNA or RNA (ssDNA or ssRNA) molecules that can selectively bind to a specific target, including proteins, peptides, carbohydrates, small molecules, toxins, and even live cells.

In some embodiments, the target sequence encodes a ribozyme, which is a ribonucleic acid (RNA) enzyme that can catalyse a chemical reaction.

In some embodiments, the target sequence encodes an antisense oligonucleotides (ASOs), which bind sequence specifically to the target RNA and modulate protein expression through several different mechanisms.

In some embodiments, the target sequence encodes a Decoy, which is a short stretch of sequence sharing same or homology to miRNA-binding sites or protein binding sites in endogenous targets.

In some embodiments, the target sequence encodes an RNA scaffold, which is an RNA sequence designed to co-localize enzymes in engineered biological pathways through interactions between scaffold's protein docking domains and their affinity protein-enzyme fusions, in vivo.

### 6.6. Vectors, Linear RNA, Precursor RNA and Circular RNA

In some embodiments, the RNA polynucleotide provided herein is a single stranded RNA. In some embodiments, the polynucleotide is a linear RNA. In some embodiments, provided herein is a precursor RNA. In some embodiments, provided the RNA polynucleotide is encoded by a vector. In some embodiments, the precursor RNA is a linear RNA produced by in vitro transcription of a vector provided herein.

In some embodiments, the RNA polynucleotide is circular RNA or is useful for making a circular RNA polynucleotide. In some embodiments, provided herein is a circular RNA. In some embodiments, the circular RNA is a circular RNA produced by a vector provided herein. In some embodiments, the circular RNA is circular RNA produced by circularization of a precursor RNA provided herein.

### Circular RNAs

Circular RNAs (also referred to as “circRNAs” or “cRNAs”) are single-stranded RNAs that are joined head to tail. circRNAs have been recognized as a pervasive class of noncoding RNAs in eukaryotic cells. Typically generated through back splicing, circRNAs are found to be very stable.

In some embodiments, splint ligation may be used to generate circular RNAs. Splint ligation involves the use of an oligonucleotide splint that hybridizes with the two ends of a linear RNA to bring the ends of the linear RNA together for ligation. Hybridization of the splint, which can be either a deoxyribo-oligonucleotide or a ribooligonucleotide, orients the 5-phosphate and 3-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, is more efficient in some cases than enzymatic ligation if the structure of the hybridized splint-RNA complex interferes with enzymatic activity (see, e.g., Dolinnaya et al. Nucleic Acids Res, 2/(23): 5403-5407 (1993); Petkovic et al., Nucleic Acids Res, 43(4): 2454-2465 (2015)).

In some embodiments, the RNA polynucleotide (e.g., circular RNA) may be of any length or size. In some embodiments the RNA polynucleotide is between 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length.

In some embodiments, the RNA polynucleotide (e.g., circular RNA) is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, or 5000 nt in length. In some embodiments, the RNA polynucleotide is no more than 3000 nt, 3500 nt, 4000 nt, 4500 nt, 5000 nt, 6000 nt, 7000 nt, 8000 nt, 9000 nt, or 10000 nt in length.

In some embodiments, the RNA polynucleotide (e.g., circular RNA) is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, or 10000 nt in length.

In some embodiments, the RNA polynucleotide (e.g., circular RNA) is at least 500, 600, 700, 800, 900, 1000, 1500, 2000, 2500, 3000, 3500, 4000, 4500, 5000, 5500, 6000, 6500, 7000, 7500, 8000, 8500, 9000, 9500 or 10000 nt in length. The RNA polynucleotide (e.g., circular RNA) can be unmodified, partially modified or completely modified.

In some embodiments, the circular RNA provided herein has higher functional stability than mRNA comprising the same expression sequence. In some embodiments, the circular RNA provided herein has higher functional stability than mRNA comprising the same expression sequence, 5moU modifications, an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail. In some embodiments, the circular RNA provided herein may have higher stability than an equivalent linear mRNA. In some embodiments, this may be shown by measuring receptor presence and density in vitro or in vivo post electroporation, with time points measured over 1 week. In some embodiments, this may be shown by measuring RNA presence via qPCR or ISH.

In some embodiments, a circular RNA polynucleotide provided herein comprises modified RNA nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Y (pseudouridine). In another embodiment, the modified nucleoside is Um (2′-O-methyluridine). In other embodiments, the modified nucleoside is m^{!}A (1-methyladenosine); m²A (2-methyladenosine); Am (2′-O-methyladenosine); ms2 m⁶A (2-methylthio-N⁶-methyladenosine); i⁶A (N⁶-isopentenyladenosine); ms2i6A (2-methylthio-N⁶ isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladeno sine); ms²t6A (2-methylthio-N⁶-threonyl carbamoyladenosine); m t A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); miI (1-methylinosine); mihn (1,2′-O-dimethylinosine); m³C (3-methylcytidine); Cm (2′-O-methylcytidine); s²C (2-thiocytidine); ac⁴C (N⁴-acetylcytidine); (5-formylcytidine); m⁵Cm (5,2′-O-dimethylcytidine); ac⁴Cm (N⁴-acetyl-2′-O-methylcytidine); k²C (lysidine); m!G (1-methylguanosine); m²G (N²-methylguanosine); m⁷G (7-methylguanosine); Gm (2′-O-methylguanosine); m² 2G (N²,N²-dimethylguanosine); m²Gm (N²,2′-O-dimethylguanosine); m2 aGm (N²,N²,2′-O-trimethylguanosine); Gr(p) (2′-O-ribosylguanosine(phosphate)); yW (wybutosine); oayW (peroxywybutosine); OHyW (hydroxy wybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosine); G⁺ (archaeosine); D (dihydrouridine); m⁵Um (5,2′-O-dimethyluridine); s⁴U (4-thiouridine); m⁵s2U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxy acetic acid); mcmo⁵U (uridine 5-oxy acetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s2U (5-methoxycarbonylmethyl-2-thiouridine); nm⁵S2U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶ ₂A (N⁶,N⁶-dimethyladenosine); Im (2′-O-methylinosine); m⁴C (N⁴-methylcytidine); m⁴Cm (N4,2′-O-dimethylcytidine); hm⁵C (5-hydraxymethylcytidine); m³U (3-methyluridine); cm⁵U (5-carboxymethyluridine); m⁶Am (N6,2′-O-dimethyladenosine); m⁶ ₂Am (N6,N6,0-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m′Gm (1,2′-O-dimethylguanosine); m′Am (1,2′-O-dimethyladenosine); rm ⁵U (5-taurinomethyluridine); τm5s2U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, l-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, polynucleotides may be codon-optimized. A codon optimized sequence may be one in which codons in a polynucleotide encoding a therapeutic product have been substituted in order to increase the expression, stability and/or activity of the therapeutic product. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

In some embodiments, a polynucleotide construct with self-splicing activity, comprising the following operably linked elements from 5′ to 3′: (a) a 3′ intron fragment; (b) an exon fragment 2 (E2); (c) a target sequence; (d) an exon fragment 1 (E1); and (d) a 5′ intron fragment. In one embodiment, the 5′ intron fragment and the 3′ intron fragment are each a fragment of a group II intron. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, a polynucleotide construct with self-splicing activity, comprising the following operably linked elements from 5′ to 3′: (a) a 3′ intron fragment; (b) an exon fragment 2 (E2); (c) a linker sequence; (d) a target sequence; (e) a linker sequence; (f) an exon fragment 1 (E1); and (g) a 5′ intron fragment. In one embodiment, the 5′ intron fragment and the 3′ intron fragment are each a fragment of a group II intron. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, a polynucleotide construct with self-splicing activity, comprising the following operably linked elements from 5′ to 3′: (a) a 5′ homology arm; (b) a 3′ intron fragment; (c) an exon fragment 2 (E2); (d) a target sequence; (e) an exon fragment 1 (E1); (f) a 5′ intron fragment; and (g) a 3′ homology arm. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, a polynucleotide construct with self-splicing activity, comprising the following operably linked elements from 5′ to 3′: (a) a 5′ homology arm; (b) a 3′ intron fragment; (c) an exon fragment 2 (E2); (d) a linker sequence; (e) a target sequence; (f) a linker sequence; (g) an exon fragment 1 (E1); (h) a 5′ intron fragment; and (i) a 3′ homology arm. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, the polynucleotide construct has self-splicing activity in vitro.

In some embodiments, the E1 and/or the E2 is 0 to 20 nucleotides in length. In a preferred embodiment, the E1 and/or the E2 is 0 to 10 nucleotides in length. In one embodiment, the E1 and/or the E2 is 0, 1, 2, 3, 4, 5, 6, 7, 8, 9 or 10 nucleotides in length.

In some embodiments, the 5′ intron fragment and the 3′ intron fragment are obtained by segmenting a group II intron at an unpaired region into two fragments, for example, an unpaired region which is a linear region between two adjacent domains of the group II intron.

In some embodiments, the 5′ intron fragment and the 3′ intron fragment are obtained by segmenting a group II intron at a loop region of a stem-loop structure of domain 1.

In some embodiments, the 5′ intron fragment and the 3′ intron fragment are obtained by segmenting a group II intron at a loop region of a stem-loop structure of domain 2.

In some embodiments, the 5′ intron fragment and the 3′ intron fragment are obtained by segmenting a group II intron at a loop region of a stem-loop structure of domain 3.

In some embodiments, the 5′ intron fragment and the 3′ intron fragment are obtained by segmenting a group II intron at a loop region of a stem-loop structure of domain 4.

In some embodiments, the 5′ intron fragment and the 3′ intron fragment are obtained by segmenting a group II intron at a loop region of a stem-loop structure of domain 5.

In some embodiments, the 5′ intron fragment and the 3′ intron fragment are obtained by segmenting a group II intron at a loop region of a stem-loop structure of domain 6.

In some embodiments, the 5′ intron fragment and the 3′ intron fragment are obtained by segmenting a group II intron at a linear region between domain 1 and domain 2.

In some embodiments, the 5′ intron fragment and the 3′ intron fragment are obtained by segmenting a group II intron at a linear region between domain 2 and domain 3.

In some embodiments, the 5′ intron fragment and the 3′ intron fragment are obtained by segmenting a group II intron at a linear region between domain 3 and domain 4.

In some embodiments, the 5′ intron fragment and the 3′ intron fragment are obtained by segmenting a group II intron at a linear region between domain 4 and domain 5.

In some embodiments, the 5′ intron fragment and the 3′ intron fragment are obtained by segmenting a group II intron at a linear region between domain 5 and domain 6.

In some embodiments, the group II intron comprises a modification of one or more nucleotides relative to its wild-type form, and the modification is selected from one or more of a deletion, a substitution, and an addition.

In some embodiments, the modification comprises a modification of one or more EBS sequences of the group II intron, wherein the EBS sequences are complementarily paired with one or more regions of a corresponding length in a target sequence on at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotide positions respectively.

In some embodiments, the modification is a modification of the two EBS sequences of the group II intron, such as EBS1 and EBS3, wherein the EBS sequences are complementarily paired with two regions of a corresponding length in a target sequence on at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotide positions respectively; preferably, the two regions are located at both ends of the target sequence, respectively.

In some embodiments, the modification is a modification of the two EBS sequences of the group II intron, such as EBS1′ and EBS3′, wherein the EBS sequences are complementarily paired with two regions of a corresponding length in a target sequence on at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotide positions respectively; preferably, the two regions are located at both ends of the target sequence, respectively.

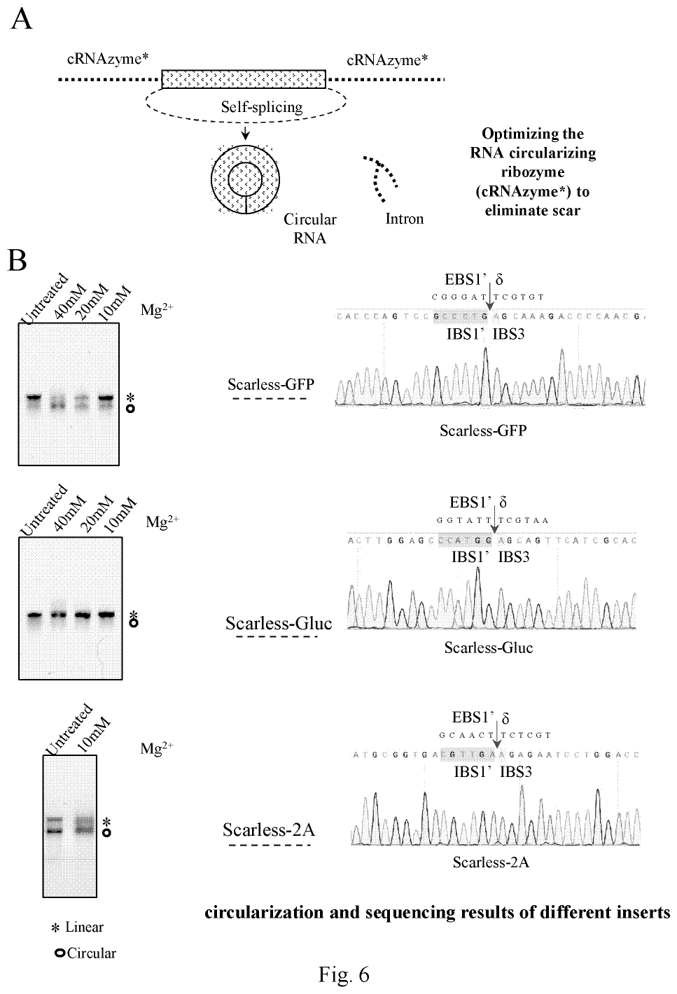

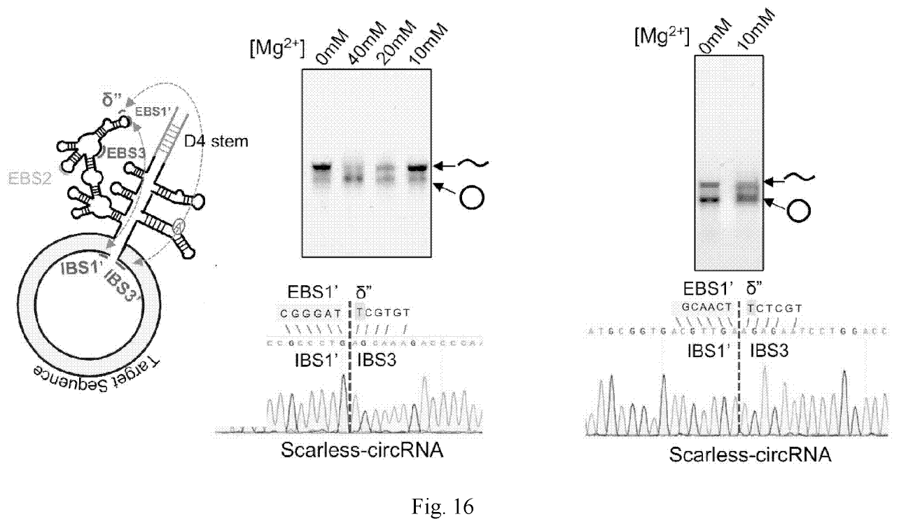

In some embodiments, the modification is a modification of EBS1 and/or δ sequence of the group II intron, or a modification of EBS1′ and/or δ″ sequence, wherein the EBS1 and/or δ sequence is complementarily paired with a region of a corresponding length in a target sequence on at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotide, optionally the modification is a modification of EBS1 and/or δ sequence and its upstream sequence, wherein the EBS1 and/or δ sequence and its upstream sequence is complementarily paired with a region of a corresponding length in a target sequence on at least 60% of the nucleotide. In some embodiments, the region of a corresponding length in a target sequence is IBS3, IBS3′, IBS3 with downstream sequence, or IBS3′ with downstream sequence. In some embodiments, the δ sequence and its upstream comprises a nucleic acid sequence selected from the group consisting: (a) SEQ ID NO: 127, (b) SEQ ID NO:128, (c) SEQ ID NO:129, and (d) SEQ ID NO 130. In some embodiments, the IBS3 and its downstream comprises a nucleic acid sequence selected from the group consisting: (a) SEQ ID NO: 131, (b) SEQ ID NO:132, (c) SEQ ID NO:133, and (d) SEQ ID NO 134. See FIGS. 6 and 16.

In some embodiments, the modification comprises a deletion of part or all of domain 4, such as a deletion of an intron-encoded protein (IEP) sequence in domain 4, preferably a deletion of all of domain 4.

In some embodiments, the modification comprises a deletion of an open reading frame (ORF).

In some embodiments, the polynucleotide construct is capable of forming a near-scarless circular RNA of the target sequence.

In some embodiments, the near-scarless circular RNA has a scar region equal to or less than 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides in length.

In some embodiments, the polynucleotide construct is capable of forming a scarless circular RNA of the target sequence.

In some embodiments, E1 and E2 are each 0 nucleotide in length. In some embodiments, E1 is 0 nucleotide in length. In some embodiments, E2 is 0 nucleotide in length.

In some embodiments, the group II intron is a group II intron derived from a microorganism (such as Clostridium tetani, or Bacillus, such as Bacillus thuringiensis).

In some embodiments, the noncoding sequence is selected from the group consisting of: a spacer sequence of SEQ ID NOs: 4-6, a polyA sequence, a poly-A-C sequence, a poly-C sequence, a poly-U sequence, an IRES, a ribosome binding site, an aptamer sequence, an RNA scaffold, a riboswitch, a ribozyme other than a self-splicing ribozyme, an antisense oligonucleotide (ASO), a scaffold, a small RNA binding site, a translational regulatory sequence, and a protein binding site.

In some embodiments, the group II intron comprises a nucleic acid sequence selected from the group consisting of: SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; and SEQ ID NO: 41.

In some embodiments, the group II intron comprises a nucleic acid sequence selected from the group consisting of: a nucleic acid sequence 95% identical to SEQ ID NO: 33; a nucleic acid sequence 95% identical to SEQ ID NO: 34; a nucleic acid sequence 95% identical to SEQ ID NO: 35; a nucleic acid sequence 95% identical to SEQ ID NO: 36; a nucleic acid sequence 95% identical to SEQ ID NO: 37; a nucleic acid sequence 95% identical to SEQ ID NO: 38; a nucleic acid sequence 95% identical to SEQ ID NO: 39; a nucleic acid sequence 95% identical to SEQ ID NO: 40; and a nucleic acid sequence 95% identical to SEQ ID NO: 41.

In some embodiments, the group II intron comprises a nucleic acid sequence selected from the group consisting of: a nucleic acid sequence 98% identical to SEQ ID NO: 33; a nucleic acid sequence 98% identical to SEQ ID NO: 34; a nucleic acid sequence 98% identical to SEQ ID NO: 35; a nucleic acid sequence 98% identical to SEQ ID NO: 36; a nucleic acid sequence 98% identical to SEQ ID NO: 37; a nucleic acid sequence 98% identical to SEQ ID NO: 38; a nucleic acid sequence 98% identical to SEQ ID NO: 39; a nucleic acid sequence 98% identical to SEQ ID NO: 40; and a nucleic acid sequence 98% identical to SEQ ID NO: 41.

In some embodiments, the group II intron comprises a nucleic acid sequence selected from the group consisting of: a nucleic acid sequence 99% identical to SEQ ID NO: 33; a nucleic acid sequence 99% identical to SEQ ID NO: 34; a nucleic acid sequence 99% identical to SEQ ID NO: 35; a nucleic acid sequence 99% identical to SEQ ID NO: 36; a nucleic acid sequence 99% identical to SEQ ID NO: 37; a nucleic acid sequence 99% identical to SEQ ID NO: 38; a nucleic acid sequence 99% identical to SEQ ID NO: 39; a nucleic acid sequence 99% identical to SEQ ID NO: 40; and a nucleic acid sequence 99% identical to SEQ ID NO: 41.

In some embodiments, the group II intron comprises a nucleic acid sequence selected from Table 16-24.

In some embodiments, the group II intron consists essentially of a nucleic acid sequence selected from the group consisting of SEQ ID NO: 33-SEQ ID NO: 41.

In some embodiments, the group II intron consists of a nucleic acid sequence selected from the group consisting of SEQ ID NO: 33-SEQ ID NO: 41.

In some embodiments, the group II intron consists of a nucleic acid sequence selected from the group consisting of SEQ ID NO: 33-SEQ ID NO: 41.

In some embodiments, the polynucleotide construct is an RNA polynucleotide construct.

In some embodiments, the 3′ intron fragment comprises a nucleic acid sequence selected from the group consisting of: (a) a nucleic acid sequence 95% identical to SEQ ID NO: 42; (b) a nucleic acid sequence 98% identical to SEQ ID NO: 42; (c) a nucleic acid sequence 99% identical to SEQ ID NO: 42; (d) SEQ ID NO: 42; (e) a nucleic acid sequence 95% identical to SEQ ID NO: 43; (f) a nucleic acid sequence 98% identical to SEQ ID NO: 43; (g) a nucleic acid sequence 99% identical to SEQ ID NO: 43; (h) SEQ ID NO: 43; (i) a nucleic acid sequence 95% identical to SEQ ID NO: 44; (j) a nucleic acid sequence 98% identical to SEQ ID NO: 44; (k) a nucleic acid sequence 99% identical to SEQ ID NO: 44; (1) SEQ ID NO: 44; (m) a nucleic acid sequence 95% identical to SEQ ID NO: 45; (n) a nucleic acid sequence 98% identical to SEQ ID NO: 45; (o) a nucleic acid sequence 99% identical to SEQ ID NO: 45; (p) SEQ ID NO: 45; (q) a nucleic acid sequence 95% identical to SEQ ID NO: 46; (r) a nucleic acid sequence 98% identical to SEQ ID NO: 46; (s) a nucleic acid sequence 99% identical to SEQ ID NO: 46; (t) SEQ ID NO: 46; (u) a nucleic acid sequence 95% identical to SEQ ID NO: 47; (v) a nucleic acid sequence 98% identical to SEQ ID NO: 47; (w) a nucleic acid sequence 99% identical to SEQ ID NO: 47; (x) SEQ ID NO: 47; (y) a nucleic acid sequence 95% identical to SEQ ID NO: 48; (z) a nucleic acid sequence 98% identical to SEQ ID NO: 48; (aa) a nucleic acid sequence 99% identical to SEQ ID NO: 48; (bb) a nucleic acid sequence SEQ ID NO: 48; (cc) a nucleic acid sequence 95% identical to SEQ ID NO: 49; (dd) a nucleic acid sequence 98% identical to SEQ ID NO: 49; (ee) a nucleic acid sequence 99% identical to SEQ ID NO: 49; (ff) SEQ ID NO: 49; (gg) a nucleic acid sequence 95% identical to SEQ ID NO: 50; (hh) a nucleic acid sequence 98% identical to SEQ ID NO: 50; (ii) a nucleic acid sequence 99% identical to SEQ ID NO: 50; (jj) SEQ ID NO: 50; (kk) a nucleic acid sequence 95% identical to SEQ ID NO: 51; (ll) a nucleic acid sequence 98% identical to SEQ ID NO: 51; (mm) a nucleic acid sequence 99% identical to SEQ ID NO: 51; (nn) SEQ ID NO: 51; (oo) a nucleic acid sequence 95% identical to SEQ ID NO: 52; (pp) a nucleic acid sequence 98% identical to SEQ ID NO: 52; (qq) a nucleic acid sequence 99% identical to SEQ ID NO: 52; and (rr) SEQ ID NO: 52.

In some embodiments, the 3′ intron fragment consists essentially of a nucleic acid sequence selected from the group consisting of a nucleic acid sequence 95% identical to any one of SEQ ID NO: 42-SEQ ID NO: 52, a nucleic acid sequence 98% identical to any one of SEQ ID NO: 42-SEQ ID NO: 52, a nucleic acid sequence 99% identical to any one of SEQ ID NO: 42-SEQ ID NO: 52, and any one of SEQ ID NO: 42-SEQ ID NO: 52.

In some embodiments, the 3′ intron fragment consists of a nucleic acid sequence selected from the group consisting of a nucleic acid sequence 95% identical to any one of SEQ ID NO: 42-SEQ ID NO: 52, a nucleic acid sequence 98% identical to any one of SEQ ID NO: 42-SEQ ID NO: 52, a nucleic acid sequence 99% identical to any one of SEQ ID NO: 42-SEQ ID NO: 52, and any one of SEQ ID NO: 42-SEQ ID NO: 52.

In some embodiments, the E2 comprises a nucleic acid sequence selected from the group consisting of: (a) SEQ ID NO: 53; (b) SEQ ID NO: 54; (c) SEQ ID NO: 55; (d) SEQ ID NO: 56; (e) SEQ ID NO: 57; (f) SEQ ID NO: 58; (g) SEQ ID NO: 59; (h) SEQ ID NO: 60; (i) SEQ ID NO: 61; (j) SEQ ID NO: 62; and (k) SEQ ID NO: 63.

In some embodiments, the E2 consists essentially of a nucleic acid sequence selected from the group consisting of SEQ ID NO: 53-SEQ ID NO: 63.

In some embodiments, the E2 consists of a nucleic acid sequence selected from the group consisting of SEQ ID NO: 53-SEQ ID NO: 63.

In some embodiments, the E1 comprises a nucleic acid sequence selected from the group consisting of: SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71. SEQ ID NO: 72; SEQ ID NO: 73; and SEQ ID NO: 74.

In some embodiments, wherein the E1 consists essentially of a nucleic acid sequence selected from the group consisting of SEQ ID NO: 64-SEQ ID NO: 74.

In some embodiments, the E1 consists of a nucleic acid sequence selected from the group consisting of SEQ ID NO: 64-SEQ ID NO: 74.

In some embodiments, the 5′ intron fragment comprises a nucleic acid sequence selected from the group consisting of: (a) a nucleic acid sequence 95% identical to SEQ ID NO: 75; (b) a nucleic acid sequence 98% identical to SEQ ID NO: 75;

(c) a nucleic acid sequence 99% identical to SEQ ID NO: 75; (d) SEQ ID NO: 75; (e) a nucleic acid sequence 95% identical to SEQ ID NO: 76; (f) a nucleic acid sequence 98% identical to SEQ ID NO: 76; (g) a nucleic acid sequence 99% identical to SEQ ID NO: 76; (h) SEQ ID NO: 76; (i) a nucleic acid sequence 95% identical to SEQ ID NO: 77; (j) a nucleic acid sequence 98% identical to SEQ ID NO: 77; (k) a nucleic acid sequence 99% identical to SEQ ID NO: 77; (1) SEQ ID NO: 77; (m) a nucleic acid sequence 95% identical to SEQ ID NO: 78; (n) a nucleic acid sequence 98% identical to SEQ ID NO: 78; (o) a nucleic acid sequence 99% identical to SEQ ID NO: 78; (p) SEQ ID NO: 78; (q) a nucleic acid sequence 95% identical to SEQ ID NO: 79; (r) a nucleic acid sequence 98% identical to SEQ ID NO: 79; (s) a nucleic acid sequence 99% identical to SEQ ID NO: 79; (t) SEQ ID NO: 79; (u) a nucleic acid sequence 95% identical to SEQ ID NO: 80; (v) a nucleic acid sequence 98% identical to SEQ ID NO: 80; (w) a nucleic acid sequence 99% identical to SEQ ID NO: 80; (x) SEQ ID NO: 80; (y) a nucleic acid sequence 95% identical to SEQ ID NO: 81; (z) a nucleic acid sequence 98% identical to SEQ ID NO: 81; (aa) a nucleic acid sequence 99% identical to SEQ ID NO: 81; (bb) SEQ ID NO: 81; (cc) a nucleic acid sequence 95% identical to SEQ ID NO: 82; (dd) a nucleic acid sequence 98% identical to SEQ ID NO: 82; (ee) a nucleic acid sequence 99% identical to SEQ ID NO: 82; (ff) SEQ ID NO: 82; (gg) a nucleic acid sequence 95% identical to SEQ ID NO: 83; (hh) a nucleic acid sequence 98% identical to SEQ ID NO: 83; (ii) a nucleic acid sequence 99% identical to SEQ ID NO: 83; (jj) SEQ ID NO: 83; (kk) a nucleic acid sequence 95% identical to SEQ ID NO: 84; (ll) a nucleic acid sequence 98% identical to SEQ ID NO: 84; (mm) a nucleic acid sequence 99% identical to SEQ ID NO: 84; (nn) SEQ ID NO: 84; (oo) a nucleic acid sequence 95% identical to SEQ ID NO: 85; (pp) a nucleic acid sequence 98% identical to SEQ ID NO: 85; (qq) a nucleic acid sequence 99% identical to SEQ ID NO: 85; (rr) SEQ ID NO: 85; (ss) a nucleic acid sequence 95% identical to SEQ ID NO: 86; (tt) a nucleic acid sequence 98% identical to SEQ ID NO: 86; (uu) a nucleic acid sequence 99% identical to SEQ ID NO: 86; (vv) SEQ ID NO: 86; (ww) a nucleic acid sequence 95% identical to SEQ ID NO: 87; (xx) a nucleic acid sequence 98% identical to SEQ ID NO: 87; (yy) a nucleic acid sequence 99% identical to SEQ ID NO: 87; (zz) SEQ ID NO: 87; (aaa) a nucleic acid sequence 95% identical to SEQ ID NO: 88; (bbb) a nucleic acid sequence 98% identical to SEQ ID NO: 88; (ccc) a nucleic acid sequence 99% identical to SEQ ID NO: 88; and (ddd) SEQ ID NO: 88.

In some embodiments, the 5′ intron fragment consists essentially of a nucleic acid sequence selected from the group consisting of a nucleic acid sequence 95% identical to any one of SEQ ID NO: 75-SEQ ID NO: 88, a nucleic acid sequence 98% identical to any one of SEQ ID NO: 75-SEQ ID NO: 88, a nucleic acid sequence 99% identical to any one of SEQ ID NO: 75-SEQ ID NO: 88, and any one of SEQ ID NO: 75-SEQ ID NO: 88.

In some embodiments, the 5′ intron fragment consists of a nucleic acid sequence selected from the group consisting of a nucleic acid sequence 95% identical to any one of SEQ ID NO: 75-SEQ ID NO: 88, a nucleic acid sequence 98% identical to any one of SEQ ID NO: 75-SEQ ID NO: 88, a nucleic acid sequence 99% identical to any one of SEQ ID NO: 75-SEQ ID NO: 88, and any one of SEQ ID NO: 75-SEQ ID NO: 88.

In some embodiments, the 5′ homology arm comprises the nucleic acid sequence of SEQ ID NO: 105. In some embodiments, the 5′ homology arm comprises the nucleic acid sequence 95% identical to of SEQ ID NO: 105. In some embodiments, the 5′ homology arm comprises the nucleic acid sequence 98% identical to of SEQ ID NO: 105. In some embodiments, the 5′ homology arm comprises the nucleic acid sequence 99% identical to of SEQ ID NO: 105.

In some embodiments, the 5′ homology arm consists essentially of the nucleic acid sequence of SEQ ID NO: 105.

In some embodiments, the 5′ homology arm consists of the nucleic acid sequence of SEQ ID NO: 105.

In some embodiments, the 3′ homology arm comprises the nucleic acid sequence of SEQ ID NO: 106. In some embodiments, the 3′ homology arm comprises the nucleic acid sequence 95% identical to of SEQ ID NO: 106. In some embodiments, the 3′ homology arm comprises the nucleic acid sequence 98% identical to of SEQ ID NO: 106. In some embodiments, the 3′ homology arm comprises the nucleic acid sequence 99% identical to of SEQ ID NO: 106.

In some embodiments, the 3′ homology arm consists essentially of the nucleic acid sequence of SEQ ID NO: 106.

In some embodiments, the 3′ homology arm consists of the nucleic acid sequence of SEQ ID NO: 106.

In some embodiments, the 5′ homology arm or 3′ homology arm is 15 to 60 nucleotides in length. In some embodiments, the 5′ homology arm or 3′ homology arm is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In some embodiments, the 5′ homology arm or 3′ homology arm sequence has up to 1%, 2%, 3%, 4%, 5%, 6%, 7%, 8%, 9%, or 10% base mismatches.

In some embodiments, the target sequence comprises a 5′ arm sequence selected from the group consisting of: (a) SEQ ID NO: 89; (b) SEQ ID NO: 90; (c) SEQ ID NO: 91; (d) SEQ ID NO: 92; (e) SEQ ID NO: 93; (f) SEQ ID NO: 94; (g) SEQ ID NO: 95; and (h) SEQ ID NO: 96.

In some embodiments, the target sequence comprises a 3′ arm sequence selected from the group consisting of: (a) SEQ ID NO: 97; (b) SEQ ID NO: 98; (c) SEQ ID NO: 99; (d) SEQ ID NO: 100; (e) SEQ ID NO: 101; (f) SEQ ID NO: 102; (g) SEQ ID NO: 103; and (h) SEQ ID NO: 104.

In some embodiments, the target sequence comprises Formula I: TI-(L)n-Z1 (I), wherein: TI is an engineered translation initiation element comprising an internal ribosome entry site (IRES)-like polynucleotide sequence or a natural IRES sequence, Z1 is an expression sequence encoding a therapeutic product; L is a linker sequence; A1 and B1 are a pair of sequences capable of circularization of the RNA polynucleotide; and n is an integer selected from 0 to 2.

In some embodiments, Z1 comprises a nucleic acid sequence selected from the group consisting of: a nucleic acid sequence 95% identical to SEQ ID NO: 107; a nucleic acid sequence 98% identical to SEQ ID NO: 107; a nucleic acid sequence 99% identical to SEQ ID NO: 107; SEQ ID NO: 107; a nucleic acid sequence 95% identical to SEQ ID NO: 108; a nucleic acid sequence 98% identical to SEQ ID NO: 108; a nucleic acid sequence 99% identical to SEQ ID NO: 108; SEQ ID NO: 108; a nucleic acid sequence 95% identical to SEQ ID NO: 109; a nucleic acid sequence 98% identical to SEQ ID NO: 109; a nucleic acid sequence 99% identical to SEQ ID NO: 109; SEQ ID NO: 109; a nucleic acid sequence 95% identical to SEQ ID NO: 110; a nucleic acid sequence 98% identical to SEQ ID NO: 110; a nucleic acid sequence 99% identical to SEQ ID NO: 110; SEQ ID NO: 110; a nucleic acid sequence 95% identical to SEQ ID NO: 111; a nucleic acid sequence 98% identical to SEQ ID NO: 111; a nucleic acid sequence 99% identical to SEQ ID NO: 111; SEQ ID NO: 111; a nucleic acid sequence 95% identical to SEQ ID NO: 112; a nucleic acid sequence 98% identical to SEQ ID NO: 112; a nucleic acid sequence 99% identical to SEQ ID NO: 112; SEQ ID NO: 112.

In some embodiments, Z1 consists essentially of a nucleic acid sequence selected from the group consisting of a nucleic acid sequence 95% identical to any one of SEQ ID NO: 107-SEQ ID NO: 112, a nucleic acid sequence 98% identical to any one of SEQ ID NO: 107-SEQ ID NO: 112, a nucleic acid sequence 99% identical to any one of SEQ ID NO: 107-SEQ ID NO: 112, and any one of SEQ ID NO: 107-SEQ ID NO: 112.

In some embodiments, Z1 consists of a nucleic acid sequence selected from the group consisting of a nucleic acid sequence 95% identical to any one of SEQ ID NO: 107-SEQ ID NO: 112, a nucleic acid sequence 98% identical to any one of SEQ ID NO: 107-SEQ ID NO: 112, a nucleic acid sequence 99% identical to any one of SEQ ID NO: 107-SEQ ID NO: 112, and any one of SEQ ID NO: 107-SEQ ID NO: 112.

In some embodiments, Z1 comprises a nucleic acid sequence encoding the amino acid sequence selected from the group consisting of: (a) SEQ ID NO: 113; (b) SEQ ID NO: 114; (c) SEQ ID NO: 115; (d) SEQ ID NO: 116; (e) SEQ ID NO: 117; and (f) SEQ ID NO: 118.

In some embodiments, Z1 consists essentially of a nucleic acid sequence encoding the amino acid sequence selected from the group consisting of SEQ ID NO: 113-SEQ ID NO: 118.

In some embodiments, the Z1 consists of a nucleic acid sequence encoding the amino acid sequence selected from the group consisting of SEQ ID NO: 113-SEQ ID NO: 118.

In some embodiments, the polynucleotide construct comprising a modified RNA nucleotide and/or modified nucleoside.

In some embodiments, the polynucleotide construct comprising 10% to 100% modified RNA nucleotide and/or modified nucleoside. In some embodiments, the polynucleotide construct comprising 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% modified RNA nucleotide and/or modified nucleoside.

In some embodiments, the modified RNA nucleotide and/or modified nucleoside is m5C (5-methylcytidine). In some embodiments, the polynucleotide construct of any one of Embodiments 47-48, wherein at least one of the modified RNA nucleotide and/or modified nucleoside is m5U (5-methyluridine).

In some embodiments, the modified RNA nucleotide and/or modified nucleoside is m6A (N6-methyladenosine).

In some embodiments, the modified RNA nucleotide and/or modified nucleoside is Y (pseudouridine).

In some embodiments, the modified RNA nucleotide and/or modified nucleoside is m1A (1-methyladenosine).

In some embodiments, the modified RNA nucleotide and/or modified nucleoside is introduced at in vitro transcription (IVT).

In some embodiments, the modified nucleoside is selected from the group consisting of: m5C (5-methylcytidine), m5U (5-methyluridine), m6A (N6-methyladenosine), s2U (2-thiouridine), Y (pseudouridine), Um (2′-O-methyluridine), m1A (1-methyladenosine), m2A (2-methyladenosine), Am (2′-O-methyladenosine), ms2 m6A (2-methylthio-N6-methyladenosine), i6A (N6-isopentenyladenosine), ms2i6A (2-methylthio-N6 isopentenyladenosine), io6A (N6-(cis-hydroxyisopentenyl)adenosine), ms2io6A (2-methylthio-N6-(cis-hydroxyisopentenyl)adenosine), g6A (N6-glycinylcarbamoyladenosine), t6A (N6-threonylcarbamoyladeno sine), ms2t6A (2-methylthio-N6-threonyl carbamoyladenosine), m6t6A (N6-methyl-N6-threonylcarbamoyladenosine), hn6A (N6-hydroxynorvalylcarbamoyladenosine), ms2hn6A (2-methylthio-N6-hydroxynorvalyl carbamoyladenosine), Ar(p) (2′-O-ribosyladenosine (phosphate)), I (inosine), m1I (1-methylinosine), mlhn (1,2′-O-dimethylinosine), m3C (3-methylcytidine), Cm (2′-O-methylcytidine), s2C (2-thiocytidine), ac4C (N4-acetylcytidine), (5-formylcytidine), m5Cm (5,2′-O-dimethylcytidine), ac4Cm (N4-acetyl-2′-O-methylcytidine), k2C (lysidine), m!G (1-methylguanosine), m2G (N2-methylguanosine), m7G (7-methylguanosine), Gm (2′-O-methylguanosine), m2 2G (N2,N2-dimethylguanosine), m2Gm (N2,2′-O-dimethylguanosine), m2 aGm (N2,N2,2′-O-trimethylguanosine), Gr(p) (2′-O-ribosylguanosine(phosphate)), yW (wybutosine), oayW (peroxywybutosine), OHyW (hydroxy wybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQo (7-cyano-7-deazaguanosine), preQi (7-aminomethyl-7-deazaguanosine), G+(archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxy acetic acid), mcmo5U (uridine 5-oxy acetic acid methyl ester), chm5U (5-(carboxyhydroxymethyl)uridine)), mchm5U (5-(carboxyhydroxymethyl)uridine methyl ester), mcm5U (5-methoxycarbonylmethyluridine), mcm5Um (5-methoxycarbonylmethyl-2′-O-methyluridine), mcm5s2U (5-methoxycarbonylmethyl-2-thiouridine), nm5S2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyluridine), ncm5Um (5-carbamoylmethyl-2′-O-methyluridine), cmnm5U (5-carboxymethylaminomethyluridine), cmnm5Um (5-carboxymethylaminomethyl-2′-O-methyluridine), cmnm5s2U (5-carboxymethylaminomethyl-2-thiouridine), m6 2A (N6,N6-dimethyladenosine), Im (2′-O-methylinosine), m4C (N4-methylcytidine), m4Cm (N4,2′-O-dimethylcytidine), hm5C (5-hydraxymethylcytidine), m3U (3-methyluridine), cm5U (5-carboxymethyluridine), m6Am (N6,2′-O-dimethyladenosine), m6 2Am (N6,N6,0-2′-trimethyladenosine), m2,7G (N2,7-dimethylguanosine), m2,2,7G (N2,N2,7-trimethylguanosine), m3Um (3,2′-O-dimethyluridine), m5D (5-methyldihydrouridine), f5Cm (5-formyl-2′-O-methylcytidine), m′Gm (1,2′-O-dimethylguanosine), m′Am (1,2′-O-dimethyladenosine), rm 5U (5-taurinomethyluridine), rm5s2U (5-taurinomethyl-2-thiouridine)), imG-14 (4-demethylwyosine), imG2 (isowyosine), or ac6A (N6-acetyladenosine), pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5-methylcytosine, pseudouridine, and 1-methylpseudouridine.

In some embodiments, the circular RNA is at least 500 nucleotides in length, at least 1,000 nucleotides in length, or at least 1,500 nucleotides in length.

In some embodiments, the circular RNA does not comprise any other sequences that do not belong to the target sequence, such as not comprising all or part of an E2 sequence and an E1 sequence.

In some embodiments, presented herein is a method of making a circular RNA, said method comprising: preparing a vector comprising the following operably linked elements from 5′ to 3′: (a) a 3′ intron fragment; (b) an exon fragment 2 (E2); (c) a target sequence; (d) an exon fragment 1 (E1); and (d) a 5′ intron fragment. In one embodiment, the 5′ intron fragment and the 3′ intron fragment are each a fragment of a group II intron. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, presented herein is a method of making a circular RNA, said method comprising: preparing a vector comprising the following operably linked elements from 5′ to 3′: (a) 3′ intron fragment; (b) an exon fragment 2 (E2); (c) a linker sequence; (d) a target sequence; (e) a linker sequence; (f) an exon fragment 1 (E1); and (g) a 5′ intron fragment, In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, presented herein is a method of making a circular RNA, said method comprising: preparing a vector comprising the following operably linked elements from 5′ to 3′: (a) a 5′ homology arm; (b) a 3′ intron fragment; (c) an exon fragment 2 (E2); (d) a target sequence; (e) an exon fragment 1 (E1); (f) a 5′ intron fragment; and (g) a 3′ homology arm. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, presented herein is a method of making a circular RNA, said method comprising: preparing a vector comprising the following operably linked elements from 5′ to 3′: (a) a 5′ homology arm; (b) a 3′ intron fragment; (c) an exon fragment 2 (E2); (d) a linker sequence; (e) a target sequence; (f) a linker sequence; (g) an exon fragment 1 (E1); (h) a 5′ intron fragment; and (i) a 3′ homology arm. In one embodiment, the 5′ intron fragment is located on the 5′ side of the 3′ intron fragment in the group II intron. In one embodiment, the E1 is a 5′ adjacent exon fragment of the group II intron, which is ≥0 nucleotides in length. In one embodiment, the E2 is a 3′ adjacent exon fragment of the group II intron, which is ≥0 nucleotides in length, and the target sequence is absent, or is a protein coding sequence, a noncoding sequence, or a combination thereof.

In some embodiments, presented herein is a method for expressing a protein in a cell, comprising (a) transfecting the cell with the circular RNA of any one of Embodiments 58-61, or (b) subjecting the polynucleotide construct of any of Embodiments 1-57 to a self-splicing circularization reaction to form a circular RNA, and transfecting the cell with the circular RNA; wherein, preferably the cell is a eukaryotic cell.

In some embodiments, presented herein is a method for expressing a protein in a cell, comprising (a) transfecting the cell with the circular RNA of any one of Embodiments 58-61, or (b) subjecting the construct of any of Embodiments 1-57 to a self-splicing circularization reaction to form a circular RNA, and transfecting the cell with the circular RNA; wherein, preferably the cell is a hepatocyte, epithelial cell, hematopoietic cell, epithelial cell, endothelial cell, lung cell, bone cell, stem cell, mesenchymal cell, neural cell (e.g., meninge, astrocyte, motor neuron, cell of the dorsal root ganglia and anterior horn motor neuron), photoreceptor cell (e.g., rod and cone), retinal pigmented epithelial cell, secretory cell, cardiac cell, adipocyte, vascular smooth muscle cell, cardiomyocyte, skeletal muscle cell, beta cell, pituitary cell, synovial lining cell, ovarian cell, testicular cell, fibroblast, B cell, T cell, dendritic cell, macrophage, reticulocyte, leukocyte, granulocyte, tumor cell, NK cell, liver starlet cell, HEK293, HEK293T, HeLa, MCF7, PC3, A549, NCI-H727, HCT-116, MCF10A, HPReC, FHC, immortalized cell lines, primary cell, yeast cell, Saccharomyces cerevisiae, Pichia pastoris, bacteria cell, Escherichia coli, insect cell, Spodoptera frugiperda sf9, Mimic Sf9, sf21, or Drosophila S2.

In some embodiments, presented herein is a method for generating a sequence with self-splicing activity using a group II intron, the method comprising the steps of: defining the sequence of the group II intron; optionally examining the in vitro self-splicing activity of the group II intron using a splicing assay (linear splicing); splitting the group II intron into two fragments, reversing the order of the two intron fragments, and confirming the in vitro circularization of RNA using a splicing assay.

In some embodiments, the polynucleotide construct, circular RNA, or method of any one of the preceding Embodiments, wherein the 5′ intron fragment and the 3′ intron fragment respectively comprise one or more pairs of paired sequences that are complementary to each other. In a preferred embodiment, the complementary paired sequence is greater than 20 nucleotides in length.

In some embodiments, the polynucleotide construct, circular RNA, or method of any one of the preceding Embodiments, wherein the 5′ intron fragment and/or the 3′ intron fragment comprises one or more affinity tag sequences selected from the group consisting of: a probe binding sequence, an MS2 binding site, a PP7 binding site, and a streptavidin binding site.

In some embodiments, the polynucleotide construct, circular RNA, or method of any one of the preceding Embodiments, wherein the EBS sequence is selected from one or more of EBS1, EBS2 and EBS3, preferably two of them, more preferably EBS1 and EBS3.

In some embodiments, the polynucleotide construct, circular RNA, or method of any one of the preceding Embodiments, wherein one or more EBS sequences of the group II intron, preferably EBS1 and EBS3, are modified, wherein the EBS sequences are complementarily paired with two regions of a corresponding length in a target sequence on at least 60% of the nucleotide positions respectively.

In a preferred embodiment, the two regions of a corresponding length in a target sequence are located at both ends of the target sequence, respectively.

In a preferred embodiment, the polynucleotide construct, circular RNA, or method of any one of the preceding Embodiments, wherein the polynucleotide construct is capable of forming a circular RNA of a target sequence in vitro.

In a preferred embodiment, the polynucleotide construct, circular RNA, or method of any one of the preceding Embodiments, wherein the polynucleotide construct is capable of forming a circular RNA of a target sequence in vivo.

In some embodiments, the group II intron comprises a nucleic acid sequence selected from Table 16-24.

### 6.6.1. Purification

In some embodiments, the polynucleotides comprise a purification tag. In a preferred embodiment, the purification tag is a 15-40 nt polynucleotides anneal to the oligos that conjugated to a purification matrix. A purification matrix includes but not limited to magnetic resin or beads, silicone resin, Sephadex resin, affinity resin, nanoparticles, and nanomaterial surface or coated surfaces.

In some embodiments, the purification tag is an intron tag. In some embodiments, the purification tag is a 5′ intron tag. In some embodiments, the purification tag is a 3′ intron tag.

The circular RNA produced by the construct or method of the present invention may be purified. For example, the purification means is selected from one or more of a group of: enzymatic treatment; chromatography, including but not limited to affinity column chromatography, reversed-phase silica gel column liquid chromatography, and gel exclusion liquid chromatography; and electrophoresis, including but not limited to gel electrophoresis such as agarose gel electrophoresis, and capillary electrophoresis.

Prior to transfecting the cell with the circular RNA product, non-circularized linear RNAs, dsRNAs, and other unwanted components are preferably removed as much as possible by a purification process. The phosphate groups at both ends of a linear RNA and some dsRNAs would activate the RIG-1 signaling pathway, causing a strong immune response in cells, leading to the degradation of exogenous RNAs, and affecting the function of circular RNAs in cells. Methods for removing linear RNAs comprise enzymatic treatment, such as treatment with RNase R; and chromatography, such as high performance liquid chromatography (HPLC). Methods for removing terminal phosphate groups comprise treatment with alkaline phosphatases, such as calf intestinal alkaline phosphatase (CIP) Administration and Delivery

The circular RNA produced by the construct or method of the present invention may be delivered into cells or animals using any of a variety of delivery systems. For example, the delivery system is selected from one or more of a group of: liposomes, polyethyleneimine (PEI), metal-organic frameworks (MOFs), lipid nanoparticles (LNPs), polycations, blood glycoproteins, red blood cell transport vehicles, Au nanoparticle (AuNP) vehicles, magnetic nanoparticle vehicles, carbon nanotubes, graphene molecular vehicles, quantum dot material vehicles, upconversion nanoparticles, layered double hydroxide material vehicles, silica nanoparticles, and calcium phosphate. In some embodiments, the circular RNA can be transfected into a cell using, for example, lipofection or electroporation.

### 6.6.2. Target Cells

In some embodiments, the target cells are deficient in a protein or enzyme of interest. For example, where it is desired to deliver a nucleic acid to a hepatocyte, the hepatocyte represents the target cell. In some embodiments, the compositions of the disclosure transfect the target cells on a discriminatory basis (i.e., do not transfect non-target cells). The compositions of the disclosure may also be prepared to preferentially target and/or expressed in a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, dendritic cells, macrophages, reticulocytes, leukocytes, granulocytes and tumor cells, NK cells, liver starlet cells, HEK293, HEK293T, HeLa, MCF7, PC3, A549, NCI-H727, HCT-116, MCF10A, HPReC, FHC and other immortalized cell lines and primary cell lines.

In some embodiments, the compositions of the disclosure may also be optimized for a variety of yeast cells, which include, but not limited to, Saccharomyces cerevisiae, Pichia pastoris.

In some embodiments, the compositions of the disclosure may also be optimized for a variety of bacteria cells, which include, but not limited to, Escherichia coli.

In some embodiments, the compositions of the disclosure may also be optimized for a variety of insect cells, which include, but not limited to, Spodoptera frugiperda sf9, Mimic Sf9, sf21, Drosophila S2.

The compositions of the disclosure may be prepared to preferentially distribute to and/or optimized for target cells such as in the heart, lungs, kidneys, liver, and spleen. In some embodiments, the compositions of the disclosure distribute into the cells of the liver to facilitate the delivery and the subsequent expression of the circRNA comprised therein by the cells of the liver (e.g., hepatocytes). The targeted cells may function as a biological “reservoir” or “depot” capable of producing, and systemically excreting a functional protein or enzyme. Accordingly, in one embodiment of the disclosure the transfer vehicle may target hepatocytes and/or preferentially distribute to the cells of the liver upon delivery. In an embodiment, following transfection of the target hepatocytes, the circRNA loaded in the vehicle are translated and a functional protein product is produced, excreted and systemically distributed. In other embodiments, cells other than hepatocytes (e.g., lung, spleen, heart, ocular, or cells of the central nervous system) can serve as a depot location for protein production.

In one embodiment, the compositions of the disclosure facilitate a subject's endogenous production of one or more functional proteins and/or enzymes. In an embodiment of the present disclosure, the transfer vehicles comprise circRNA which encode a deficient protein or enzyme. Upon distribution of such compositions to the target tissues and the subsequent transfection of such target cells, the exogenous circRNA loaded into the transfer vehicle (e.g., a lipid nanoparticle) may be translated in vivo to produce a functional protein or enzyme encoded by the exogenously administered circRNA (e.g., a protein or enzyme in which the subject is deficient). Accordingly, the compositions of the present disclosure exploit a subject's ability to translate exogenously- or recombinantly-prepared circRNA to produce an endogenously-translated protein or enzyme, and thereby produce (and where applicable excrete) a functional protein or enzyme. The expressed or translated proteins or enzymes may also be characterized by the in vivo inclusion of native post-translational modifications which may often be absent in recombinantly-prepared proteins or enzymes, thereby further reducing the immunogenicity of the translated protein or enzyme.

The administration of circRNA encoding a deficient protein or enzyme avoids the need to deliver the nucleic acids to specific organelles within a target cell. Rather, upon transfection of a target cell and delivery of the nucleic acids to the cytoplasm of the target cell, the circRNA contents of a transfer vehicle may be translated and a functional protein or enzyme expressed.

In some embodiments, a circular RNA comprises one or more miRNA binding sites. In some embodiments, a circular RNA comprises one or more miRNA binding sites recognized by miRNA present in one or more non-target cells or non-target cell types (e.g., Kupffer cells) and not present in one or more target cells or target cell types (e.g., hepatocytes). In some embodiments, a circular RNA comprises one or more miRNA binding sites recognized by miRNA present in an increased concentration in one or more non-target cells or non-target cell types (e.g., Kupffer cells) compared to one or more target cells or target cell types (e.g., hepatocytes). miRNAs are thought to function by pairing with complementary sequences within RNA molecules, resulting in gene silencing.

### 6.6.3. Pharmaceutical Compositions/Administration

In some embodiments, provided herein are compositions (e.g., pharmaceutical compositions) comprising a therapeutic agent provided herein. In some embodiments, the therapeutic agent is a circular RNA polynucleotide provided herein. In some embodiments the therapeutic agent is a vector provided herein. In some embodiments, the therapeutic agent is a cell comprising a circular RNA or vector provided herein. In some embodiments, the composition further comprises a pharmaceutically acceptable carrier. In some embodiments, the compositions provided herein comprise a therapeutic agent provided herein in combination with other pharmaceutically active agents or drugs. In a preferred embodiment, the pharmaceutical composition comprises a cell provided herein or populations thereof.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein.

In some embodiments, the pharmaceutical composition comprises a preservative. In some embodiments, suitable preservatives may include, for example, methylparaben, propylparaben, sodium benzoate, and benzalkonium chloride. Optionally, a mixture of two or more preservatives may be used. The preservative or mixtures thereof are typically present in an amount of about 0.0001% to about 2% by weight of the total composition.

In some embodiments, the pharmaceutical composition comprises a buffering agent. In some embodiments, suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

In some embodiments, the concentration of therapeutic agent in the pharmaceutical composition can vary, e.g., less than about 1%, or at least about 1%, 2%, 3%, 4%, 5%, 6%, 7%, 8%, 9% 10%, 15%, 20%, 25%, 30%, 35%, 40%, 45%, or about 50% or more by weight, and can be selected primarily by fluid volumes, and viscosities, in accordance with the particular mode of administration selected.

The following formulations for oral, aerosol, parenteral (e.g., subcutaneous, intravenous, intraarterial, intramuscular, intradermal, intraperitoneal, and intrathecal), and topical administration are merely exemplary and are in no way limiting. More than one route can be used to administer the therapeutic agents provided herein, and in some instances, a particular route can provide a more immediate and more effective response than another route.

Formulations suitable for oral administration can comprise or consist of (a) liquid solutions, such as an effective amount of the therapeutic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic agent with a flavorant, usually sucrose, acacia or tragacanth. Pastilles can comprise the therapeutic agent with an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In some embodiments, the therapeutic agents provided herein can be administered in a physiologically acceptable diluent in a pharmaceutical carrier, such as a sterile liquid or mixture of liquids including water, saline, aqueous dextrose and related sugar solutions, an alcohol such as ethanol or hexadecyl alcohol, a glycol such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agents and other pharmaceutical adjuvants.

Oils, which can be used in parenteral formulations in some embodiments, include petroleum, animal oils, vegetable oils, or synthetic oils. Specific examples of oils include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in some embodiments of parenteral formulations include fatty alkali metal, ammonium, and triethanolamine salts, and suitable detergents include (a) cationic detergents such as, for example, dimethyl dialkyl ammonium halides and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

In some embodiments, the parenteral formulations will contain, for example, from about 0.5% to about 25% by weight of the therapeutic agent in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol, sorbitan fatty acid esters such as sorbitan monooleate, and high molecular weight adducts of ethylene oxide with a hydrophobic base formed by the condensation of propylene oxide with propylene glycol. The parenteral formulations can be presented in unit-dose or multi-dose sealed containers, such as ampoules or vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In some embodiments, injectable formulations are provided herein. The requirements for effective pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Toissel, 4th ed, pages 622-630 (1986)).

In some embodiments, topical formulations are provided herein. Topical formulations, including those that are useful for transdermal drug release, are suitable in the context of certain embodiments provided herein for application to skin. In some embodiments, the therapeutic agent alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

In some embodiments, the therapeutic agents provided herein can be formulated as inclusion complexes, such as cyclodextrin inclusion complexes, or liposomes. Liposomes can serve to target the therapeutic agents to a particular tissue. Liposomes also can be used to increase the half-life of the therapeutic agents. Many methods are available for preparing liposomes, as described in, for example, Szoka et al, Ann. Rev. Biophys. Bioeng., 9, 467 (1980) and U.S. Pat. Nos. 4,235,871, 4,501,728, 4,837,028, and 5,019,369.

In some embodiments, the therapeutic agents provided herein are formulated in time-released, delayed release, or sustained release delivery systems such that the delivery of the composition occurs prior to, and with sufficient time to cause, sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the disclosure are formulated such that they are suitable for extended-release of the circRNA contained therein. Such extended-release compositions may be conveniently administered to a subject at extended dosing intervals. For example, in one embodiment, the compositions of the present disclosure are administered to a subject twice a day, daily or every other day. In an embodiment, the compositions of the present disclosure are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by a polynucleotide described herein is produced by a target cell for sustained amounts of time. For example, the protein may be produced for more than one hour, more than four, more than six, more than 12, more than 24, more than 48 hours, or more than 72 hours after administration. In some embodiments the therapeutic product is expressed at a peak level about six hours after administration. In some embodiments the expression of the therapeutic product is sustained at least at a therapeutic level. In some embodiments the therapeutic product is expressed at least at a therapeutic level for more than one, more than four, more than six, more than 12, more than 24, more than 48, or more than 72 hours after administration. In some embodiments, the therapeutic product is detectable at a therapeutic level in patient serum or tissue (e.g., liver or lung). In some embodiments, the level of detectable therapeutic product is from continuous expression from the circRNA composition over periods of time of more than one, more than four, more than six, more than 12, more than 24, more than 48, or more than 72 hours after administration.

In some embodiments, a protein encoded by a polynucleotide described herein is produced at levels above normal physiological levels. The level of protein may be increased as compared to a control. In some embodiments, the control is the baseline physiological level of the therapeutic product in a normal individual or in a population of normal individuals. In other embodiments, the control is the baseline physiological level of the therapeutic product in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the therapeutic product upon other therapeutic intervention, e.g., upon direct injection of the corresponding therapeutic product, at one or more comparable time points.

In some embodiments, the levels of a protein encoded by a polynucleotide described herein are detectable at 3 days, 4 days, 5 days, or 1 week or more after administration. Increased levels of secreted protein may be observed in the serum and/or in a tissue (e.g., liver or lung).

In some embodiments, the method yields a sustained circulation half-life of a protein encoded by a polynucleotide described herein. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

Many types of release delivery systems are available and known to those of ordinary skill in the art. They include polymer based systems such as poly (lactide-glycolide), copolyoxalates, polycaprolactones, polyesteramides, polyorthoesters, polyhydroxybutyiic acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In some embodiments, the therapeutic agent can be conjugated either directly or indirectly through a linking moiety to a targeting moiety. Methods for conjugating therapeutic agents to targeting moieties is known in the art. See, for instance, Wadwa et al, J, Drug Targeting 3:111 (1995) and U.S. Pat. No. 5,087,616.

In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150). Depot forms of therapeutic agents can be, for example, an implantable composition comprising the therapeutic agents and a porous or non-porous material, such as a polymer, wherein the therapeutic agents are encapsulated by or diffused throughout the material and/or degradation of the non-porous material. The depot is then implanted into the desired location within the body and the therapeutic agents are released from the implant at a predetermined rate.

### 6.6.4. Use

The circular RNA produced by the construct or method of the present invention may be used for a variety of purposes, depending on the variety of target sequences. For example, where the target sequence comprises or consists of a protein coding sequence, the resulting circular RNA may be used for protein expression. The circular RNA of the present invention may also be used for various functions such as regulating miRNA activity, neutralizing binding of RNA-binding proteins, and expressing aptamers.

### 7. Assays

Various IRES-like sequence variants, endogenous IRES sequence variants, or a combination thereof, may be tested for their ability to attracts a eukaryotic ribosomal translation initiation complex and/or promote translation initiation. The assays below are described for IRES-like sequences but can be performed analogously for endogenous IRES sequence, a combination of IRES-like sequence and endogenous IRES sequence, a sequence comprising one or more IRES-like sequences or endogenous IRES sequences.

### 7.1. Determining Group II Intron's Secondary Structure and Split Site

Stem-loop structure is a type of an RNA secondary structure, which can be determined by any suitable polynucleotide folding algorithm. Some programs are based on the calculation of the minimum Gibbs free energy. An example of one such algorithm is mFold and is described by Zuker and Stiegler (Nucleic Acids Res. 9 (1981), 133-148). Another exemplary folding algorithm is the online web server RNAfold developed by the Institute for Theoretical Chemistry at the University of Vienna using a centroid structure prediction algorithm (e.g. AR Gruber et al., 2008, Cell 106). (1): 23-24; and PA Carr and GM Church, 2009, Nature Biotechnology 27 (12): 1151-62). Additional algorithms can be found in US Provisional Patent Application No. 61/836,080 (Attorney Docket No. 44790.11.2022; Broad reference number BI-2013/004A), which is incorporated herein by reference. Group II intron mainly comprises 6 stem-loop structures, called domains 1 to 6 (D1 to D6), and the 6 domains are arranged in sequence, comprising multiple exon binding sequences (EBSs), such as EBS1, EBS2, and EBS3. These EBS sequences interact, such as complementarily pair, with the intron binding sequences (IBSs) in exon regions, triggering splicing by virtue of their own hydroxyl groups within the EBS nucleic acid sequences. An exemplary structure of a group II intron's secondary structure is shown in FIG. 9. In one embodiment, group II intron are identified using an online predicting tool or a predicting software. An example of such online predicting tool is the online web server “http://webapps2.ucalgary.ca/” created by Zimmerly lab, University of Calgary.

In one embodiment, the autocatalytic self-splicing group II intron is split into two fragments at the D1 domain, and a target sequence is inserted between the split intron fragments. In one embodiment, the autocatalytic self-splicing group II intron is split into two fragments at the D2 domain, and a target sequence is inserted between the split intron fragments. In one embodiment, the autocatalytic self-splicing group II intron is split into two fragments at the D3 domain, and a target sequence is inserted between the split intron fragments.

In a preferred embodiment, the autocatalytic self-splicing group II intron is split into two fragments at the D4 domain, and a target sequence is inserted between the split intron fragments, shown in FIG. 12.

In one embodiment, the autocatalytic self-splicing group II intron is split into two fragments at the D5 domain, and a target sequence is inserted between the split intron fragments. In one embodiment, the autocatalytic self-splicing group II intron is split into two fragments at the D6 domain, and a target sequence is inserted between the split intron fragments

### A. In Vitro Circrna Production

Precursor RNAs are produced by in vitro transcription, and then circularized through cRNAzyme system.

The vectors provided herein can be made using standard techniques of molecular biology. For example, the various elements of the vectors provided herein can be obtained using recombinant methods, such as by screening cDNA and genomic libraries from cells, or by deriving the polynucleotides from a vector known to include the same.

The various elements of the vectors provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair et al, Science (1984) 223 1299; and Jay et al, J. Biol. Chem. (1984) 259:631 1.

Thus, particular nucleotide sequences can be obtained from vectors harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired vector elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al, Proc. Natl. Acad. Sci. USA (1991) 88:4084-4088. Additionally, oligonucleotide-directed synthesis (Jones et al, Nature (1986) 54:75-82), oligonucleotide directed mutagenesis of preexisting nucleotide regions (Riechmann et al, Nature (1988) 332:323-327 and Verhoeyen et al., Science (1988) 239: 1534-1536), and enzymatic filling-in of gapped oligonucleotides using T4 DNA polymerase (Queen et al, Proc. Natl. Acad. Sci. USA (1989) 86: 10029-10033) can be used.

The precursor RNA provided herein can be generated by incubating a vector provided herein under conditions permissive of transcription of the precursor RNA encoded by the vector. For example, in some embodiments a precursor RNA is synthesized by incubating a vector provided herein that comprises an RNA polymerase promoter upstream of its 5′ duplex forming region and/or expression sequence with a compatible RNA polymerase enzyme under conditions permissive of in vitro transcription. In some embodiments, the vector is incubated inside of a cell by a bacteriophage RNA polymerase or in the nucleus of a cell by host RNA polymerase P.

In some embodiments, provided herein is a method of generating precursor RNA by performing in vitro transcription using a vector provided herein as a template (e.g., a vector provided herein with an RNA polymerase promoter positioned upstream of the 5′ homology region).

In some embodiments, the resulting precursor RNA can be used to generate circular RNA (e.g., a circular RNA polynucleotide provided herein).

Thus, in some embodiments provided herein is a method of making circular RNA. In some embodiments, the method comprises synthesizing precursor RNA by transcription (e.g., run-off transcription) using a vector provided herein as a template, and incubating the resulting precursor RNA in conditions suitable for circularization, to form circular RNA.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA.

7.2. Assessing Therapeutic Product Expression Levels and/or Activities

The level of a therapeutic product, such as a polypeptide, a protein, an antibody, or an enzyme, can be determined by any method known in the art or described herein. For example, the level of a therapeutic product, such as a polypeptide, a protein, an antibody, or an enzyme, in a tissue sample can be determined by assessing (e.g., quantifying) transcribed RNA of the protein in the sample using, e.g., Northern blotting, PCR analysis, real time PCR analysis, or any other technique known in the art or described herein. In one embodiment, the level of a therapeutic product, such as a polypeptide, a protein, an antibody, or an enzyme in a tissue sample can be determined by assessing (e.g., quantifying) mRNA of the protein in the sample. The level of a therapeutic product, such as a polypeptide, a protein, an antibody, or an enzyme, in a tissue sample can also be determined by assessing (e.g., quantifying) the level of polypeptide or protein expression of the therapeutic product in the sample using, e.g., immunohistochemical analysis, Western blotting, ELISA, immunoprecipitation, flow cytometry analysis, or any other technique known in the art or described herein. In particular embodiments, the level of the protein is determined by a method capable of quantifying the amount of the therapeutic product present in a tissue sample of a patient (e.g., in human serum), and/or capable of detecting the correction of the level of protein following treatment with a circRNA or a formulation comprising a circRNA.

### 7.3. Screening Assays

Various IRES-like sequence variants, endogenous IRES sequence variants, or a combination thereof, may be tested for their ability to attracts a eukaryotic ribosomal translation initiation complex and/or promote translation initiation. The assays below are described for IRES-like sequences but can be performed analogously for endogenous IRES sequence, a combination of IRES-like sequence and endogenous IRES sequence, a sequence comprising one or more IRES-like sequences or endogenous IRES sequences.

### 7.3.1. Expression Level

For example, the effect of an IRES-like sequence, endogenous IRES sequence or a variant thereof, or a combination thereof disclosed herein on the expression of level of a therapeutic product may be assessed. In some embodiments, the effect may be assessed through in vitro translation of a RNA polynucleotide or circular RNA comprising an IRES-like sequence, endogenous IRES sequence or a variant thereof, or a combination thereof disclosed herein in a cell free system. In some embodiments, the effect may be assessed through transfection/transformation of a cell with a RNA polynucleotide or circular RNA comprising an IRES-like sequence, endogenous IRES sequence or a variant thereof, or a combination thereof disclosed herein and expression of the RNA polynucleotide or circular RNA. The expression level of the therapeutic product may be assessed according to any method known in the art and/or described herein, e.g., immunohistochemical analysis, Western blotting, ELISA, immunoprecipitation, and flow cytometry analysis.

In some embodiments, IRES-like sequences, endogenous IRES sequences or variants thereof, or combinations thereof identified based on their effect on the expression of level of a therapeutic product (e.g., an expression marker such as a fluorescence protein or a luciferase) may be further assessed for their ability to promote, facilitate, or regulate (e.g., increase or decrease) the therapeutic product expression in vitro. In some embodiments, IRES-like sequences, endogenous IRES sequences or variants thereof, or combinations thereof may be further assessed in animal models for their ability to promote, facilitate, or regulate (e.g., increase or decrease) the therapeutic product expression in vivo. In some embodiments, IRES-like sequences, endogenous IRES sequences or variants thereof, or combinations thereof may be further assessed in animal models for their ability to promote, facilitate, or regulate (e.g., increase or decrease) the therapeutic product expression in a specific tissue or organ.

Non-limiting illustrative examples of the various assays or methods are provided below.

### In Vitro Transfection and Translation

A desirable amount of a circular RNA can be transfected into cells (e.g., prokaryotic cells or eukaryotic cells) using a transfecting agent, such as Lipofectamine 3000 (Invitrogen). In vitro translation of a desirable amount of a circular RNA may be performed in a cell free lysate. The cell lysate may be collected to analyze the protein expression after incubation.

### Western Blot

Cells transfected with a RNA polynucleotide or circular RNA comprising an IRES-like sequence, endogenous IRES sequence or a variant thereof, or a combination thereof of the present disclosure according to the methods or techniques described herein may be lysed. The total cell lysates are resolved, e.g., through electrophoresis, such as with 4-20% ExpressPlus™ PAGE Gel (GeneScript®). The proteins are transferred to a membrane (e.g., PVDF membrane) to be probed with an antibody against the protein. A secondary antibody binding to the first antibody may be used to stain the membrane for detection. Positive controls such as such as known Gtx, Rsv, CrPV, PSIV, or TSV IRES may be used.

### Luciferase Assay

Cells transfected with a RNA polynucleotide or circular RNA comprising an IRES-like sequence, endogenous IRES sequence or a variant thereof, or a combination thereof of the present disclosure according to the methods or techniques described herein may be lysed. A luciferase reporter assay, such as the dual-luciferase reporter assay system from Promega™, is used to generate the luminescent signal. The luminescence is measured, for example, by Bio-Tek synergy HI.

### Flow Cytometry

Cells transfected with a RNA polynucleotide or circular RNA comprising an IRES-like sequence, endogenous IRES sequence or a variant thereof, or a combination thereof of the present disclosure according to the methods or techniques described herein are collected for flow cytometry, for example, by using BD FACSAria II. To select the singlets, SSC-A vs FSC-A may be used to select 293T cells. Two round selections of singlets may be used by SSC-W vs FSC-H and FSC-W vs FSC-H. FITC-A vs FSC-A may be used to select GFP-positive cells, and the expression level may determined by the level of fluorescence.

### ELISA

ELISA may be carried out according to Bull World Health Organ. 54(2):129-39 (1976) (PMID: 798633).

Optionally, the therapeutic product may be derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatable crosslinkers, or any combinations thereof. Optionally, the expression level of the therapeutic product may be assessed by measuring the level of the derivatizing groups.

### 7.3.2. Biological Activities

The biological activities or functions of an IRES-like sequence, endogenous IRES sequence or a variant thereof, or a combination thereof disclosed herein may be assessed according to any method known in the art and/or described herein, e.g., in vivo imaging and PET. For example, the biological activity may include inhibition of tumor growth, and may be assessed in animal models, such as cell-line-derived xenograft (CDX) and patient-derived xenograft (PDX) model.

Non-limiting illustrative examples of the various assays or methods are provided below.

### In Vivo Imaging

For detection of in vivo expression, female BALB/c mice aged 6-8 weeks may be used for administration with an RNA polynucleotide or circular RNA comprising an IRES-like sequence, endogenous IRES sequence or a variant thereof, or a combination thereof of the present disclosure (e.g., a luciferase-encoding circular RNA). The administration may be via intramuscular (i.m.), subcutaneous (s.c.), or intranasal (i.n.) routes. At certain times post administration, animals are injected intraperitoneally (i.p.) with luciferase substrate. Fluorescence signals are collected, for example, by IVIS Spectrum instrument (PerkinElmer). For in vitro imaging, tissues including brain, heart, liver, spleen, lung, kidney, and muscle from the animals are collected immediately, and fluorescence signals of each tissue are measured, for example, by IVIS imager. The fluorescence signals in regions of interest (ROIs) are quantified, e.g., by using Living Image 3.0.

### PET

Mouse tumor xenografts are formed with tumor cell lines. PET scans are performed before and after administration of the animals with an RNA polynucleotide or circular RNA comprising an IRES-like sequence, endogenous IRES sequence or a variant thereof, or a combination thereof of the present disclosure. For example, PET scans may be performed 1 h after a 3.7- to 7.4-MVBq administration. A second PET scan may be performed at suitable time points after further administrations.

For example, the assay can be used to access the competitive binding ability of the expressed therapeutic product in a biological system.

For example, the assay can be used to access the enzymatic ability of the expressed therapeutic product in a biological system.

For example, the assay can be used to access the inhibition activities of the expressed therapeutic product in a biological system.

Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the therapeutic product on the molecular target.

### Sequences

TABLE 1
Sequence Listing part 1

| SEQ ID | | |
|---|---|---|
| NO | Name | SEQUENCE: |
| 1 | Group II | CAAAGGCTTACCTATCACTAGCGCGACACGTTCCTAAGTGAAAAGCTTAG |
| | intron | GCACTGTCGAACTCAACAGTTCAGCAGTGAACTGTCATTCTAAGAAGTCA |
| | Bth | AATGAAGGAGTAACGTCTGGAAGGGCTTCCCTTAATCCTCCGACATGCAG |
| | | GAAAGTAGGCAAGTACTGAACTGTGTGAAGCTCGGTGAAGTCGGTTGAA |
| | | GGTTACCGTAAATTAGTATCTCTAATACGAAAGCTATCCAGCGGTGGATGG |
| | | TGTAACTGATAGACCGGAGGTCTATAAAACACTCAAGGTTAGGATGCGCG |
| | | ATGAACTAGAGGCGATCGCTAGTAAGCGCAGACGAATCCCTGATGGTACG |
| | | GGTCTATATCGGGAGGGAATCGAAAGGTTCTCTGACACAAATAAGTGTCG |
| | | CTACTGTGGGTGAGTAAAACTCTCCTTTATGAAAGCCCATATATCGTTACA |
| | | GGCGTTATTAAGGTAGCAGGCTCATAGGGGAAACCTAAAAGTGTATGTAC |
| | | AGATAAGAATGACGGAACGTGGTAAGCTGCCGACATGGAGGGCTTGTTCT |
| | | CTTTGAAGTGTTGCCAAGGAAAGTCACAATGAGATTAGTTGTCGATATAA |
| | | CTTGGTTTAACGGCAGTGAAAGTGGTGGCACAGTACCGATGAAACGTGTA |
| | | ATGAACGTGGAGGGATAGCCACTAGTCGATTGAAGATTGAAGGTTACTATT |
| | | GGTTAACATGGTTTCGAGTAAGACTAAGAGATGTAATGCTCCAAAGTAATA |
| | | AGGAGGTTACAGCCCATGTTAAAGAAAACCAAGCTAAGACATAACGAATA |
| | | TTATGATACACAAAAAAAGTGTATGACAATTTATACTCGAACAGTCTTAAC |
| | | GGTAACAATTTCTTTCAATTGGAAACGATGGAACGCCGTATGCCCGGAAA |
| | | CGGGCGCGTACGGTGTGGAGTGGGGGAAAAGCTGGAGATAATCTCAAAG |
| | | GCTTACCTATCACTATCGCGACACGTTCCTAAGTGA |
| 2 | Group II | GCCATAcaataaaagtgcgaaacgttatcctataagtaagaaagttttaaaattttcttacgaaaaggata |
| | intron | gaacttaaaagttctaactgttctactaaagtaataagtgaaaatcttatttaaagcaaacaaccaagtag |
| | Cte_original | ctttaagtctaagtcccctacacaagttttatactactatgcaaaacttgtgaagctaggtaaggtcgtaa |
| | (*See | tccgtgaaagtcggatgcggggctccttaaaagattactatggtaaacataagctaatccattaagatgcg |
| | footnotes | atttatatgtattttatactgttaaatatttttgtgcttgtggcttggtataaaacagttaagatgaagta |
| | to Table 1) | cttaactggttttggaataattggttgttaaactaaaacattataaatcgttagtggatacctaaggtaat |
| | | caaaaatagggataggtagaatggaacgtttgatgctgtatatgaagaggtttagtagaacctaggacaca |
| | | tatacgggctcagcaggttcatagtagctatgatactcagccggaagtcaattaattttgaaatacttcta |
| | | tggtaacataggagaaggataaaactgagtgagccaaggaacctagtcggtaatagaaaagtggaagttaa |
| | | aacaaatataagattttagaattaatttaattaatgaacggaattaatttaatgatatttaaagttagacg |
| | | gttataaattaaacatttcaaaattaaaccatatccaaattcataaatatagctagatcatatcactagtt |
| | | taaaaataaataaatcatttcaaattactattaagtaaggtattaataccttacttaatagtaatctcatt |
| | | acataagagaattactagattagcagacagattcataaaaactatatcaactaggacaatagaaaatatat |
| | | ttatacacttcctattatcgagcgaacgccttatgcgatgaaagtcgcacgtagggtgtagaccaagcgaa |
| | | atcctatgcatttaggatagtgaggtatAGCAAA |
| 3 | Group II | GCAAGACAATAAAAGTTTGACACGTGATCCTATAAGTAAGAAAGTTTTAA |
| | intron | AATTTTCTTACGAAAAGGATAGAACTTAAAAGTTCTAACTGTTCTACTAAA |
| | Cte_mut | GTAATAAGTGAAAATCTTATTTAAAGCAAACAACCAAGTAGCTTTAAGTCT |
| | | AAGTCCCCTACACAAGTTTTATACTACTATGCAAAACTTGTGAAGCTAGGT |
| | | AAGGTCGTAATCCGTGAAAGTCGGATGCGGGGCTCCTTAAAAGATTACTA |
| | | TGGTAAACATAAGCTAATCCATTAAGATGCGATTTATATGTATTTTATACTGT |
| | | TAAATATTTTTGTGCTTGTGGCTTGGTATAAAACAGTTAAGATGAAGTACTT |
| | | AACTGGTTTTGGAATAATTGGTTGTTAAACTAAAACATTATAAATCGTTAGT |
| | | GGATACCTAAGGTAATCAAAAATAGGGATAGGTAGAATGGAACGTTTGAT |
| | | GCTGTATATGAAGAGGTTTAGTAGAACCTAGGACACATATACGGGCTCAGC |
| | | AGGTTCATAGTAGCTATGATACTCAGCCGGAAGTCAATTAATTTTGAAATA |
| | | CTTCTATGGTAACATAGGAGAAGGATAAAACTGAGTGAGCCAAGGAACCT |
| | | AGTCGGTAATAGAAAAGTGGAAGTTAAAACAAATATAAGATTTTAGAATTA |
| | | ATTTAATTAATGAACGGAATTAATTTAATGATATTTAAAGTTAGACGGTTAT |
| | | AAATTAAACATTTCAAAATTAAACCATATCCAAATTCATAAATATAGCTAGA |
| | | TCATATCACTAGTTTAAAAATAAATAAATCATTTCAAATTACTATTAAGTAA |
| | | GGTATTAATACCTTACTTAATAGTAATCTCATTACATAAGAGAATTACTAGAT |
| | | TAGCAGACAGATTCATAAAAACTATATCAACTAGGACAATAGAAAATATAT |
| | | TTATACACTTCCTATTATCGAGCGAACGCCTTATGCGATGAAAGTCGCACG |
| | | TAGGGTGTAGACCAAGCGAAATCCTATGCATTTAGGATAGTGAGGTATAGC |
| | | AAA |
| 4 | Spacer | GCAATAGCCGAAAAACAAAAAACAAAAAAAACAAAAAAAAAACCAAAA |
| | sequence | AAACAAAACACA |
| | 1 | |
| 5 | Spacer | AAATTATAATAATTATAATA |
| | sequence | |
| | 2 | |
| 6 | Spacer | ATGAAACCGGCTCGGATTCCGCCCGCGTGCGCCATCCCCTCAGCTAGCAG |
| | sequence | GTGTGAGCGGCTTTCTGCCCGCAGTCTCTACACAGCTCAGCATCCTGACG |
| | 3 | CCTCCTCCCCTTGCAGGGGCGTGAAGCTACTTCAGACTCTGCTGTGACGA |
| | | CTTGGCCGCCAGGCACCGATCCTCCCCGGTGAGAAGGTCCACGAATCTTA |
| | | CTGCAGACAGATTTGCTCAGCGCG |
| 7 | Probe1 | CTTTCACTACTCCTACGAGCACCA |
| 8 | Probe2 | GACCATGCTCCCAAGCAAGATCATG |
| 9 | Gluc | ATGGGAGTCAAAGTTCTGTTTGCCCTGATCTGCATCGCTGTGGCCGAGGC |
| | | CAAGCCCACCGAGAACAACGAAGACTTCAACATCGTGGCCGTGGCCAGC |
| | | AACTTCGCGACCACGGATCTCGATGCTGACCGCGGGAAGTTGCCCGGCA |
| | | AGAAGCTGCCGCTGGAGGTGCTCAAAGAGATGGAAGCCAATGCCCGGAA |
| | | AGCTGGCTGCACCAGGGGCTGTCTGATCTGCCTGTCCCACATCAAGTGCA |
| | | CGCCCAAGATGAAGAAGTTCATCCCAGGACGCTGCCACACCTACGAAGG |
| | | CGACAAAGAGTCCGCACAGGGCGGCATAGGCGAGGCGATCGTCGACATT |
| | | CCTGAGATTCCTGGGTTCAAGGACTTGGAGCCCATGGAGCAGTTCATCGC |
| | | ACAGGTCGATCTGTGTGTGGACTGCACAACTGGCTGCCTCAAAGGGCTTG |
| | | CCAACGTGCAGTGTTCTGACCTGCTCAAGAAGTGGCTGCCGCAACGCTGT |
| | | GCGACCTTTGCCAGCAAGATCCAGGGCCAGGTGGACAAGATCAAGGGGG |
| | | CCGGTGGTGAC |
| 10 | Rluc 1 | ATGGCTTCCAAGGTGTACGACCCCGAGCAACGCAAACGCATGATCACTGG |
| | | GCCTCAGTGGTGGGCTCGCTGCAAGCAAATGAACGTGCTGGACTCCTTCA |
| | | TCAACTACTATGATTCCGAGAAGCACGCCGAGAACGCCGTGATTTTTCTG |
| | | CATGGTAACGCTGCCTCCAGCTACCTGTGGAGGCACGTCGTGCCTCACAT |
| | | CGAGCCCGTGGCTAGATGCATCATCCCTGATCTGATCGGAATGGGTAAGTC |
| | | CGGCAAGAGCGGGAATGGCTCATATCGCCTCCTGGATCACTACAAGTACC |
| | | TCACCGCTTGGTTCGAGCTGCTGAACCTTCCAAAGAAAATCATCTTTGTG |
| | | GGCCACGACTGGGGGGCTTGTCTGGCCTTTCACTACTCCTACGAGCACCA |
| | | AGACAAGATCAAGGCCATCGTCCATGCTGAGAGTGTCGTGGACGTGATCG |
| | | AGTCCTGGGACGAGTGGCCTGACATCGAGGAGGATATCGCCCTGATCAAG |
| | | AGCGAAGAGGGCGAGAAAATGGTGCTTGAGAATAACTTCTTCGTCGAGA |
| | | CCATGCTCCCAAGCAAGATCATGCGGAAACTGGAGCCTGAGGAGTTCGCT |
| | | GCCTACCTGGAGCCATTCAAGGAGAAGGGCGAGGTTAGACGGCCTACCC |
| | | TCTCCTGGCCTCGCGAGATCCCTCTCGTTAAGGGAGGCAAGCCCGACGTC |
| | | GTCCAGATTGTCCGCAACTACAACGCCTACCTTCGGGCCAGCGACGATCT |
| | | GCCTAAGATGTTCATCGAGTCCGACCCTGGGTTCTTTTCCAACGCTATTGT |
| | | CGAGGGAGCTAAGAAGTTCCCTAACACCGAGTTCGTGAAGGTGAAGGGC |
| | | CTCCACTTCAGCCAGGAGGACGCTCCAGATGAAATGGGTAAGTACATCAA |
| | | GAGCTTCGTGGAGCGCGTGCTGAAGAACGAGCAGTAA |
| 11 | Rluc2 | ATGAAACCGGCTCGGATTCCGCCCGCGTGCGCCATCCCCTCAGCTAGCAG |
| | IRES- | GTGTGAGCGGCTTTCTGCCCGCAGTCTCTACACAGCTCAGCATCCTGACG |
| | | CCTCCTCCCCTTGCAGGGGCGTGAAGCTACTTCAGACTCTGCTGTGACGA |
| | | CTTGGCCGCCAGGCACCGATCCTCCCCGGTGAGAAGGTCCACGAATCTTA |
| | | CTGCAGACAGATTTGCTCAGCGCGATGGCTTCCAAGGTGTACGACCCCGA |
| | | GCAACGCAAACGCATGATCACTGGGCCTCAGTGGTGGGCTCGCTGCAAG |
| | | CAAATGAACGTGCTGGACTCCTTCATCAACTACTATGATTCCGAGAAGCA |
| | | CGCCGAGAACGCCGTGATTTTTCTGCATGGTAACGCTGCCTCCAGCTACCT |
| | | GTGGAGGCACGTCGTGCCTCACATCGAGCCCGTGGCTAGATGCATCATCC |
| | | CTGATCTGATCGGAATGGGTAAGTCCGGCAAGAGCGGGAATGGCTCATAT |
| | | CGCCTCCTGGATCACTACAAGTACCTCACCGCTTGGTTCGAGCTGCTGAA |
| | | CCTTCCAAAGAAAATCATCTTTGTGGGCCACGACTGGGGGGCTTGTCTGG |
| | | CCTTTCACTACTCCTACGAGCACCAAGACAAGATCAAGGCCATCGTCCAT |
| | | GCTGAGAGTGTCGTGGACGTGATCGAGTCCTGGGACGAGTGGCCTGACA |
| | | TCGAGGAGGATATCGCCCTGATCAAGAGCGAAGAGGGCGAGAAAATGGT |
| | | GCTTGAGAATAACTTCTTCGTCGAGACCATGCTCCCAAGCAAGATCATGC |
| | | GGAAACTGGAGCCTGAGGAGTTCGCTGCCTACCTGGAGCCATTCAAGGA |
| | | GAAGGGCGAGGTTAGACGGCCTACCCTCTCCTGGCCTCGCGAGATCCCTC |
| | | TCGTTAAGGGAGGCAAGCCCGACGTCGTCCAGATTGTCCGCAACTACAAC |
| | | GCCTACCTTCGGGCCAGCGACGATCTGCCTAAGATGTTCATCGAGTCCGA |
| | | CCCTGGGTTCTTTTCCAACGCTATTGTCGAGGGAGCTAAGAAGTTCCCTA |
| | | ACACCGAGTTCGTGAAGGTGAAGGGCCTCCACTTCAGCCAGGAGGACGC |
| | | TCCAGATGAAATGGGTAAGTACATCAAGAGCTTCGTGGAGCGCGTGCTGA |
| | | AGAACGAGCAGTAA |
| 12 | EgFP | AGCAAAGACCCCAACGAGAAGCGCGATCACATGGTCCTGCTGGAGTTCG |
| | | TGACCGCCGCCGGGATCACTCTCGGCATGGACGAGCTGTACAAGTAACAA |
| | | ACAAACAAAACAAAAACACTCCCCTGTGAGGAACTACTGTCTTCACGCA |
| | | GAAAGCGTCTAGCCATGGCGTTAGTATGAGTGTCGTGCAGCCTCCAGGAC |
| | | CCCCCCTCCCGGGAGAGCCATAGTGGTCTGCGGAACCGGTGAGTACACCG |
| | | GAATTGCCAGGACGACCGGGTCCTTTCTTGGATAAACCCGCTCAATGCCT |
| | | GGAGATTTGGGCGTGCCCCCGCAAGACTGCTAGCCGAGTAGTGTTGGGTC |
| | | GCGAAAGGCCTTGTGGTACTGCCTGATAGGGTGCTTGCGAGTGCCCCGGG |
| | | AGGTCTCGTAGACCGTGCACCATGAGCACGAATCCTAAAATGGTGAGCAA |
| | | GGGCGAGGAGCTGTTCACCGGGGTGGTGCCCATCCTGGTCGAGCTGGAC |
| | | GGCGACGTAAACGGCCACAAGTTCAGCGTGTCCGGCGAGGGCGAGGGCG |
| | | ATGCCACCTACGGCAAGCTGACCCTGAAGTTCATCTGCACCACCGGCAAG |
| | | CTGCCCGTGCCCTGGCCCACCCTCGTGACCACCCTGACCTACGGCGTGCA |
| | | GTGCTTCAGCCGCTACCCCGACCACATGAAGCAGCACGACTTCTTCAAGT |
| | | CCGCCATGCCCGAAGGCTACGTCCAGGAGCGCACCATCTTCTTCAAGGAC |
| | | GACGGCAACTACAAGACCCGCGCCGAGGTGAAGTTCGAGGGCGACACCC |
| | | TGGTGAACCGCATCGAGCTGAAGGGCATCGACTTCAAGGAGGACGGCAA |
| | | CATCCTGGGGCACAAGCTGGAGTACAACTACAACAGCCACAACGTCTATA |
| | | TCATGGCCGACAAGCAGAAGAACGGCATCAAGGTGAACTTCAAGATCCG |
| | | CCACAACATCGAGGACGGCAGCGTGCAGCTCGCCGACCACTACCAGCAG |
| | | AACACCCCCATCGGCGACGGCCCCGTGCTGCTGCCCGACAACCACTACCT |
| | | GAGCACCCAGTCCGCCCTG |
| 13 | IRES- | AGCAGTTCATCGCACAGGTCGATCTGTGTGTGGACTGCACAACTGGCTGC |
| | gluc | CTCAAAGGGCTTGCCAACGTGCAGTGTTCTGACCTGCTCAAGAAGTGGCT |
| | | GCCGCAACGCTGTGCGACCTTTGCCAGCAAGATCCAGGGCCAGGTGGAC |
| | | AAGATCAAGGGGGCCGGTGGTGACTAACAAACAAACAAAACAAAAACA |
| | | CTCCCCTGTGAGGAACTACTGTCTTCACGCAGAAAGCGTCTAGCCATGGC |
| | | GTTAGTATGAGTGTCGTGCAGCCTCCAGGACCCCCCCTCCCGGGAGAGCC |
| | | ATAGTGGTCTGCGGAACCGGTGAGTACACCGGAATTGCCAGGACGACCG |
| | | GGTCCTTTCTTGGATAAACCCGCTCAATGCCTGGAGATTTGGGCGTGCCCC |
| | | CGCAAGACTGCTAGCCGAGTAGTGTTGGGTCGCGAAAGGCCTTGTGGTAC |
| | | TGCCTGATAGGGTGCTTGCGAGTGCCCCGGGAGGTCTCGTAGACCGTGCA |
| | | CCATGAGCACGAATCCTAAAATGGGAGTCAAAGTTCTGTTTGCCCTGATC |
| | | TGCATCGCTGTGGCCGAGGCCAAGCCCACCGAGAACAACGAAGACTTCA |
| | | ACATCGTGGCCGTGGCCAGCAACTTCGCGACCACGGATCTCGATGCTGAC |
| | | CGCGGGAAGTTGCCCGGCAAGAAGCTGCCGCTGGAGGTGCTCAAAGAGA |
| | | TGGAAGCCAATGCCCGGAAAGCTGGCTGCACCAGGGGCTGTCTGATCTG |
| | | CCTGTCCCACATCAAGTGCACGCCCAAGATGAAGAAGTTCATCCCAGGAC |
| | | GCTGCCACACCTACGAAGGCGACAAAGAGTCCGCACAGGGCGGCATAGG |
| | | CGAGGCGATCGTCGACATTCCTGAGATTCCTGGGTTCAAGGACTTGGAGC |
| | | CCATGG |
| 14 | arm1 | AATACCTTACTTAATAGTAACAATAGAAAATC |
| 15 | arm2 | AAGCTAGATCATATTACTATTAAGTAAGGTATT |
| 16 | CRNAzy | AATACCTTACTTAATAGTAACAATAGAAAATCCTATTATCGAGCGAACGCC |
| | me (Cte- | TTATGCGATGAAAGTCGCACGTAGGGTGTAGACCAAGCGAAATCCTATGC |
| | Rluc) | ATTTAGGATAGTGAGGTATAGCAAAATGGCTTCCAAGGTGTACGACCCCG |
| | | AGCAACGCAAACGCATGATCACTGGGCCTCAGTGGTGGGCTCGCTGCAA |
| | | GCAAATGAACGTGCTGGACTCCTTCATCAACTACTATGATTCCGAGAAGC |
| | | ACGCCGAGAACGCCGTGATTTTTCTGCATGGTAACGCTGCCTCCAGCTAC |
| | | CTGTGGAGGCACGTCGTGCCTCACATCGAGCCCGTGGCTAGATGCATCAT |
| | | CCCTGATCTGATCGGAATGGGTAAGTCCGGCAAGAGCGGGAATGGCTCAT |
| | | ATCGCCTCCTGGATCACTACAAGTACCTCACCGCTTGGTTCGAGCTGCTGA |
| | | ACCTTCCAAAGAAAATCATCTTTGTGGGCCACGACTGGGGGGCTTGTCTG |
| | | GCCTTTCACTACTCCTACGAGCACCAAGACAAGATCAAGGCCATCGTCCA |
| | | TGCTGAGAGTGTCGTGGACGTGATCGAGTCCTGGGACGAGTGGCCTGAC |
| | | ATCGAGGAGGATATCGCCCTGATCAAGAGCGAAGAGGGCGAGAAAATGG |
| | | TGCTTGAGAATAACTTCTTCGTCGAGACCATGCTCCCAAGCAAGATCATG |
| | | CGGAAACTGGAGCCTGAGGAGTTCGCTGCCTACCTGGAGCCATTCAAGG |
| | | AGAAGGGCGAGGTTAGACGGCCTACCCTCTCCTGGCCTCGCGAGATCCCT |
| | | CTCGTTAAGGGAGGCAAGCCCGACGTCGTCCAGATTGTCCGCAACTACAA |
| | | CGCCTACCTTCGGGCCAGCGACGATCTGCCTAAGATGTTCATCGAGTCCG |
| | | ACCCTGGGTTCTTTTCCAACGCTATTGTCGAGGGAGCTAAGAAGTTCCCT |
| | | AACACCGAGTTCGTGAAGGTGAAGGGCCTCCACTTCAGCCAGGAGGACG |
| | | CTCCAGATGAAATGGGTAAGTACATCAAGAGCTTCGTGGAGCGCGTGCTG |
| | | AAGAACGAGCAGTAAGCCATACAATAAAAGTGCGAAACGTTATCCTATAA |
| | | GTAAGAAAGTTTTAAAATTTTCTTACGAAAAGGATAGAACTTAAAAGTTC |
| | | TAACTGTTCTACTAAAGTAATAAGTGAAAATCTTATTTAAAGCAAACAACC |
| | | AAGTAGCTTTAAGTCTAAGTCCCCTACACAAGTTTTATACTACTATGCAAA |
| | | ACTTGTGAAGCTAGGTAAGGTCGTAATCCGTGAAAGTCGGATGCGGGGCT |
| | | CCTTAAAAGATTACTATGGTAAACATAAGCTAATCCATTAAGATGCGATTTA |
| | | TATGTATTTTATACTGTTAAATATTTTTGTGCTTGTGGCTTGGTATAAAACAG |
| | | TTAAGATGAAGTACTTAACTGGTTTTGGAATAATTGGTTGTTAAACTAAAA |
| | | CATTATAAATCGTTAGTGGATACCTAAGGTAATCAAAAATAGGGATAGGTA |
| | | GAATGGAACGTTTGATGCTGTATATGAAGAGGTTTAGTAGAACCTAGGAC |
| | | ACATATACGGGCTCAGCAGGTTCATAGTAGCTATGATACTCAGCCGGAAGT |
| | | CAATTAATTTTGAAATACTTCTATGGTAACATAGGAGAAGGATAAAACTGA |
| | | GTGAGCCAAGGAACCTAGTCGGTAATAGAAGCTAGATCATATTACTATTAA |
| | | GTAAGGTATT |
| 17 | Group II | CAAAGGCUUACCUAUCACUAGCGCGACACGUUCCUAAGUGAAAAGCUU |
| | intron | AGGCACUGUCGAACUCAACAGUUCAGCAGUGAACUGUCAUUCUAAGAA |
| | Bth | GUCAAAUGAAGGAGUAACGUCUGGAAGGGCUUCCCUUAAUCCUCCGAC |
| | | AUGCAGGAAAGUAGGCAAGUACUGAACUGUGUGAAGCUCGGUGAAGU |
| | | CGGUUGAAGGUUACCGUAAAUUAGUAUCUCUAAUACGAAAGCUAUCC |
| | | AGCGGUGGAUGGUGUAACUGAUAGACCGGAGGUCUAUAAAACACUCA |
| | | AGGUUAGGAUGCGCGAUGAACUAGAGGCGAUCGCUAGUAAGCGCAGA |
| | | CGAAUCCCUGAUGGUACGGGUCUAUAUCGGGAGGGAAUCGAAAGGUU |
| | | CUCUGACACAAAUAAGUGUCGCUACUGUGGGUGAGUAAAACUCUCCUU |
| | | UAUGAAAGCCCAUAUAUCGUUACAGGCGUUAUUAAGGUAGCAGGCUC |
| | | AUAGGGGAAACCUAAAAGUGUAUGUACAGAUAAGAAUGACGGAACGU |
| | | GGUAAGCUGCCGACAUGGAGGGCUUGUUCUCUUUGAAGUGUUGCCAA |
| | | GGAAAGUCACAAUGAGAUUAGUUGUCGAUAUAACUUGGUUUAACGGC |
| | | AGUGAAAGUGGUGGCACAGUACCGAUGAAACGUGUAAUGAACGUGGA |
| | | GGGAUAGCCACUAGUCGAUUGAAGAUUGAAGGUUACUAUUGGUUAAC |
| | | AUGGUUUCGAGUAAGACUAAGAGAUGUAAUGCUCCAAAGUAAUAAGG |
| | | AGGUUACAGCCCAUGUUAAAGAAAACCAAGCUAAGACAUAACGAAUA |
| | | UUAUGAUACACAAAAAAAGUGUAUGACAAUUUAUACUCGAACAGUCU |
| | | UAACGGUAACAAUUUCUUUCAAUUGGAAACGAUGGAACGCCGUAUGC |
| | | CCGGAAACGGGCGCGUACGGUGUGGAGUGGGGGAAAAGCUGGAGAUA |
| | | AUCUCAAAGGCUUACCUAUCACUAUCGCGACACGUUCCUAAGUGA |
| 18 | Group II | GCCAUACAAUAAAAGUGCGAAACGUUAUCCUAUAAGUAAGAAAGUUU |
| | intron | UAAAAUUUUCUUACGAAAAGGAUAGAACUUAAAAGUUCUAACUGUUC |
| | Cte_original | UACUAAAGUAAUAAGUGAAAAUCUUAUUUAAAGCAAACAACCAAGUA |
| | | GCUUUAAGUCUAAGUCCCCUACACAAGUUUUAUACUACUAUGCAAAAC |
| | | UUGUGAAGCUAGGUAAGGUCGUAAUCCGUGAAAGUCGGAUGCGGGGC |
| | | UCCUUAAAAGAUUACUAUGGUAAACAUAAGCUAAUCCAUUAAGAUGC |
| | | GAUUUAUAUGUAUUUUAUACUGUUAAAUAUUUUUGUGCUUGUGGCUU |
| | | GGUAUAAAACAGUUAAGAUGAAGUACUUAACUGGUUUUGGAAUAAUU |
| | | GGUUGUUAAACUAAAACAUUAUAAAUCGUUAGUGGAUACCUAAGGUA |
| | | AUCAAAAAUAGGGAUAGGUAGAAUGGAACGUUUGAUGCUGUAUAUGA |
| | | AGAGGUUUAGUAGAACCUAGGACACAUAUACGGGCUCAGCAGGUUCA |
| | | UAGUAGCUAUGAUACUCAGCCGGAAGUCAAUUAAUUUUGAAAUACUU |
| | | CUAUGGUAACAUAGGAGAAGGAUAAAACUGAGUGAGCCAAGGAACCU |
| | | AGUCGGUAAUAGAAAAGUGGAAGUUAAAACAAAUAUAAGAUUUUAGA |
| | | AUUAAUUUAAUUAAUGAACGGAAUUAAUUUAAUGAUAUUUAAAGUUA |
| | | GACGGUUAUAAAUUAAACAUUUCAAAAUUAAACCAUAUCCAAAUUCA |
| | | UAAAUAUAGCUAGAUCAUAUCACUAGUUUAAAAAUAAAUAAAUCAUU |
| | | UCAAAUUACUAUUAAGUAAGGUAUUAAUACCUUACUUAAUAGUAAUC |
| | | UCAUUACAUAAGAGAAUUACUAGAUUAGCAGACAGAUUCAUAAAAAC |
| | | UAUAUCAACUAGGACAAUAGAAAAUAUAUUUAUACACUUCCUAUUAU |
| | | CGAGCGAACGCCUUAUGCGAUGAAAGUCGCACGUAGGGUGUAGACCAA |
| | | GCGAAAUCCUAUGCAUUUAGGAUAGUGAGGUAUAGCAAA |
| 19 | Group II | GCAAGACAAUAAAAGUUUGACACGUGAUCCUAUAAGUAAGAAAGUUU |
| | intron | UAAAAUUUUCUUACGAAAAGGAUAGAACUUAAAAGUUCUAACUGUUC |
| | Cte_mut | UACUAAAGUAAUAAGUGAAAAUCUUAUUUAAAGCAAACAACCAAGUA |
| | | GCUUUAAGUCUAAGUCCCCUACACAAGUUUUAUACUACUAUGCAAAAC |
| | | UUGUGAAGCUAGGUAAGGUCGUAAUCCGUGAAAGUCGGAUGCGGGGC |
| | | UCCUUAAAAGAUUACUAUGGUAAACAUAAGCUAAUCCAUUAAGAUGC |
| | | GAUUUAUAUGUAUUUUAUACUGUUAAAUAUUUUUGUGCUUGUGGCUU |
| | | GGUAUAAAACAGUUAAGAUGAAGUACUUAACUGGUUUUGGAAUAAUU |
| | | GGUUGUUAAACUAAAACAUUAUAAAUCGUUAGUGGAUACCUAAGGUA |
| | | AUCAAAAAUAGGGAUAGGUAGAAUGGAACGUUUGAUGCUGUAUAUGA |
| | | AGAGGUUUAGUAGAACCUAGGACACAUAUACGGGCUCAGCAGGUUCA |
| | | UAGUAGCUAUGAUACUCAGCCGGAAGUCAAUUAAUUUUGAAAUACUU |
| | | CUAUGGUAACAUAGGAGAAGGAUAAAACUGAGUGAGCCAAGGAACCU |
| | | AGUCGGUAAUAGAAAAGUGGAAGUUAAAACAAAUAUAAGAUUUUAGA |
| | | AUUAAUUUAAUUAAUGAACGGAAUUAAUUUAAUGAUAUUUAAAGUUA |
| | | GACGGUUAUAAAUUAAACAUUUCAAAAUUAAACCAUAUCCAAAUUCA |
| | | UAAAUAUAGCUAGAUCAUAUCACUAGUUUAAAAAUAAAUAAAUCAUU |
| | | UCAAAUUACUAUUAAGUAAGGUAUUAAUACCUUACUUAAUAGUAAUC |
| | | UCAUUACAUAAGAGAAUUACUAGAUUAGCAGACAGAUUCAUAAAAAC |
| | | UAUAUCAACUAGGACAAUAGAAAAUAUAUUUAUACACUUCCUAUUAU |
| | | CGAGCGAACGCCUUAUGCGAUGAAAGUCGCACGUAGGGUGUAGACCAA |
| | | GCGAAAUCCUAUGCAUUUAGGAUAGUGAGGUAUAGCAAA |
| 20 | Spacer | GCAAUAGCCGAAAAACAAAAAACAAAAAAAACAAAAAAAAAACCAAA |
| | sequence | AAAACAAAACACA |
| | 1 | |
| 21 | Spacer | AAAUUAUAAUAAUUAUAAUA |
| | sequence | |
| | 2 | |
| 22 | Spacer | AUGAAACCGGCUCGGAUUCCGCCCGCGUGCGCCAUCCCCUCAGCUAGC |
| | sequence | AGGUGUGAGCGGCUUUCUGCCCGCAGUCUCUACACAGCUCAGCAUCCU |
| | 3 | GACGCCUCCUCCCCUUGCAGGGGCGUGAAGCUACUUCAGACUCUGCUG |
| | | UGACGACUUGGCCGCCAGGCACCGAUCCUCCCCGGUGAGAAGGUCCAC |
| | | GAAUCUUACUGCAGACAGAUUUGCUCAGCGCG |
| 23 | Probe1 | CUUUCACUACUCCUACGAGCACCA |
| 24 | Probe2 | GACCAUGCUCCCAAGCAAGAUCAUG |
| 25 | Gluc | AUGGGAGUCAAAGUUCUGUUUGCCCUGAUCUGCAUCGCUGUGGCCGAG |
| | | GCCAAGCCCACCGAGAACAACGAAGACUUCAACAUCGUGGCCGUGGCC |
| | | AGCAACUUCGCGACCACGGAUCUCGAUGCUGACCGCGGGAAGUUGCCC |
| | | GGCAAGAAGCUGCCGCUGGAGGUGCUCAAAGAGAUGGAAGCCAAUGCC |
| | | CGGAAAGCUGGCUGCACCAGGGGCUGUCUGAUCUGCCUGUCCCACAUC |
| | | AAGUGCACGCCCAAGAUGAAGAAGUUCAUCCCAGGACGCUGCCACACC |
| | | UACGAAGGCGACAAAGAGUCCGCACAGGGCGGCAUAGGCGAGGCGAUC |
| | | GUCGACAUUCCUGAGAUUCCUGGGUUCAAGGACUUGGAGCCCAUGGAG |
| | | CAGUUCAUCGCACAGGUCGAUCUGUGUGUGGACUGCACAACUGGCUGC |
| | | CUCAAAGGGCUUGCCAACGUGCAGUGUUCUGACCUGCUCAAGAAGUGG |
| | | CUGCCGCAACGCUGUGCGACCUUUGCCAGCAAGAUCCAGGGCCAGGUG |
| | | GACAAGAUCAAGGGGGCCGGUGGUGAC |
| 26 | Rluc 1 | AUGGCUUCCAAGGUGUACGACCCCGAGCAACGCAAACGCAUGAUCACU |
| | | GGGCCUCAGUGGUGGGCUCGCUGCAAGCAAAUGAACGUGCUGGACUCC |
| | | UUCAUCAACUACUAUGAUUCCGAGAAGCACGCCGAGAACGCCGUGAUU |
| | | UUUCUGCAUGGUAACGCUGCCUCCAGCUACCUGUGGAGGCACGUCGUG |
| | | CCUCACAUCGAGCCCGUGGCUAGAUGCAUCAUCCCUGAUCUGAUCGGA |
| | | AUGGGUAAGUCCGGCAAGAGCGGGAAUGGCUCAUAUCGCCUCCUGGAU |
| | | CACUACAAGUACCUCACCGCUUGGUUCGAGCUGCUGAACCUUCCAAAG |
| | | AAAAUCAUCUUUGUGGGCCACGACUGGGGGGCUUGUCUGGCCUUUCAC |
| | | UACUCCUACGAGCACCAAGACAAGAUCAAGGCCAUCGUCCAUGCUGAG |
| | | AGUGUCGUGGACGUGAUCGAGUCCUGGGACGAGUGGCCUGACAUCGA |
| | | GGAGGAUAUCGCCCUGAUCAAGAGCGAAGAGGGCGAGAAAAUGGUGC |
| | | UUGAGAAUAACUUCUUCGUCGAGACCAUGCUCCCAAGCAAGAUCAUGC |
| | | GGAAACUGGAGCCUGAGGAGUUCGCUGCCUACCUGGAGCCAUUCAAGG |
| | | AGAAGGGCGAGGUUAGACGGCCUACCCUCUCCUGGCCUCGCGAGAUCC |
| | | CUCUCGUUAAGGGAGGCAAGCCCGACGUCGUCCAGAUUGUCCGCAACU |
| | | ACAACGCCUACCUUCGGGCCAGCGACGAUCUGCCUAAGAUGUUCAUCG |
| | | AGUCCGACCCUGGGUUCUUUUCCAACGCUAUUGUCGAGGGAGCUAAGA |
| | | AGUUCCCUAACACCGAGUUCGUGAAGGUGAAGGGCCUCCACUUCAGCC |
| | | AGGAGGACGCUCCAGAUGAAAUGGGUAAGUACAUCAAGAGCUUCGUG |
| | | GAGCGCGUGCUGAAGAACGAGCAGUAA |
| 27 | Rluc2 | AUGAAACCGGCUCGGAUUCCGCCCGCGUGCGCCAUCCCCUCAGCUAGC |
| | | AGGUGUGAGCGGCUUUCUGCCCGCAGUCUCUACACAGCUCAGCAUCCU |
| | | GACGCCUCCUCCCCUUGCAGGGGCGUGAAGCUACUUCAGACUCUGCUG |
| | | UGACGACUUGGCCGCCAGGCACCGAUCCUCCCCGGUGAGAAGGUCCAC |
| | | GAAUCUUACUGCAGACAGAUUUGCUCAGCGCGAUGGCUUCCAAGGUGU |
| | | ACGACCCCGAGCAACGCAAACGCAUGAUCACUGGGCCUCAGUGGUGGG |
| | | CUCGCUGCAAGCAAAUGAACGUGCUGGACUCCUUCAUCAACUACUAUG |
| | | AUUCCGAGAAGCACGCCGAGAACGCCGUGAUUUUUCUGCAUGGUAACG |
| | | CUGCCUCCAGCUACCUGUGGAGGCACGUCGUGCCUCACAUCGAGCCCG |
| | | UGGCUAGAUGCAUCAUCCCUGAUCUGAUCGGAAUGGGUAAGUCCGGCA |
| | | AGAGCGGGAAUGGCUCAUAUCGCCUCCUGGAUCACUACAAGUACCUCA |
| | | CCGCUUGGUUCGAGCUGCUGAACCUUCCAAAGAAAAUCAUCUUUGUGG |
| | | GCCACGACUGGGGGGCUUGUCUGGCCUUUCACUACUCCUACGAGCACC |
| | | AAGACAAGAUCAAGGCCAUCGUCCAUGCUGAGAGUGUCGUGGACGUG |
| | | AUCGAGUCCUGGGACGAGUGGCCUGACAUCGAGGAGGAUAUCGCCCUG |
| | | AUCAAGAGCGAAGAGGGCGAGAAAAUGGUGCUUGAGAAUAACUUCUU |
| | | CGUCGAGACCAUGCUCCCAAGCAAGAUCAUGCGGAAACUGGAGCCUGA |
| | | GGAGUUCGCUGCCUACCUGGAGCCAUUCAAGGAGAAGGGCGAGGUUA |
| | | GACGGCCUACCCUCUCCUGGCCUCGCGAGAUCCCUCUCGUUAAGGGAG |
| | | GCAAGCCCGACGUCGUCCAGAUUGUCCGCAACUACAACGCCUACCUUC |
| | | GGGCCAGCGACGAUCUGCCUAAGAUGUUCAUCGAGUCCGACCCUGGGU |
| | | UCUUUUCCAACGCUAUUGUCGAGGGAGCUAAGAAGUUCCCUAACACCG |
| | | AGUUCGUGAAGGUGAAGGGCCUCCACUUCAGCCAGGAGGACGCUCCAG |
| | | AUGAAAUGGGUAAGUACAUCAAGAGCUUCGUGGAGCGCGUGCUGAAG |
| | | AACGAGCAGUAA |
| 28 | IRES- | AGCAAAGACCCCAACGAGAAGCGCGAUCACAUGGUCCUGCUGGAGUUC |
| | EgFP | GUGACCGCCGCCGGGAUCACUCUCGGCAUGGACGAGCUGUACAAGUAA |
| | | CAAACAAACAAAACAAAAACACUCCCCUGUGAGGAACUACUGUCUUCA |
| | | CGCAGAAAGCGUCUAGCCAUGGCGUUAGUAUGAGUGUCGUGCAGCCUC |
| | | CAGGACCCCCCCUCCCGGGAGAGCCAUAGUGGUCUGCGGAACCGGUGA |
| | | GUACACCGGAAUUGCCAGGACGACCGGGUCCUUUCUUGGAUAAACCCG |
| | | CUCAAUGCCUGGAGAUUUGGGCGUGCCCCCGCAAGACUGCUAGCCGAG |
| | | UAGUGUUGGGUCGCGAAAGGCCUUGUGGUACUGCCUGAUAGGGUGCU |
| | | UGCGAGUGCCCCGGGAGGUCUCGUAGACCGUGCACCAUGAGCACGAAU |
| | | CCUAAAAUGGUGAGCAAGGGCGAGGAGCUGUUCACCGGGGUGGUGCCC |
| | | AUCCUGGUCGAGCUGGACGGCGACGUAAACGGCCACAAGUUCAGCGUG |
| | | UCCGGCGAGGGCGAGGGCGAUGCCACCUACGGCAAGCUGACCCUGAAG |
| | | UUCAUCUGCACCACCGGCAAGCUGCCCGUGCCCUGGCCCACCCUCGUG |
| | | ACCACCCUGACCUACGGCGUGCAGUGCUUCAGCCGCUACCCCGACCAC |
| | | AUGAAGCAGCACGACUUCUUCAAGUCCGCCAUGCCCGAAGGCUACGUC |
| | | CAGGAGCGCACCAUCUUCUUCAAGGACGACGGCAACUACAAGACCCGC |
| | | GCCGAGGUGAAGUUCGAGGGCGACACCCUGGUGAACCGCAUCGAGCUG |
| | | AAGGGCAUCGACUUCAAGGAGGACGGCAACAUCCUGGGGCACAAGCUG |
| | | GAGUACAACUACAACAGCCACAACGUCUAUAUCAUGGCCGACAAGCAG |
| | | AAGAACGGCAUCAAGGUGAACUUCAAGAUCCGCCACAACAUCGAGGAC |
| | | GGCAGCGUGCAGCUCGCCGACCACUACCAGCAGAACACCCCCAUCGGC |
| | | GACGGCCCCGUGCUGCUGCCCGACAACCACUACCUGAGCACCCAGUCC |
| | | GCCCUG |
| 29 | IRES- | AGCAGUUCAUCGCACAGGUCGAUCUGUGUGUGGACUGCACAACUGGCU |
| | gluc | GCCUCAAAGGGCUUGCCAACGUGCAGUGUUCUGACCUGCUCAAGAAGU |
| | | GGCUGCCGCAACGCUGUGCGACCUUUGCCAGCAAGAUCCAGGGCCAGG |
| | | UGGACAAGAUCAAGGGGGCCGGUGGUGACUAACAAACAAACAAAACA |
| | | AAAACACUCCCCUGUGAGGAACUACUGUCUUCACGCAGAAAGCGUCUA |
| | | GCCAUGGCGUUAGUAUGAGUGUCGUGCAGCCUCCAGGACCCCCCCUCC |
| | | CGGGAGAGCCAUAGUGGUCUGCGGAACCGGUGAGUACACCGGAAUUGC |
| | | CAGGACGACCGGGUCCUUUCUUGGAUAAACCCGCUCAAUGCCUGGAGA |
| | | UUUGGGCGUGCCCCCGCAAGACUGCUAGCCGAGUAGUGUUGGGUCGCG |
| | | AAAGGCCUUGUGGUACUGCCUGAUAGGGUGCUUGCGAGUGCCCCGGGA |
| | | GGUCUCGUAGACCGUGCACCAUGAGCACGAAUCCUAAAAUGGGAGUCA |
| | | AAGUUCUGUUUGCCCUGAUCUGCAUCGCUGUGGCCGAGGCCAAGCCCA |
| | | CCGAGAACAACGAAGACUUCAACAUCGUGGCCGUGGCCAGCAACUUCG |
| | | CGACCACGGAUCUCGAUGCUGACCGCGGGAAGUUGCCCGGCAAGAAGC |
| | | UGCCGCUGGAGGUGCUCAAAGAGAUGGAAGCCAAUGCCCGGAAAGCUG |
| | | GCUGCACCAGGGGCUGUCUGAUCUGCCUGUCCCACAUCAAGUGCACGC |
| | | CCAAGAUGAAGAAGUUCAUCCCAGGACGCUGCCACACCUACGAAGGCG |
| | | ACAAAGAGUCCGCACAGGGCGGCAUAGGCGAGGCGAUCGUCGACAUUC |
| | | CUGAGAUUCCUGGGUUCAAGGACUUGGAGCCCAUGG |
| 30 | arm1 | AAUACCUUACUUAAUAGUAACAAUAGAAAAUC |
| 31 | arm2 | AAGCUAGAUCAUAUUACUAUUAAGUAAGGUAUU |
| 32 | CRNAzy | AAUACCUUACUUAAUAGUAACAAUAGAAAAUCCUAUUAUCGAGCGAA |
| | me (Cte- | CGCCUUAUGCGAUGAAAGUCGCACGUAGGGUGUAGACCAAGCGAAAUC |
| | Rluc) | CUAUGCAUUUAGGAUAGUGAGGUAUAGCAAAAUGGCUUCCAAGGUGU |
| | | ACGACCCCGAGCAACGCAAACGCAUGAUCACUGGGCCUCAGUGGUGGG |
| | | CUCGCUGCAAGCAAAUGAACGUGCUGGACUCCUUCAUCAACUACUAUG |
| | | AUUCCGAGAAGCACGCCGAGAACGCCGUGAUUUUUCUGCAUGGUAACG |
| | | CUGCCUCCAGCUACCUGUGGAGGCACGUCGUGCCUCACAUCGAGCCCG |
| | | UGGCUAGAUGCAUCAUCCCUGAUCUGAUCGGAAUGGGUAAGUCCGGCA |
| | | AGAGCGGGAAUGGCUCAUAUCGCCUCCUGGAUCACUACAAGUACCUCA |
| | | CCGCUUGGUUCGAGCUGCUGAACCUUCCAAAGAAAAUCAUCUUUGUGG |
| | | GCCACGACUGGGGGGCUUGUCUGGCCUUUCACUACUCCUACGAGCACC |
| | | AAGACAAGAUCAAGGCCAUCGUCCAUGCUGAGAGUGUCGUGGACGUG |
| | | AUCGAGUCCUGGGACGAGUGGCCUGACAUCGAGGAGGAUAUCGCCCUG |
| | | AUCAAGAGCGAAGAGGGCGAGAAAAUGGUGCUUGAGAAUAACUUCUU |
| | | CGUCGAGACCAUGCUCCCAAGCAAGAUCAUGCGGAAACUGGAGCCUGA |
| | | GGAGUUCGCUGCCUACCUGGAGCCAUUCAAGGAGAAGGGCGAGGUUA |
| | | GACGGCCUACCCUCUCCUGGCCUCGCGAGAUCCCUCUCGUUAAGGGAG |
| | | GCAAGCCCGACGUCGUCCAGAUUGUCCGCAACUACAACGCCUACCUUC |
| | | GGGCCAGCGACGAUCUGCCUAAGAUGUUCAUCGAGUCCGACCCUGGGU |
| | | UCUUUUCCAACGCUAUUGUCGAGGGAGCUAAGAAGUUCCCUAACACCG |
| | | AGUUCGUGAAGGUGAAGGGCCUCCACUUCAGCCAGGAGGACGCUCCAG |
| | | AUGAAAUGGGUAAGUACAUCAAGAGCUUCGUGGAGCGCGUGCUGAAG |
| | | AACGAGCAGUAAGCCAUACAAUAAAAGUGCGAAACGUUAUCCUAUAA |
| | | GUAAGAAAGUUUUAAAAUUUUCUUACGAAAAGGAUAGAACUUAAAAG |
| | | UUCUAACUGUUCUACUAAAGUAAUAAGUGAAAAUCUUAUUUAAAGCA |
| | | AACAACCAAGUAGCUUUAAGUCUAAGUCCCCUACACAAGUUUUAUACU |
| | | ACUAUGCAAAACUUGUGAAGCUAGGUAAGGUCGUAAUCCGUGAAAGU |
| | | CGGAUGCGGGGCUCCUUAAAAGAUUACUAUGGUAAACAUAAGCUAAU |
| | | CCAUUAAGAUGCGAUUUAUAUGUAUUUUAUACUGUUAAAUAUUUUUG |
| | | UGCUUGUGGCUUGGUAUAAAACAGUUAAGAUGAAGUACUUAACUGGU |
| | | UUUGGAAUAAUUGGUUGUUAAACUAAAACAUUAUAAAUCGUUAGUGG |
| | | AUACCUAAGGUAAUCAAAAAUAGGGAUAGGUAGAAUGGAACGUUUGA |
| | | UGCUGUAUAUGAAGAGGUUUAGUAGAACCUAGGACACAUAUACGGGC |
| | | UCAGCAGGUUCAUAGUAGCUAUGAUACUCAGCCGGAAGUCAAUUAAU |
| | | UUUGAAAUACUUCUAUGGUAACAUAGGAGAAGGAUAAAACUGAGUGA |
| | | GCCAAGGAACCUAGUCGGUAAUAGAAGCUAGAUCAUAUUACUAUUAA |
| | | GUAAGGUAUU |

*As to the sequence of SEQ ID NO: 2 (Group II intron Cte_original), the nucleotides from exon are represented by UPPER LETTERS. The nucleotides from exon and interacting with an EBS region are represented by UNDERLINED UPPER LETTERS. The six domains of the group II intron are represented by underlined lower letters. The EBS regions are indicated by italic bold underlined lower letters.

TABLE 2
Group II Intron Sequences

| SEQ ID | | |
|---|---|---|
| NO | Name | Sequence: |
| 33 | Group II | CUCUCUaaauagcaauauuuaccuuuggagggaaaaguuaucaggcaugcaccugguagcuaguc |
| | Intron (1) | uuuaaaccaauagauugcaucgguuuaaaaggcaagaccgucaaauugcgggaaaggggucaacagcc |
| | | guucaguaccaagucucaggggaaacuuugagauggccuugcaaaggguaugguaauaagcugacgg |
| | | acaugguccuaaccacgcagccaaguccuaagucaacagaucuucuguugauauggaugcaguucaca |
| | | gacuaaaugucggucggggaagauguauucuucucauaagauauagucggaccucuccuuaauggga |
| | | gcuagcggaugaagugaugcaacacuggagccgcugggaacuaauuuguaugcgaaaguauauugau |
| | | uaguuuuggaguacucgUAAGGUA |
| 34 | Group II | UGCAUGUGAUGCAGGUCGUGgugcgaaucguuccuaagugaaaagcuuaggcaucuu |
| | Intron (2) | agacaagggaaggccccauuaagauagaacuaaugauucgagagacagaaugauagaguaacgucuug |
| | | aaauguuuccccuaagucuccgacaugcauugaaagguaggauguuuaaaugugugaagcucgguga |
| | | agacggcugacagauaccguagugaaaaauguguuuuaaccgaaaguccuuuuaguaagaggaugua |
| | | ucccauaggccggggguccguaaaguaucuauggugagaauguuauaugaaauaacugacgaacuuu |
| | | cgaauuaacgagucuauaaccauacaaucagaaaugauaguaaaccauauuuuguguugugugugag |
| | | guuaaguaaauucgcggcuaugaacaaccuuacaguauuauagguacugucuagcacgcaggcucaua |
| | | gaaggcaccuaaggguaccauauaguggauagaaucauuggaacgaugaaagcucaggacgcagagaa |
| | | ccuacauucugugaagcggugguaaggaaaaggaggaauccuauaacuuaucuuuauuugagugaua |
| | | gcgaugucauaguagcguggaauuuauggaaacauaaaggagcgaagggcauuagucaauauuguga |
| | | ggaaaaagcaauauugauggcaagccguaugaagggaaacuuucauguacgguuuagugugggggaa |
| | | aaagcagagauuauaucaaaguuuuaccuaucacaauAAGGAGAGAAUAUUUUUAAU |
| 35 | Group II | CAAAGGCUUACCUAUCACUAgcgcgacacguuccuaagugaaaagcuuaggcacuguc |
| | Intron (3) | gaacucaacaguucagcagugaacugucauucuaagaagucaaaugaaggaguaacgucuggaagggc |
| | | uucccuuaauccuccgacaugcaggaaaguaggcaaguacugaacugugugaagcucggugaagucg |
| | | guugaagguuaccguaaauuaguaucucuaauacgaaagcuauccagcgguggaugguguaacugau |
| | | agaccggaggucuauaaaacacucaagguuaggaugcgcgaugaacuagaggcgaucgcuaguaagcg |
| | | cagacgaaucccugaugguacgggucuauaucgggagggaaucgaaagguucucugacacaaauaag |
| | | ugucgcuacugugggugaguaaaacucuccuuuaugaaagcccauauaucguuacaggcguuauuaa |
| | | gguagcaggcucauaggggaaaccuaaaaguguauguacagauaagaaugacggaacgugguaagcu |
| | | gccgacauggagggcuuguucucuuugaaguguugccaaggaaagucacaaugagauuaguugucga |
| | | uauaacuugguuuaacggcagugaaagugguggcacaguaccgaugaaacguguaaugaacguggag |
| | | ggauagccacuagucgauugaagauugaagguuacuauugguuaacaugguuucgaguaagacuaag |
| | | agauguaaugcuccaaaguaauaaggagguuacagcccauguuaaagaaaaccaagcuaagacauaac |
| | | gaauauuaugauacacaaaaaaaguguaugacaauuuauacucgaacagucuuaacgguaacaauuuc |
| | | uuucaauuggaaacgauggaacgccguaugcccggaaacgggcgcguacgguguggagugggggaaa |
| | | agcuggagauaaucucaaaggcuuaccuaucacuauCGCGACACGUUCCUAAGUGA |
| 36 | Group II | UUAUAACAAAGUAGUUAUUUgugcgacacguuucuuuauaagugugcaaacacgaag |
| | Intron Bth | uaggaggguuaucaauuugaugacaacaacggauugacugcaaguaggaaugaaagccgucagguug |
| | | agcugaaacuacuuaucugauacuccuauaugcaaggcgugauaguagucacaaauuguaugaagcua |
| | | ggugaagucggcugaacaaaaccuaagugagaaaucauaugguaauggauaggucgggaugcuacaa |
| | | aacaucuauggugagaauguccuaacggacuggcgaauguacagguuuaaaggauuaauucauuaga |
| | | aauguguauauugucaacgacgacgcuaucuaccgaaaaguaagaguaaauaauaugaaauucggaag |
| | | aucuaacgaugaggauguaaagauaacaggcuuacagcaagcaccuaaagauauauguauagcuaagu |
| | | cauucagaacgugguaagcaagagacugucacaaaugccuacuaacagacaaggugcauauaagguuc |
| | | uaacgaaccaaaauugcuuuauucuugugaagguggggacacaguaccgacgaagcauguaacaaaug |
| | | uggagggauagucccuagucuuguucguugaaaacuaaaucaacuggauauaacucacaggaucgag |
| | | uaagaugaugugacuuuucguaagaaaagggaaugaauacguuggugaguacaacaugguuuguaau |
| | | cuaguuguuuuaguauaaaaggauaagauggggcgcuguaugcgaugaaagucgcacguacaguguc |
| | | aagcgggggaaaagauggagauaacuuuaaagucuuaccuaucgcaacGGAUAAAAAGAAU |
| | | CCCUGCU |
| 37 | Group II | GAAUAUACUAUAGCCAUAcaauaaaagugcgaaacguuauccuauaaguaagaaaguuu |
| | Intron Cte | uaaaauuuucuuacgaaaaggauagaacuuaaaaguucuaacuguucuacuaaaguaauaagugaaaa |
| | | ucuuauuuaaagcaaacaaccaaguagcuuuaagucuaaguccccuacacaaguuuuauacuacuaug |
| | | caaaacuugugaagcuagguaaggucguaauccgugaaagucggaugcggggcuccuuaaaagauua |
| | | gugcuuguggcuugguauaaaacaguuaagaugaaguacuuaacugguuuuggaauaauugguugu |
| | | uaaacuaaaacauuauaaaucguuaguggauaccuaagguaaucaaaaauagggauagguagaaugga |
| | | acguuugaugcuguauaugaagagguuuaguagaaccuaggacacauauacgggcucagcagguuca |
| | | uaguagcuaugauacucagccggaagucaauuaauuuugaaauacuucuaugguaacauaggagaag |
| | | gauaaaacugagugagccaaggaaccuagucgguaauagaaaaguggaaguuaaaacaaauauaagau |
| | | uuuagaauuaauuuaauuaaugaacggaauuaauuuaaugauauuuaaaguuagacgguuauaaauu |
| | | aaacauuucaaaauuaaaccauauccaaauucauaaauauagcuagaucauaucacuaguuuaaaaaua |
| | | aauaaaucauuucaaauuacuauuaaguaagguauuaauaccuuacuuaauaguaaucucauuacaua |
| | | agagaauuacuagauuagcagacagauucauaaaaacuauaucaacuaggacaauagaaaauauauuu |
| | | auacacuuccuauuaucgagcgaacgccuuaugcgaugaaagucgcacguaggguguagaccaagcga |
| | | aauccuaugcauuuaggauagugagguauAGCAAAGGAGAA |
| 38 | Group II | ACUCUAGGUAGACGAGAgugcgacuaguaaagugcuuaauaacaauguggugaaagccc |
| | Intron (6) | accagauaacccauuaucugagcucagacgguaugcauuagaaguaauuuuuuguguaaagcccgu |
| | | uuaauuugguaaacagaccaaccaaccuauauauaaggauggugagcuauauuacuauggauaaaauu |
| | | uuuaugaauucacguucgaagcguauuagagugaguuuuauuuagaaggaaaaaaguaaauaaaauu |
| | | cuaauuaauuguauaaacaauuuuucguuuguuuuuaaugggucuuauauaauguacguauagu |
| | | gaaauccuaagguaguaaauaagguguuauuaaguaaacuagguaagcccaauaauaucuucauauga |
| | | uaguaugaagaaguucaaguguaaauuugaauauauauuaguggguaaaggauauuuuaaaaagcga |
| | | augucucauauuaauagugagaauagguuuaugacuaauucgaaagaaugcugacuuaaaauuaaua |
| | | uuaauauuucgauauuaauauuugagccguaugcgaugaaagucgcacguacgguucuuagaggggg |
| | | aaaguccaagagggccuaccuaucucaacA |
| 39 | Group II | CUCUAGAUAGACGAGAgugcgacuagauaaguacuuaauagcaauguggugaaagucca |
| | Intron (7) | ccugauaacccauagucugagcucagacgguaugcauuagaaguaauuuuuuguguaaagcccguu |
| | | uaaucuggucuaaaggaccuuccacccuaaauauauagggggagagcuauaauacuaaggauaaaauu |
| | | uuuuugaauucauguuuaaagcguauuagaggaaauuuauuucuaugaaaaagaagaaaaaauuuu |
| | | uaauuaacuguauaaaaaguuuucguuuuuuuuagugggucuuauguuaauguaguauagug |
| | | gaauccuaagguauuaauuaagguguuauuaaguaaacuagguaagcccaauagugucuucauauua |
| | | uaauaugaagaaguucaauugugaaauugaauaugcauuaguggguaaaggauauuuuaaaaagcga |
| | | augucucauauuaauagugagaauaggucuaugacuaauucgaaagaaugcugacuuaaaauuggua |
| | | uuaauauaugcguuucgauguauauauuaauauuugagccguaugcgaugaaagucgcacguacggu |
| | | ucuuagagggggaaaucuuaguaauaagacgaccuaucucgacA |
| 40 | Group II | GGGUAGCAAUAAGGAUgugcgacuuguuaaguuuuaacaaaaauuguauaacguuuauu |
| | Intron (8) | uauaaagcucuaauuauaaguguaaauacacuuuuaggcuucuucuaugguuagagaaaucgaaccaa |
| | | uguaauuaaaacuuugauguauuaggcauuuaacguguccuugguuaaaugaagaugaacauaagua |
| | | uacaaaguaaaauuggaaccuaaggaagaauuguuuuuguuaagaaacaagguaauaccuauaacugg |
| | | cuaauauaaauuugcaagguuuauuguaaaauaaacuauagguuagagguaaaaggauaauguaaaaa |
| | | gcgaaugcaauucuguaauggaauugauaggguauauaccuaacuugaaagagugcugacuuacaua |
| | | uagauguuauuuacguuucgacguaaauaauguuugagccguaugcuaugaaaguagcacguacggu |
| | | ucuaagagggggaaaguccgagaggaccuaccuaucucaacU |
| 41 | Group II | ACUCUAGGUAGACGAGAgugcgacuaguaaaguguuuaauaauaauguggugaaaaccc |
| | Intron (9) | accagauaacccauuaucugagcucauacggugugcauuaaaaguaauuuuuuuguauaaagcccgu |
| | | uuaaucagguaaauaauccuuccauccuaauuauaucuauagauauaaaaggauggugagcuauauua |
| | | cuauggauaagauuuuuuugaauucacguuugaagcguauuagaguaaguuuuauuuaaaaggaaaa |
| | | aaaaaaauuaaaaaaauuaaauuaauuguauaaaaaguuuucguuuuuuuuaaugggucuuau |
| | | auuaauguacguauagugaaauccuaauguaguaauuaagguguuauuaaauaaacuagguaagccc |
| | | aauaaugucuucauaauaugaagaaguucaaguguaaauuugaauauacauuaguggguaaaggaua |
| | | uuuuaaaaagcgaaugucucauauuaauagugagaauaggucuaugacuaauucgaaagaaugcuga |
| | | cuuaaaauuaauauuaauauuaauauauauauauuaauauuugagccguaugcgaugaaaaucgcacg |
| | | uacgguucuuagagggggaaagcucgagagggccuaccuaucuccacAGACCCUAUGCAG |
| | | CU |

TABLE 3
3' intron fragment

| SEQ | | |
|---|---|---|
| ID | | |
| NO: | Name | Sequence: |
| 42 | 3' intron | CUUUGAGAUGGCCUUGCAAAGGGUAUGGUAAUAAGCUGACGG |
| | fragment 1 | ACAUGGUCCUAACCACGCAGCCAAGUCCUAAGUCAACAGAUCU |
| | (obtained by | UCUGUUGAUAUGGAUGCAGUUCACAGACUAAAUGUCGGUCGGG |
| | segmenting a | GAAGAUGUAUUCUUCUCAUAAGAUAUAGUCGGACCUCUCCUUA |
| | group II intron | AUGGGAGCUAGCGGAUGAAGUGAUGCAACACUGGAGCCGCUGG |
| | at domain 4) | GAACUAAUUUGUAUGCGAAAGUAUAUUGAUUAGUUUUGGAGU |
| | | ACUCG |
| 43 | 3' intron | GCAAUAUUGAUGGCAAGCCGUAUGAAGGGAAACUUUCAUGUAC |
| | fragment 2 | GGUUUAGUGUGGGGGAAAAAGCAGAGAUUAUAUCAAAGUUUU |
| | (obtained by | ACCUAUCACAAU |
| | segmenting a | |
| | group II intron | |
| | at domain 4) | |
| 44 | 3' intron | AUUGGAAACGAUGGAACGCCGUAUGCCCGGAAACGGGCGCGUA |
| | fragment 3 | CGGUGUGGAGUGGGGGAAAAGCUGGAGAUAAUCUCAAAGGCU |
| | (obtained by | UACCUAUCACUAU |
| | segmenting a | |
| | group II intron | |
| | at domain 4) | |
| 45 | 3' intron | GGAUAAGAUGGGGCGCUGUAUGCGAUGAAAGUCGCACGUACAG |
| | fragment 4 | UGUCAAGCGGGGGAAAAGAUGGAGAUAACUUUAAAGUCUUAC |
| | (obtained by | CUAUCGCAAC |
| | segmenting a | |
| | group II intron | |
| | at domain 4) | |
| 46 | 3' intron | CUAUUAUCGAGCGAACGCCUUAUGCGAUGAAAGUCGCACGUAG |
| | fragment 5 | GGUGUAGACCAAGCGAAAUCCUAUGCAUUUAGGAUAGUGAGG |
| | (obtained by | UAU |
| | segmenting a | |
| | group II intron | |
| | at domain 4) | |
| 47 | 3' intron | AUAUUAAUAUUUGAGCCGUAUGCGAUGAAAGUCGCACGUACGG |
| | fragment 6 | UUCUUAGAGGGGGAAAGUCCAAGAGGGCCUACCUAUCUCAAC |
| | (obtained by | |
| | segmenting a | |
| | group II intron | |
| | at domain 4) | |
| 48 | 3' intron | AUGUAUAUAUUAAUAUUUGAGCCGUAUGCGAUGAAAGUCGCA |
| | fragment 7 | CGUACGGUUCUUAGAGGGGGAAAUCUUAGUAAUAAGACGACCU |
| | (obtained by | AUCUCGAC |
| | segmenting a | |
| | group II intron | |
| | at domain 4) | |
| 49 | 3' intron | ACGUAAAUAAUGUUUGAGCCGUAUGCUAUGAAAGUAGCACGU |
| | fragment 8 | ACGGUUCUAAGAGGGGGAAAGUCCGAGAGGACCUACCUAUCUC |
| | (obtained by | AAC |
| | segmenting a | |
| | group II intron | |
| | at domain 4) | |
| 50 | 3 intron | AUAUUAAUAUUUGAGCCGUAUGCGAUGAAAAUCGCACGUACGG |
| | fragment 9 | UUCUUAGAGGGGGAAAGCUCGAGAGGGCCUACCUAUCUCCAC |
| | (obtained by | |
| | segmenting a | |
| | group II intron | |
| | at domain 4) | |
| 51 | 3' intron | GAAUAAUUGGUUGUUAAACUAAAACAUUAUAAAUCGUUAGUG |
| | fragment 10 | GAUACCUAAGGUAAUCAAAAAUAGGGAUAGGUAGAAUGGAAC |
| | (obtained by | GUUUGAUGCUGUAUAUGAAGAGGUUUAGUAGAACCUAGGACA |
| | segmenting a | CAUAUACGGGCUCAGCAGGUUCAUAGUAGCUAUGAUACUCAGC |
| | group II intron | CGGAAGUCAAUUAAUUUUGAAAUACUUCUAUGGUAACAUAGG |
| | at domain 1) | AGAAGGAUAAAACUGAGUGAGCCAAGGAACCUAGUCGGUAAU |
| | | AGAAGCUAGAUCAUAUUACUAUUAAGUAAGGUAUUAAUACCU |
| | | UACUUAAUAGUAACAAUAGAAAAUCCUAUUAUCGAGCGAACGC |
| | | CUUAUGCGAUGAAAGUCGCACGUAGGGUGUAGACCAAGCGAAA |
| | | UCCUAUGCAUUUAGGAUAGUGAGGUAU |
| 52 | 3' intron | UACUUCUAUGGUAACAUAGGAGAAGGAUAAAACUGAGUGAGC |
| | fragment 11 | CAAGGAACCUAGUCGGUAAUAGAAGCUAGAUCAUAUUACUAUU |
| | (obtained by | AAGUAAGGUAUUAAUACCUUACUUAAUAGUAACAAUAGAAAA |
| | segmenting a | UCCUAUUAUCGAGCGAACGCCUUAUGCGAUGAAAGUCGCACGU |
| | group II intron | AGGGUGUAGACCAAGCGAAAUCCUAUGCAUUUAGGAUAGUGA |
| | at domain 3) | GGUAU |

TABLE 4
E2

| SEQ | |
|---|---|
| ID NO: | Sequence: |
| 53 | UAAGGUA |
| 54 | AAGGAG |
| 55 | AAGUGA |
| 56 | CCUGCU |
| 57 | AGCAGU |
| 58 | AGCAAA |
| 59 | AGAGAA |
| 60 | AGCAAA |
| 61 | A |
| 62 | U |
| 63 | AGACCC |

TABLE 5
E1

| SEQ | |
|---|---|
| ID NO: | Sequence: |
| 64 | CUCUCU |
| 65 | GUCGUG |
| 66 | CAAAGG |
| 67 | UUAUUU |
| 68 | CCAUGG |
| 69 | GCCCUG |
| 70 | CGUUGA |
| 71 | GCCAUA |
| 72 | ACGAGA |
| 73 | AAGGAU |
| 74 | AGACGAGA |

TABLE 6
5′ intron fragment

| SEQ | | |
|---|---|---|
| ID | | |
| NO: | Name | Sequence: |
| 75 | 5' intron | AAAUAGCAAUAUUUACCUUUGGAGGGAAAAGUUAUCAGGCAUGCA |
| | fragment 1 | CCUGGUAGCUAGUCUUUAAACCAAUAGAUUGCAUCGGUUUAAAAG |
| | (obtained by | GCAAGACCGUCAAAUUGCGGGAAAGGGGUCAACAGCCGUUCAGUAC |
| | segmenting | CAAGUCUCAGGG |
| | a group II | |
| | intron at | |
| | domain 4) | |
| 76 | 5' intron | GUGCGAAUCGUUCCUAAGUGAAAAGCUUAGGCAUCUUAGACAAGG |
| | fragment 2 | GAAGGCCCCAUUAAGAUAGAACUAAUGAUUCGAGAGACAGAAUGA |
| | (obtained by | UAGAGUAACGUCUUGAAAUGUUUCCCCUAAGUCUCCGACAUGCAUU |
| | segmenting | GAAAGGUAGGAUGUUUAAAUGUGUGAAGCUCGGUGAAGACGGCUG |
| | a group II | ACAGAUACCGUAGUGAAAAAUGUGUUUUAACCGAAAGUCCUUUUA |
| | intron at | GUAAGAGGAUGUAUCCCAUAGGCCGGGGGUCCGUAAAGUAUCUAU |
| | domain 4) | GGUGAGAAUGUUAUAUGAAAUAACUGACGAACUUUCGAAUUAACG |
| | | AGUCUAUAACCAUACAAUCAGAAAUGAUAGUAAACCAUAUUUUGU |
| | | GUUGUGUGUGAGGUUAAGUAAAUUCGCGGCUAUGAACAACCUUAC |
| | | AGUAUUAUAGGUACUGUCUAGCACGCAGGCUCAUAGAAGGCACCUA |
| | | AGGGUACCAUAUAGUGGAUAGAAUCAUUGGAACGAUGAAAGCUCA |
| | | GGACGCAGAGAACCUACAUUCUGUGAAGCGGUGGUAAGGAAAAGG |
| | | AGGAAUCCUAUAACUUAUCUUUAUUUGAGUGAUAGCGAUGUCAUA |
| | | GUAGCGUGGAAUUUAUGGAAACAUAAAGGAGCGAAGGGCAUUAGU |
| | | CAAUAUUGU |
| 77 | 5' intron | GCGCGACACGUUCCUAAGUGAAAAGCUUAGGCACUGUCGAACUCAA |
| | fragment 3 | CAGUUCAGCAGUGAACUGUCAUUCUAAGAAGUCAAAUGAAGGAGU |
| | (obtained by | AACGUCUGGAAGGGCUUCCCUUAAUCCUCCGACAUGCAGGAAAGUA |
| | segmenting | GGCAAGUACUGAACUGUGUGAAGCUCGGUGAAGUCGGUUGAAGGU |
| | a group II | UACCGUAAAUUAGUAUCUCUAAUACGAAAGCUAUCCAGCGGUGGA |
| | intron at | UGGUGUAACUGAUAGACCGGAGGUCUAUAAAACACUCAAGGUUAG |
| | domain 4) | GAUGCGCGAUGAACUAGAGGCGAUCGCUAGUAAGCGCAGACGAAUC |
| | | CCUGAUGGUACGGGUCUAUAUCGGGAGGGAAUCGAAAGGUUCUCU |
| | | GACACAAAUAAGUGUCGCUACUGUGGGUGAGUAAAACUCUCCUUU |
| | | AUGAAAGCCCAUAUAUCGUUACAGGCGUUAUUAAGGUAGCAGGCU |
| | | CAUAGGGGAAACCUAAAAGUGUAUGUACAGAUAAGAAUGACGGAA |
| | | CGUGGUAAGCUGCCGACAUGGAGGGCUUGUUCUCUUUGAAGUGUU |
| | | GCCAAGGAAAGUCACAAUGAGAUUAGUUGUCGAUAUAACUUGGUU |
| | | UAACGGCAGUGAAAGUGGUGGCACAGUACCGAUGAAACGUGUAAU |
| | | GAACGUGGAGGGAUAGCCACUAGUCGAUUGAAG |
| 78 | in intron | GUGCGACACGUUUCUUUAUAAGUGUGCAAACACGAAGUAGGAGGG |
| | fragment 4 | UUAUCAAUUUGAUGACAACAACGGAUUGACUGCAAGUAGGAAUGA |
| | (obtained by | AAGCCGUCAGGUUGAGCUGAAACUACUUAUCUGAUACUCCUAUAUG |
| | segmenting | CAAGGCGUGAUAGUAGUCACAAAUUGUAUGAAGCUAGGUGAAGUC |
| | a group II | GGCUGAACAAAACCUAAGUGAGAAAUCAUAUGGUAAUGGAUAGGU |
| | intron at | CGGGAUGCUACAAAACAUCUAUGGUGAGAAUGUCCUAACGGACUG |
| | domain 4) | GCGAAUGUACAGGUUUAAAGGAUUAAUUCAUUAGAAAUGUGUAUA |
| | | UUGUCAACGACGACGCUAUCUACCGAAAAGUAAGAGUAAAUAAUA |
| | | UGAAAUUCGGAAGAUCUAACGAUGAGGAUGUAAAGAUAACAGGCU |
| | | UACAGCAAGCACCUAAAGAUAUAUGUAUAGCUAAGUCAUUCAGAA |
| | | CGUGGUAAGCAAGAGACUGUCACAAAUGCCUACUAACAGACAAGGU |
| | | GCAUAUAAGGUUCUAACGAACCAAAAUUGCUUUAUUCUUGUGAAG |
| | | GUGGGGACACAGUACCGACGAAGCAUGUAACAAAUGUGGAGGGAU |
| | | AGUCCCUAGUCUUGUUC |
| 79 | 5' intron | CAAUAAAAGUGCGAAACGUUAUCCUAUAAGUAAGAAAGUUUUAAA |
| | fragment 5 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 4) | AGAUGCGAUUUAUAUGUAUUUUAUACUGUUAAAUAUUUUaaUGCUU |
| | | UaUGgUUGGUAUAAAACAGUUAAGAUGAAGUACUUAACUGGUUUUG |
| | | GAAUAAUUGGUUGUUAAACUAAAACAUUAUAAAUCGUUAGUGGAU |
| | | ACCUAAGGUAAUCAAAAAUAGGGAUAGGUAGAAUGGAACGUUUGA |
| | | UGCUGUAUAUGAAGAGGUUUAGUAGAACCUAGGACACAUAUACGG |
| | | GCUCAGCAGGUUCAUAGUAGCUAUGAUACUCAGCCGGAAGUCAAUU |
| | | AAUUUUGAAAUACUUCUAUGGUAACAUAGGAGAAGGAUAAAACUG |
| | | AGUGAGCCAAGGAACCUAGUCGGUAAUAG |
| 80 | 5' intron | CAAUAAAAGUGCGAAACGUUAUCCUAUAAGUAAGAAAGUUUUAAA |
| | fragment 6 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 4) | AGAUGCGAUUUAUAUGUAUUUUAUACUGUUAAAUAUUUUUGUGCU |
| | | UagGGCUUGGUAUAAAACAGUUAAGAUGAAGUACUUAACUGGUUUU |
| | | GGAAUAAUUGGUUGUUAAACUAAAACAUUAUAAAUCGUUAGUGGA |
| | | UACCUAAGGUAAUCAAAAAUAGGGAUAGGUAGAAUGGAACGUUUG |
| | | AUGCUGUAUAUGAAGAGGUUUAGUAGAACCUAGGACACAUAUACG |
| | | GGCUCAGCAGGUUCAUAGUAGCUAUGAUACUCAGCCGGAAGUCAAU |
| | | UAAUUUUGAAAUACUUCUAUGGUAACAUAGGAGAAGGAUAAAACU |
| | | GAGUGAGCCAAGGAACCUAGUCGGUAAUAG |
| 81 | 5' intron | CAAUAAAAGUGCGAAACGUUAUCCUAUAAGUAAGAAAGUUUUAAA |
| | fragment 7 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 4) | AGAUGCGAUUUAUAUGUAUUUUAUACUGUUAAAUAUUUUUGcUcUU |
| | | caacgUUGGUAUAAAACAGUUAAGAUGAAGUACUUAACUGGUUUUGG |
| | | AAUAAUUGGUUGUUAAACUAAAACAUUAUAAAUCGUUAGUGGAUA |
| | | CCUAAGGUAAUCAAAAAUAGGGAUAGGUAGAAUGGAACGUUUGAU |
| | | GCUGUAUAUGAAGAGGUUUAGUAGAACCUAGGACACAUAUACGGG |
| | | CUCAGCAGGUUCAUAGUAGCUAUGAUACUCAGCCGGAAGUCAAUUA |
| | | AUUUUGAAAUACUUCUAUGGUAACAUAGGAGAAGGAUAAAACUGA |
| | | GUGAGCCAAGGAACCUAGUCGGUAAUAG |
| 82 | 5' intron | CAAUAAAAGUGCGAAACGUUAUCCUAUAAGUAAGAAAGUUUUAAA |
| | fragment 8 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 4) | AGAUGCGAUUUAUAUGUAUUUUAUACUGUUAAAUAUUUUUGUGCU |
| | | UGUGGCUUGGUAUAAAACAGUUAAGAUGAAGUACUUAACUGGUUU |
| | | UGGAAUAAUUGGUUGUUAAACUAAAACAUUAUAAAUCGUUAGUGG |
| | | AUACCUAAGGUAAUCAAAAAUAGGGAUAGGUAGAAUGGAACGUUU |
| | | GAUGCUGUAUAUGAAGAGGUUUAGUAGAACCUAGGACACAUAUAC |
| | | GGGCUCAGCAGGUUCAUAGUAGCUAUGAUACUCAGCCGGAAGUCAA |
| | | UUAAUUUUGAAAUACUUCUAUGGUAACAUAGGAGAAGGAUAAAAC |
| | | UGAGUGAGCCAAGGAACCUAGUCGGUAAUAG |
| 83 | 5' intron | GUGCGACUAGUAAAGUGCUUAAUAACAAUGUGGUGAAAGCCCACC |
| | fragment 9 | AGAUAACCCAUUAUCUGAGCUCAGACGGUAUGCAUUAGAAGUAAU |
| | (obtained by | UCUUUUGUGUAAAGCCCGUUUAAUUUGGUAAACAGACCAACCAACC |
| | segmenting | UAUAUAUAAGGAUGGUGAGCUAUAUUACUAUGGAUAAAAUUUUUA |
| | a group II | UGAAUUCACGUUCGAAGCGUAUUAGAGUGAGUUUUAUUUAGAAGG |
| | intron at | AAAAAAGUAAAUAAAAUUCUAAUUAAUUGUAUAAACAAUUUUUCG |
| | domain 4) | UUUGUUUAUUAAUGGGUCUUAUAUUAAUGUACGUAUAGUGAAAUC |
| | | CUAAGGUAGUAAAUAAGGUGUUAUUAAGUAAACUAGGUAAGCCCA |
| | | AUAAUAUCUUCAUAUGAUAGUAUGAAGAAGUUCAAGUGUAAAUUU |
| | | GAAUAUAUAUUAGUGGGUAAAGGAUAUUUUAAAAAGCGAAUGUCU |
| | | CAUAUUAAUAGUGAGAAUAGGUUUAUGACUAAUUCGAAAGAAUGC |
| | | UGACUUAAAAUUAAUAUUAAUAU |
| 84 | 5' intron | GUGCGACUAGAUAAGUACUUAAUAGCAAUGUGGUGAAAGUCCACC |
| | fragment 10 | UGAUAACCCAUAGUCUGAGCUCAGACGGUAUGCAUUAGAAGUAAU |
| | (obtained by | UCUUUUGUGUAAAGCCCGUUUAAUCUGGUCUAAAGGACCUUCCACC |
| | segmenting | CUAAAUAUAUAGGGGGAGAGCUAUAAUACUAAGGAUAAAAUUUUU |
| | a group II | UUGAAUUCAUGUUUAAAGCGUAUUAGAGGAAAUUUAUUUCUAUGA |
| | intron at | AAAAGAAGAAAUAAAUUUUUAAUUAACUGUAUAAACAAGUUUUCG |
| | domain 4) | UUUGUUUAUUAGUGGGUCUUAUGUUAAUGUACGUAUAGUGGAAUC |
| | | CUAAGGUAUUAAUUAAGGUGUUAUUAAGUAAACUAGGUAAGCCCA |
| | | AUAGUGUCUUCAUAUUAUAAUAUGAAGAAGUUCAAUUGUGAAAUU |
| | | GAAUAUGCAUUAGUGGGUAAAGGAUAUUUUAAAAAGCGAAUGUCU |
| | | CAUAUUAAUAGUGAGAAUAGGUCUAUGACUAAUUCGAAAGAAUGC |
| | | UGACUUAAAAUUGGUAUUAAUAUAUGCGU |
| 85 | 5' intron | GUGCGACUUGUUAAGUUUUAACAAAAAUUGUAUAACGUUUAUUAA |
| | fragment 11 | UGAUUAUACAUUGUAUUUCAUCUUACAAUAGCCUAAUUAGAUAUG |
| | (obtained by | CAUUUAGGGUAACUUUUUUGUAUAAAGCUCUAAUUAUAAGUGUAA |
| | segmenting | AUACACUUUUAGGCUUCUUCUAUGGUUAGAGAAAUCGAACCAAUG |
| | a group II | UAAUUAAAACUUUGAUGUAUUAGGCAUUUAACGUGUCCUUGGUUA |
| | intron at | AAUGAAGAUGAACAUAAGUAUACAAAGUAAAAUUGGAACCUAAGG |
| | domain 4) | AAGAAUUGUUUUUGUUAAGAAACAAGGUAAUACCUAUAACUGGCU |
| | | AAUAUAAAUUUGCAAGGUUUAUUGUAAAAUAAACUAUAGGUUAGA |
| | | GGUAAAAGGAUAAUGUAAAAAGCGAAUGCAAUUCUGUAAUGGAAU |
| | | UGAUAGGGUAUAUACCUAACUUGAAAGAGUGCUGACUUACAUAUA |
| | | GAUGUUAUUUACGU |
| 86 | 5' intron | GUGCGACUAGUAAAGUGUUUAAUAAUAAUGUGGUGAAAACCCACC |
| | fragment 12 | AGAUAACCCAUUAUCUGAGCUCAUACGGUGUGCAUUAAAAGUAAU |
| | (obtained by | UUUUUUGUAUAAAGCCCGUUUAAUCAGGUAAAUAAUCCUUCCAUCC |
| | segmenting | UAAUUAUAUCUAUAGAUAUAAAAGGAUGGUGAGCUAUAUUACUAU |
| | a group II | GGAUAAGAUUUUUUUGAAUUCACGUUUGAAGCGUAUUAGAGUAAG |
| | intron at | UUUUAUUUAAAAGGAAAAAAAAAAAUUAAAUAAAAUUAAAUUAAU |
| | domain 4) | UGUAUAAAUAAGUUUUCGUUUAUUUAUUAAUGGGUCUUAUAUUAA |
| | | UGUACGUAUAGUGAAAUCCUAAUGUAGUAAUUAAGGUGUUAUUAA |
| | | AUAAACUAGGUAAGCCCAAUAAUGUCUUCAUAAUAUGAAGAAGUU |
| | | CAAGUGUAAAUUUGAAUAUACAUUAGUGGGUAAAGGAUAUUUUAA |
| | | AAAGCGAAUGUCUCAUAUUAAUAGUGAGAAUAGGUCUAUGACUAA |
| | | UUCGAAAGAAUGCUGACUUAAAAUUAAUAUUAAUAUUAAUAU |
| 87 | 5' intron | CAAUAAAAGUGCGAAACGUUAUCCUAUAAGUAAGAAAGUUUUAAA |
| | fragment 12 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 1) | AGAUGCGAUUUAUAUGUAUUUUAUACUGUUAAAUAUUUUUGUGCU |
| | | UGUGGCUUGGUAUAAAACAGUUAAGAUGAAGUACUUAACUGGUUU |
| | | UG |
| 88 | 5' intron | CAAUAAAAGUGCGAAACGUUAUCCUAUAAGUAAGAAAGUUUUAAA |
| | fragment 12 | AUUUUCUUACGAAAAGGAUAGAACUUAAAAGUUCUAACUGUUCUA |
| | (obtained by | CUAAAGUAAUAAGUGAAAAUCUUAUUUAAAGCAAACAACCAAGUA |
| | segmenting | GCUUUAAGUCUAAGUCCCCUACACAAGUUUUAUACUACUAUGCAAA |
| | a group II | ACUUGUGAAGCUAGGUAAGGUCGUAAUCCGUGAAAGUCGGAUGCG |
| | intron at | GGGCUCCUUAAAAGAUUACUAUGGUAAACAUAAGCUAAUCCAUUA |
| | domain 3) | AGAUGCGAUUUAUAUGUAUUUUAUACUGUUAAAUAUUUUUGUGCU |
| | | UGUGGCUUGGUAUAAAACAGUUAAGAUGAAGUACUUAACUGGUUU |
| | | UGGAAUAAUUGGUUGUUAAACUAAAACAUUAUAAAUCGUUAGUGG |
| | | AUACCUAAGGUAAUCAAAAAUAGGGAUAGGUAGAA |

TABLE 7
5' arm of target sequence

| SEQ | | |
|---|---|---|
| ID | | |
| NO: | Name | Sequence: |
| 89 | CV1 | GCAAUAGCCGAAAAACAAAAAACAAAAAAAACAAAA |
| | (5' arm of target sequence) | AAAAAACCAAAAAAACAAAACACA |
| 90 | CV2 | CGAAAGGAGGAGGAGGAGGAAAAAAAGGAGGAGGAG |
| | (5' arm of target sequence) | GAAACCAAAAAAACAAAACACA |
| 91 | CV3 | GCAAUGGAGGAGGAGGAGGAAAAAAAGGAGGAGGAG |
| | (5' arm of target sequence) | GAAGCCGAAAAACAAAAAACAAAAAAAACAAAAAAA |
| | | AAACCAAAAAAACAAAACACA |
| 92 | CV4 | GCAAUCCUCCUCCUCCUAAAAAACCUCCUCCUCCUCC |
| | (5' arm of target sequence) | UAGCCGAAAAACAAAAAACAAAAAAAACAAAAAAAA |
| | | AACCAAAAAAACAAAACACA |
| 93 | CV5 | CGAAAGGAGGAGGAGGAGGAACCAAAAAAACAAAAC |
| | (5' arm of target sequence) | ACA |
| 94 | CV6 | CGAAACCUCCUCCUCCUCCUAACCAAAAAAACAAAAC |
| | (5' arm of target sequence) | ACA |
| 95 | CV7 | CGAAACCUCCUCCUCCUAAAAAACCUCCUCCUCCUCC |
| | (5' arm of target sequence) | UAACCAAAAAAACAAAACACA |
| 96 | CV8 | CGAAACCUCCUCCUCCUAAAAAACCUCCUCCUCCUCC |
| | (5' arm of target sequence) | UAACCAAAAAAACAAAACACA |

TABLE 8
3' arm of target sequence

| SEQ | | |
|---|---|---|
| ID NO: | Name | Sequence: |
| 97 | CV1 | AAAAAACAAAAAACAAAAC |
| | (3' arm) | |
| 98 | CV2 | AAAAAACAAAAAACACCUCCUCCUCCUAAAAAACCUCCUCCUCCUCCU |
| | (3' arm) | AAAA |
| 99 | CV3 | AAAAAACAAAAAACACCUCCUCCUCCUAAAAAACCUCCUCCUCCUCCU |
| | (3' arm) | AAAC |
| 100 | CV4 | AAAAAACAAAAAACAGGAGGAGGAGGAGGAAAAAAAGGAGGAGGAGG |
| | (3' arm) | AAAAC |
| 101 | CV5 | AAAAAACAAAAAACACCUCCUCCUCCUCCUAAAA |
| | (3' arm) | |
| 102 | CV6 | AAAAAACAAAAAACAGGAGGAGGAGGAGGAAAA |
| | (3' arm) | |
| 103 | CV7 | AAAAAACAAAAAACAGGAGGAGGAGGAGGAAAAAAAGGAGGAGGAGG |
| | (3' arm) | AAAA |
| 104 | CV8 | AUUAGAGACAAUUUGAAAUAAUUUAGAUUGGCUUAACCCUACUGUGC |
| | (3' arm) | UAACCGAACCAGAUAACGGUACAGUAGGGGUAAAUUCUCCGCAUUCG |
| | | GUGCGG |

TABLE 9
homology arm sequence

| SEQ | | |
|---|---|---|
| ID NO: | Name | Sequence: |
| 105 | 5' homology arm | AAUACCUUAC |
| | sequence | UUAAUAGUAA |
| 106 | 3' homology arm | UUACUAUUAA |
| | sequence | GUAAGGUAUU |

TABLE 10
target sequence

| SEQ | | |
|---|---|---|
| ID NO: | Name | Sequence: |
| 107 | Gluc | AUGGGAGUCAAAGUUCUGUUUGCCCUGAUCUGCAUCGCUGUGGCCG |
| | | AGGCCAAGCCCACCGAGAACAACGAAGACUUCAACAUCGUGGCCGU |
| | | GGCCAGCAACUUCGCGACCACGGAUCUCGAUGCUGACCGCGGGAAG |
| | | UUGCCCGGCAAGAAGCUGCCGCUGGAGGUGCUCAAAGAGAUGGAA |
| | | GCCAAUGCCCGGAAAGCUGGCUGCACCAGGGGCUGUCUGAUCUGCC |
| | | UGUCCCACAUCAAGUGCACGCCCAAGAUGAAGAAGUUCAUCCCAGG |
| | | ACGCUGCCACACCUACGAAGGCGACAAAGAGUCCGCACAGGGCGGC |
| | | AUAGGCGAGGCGAUCGUCGACAUUCCUGAGAUUCCUGGGUUCAAG |
| | | GACUUGGAGCCCAUGGAGCAGUUCAUCGCACAGGUCGAUCUGUGUG |
| | | UGGACUGCACAACUGGCUGCCUCAAAGGGCUUGCCAACGUGCAGUG |
| | | UUCUGACCUGCUCAAGAAGUGGCUGCCGCAACGCUGUGCGACCUUU |
| | | GCCAGCAAGAUCCAGGGCCAGGUGGACAAGAUCAAGGGGGCCGGUG |
| | | GUGACUAG |
| 108 | EGFP | AUGGUGAGCAAGGGCGAGGAGCUGUUCACCGGGGUGGUGCCCAUCC |
| | | UGGUCGAGCUGGACGGCGACGUAAACGGCCACAAGUUCAGCGUGUC |
| | | CGGCGAGGGCGAGGGCGAUGCCACCUACGGCAAGCUGACCCUGAAG |
| | | UUCAUCUGCACCACCGGCAAGCUGCCCGUGCCCUGGCCCACCCUCG |
| | | UGACCACCCUGACCUACGGCGUGCAGUGCUUCAGCCGCUACCCCGA |
| | | CCACAUGAAGCAGCACGACUUCUUCAAGUCCGCCAUGCCCGAAGGC |
| | | UACGUCCAGGAGCGCACCAUCUUCUUCAAGGACGACGGCAACUACA |
| | | AGACCCGCGCCGAGGUGAAGUUCGAGGGCGACACCCUGGUGAACCG |
| | | CAUCGAGCUGAAGGGCAUCGACUUCAAGGAGGACGGCAACAUCCUG |
| | | GGGCACAAGCUGGAGUACAACUACAACAGCCACAACGUCUAUAUCA |
| | | UGGCCGACAAGCAGAAGAACGGCAUCAAGGUGAACUUCAAGAUCCG |
| | | CCACAACAUCGAGGACGGCAGCGUGCAGCUCGCCGACCACUACCAG |
| | | CAGAACACCCCCAUCGGCGACGGCCCCGUGCUGCUGCCCGACAACC |
| | | ACUACCUGAGCACCCAGUCCGCCCUGAGCAAAGACCCCAACGAGAA |
| | | GCGCGAUCACAUGGUCCUGCUGGAGUUCGUGACCGCCGCCGGGAUC |
| | | ACUCUCGGCAUGGACGAGCUGUACAAGUAG |
| 109 | Rluc | AUGGCUUCCAAGGUGUACGACCCCGAGCAACGCAAACGCAUGAUCA |
| | | CUGGGCCUCAGUGGUGGGCUCGCUGCAAGCAAAUGAACGUGCUGGA |
| | | CUCCUUCAUCAACUACUAUGAUUCCGAGAAGCACGCCGAGAACGCC |
| | | GUGAUUUUUCUGCAUGGUAACGCUGCCUCCAGCUACCUGUGGAGGC |
| | | ACGUCGUGCCUCACAUCGAGCCCGUGGCUAGAUGCAUCAUCCCUGA |
| | | UCUGAUCGGAAUGGGUAAGUCCGGCAAGAGCGGGAAUGGCUCAUA |
| | | UCGCCUCCUGGAUCACUACAAGUACCUCACCGCUUGGUUCGAGCUG |
| | | CUGAACCUUCCAAAGAAAAUCAUCUUUGUGGGCCACGACUGGGGGG |
| | | CUUGUCUGGCCUUUCACUACUCCUACGAGCACCAAGACAAGAUCAA |
| | | GGCCAUCGUCCAUGCUGAGAGUGUCGUGGACGUGAUCGAGUCCUGG |
| | | GACGAGUGGCCUGACAUCGAGGAGGAUAUCGCCCUGAUCAAGAGCG |
| | | AAGAGGGCGAGAAAAUGGUGCUUGAGAAUAACUUCUUCGUCGAGA |
| | | CCAUGCUCCCAAGCAAGAUCAUGCGGAAACUGGAGCCUGAGGAGUU |
| | | CGCUGCCUACCUGGAGCCAUUCAAGGAGAAGGGCGAGGUUAGACGG |
| | | CCUACCCUCUCCUGGCCUCGCGAGAUCCCUCUCGUUAAGGGAGGCA |
| | | AGCCCGACGUCGUCCAGAUUGUCCGCAACUACAACGCCUACCUUCG |
| | | GGCCAGCGACGAUCUGCCUAAGAUGUUCAUCGAGUCCGACCCUGGG |
| | | UUCUUUUCCAACGCUAUUGUCGAGGGAGCUAAGAAGUUCCCUAACA |
| | | CCGAGUUCGUGAAGGUGAAGGGCCUCCACUUCAGCCAGGAGGACGC |
| | | UCCAGAUGAAAUGGGUAAGUACAUCAAGAGCUUCGUGGAGCGCGU |
| | | GCUGAAGAACGAGCAGUAA |
| 110 | Fluc | AUGGCCGAUGCUAAGAACAUUAAGAAGGGCCCUGCUCCCUUCUACC |
| | | CUCUGGAGGAUGGCACCGCUGGCGAGCAGCUGCACAAGGCCAUGAA |
| | | GAGGUAUGCCCUGGUGCCUGGCACCAUUGCCUUCACCGAUGCCCAC |
| | | AUUGAGGUGGACAUCACCUAUGCCGAGUACUUCGAGAUGUCUGUG |
| | | CGCCUGGCCGAGGCCAUGAAGAGGUACGGCCUGAACACCAACCACC |
| | | GCAUCGUGGUGUGCUCUGAGAACUCUCUGCAGUUCUUCAUGCCAGU |
| | | GCUGGGCGCCCUGUUCAUCGGAGUGGCCGUGGCCCCUGCUAACGAC |
| | | AUUUACAACGAGCGCGAGCUGCUGAACAGCAUGGGCAUUUCUCAGC |
| | | CUACCGUGGUGUUCGUGUCUAAGAAGGGCCUGCAGAAGAUCCUGA |
| | | ACGUGCAGAAGAAGCUGCCUAUCAUCCAGAAGAUCAUCAUCAUGGA |
| | | CUCUAAGACCGACUACCAGGGCUUCCAGAGCAUGUACACAUUCGUG |
| | | ACAUCUCAUCUGCCUCCUGGCUUCAACGAGUACGACUUCGUGCCAG |
| | | AGUCUUUCGACAGGGACAAAACCAUUGCCCUGAUCAUGAACAGCUC |
| | | UGGGUCUACCGGCCUGCCUAAGGGCGUGGCCCUGCCUCAUCGCACC |
| | | GCCUGUGUGCGCUUCUCUCACGCCCGCGACCCUAUUUUCGGCAACC |
| | | AGAUCAUCCCCGACACCGCUAUUCUGAGCGUGGUGCCAUUCCACCA |
| | | CGGCUUCGGCAUGUUCACCACCCUGGGCUACCUGAUUUGCGGCUUU |
| | | CGGGUGGUGCUGAUGUACCGCUUCGAGGAGGAGCUGUUCCUGCGCA |
| | | GCCUGCAAGACUACAAAAUUCAGUCUGCCCUGCUGGUGCCAACCCU |
| | | GUUCAGCUUCUUCGCUAAGAGCACCCUGAUCGACAAGUACGACCUG |
| | | UCUAACCUGCACGAGAUUGCCUCUGGCGGCGCCCCACUGUCUAAGG |
| | | AGGUGGGCGAAGCCGUGGCCAAGCGCUUUCAUCUGCCAGGCAUCCG |
| | | CCAGGGCUACGGCCUGACCGAGACAACCAGCGCCAUUCUGAUUACC |
| | | CCAGAGGGCGACGACAAGCCUGGCGCCGUGGGCAAGGUGGUGCCAU |
| | | UCUUCGAGGCCAAGGUGGUGGACCUGGACACCGGCAAGACCCUGGG |
| | | AGUGAACCAGCGCGGCGAGCUGUGUGUGCGCGGCCCUAUGAUUAUG |
| | | UCCGGCUACGUGAAUAACCCUGAGGCCACAAACGCCCUGAUCGACA |
| | | AGGACGGCUGGCUGCACUCUGGCGACAUUGCCUACUGGGACGAGGA |
| | | CGAGCACUUCUUCAUCGUGGACCGCCUGAAGUCUCUGAUCAAGUAC |
| | | AAGGGCUACCAGGUGGCCCCAGCCGAGCUGGAGUCUAUCCUGCUGC |
| | | AGCACCCUAACAUUUUCGACGCCGGAGUGGCCGGCCUGCCCGACGA |
| | | CGAUGCCGGCGAGCUGCCUGCCGCCGUCGUCGUGCUGGAACACGGC |
| | | AAGACCAUGACCGAGAAGGAGAUCGUGGACUAUGUGGCCAGCCAG |
| | | GUGACAACCGCCAAGAAGCUGCGCGGCGGAGUGGUGUUCGUGGACG |
| | | AGGUGCCCAAGGGCCUGACCGGCAAGCUGGACGCCCGCAAGAUCCG |
| | | CGAGAUCCUGAUCAAGGCUAAGAAAGGCGGCAAGAUCGCCGUGUA |
| | | A |
| 111 | RBD | AUGGACGCCAUGAAACGGGGACUGUGCUGCGUGCUGCUGCUGUGU |
| | | GGCGCCGUGUUCGUGUCACCUAGCCGGGUGCAGCCUACCGAGAGCA |
| | | UCGUGCGGUUCCCUAACAUCACAAACCUGUGUCCAUUCGGCGAGGU |
| | | GUUCAACGCCACCAGAUUCGCCAGCGUGUACGCUUGGAAUAGAAAA |
| | | AGAAUCUCUAAUUGCGUGGCCGAUUACAGCGUGCUGUACAACAGCG |
| | | CCUCCUUCAGCACCUUCAAGUGCUACGGCGUGUCCCCCACCAAGCU |
| | | GAACGACCUGUGCUUCACAAAUGUCUACGCCGAUAGCUUCGUGAUU |
| | | AGAGGCGACGAGGUGAGGCAGAUCGCUCCAGGCCAGACCGGCAAGA |
| | | UCGCUGAUUACAACUACAAGCUGCCUGAUGACUUCACAGGAUGUGU |
| | | GAUCGCCUGGAACAGCAACAACCUCGACAGCAAGGUGGGAGGCAAC |
| | | UACAAUUACCUGUAUAGACUGUUCAGAAAGUCCAACCUGAAGCCCU |
| | | UCGAGAGAGACAUCAGCACCGAAAUCUACCAGGCCGGCUCCACCCC |
| | | UUGCAACGGAGUGGAAGGCUUCAACUGCUACUUCCCCCUGCAGAGC |
| | | UACGGUUUUCAGCCUACCAACGGCGUGGGCUACCAGCCCUACCGCG |
| | | UGGUUGUGCUGAGCUUCGAGCUGCUGCACGCCCCAGCUACAGUGUG |
| | | CGGCCCUAAGAAAUCUACCAACCUGGUGAAGAACAAGGGCUAUAUC |
| | | CCCGAGGCCCCUAGAGACGGCCAAGCCUACGUGCGGAAGGACGGCG |
| | | AAUGGGUCCUGCUCAGCACAUUCCUGGGCAGCUGA |
| 112 | saCAS9 | AUGGCCCCAAAGAAGAAGCGGAAGGUCGGUAUCCACGGAGUCCCAG |
| | | CAGCCAAGCGGAACUACAUCCUGGGCCUGGACAUCGGCAUCACCAG |
| | | CGUGGGCUACGGCAUCAUCGACUACGAGACACGGGACGUGAUCGAU |
| | | GCCGGCGUGCGGCUGUUCAAAGAGGCCAACGUGGAAAACAACGAGG |
| | | GCAGGCGGAGCAAGAGAGGCGCCAGAAGGCUGAAGCGGCGGAGGC |
| | | GGCAUAGAAUCCAGAGAGUGAAGAAGCUGCUGUUCGACUACAACC |
| | | UGCUGACCGACCACAGCGAGCUGAGCGGCAUCAACCCCUACGAGGC |
| | | CAGAGUGAAGGGCCUGAGCCAGAAGCUGAGCGAGGAAGAGUUCUC |
| | | UGCCGCCCUGCUGCACCUGGCCAAGAGAAGAGGCGUGCACAACGUG |
| | | AACGAGGUGGAAGAGGACACCGGCAACGAGCUGUCCACCAAAGAGC |
| | | AGAUCAGCCGGAACAGCAAGGCCCUGGAAGAGAAAUACGUGGCCGA |
| | | ACUGCAGCUGGAACGGCUGAAGAAAGACGGCGAAGUGCGGGGCAG |
| | | CAUCAACAGAUUCAAGACCAGCGACUACGUGAAAGAAGCCAAACAG |
| | | CUGCUGAAGGUGCAGAAGGCCUACCACCAGCUGGACCAGAGCUUCA |
| | | UCGACACCUACAUCGACCUGCUGGAAACCCGGCGGACCUACUAUGA |
| | | GGGACCUGGCGAGGGCAGCCCCUUCGGCUGGAAGGACAUCAAAGAA |
| | | UGGUACGAGAUGCUGAUGGGCCACUGCACCUACUUCCCCGAGGAAC |
| | | UGCGGAGCGUGAAGUACGCCUACAACGCCGACCUGUACAACGCCCU |
| | | GAACGACCUGAACAAUCUCGUGAUCACCAGGGACGAGAACGAGAAG |
| | | CUGGAAUAUUACGAGAAGUUCCAGAUCAUCGAGAACGUGUUCAAG |
| | | CAGAAGAAGAAGCCCACCCUGAAGCAGAUCGCCAAAGAAAUCCUCG |
| | | UGAACGAAGAGGAUAUUAAGGGCUACAGAGUGACCAGCACCGGCA |
| | | AGCCCGAGUUCACCAACCUGAAGGUGUACCACGACAUCAAGGACAU |
| | | UACCGCCCGGAAAGAGAUUAUUGAGAACGCCGAGCUGCUGGAUCAG |
| | | AUUGCCAAGAUCCUGACCAUCUACCAGAGCAGCGAGGACAUCCAGG |
| | | AAGAACUGACCAAUCUGAACUCCGAGCUGACCCAGGAAGAGAUCGA |
| | | GCAGAUCUCUAAUCUGAAGGGCUAUACCGGCACCCACAACCUGAGC |
| | | CUGAAGGCCAUCAACCUGAUCCUGGACGAGCUGUGGCACACCAACG |
| | | ACAACCAGAUCGCUAUCUUCAACCGGCUGAAGCUGGUGCCCAAGAA |
| | | GGUGGACCUGUCCCAGCAGAAAGAGAUCCCCACCACCCUGGUGGAC |
| | | GACUUCAUCCUGAGCCCCGUCGUGAAGAGAAGCUUCAUCCAGAGCA |
| | | UCAAAGUGAUCAACGCCAUCAUCAAGAAGUACGGCCUGCCCAACGA |
| | | CAUCAUUAUCGAGCUGGCCCGCGAGAAGAACUCCAAGGACGCCCAG |
| | | AAAAUGAUCAACGAGAUGCAGAAGCGGAACCGGCAGACCAACGAGC |
| | | GGAUCGAGGAAAUCAUCCGGACCACCGGCAAAGAGAACGCCAAGUA |
| | | CCUGAUCGAGAAGAUCAAGCUGCACGACAUGCAGGAAGGCAAGUGC |
| | | CUGUACAGCCUGGAAGCCAUCCCUCUGGAAGAUCUGCUGAACAACC |
| | | CCUUCAACUAUGAGGUGGACCACAUCAUCCCCAGAAGCGUGUCCUU |
| | | CGACAACAGCUUCAACAACAAGGUGCUCGUGAAGCAGGAAGAAAAC |
| | | AGCAAGAAGGGCAACCGGACCCCAUUCCAGUACCUGAGCAGCAGCG |
| | | ACAGCAAGAUCAGCUACGAAACCUUCAAGAAGCACAUCCUGAAUCU |
| | | GGCCAAGGGCAAGGGCAGAAUCAGCAAGACCAAGAAAGAGUAUCU |
| | | GCUGGAAGAACGGGACAUCAACAGGUUCUCCGUGCAGAAAGACUUC |
| | | AUCAACCGGAACCUGGUGGAUACCAGAUACGCCACCAGAGGCCUGA |
| | | UGAACCUGCUGCGGAGCUACUUCAGAGUGAACAACCUGGACGUGAA |
| | | AGUGAAGUCCAUCAAUGGCGGCUUCACCAGCUUUCUGCGGCGGAAG |
| | | UGGAAGUUUAAGAAAGAGCGGAACAAGGGGUACAAGCACCACGCC |
| | | GAGGACGCCCUGAUCAUUGCCAACGCCGAUUUCAUCUUCAAAGAGU |
| | | GGAAGAAACUGGACAAGGCCAAAAAAGUGAUGGAAAACCAGAUGU |
| | | UCGAGGAAAAGCAGGCCGAGAGCAUGCCCGAGAUCGAAACCGAGCA |
| | | GGAGUACAAAGAGAUCUUCAUCACCCCCCACCAGAUCAAGCACAUU |
| | | AAGGACUUCAAGGACUACAAGUACAGCCACCGGGUGGACAAGAAGC |
| | | CUAAUAGAGAGCUGAUUAACGACACCCUGUACUCCACCCGGAAGGA |
| | | CGACAAGGGCAACACCCUGAUCGUGAACAAUCUGAACGGCCUGUAC |
| | | GACAAGGACAAUGACAAGCUGAAAAAGCUGAUCAACAAGAGCCCCG |
| | | AAAAGCUGCUGAUGUACCACCACGACCCCCAGACCUACCAGAAACU |
| | | GAAGCUGAUUAUGGAACAGUACGGCGACGAGAAGAAUCCCCUGUA |
| | | CAAGUACUACGAGGAAACCGGGAACUACCUGACCAAGUACUCCAAA |
| | | AAGGACAACGGCCCCGUGAUCAAGAAGAUUAAGUAUUACGGCAAC |
| | | AAACUGAACGCCCAUCUGGACAUCACCGACGACUACCCCAACAGCA |
| | | GAAACAAGGUCGUGAAGCUGUCCCUGAAGCCCUACAGAUUCGACGU |
| | | GUACCUGGACAAUGGCGUGUACAAGUUCGUGACCGUGAAGAAUCU |
| | | GGAUGUGAUCAAAAAAGAAAACUACUACGAAGUGAAUAGCAAGUG |
| | | CUAUGAGGAAGCUAAGAAGCUGAAGAAGAUCAGCAACCAGGCCGA |
| | | GUUUAUCGCCUCCUUCUACAACAACGAUCUGAUCAAGAUCAACGGC |
| | | GAGCUGUAUAGAGUGAUCGGCGUGAACAACGACCUGCUGAACCGG |
| | | AUCGAAGUGAACAUGAUCGACAUCACCUACCGCGAGUACCUGGAAA |
| | | ACAUGAACGACAAGAGGCCCCCCAGGAUCAUUAAGACAAUCGCCUC |
| | | CAAGACCCAGAGCAUUAAGAAGUACAGCACAGACAUUCUGGGCAAC |
| | | CUGUAUGAAGUGAAAUCUAAGAAGCACCCUCAGAUCAUCAAAAAG |
| | | GGCAAAAGGCCGGCGGCCACGAAAAAGGCCGGCCAGGCAAAAAAGA |
| | | AAAAGGGAUCCUACCCAUACGAUGUUCCAGAUUACGCUUACCCAUA |
| | | CGAUGUUCCAGAUUACGCUUACCCAUACGAUGUUCCAGAUUACGCU |
| | | UAA |

TABLE 11
amino acid sequence

| SEQ | | |
|---|---|---|
| ID NO: | Name | Sequence: |
| 113 | Gluc | MGVKVLFALICIAVAEAKPTENNEDFNIVAVASNFATTDLDADRGKLPGK |
| | | KLPLEVLKEMEANARKAGCTRGCLICLSHIKCTPKMKKFIPGRCHTYEG |
| | | DKESAQGGIGEAIVDIPEIPGFKDLEPMEQFIAQVDLCVDCTTGCLKGLA |
| | | NVQCSDLLKKWLPQRCATFASKIQGQVDKIKGAGGD |
| 114 | EGFP | MVSKGEELFTGVVPILVELDGDVNGHKFSVSGEGEGDATYGKLTLKFICT |
| | | TGKLPVPWPTLVTTLTYGVQCFSRYPDHMKQHDFFKSAMPEGYVQERTI |
| | | FFKDDGNYKTRAEVKFEGDTLVNRIELKGIDFKEDGNILGHKLEYNYNS |
| | | HNVYIMADKQKNGIKVNFKIRHNIEDGSVQLADHYQQNTPIGDGPVLLP |
| | | DNHYLSTQSALSKDPNEKRDHMVLLEFVTAAGITLGMDELYK |
| 115 | Rluc | MASKVYDPEQRKRMITGPQWWARCKQMNVLDSFINYYDSEKHAENAVI |
| | | FLHGNAASSYLWRHVVPHIEPVARCIIPDLIGMGKSGKSGNGSYRLLDHY |
| | | KYLTAWFELLNLPKKIIFVGHDWGACLAFHYSYEHQDKIKAIVHAESVV |
| | | DVIESWDEWPDIEEDIALIKSEEGEKMVLENNFFVETMLPSKIMRKLEPEE |
| | | FAAYLEPFKEKGEVRRPTLSWPREIPLVKGGKPDVVQIVRNYNAYLRASD |
| | | DLPKMFIESDPGFFSNAIVEGAKKFPNTEFVKVKGLHFSQEDAPDEMGKY |
| | | IKSFVERVLKNEQ |
| 116 | Fluc | MADAKNIKKGPAPFYPLEDGTAGEQLHKAMKRYALVPGTIAFTDAHIEV |
| | | DITYAEYFEMSVRLAEAMKRYGLNTNHRIVVCSENSLQFFMPVLGALFIG |
| | | VAVAPANDIYNERELLNSMGISQPTVVFVSKKGLQKILNVQKKLPIIQKIII |
| | | MDSKTDYQGFQSMYTFVTSHLPPGFNEYDFVPESFDRDKTIALIMNSSGS |
| | | TGLPKGVALPHRTACVRFSHARDPIFGNQIIPDTAILSVVPFHHGFGMFTTL |
| | | GYLICGFRVVLMYRFEEELFLRSLQDYKIQSALLVPTLFSFFAKSTLIDKY |
| | | DLSNLHEIASGGAPLSKEVGEAVAKRFHLPGIRQGYGLTETTSAILITPEGD |
| | | DKPGAVGKVVPFFEAKVVDLDTGKTLGVNQRGELCVRGPMIMSGYVNN |
| | | PEATNALIDKDGWLHSGDIAYWDEDEHFFIVDRLKSLIKYKGYQVAPAEL |
| | | ESILLQHPNIFDAGVAGLPDDDAGELPAAVVVLEHGKTMTEKEIVDYVAS |
| | | QVTTAKKLRGGVVFVDEVPKGLTGKLDARKIREILIKAKKGGKIAV |
| 117 | RBD | MDAMKRGLCCVLLLCGAVFVSPSRVQPTESIVRFPNITNLCPFGEVFNAT |
| | | RFASVYAWNRKRISNCVADYSVLYNSASFSTFKCYGVSPTKLNDLCFTNV |
| | | YADSFVIRGDEVRQIAPGQTGKIADYNYKLPDDFTGCVIAWNSNNLDSK |
| | | VGGNYNYLYRLFRKSNLKPFERDISTEIYQAGSTPCNGVEGENCYFPLQS |
| | | YGFQPTNGVGYQPYRVVVLSFELLHAPATVCGPKKSTNLVKNKGYIPEAP |
| | | RDGQAYVRKDGEWVLLSTFLGS |
| 118 | saCAS9 | MAPKKKRKVGIHGVPAAKRNYILGLDIGITSVGYGIIDYETRDVIDAGVR |
| | | LFKEANVENNEGRRSKRGARRLKRRRRHRIQRVKKLLFDYNLLTDHSEL |
| | | SGINPYEARVKGLSQKLSEEEFSAALLHLAKRRGVHNVNEVEEDTGNELS |
| | | TKEQISRNSKALEEKYVAELQLERLKKDGEVRGSINRFKTSDYVKEAKQL |
| | | LKVQKAYHQLDQSFIDTYIDLLETRRTYYEGPGEGSPFGWKDIKEWYEM |
| | | LMGHCTYFPEELRSVKYAYNADLYNALNDLNNLVITRDENEKLEYYEKF |
| | | QIIENVFKQKKKPTLKQIAKEILVNEEDIKGYRVTSTGKPEFTNLKVYHDI |
| | | KDITARKEIIENAELLDQIAKILTIYQSSEDIQEELTNLNSELTQEEIEQISNL |
| | | KGYTGTHNLSLKAINLILDELWHTNDNQIAIFNRLKLVPKKVDLSQQKEIP |
| | | TTLVDDFILSPVVKRSFIQSIKVINAIIKKYGLPNDIIIELAREKNSKDAQKM |
| | | INEMQKRNRQTNERIEEIIRTTGKENAKYLIEKIKLHDMQEGKCLYSLEAI |
| | | PLEDLLNNPFNYEVDHIIPRSVSFDNSFNNKVLVKQEENSKKGNRTPFQY |
| | | LSSSDSKISYETFKKHILNLAKGKGRISKTKKEYLLEERDINRFSVQKDFIN |
| | | RNLVDTRYATRGLMNLLRSYFRVNNLDVKVKSINGGFTSFLRRKWKFKK |
| | | ERNKGYKHHAEDALIIANADFIFKEWKKLDKAKKVMENQMFEEKQAES |
| | | MPEIETEQEYKEIFITPHQIKHIKDFKDYKYSHRVDKKPNRELINDTLYSTR |
| | | KDDKGNTLIVNNLNGLYDKDNDKLKKLINKSPEKLLMYHHDPQTYQKL |
| | | KLIMEQYGDEKNPLYKYYEETGNYLTKYSKKDNGPVIKKIKYYGNKLNA |
| | | HLDITDDYPNSRNKVVKLSLKPYRFDVYLDNGVYKFVTVKNLDVIKKEN |
| | | YYEVNSKCYEEAKKLKKISNQAEFIASFYNNDLIKINGELYRVIGVNNDLL |
| | | NRIEVNMIDITYREYLENMNDKRPPRIIKTIASKTQSIKKYSTDILGNLYEV |
| | | KSKKHPQIIKKGKRPAATKKAGQAKKKKGSYPYDVPDYAYPYDVPDYAY |
| | | PYDVPDYA |

TABLE 12
EBS1

| SEQ | | |
|---|---|---|
| ID NO: | Name | Sequence: |
| 119 | EBS1 sequence 1 | UAGGGC |
| 120 | EBS1 sequence 2 | UUAUGG |
| 121 | EBS1 sequence 3 | UCAACG |
| 122 | EBS1 sequence 4 | UGUGGC |

TABLE 13
IBS1

| SEQ | | |
|---|---|---|
| ID NO: | Name | Sequence: |
| 123 | IBS1 sequence 1 | GCCCUG |
| 124 | IBS1 sequence 2 | CCAUGG |
| 125 | IBS1 sequence 3 | CGUUGA |
| 126 | IBS1 sequence 4 | GCCAUA |

TABLE 14
8 and its upstream sequence

| SEQ | | |
|---|---|---|
| ID NO: | Name | Sequence: |
| 127 | 8 and its upstream | UGUGCU |
| | sequence 1 | |
| 128 | 8 and its upstream | AAUGCU |
| | sequence 2 | |
| 129 | 8 and its upstream | UGCUCU |
| | sequence 3 | |
| 130 | 8 and its upstream | UGUGCU |
| | sequence 4 | |

TABLE 15
IBS3 and its downstream sequence

| SEQ | | |
|---|---|---|
| ID NO: | Name | Sequence: |
| 131 | IBS3 and its downstream | AGCAAA |
| | sequence 1 | |
| 132 | IBS3 and its downstream | AGCAGU |
| | sequence 2 | |
| 133 | IBS3 and its downstream | AGAGAA |
| | sequence 3 | |
| 134 | IBS3 and its downstream | AGCAAA |
| | sequence 4 | |

TABLE 16
Eubacteria Introns

| Name | GenBank |
|---|---|
| A.v.I1 | AY057439.2: 1648 . . . 4444 |
| A.v.I2 | NZ_AAAD01000052.1: [3989 . . . 5847] |
| A.v.I3 | CP001157.1: 2457039 . . . 2459231 |
| A.v.I5 | CP001157.1: 2471407 . . . 2473316 |
| Ac.ca.I1 | CP001472.1: 3723390 . . . 3725327 |
| Act.pt.I1 | AF369871.1: [2878 . . . 4803] |
| Acv.sp.I1 | CP000539.1: [2353956 . . . 2355875] |
| Acy.ma.I1 | CP000840.1: [229001 . . . 230873] |
| Ag.r.I1 | AP002086.1: [134014 . . . 136195] |
| Ag.ra.I1 | CP000629.1: 1749091 . . . 1750949 |
| Al.ma.I1 | CP001103.3: [2968732 . . . 2970821] |
| Alc.vi.I1 | CP001896.1: 3347252 . . . 3349087 |
| Alc.vi.I2 | CP001896.1: 961349 . . . 963350 |
| Alk.or.I1 | CP000853.1: [2108190 . . . 2110275] |
| Als.sh.I1 | FP929032.1: [1396482 . . . 1398884] |
| Als.sh.I2 | FP929032.1: [3736965 . . . 3739343] |
| Als.sh.I3 | FP929032.1: [3352050 . . . 3354541] |
| Alx.bo.I1 | AM286690.1: 701337 . . . 704551 |
| Am.de.I1 | CP001785.1: 1080266 . . . 1082151 |
| An.pr.I1 | CP001709.1: 88835 . . . 91175 |
| An.v.I1 | CP000121.1: 50858 . . . 53348 |
| B.a.I1 | AE011190.1: [6579 . . . 9109] |
| B.a.I2 | AE011190.1: [30945 . . . 33835] |
| B.c.I1 | AE016877.1: [2627659 . . . 2630017] |
| B.c.I10 | ABDM02000062.1: [164204 . . . 166883] |
| B.c.I11 | CP001285.1: [9919 . . . 12544] |
| B.c.I12 | CP001186.1: 725163 . . . 727690 |
| B.c.I14 | ABDM02000034.1: [1922 . . . 4450] |
| B.c.I15 | ABDL02000005.1: [20000 . . . 22547] |
| B.c.I16 | CP000228.1: 228934 . . . 231746 |
| B.c.I17 | CP000228.1: 6219 . . . 8855 |
| B.c.I18 | CP000227.1: 2102949 . . . 2105487 |
| B.c.I2 | AE017194.1: [3444106 . . . 3446987] |
| B.c.I3 | AE017194.1: [3603527 . . . 3606193] |
| B.c.I4 | AE017195.1: [32766 . . . 35608] |
| B.c.I5 | AE017195.1: 84166 . . . 86938 |
| B.c.I6 | DQ889679.1: 178948 . . . 181808 |
| B.c.I7a | CP000040.1: 462018 . . . 464610 |
| B.c.I8 | CP000764.1: [3216324 . . . 3218844] |
| B.h.I1 | BA000004.3: 56387 . . . 58269 |
| B.me.I1 | AB022308.1: 3853 . . . 6569 |
| B.me.I2 | AF142677.4: 34045 . . . 36400 |
| B.me.I3 | CP001983.1: 1833173 . . . 1836068 |
| B.my.I1 | ACMV01000578.1: 1 . . . 2843 |
| B.pf.I1 | CP001879.1: [169320 . . . 172131] |
| B.pf.I2 | CP001879.1: [140094 . . . 142818] |
| B.pf.I4 | CP001879.1: [132961 . . . 135833] |
| B.ps.I1 | ACMX01000035.1: 32580 . . . 35423 |
| B.sp.I1 | NZ_AAOX01000004.1: [96386 . . . 98244] |
| B.sp.I2 | EF165030.1: 173 . . . 2955 |
| B.th.I1 | DQ025752.1: [4351 . . . 6956] |
| B.th.I3 | DQ363750.1: 30070 . . . 32039 |
| B.th.I5 | FM992108.1: 131 . . . 3040 |
| B.th.I6 | FM992109.1: 370 . . . 3180 |
| B.th.I7 | FM992111.1: 1064 . . . 3765 |
| B.th.I9 | CP000485.1: [3657622 . . . 3660287] |
| Ba.fr.I1 | AY515263.1: [38446 . . . 40893] |
| Ba.t.I1 | AE015928.1: [2871095 . . . 2873499] |
| Ba.t.I2 | AE015928.1: [3241156 . . . 3243662] |
| Ba.t.I3 | AE015928.1: [3254698 . . . 3258524] |
| Ba.t.I4 | AE015928.1: [3254752 . . . 3256655] |
| Ba.vu.I1 | CP000139.1: 2745315 . . . 2747743 |
| Ba.vu.I2 | CP000139.1: 2750217 . . . 2752618 |
| Bo.pe.I1 | AM902716.1: 1525067 . . . 1527603 |
| Br.j.I1 | BA000040.2: 2212569 . . . 2214373 |
| Br.j.I2 | BA000040.2: 2069342 . . . 2071253 |
| Br.sp.I1 | CP000494.1: [6816299 . . . 6818172] |
| Brb.br.I1 | AP008955.1: 811204 . . . 813093 |
| Bu.ce.I1 | CP000379.1: [1630309 . . . 1632162] |
| Bu.ce.I2 | AM747721.1: [1153119 . . . 1155033] |
| Bu.f.I1 | NZ_AAAC01000271.1: [24723 . . . 26575] |
| Bu.f.I2 | NZ_AAAC01000248.1: [41364 . . . 43217] |
| Bu.f.I3 | NZ_AAAC01000146.1: [5817 . . . 7672] |
| Bu.vi.I1 | CP000616.1: [1220693 . . . 1222534] |
| Bu.vi.I2 | CP000617.1: 381828 . . . 383697 |
| Bu.xe.I1 | CP000270.1: 3577975 . . . 3579828 |
| Bu.xe.I2 | CP000270.1: [3157292 . . . 3159144] |
| By.fi.I1 | FP929036.1: 1233324 . . . 1236128 |
| c-Acb.ph.I1 | CP001715.1: [3647982 . . . 3649879] |
| c-Acb.ph.I2 | CP001715.1: 2040775 . . . 2042757 |
| c-Ku.st.I1 | CT573074.1: 62738 . . . 64755 |
| c-Mb.ox.I1 | FP565575.1: 17703 . . . 19629 |
| C.a.I1 | AE001437.1: [3710916 . . . 3712835] |
| C.be.I3 | CP000721.1: [3718265 . . . 3720149] |
| C.bo.I1 | CP001083.1: [3104052 . . . 3106759] |
| C.bo.I2 | CP001581.1: [2642061 . . . 2644554] |
| C.bo.I3 | CP000963.1: [80372 . . . 82873] |
| C.ce.I1 | CP001348.1: [3312256 . . . 3314954] |
| C.cf.I1 | FP929037.1: [497155 . . . 499743] |
| C.d.I1 | AM180355.1: 596100 . . . 598746 |
| C.d.I2 | FN668944.1: 1876301 . . . 1878962 |
| C.d.I3 | FN668944.1: 4138141 . . . 4140922 |
| C.kl.I1 | AP009049.1: 559484 . . . 562141 |
| C.pe.I1 | AB236336.1: 19829 . . . 22579 |
| Cc.w.I1 | NZ_AADV01000039.1: [6112 . . . 8597] |
| Cc.w.I3 | NZ_AADV01000001.1: 413430 . . . 416144 |
| Cc.w.I5 | NZ_AADV02000007.1: [20418 . . . 23139] |
| Cc.w.I6 | NZ_AADV02000041.1: [1790 . . . 4153] |
| Cc.w.I7 | NZ_AADV02000076.1: [9489 . . . 11634] |
| Cev.ja.I1 | CP000934.1: [3788874 . . . 3790736] |
| Ch.lu.I1 | CP000096.1: [751837 . . . 753995] |
| Ch.ph.I1 | CP001101.1: 1470543 . . . 1472697 |
| Ch.ph.I2 | CP000492.1: [3012641 . . . 3014550] |
| Ci.ro.I1 | FN543502.1: 3382345 . . . 3385222 |
| Ci.ro.I2 | FN543502.1: [3820414 . . . 3823001] |
| Co.ca.I1 | FP929038.1: 3172164 . . . 3174036 |
| Cu.me.I1 | CP000352.1: 3134534 . . . 3137479 |
| Cu.me.I2 | CP000352.1: 269574 . . . 272518 |
| Cu.ta.I1 | CU633751.1: [138639 . . . 140516] |
| Cu.ta.I2 | CU633751.1: [81150 . . . 83086] |
| Cx.sp.I1 | X71404.1: 446 . . . 2898 |
| D.h.I1 | CP001336.1: 1633064 . . . 1638161 |
| D.h.I2 | CP001336.1: 1634688 . . . 1637195 |
| D.h.I3 | CP001336.1: 3041150 . . . 3043051 |
| D.h.I4 | AP008230.1: [5193183 . . . 5195085] |
| D.h.I5 | AP008230.1: 4383270 . . . 4385626 |
| D.h.I6 | AP008230.1: [5169112 . . . 5171014] |
| D.h.I7 | AP008230.1: [5171857 . . . 5174375] |
| Dch.a.I1 | CP000089.1: 759875 . . . 761862 |
| Dh.re.I1 | CP001734.1: 751396 . . . 753288 |
| Dh.re.I2 | CP001734.1: [2610697 . . . 2612577] |
| Di.da.I1 | CP001654.1: [814296 . . . 816881] |
| Di.ze.I1 | CP001655.1: 4627726 . . . 4629590 |
| Di.ze.I2 | CP001655.1: [788411 . . . 790997] |
| Dsf.p.I1 | CR522871.1: [6124 . . . 8213] |
| Dsf.p.I2 | CR522870.1: [2856969 . . . 2859062] |
| E.c.I10 | AB255435.1: 19779 . . . 21675 |
| E.c.I11 | EU935739.1: 16018 . . . 18678 |
| E.c.I2 | X77508.1: 518 . . . 2408 |
| E.c.I3 | CU928162.2: 337804 . . . 339627 |
| E.c.I4 | AB024946.1: 48555 . . . 50824 |
| E.c.I5 | AF074613.1: 58241 . . . 60646 |
| E.c.I7 | AY785243.1: [414 . . . 2383] |
| E.c.I8 | AP010910.1: 8403 . . . 10745 |
| Ef.a.I1 | AY248839.1: 1 . . . 1884 |
| En.ca.I1 | FN555436.1: 22093 . . . 24738 |
| En.f.I3 | AE016830.1: [2249712 . . . 2252481] |
| En.fm.I1 | NZ_AAAK03000007.1: 10877 . . . 13634 |
| En.fm.I2 | DQ321786.1: 12459 . . . 15138 |
| En.fm.I3 | FN424376.1: [17411 . . . 20180] |
| En.fm.I4 | AB105543.1: 1 . . . 2748 |
| Eu.cy.I1 | FP929041.1: 418271 . . . 421021 |
| Eu.re.I1 | CP001107.1: 2565508 . . . 2567889 |
| Eu.re.I2 | FP929043.1: 124807 . . . 126667 |
| Eu.re.I3 | CP001107.1: [2051477 . . . 2053382] |
| Eu.si.I1 | FP929044.1: 1418369 . . . 1421172 |
| Eu.si.I2 | FP929044.1: 1412093 . . . 1414472 |
| Fa.pr.I1 | FP929046.1: 1416454 . . . 1418833 |
| Fa.pr.I2 | FP929046.1: 829768 . . . 831634 |
| Fl.jo.I1 | CP000685.1: [4416242 . . . 4418139] |
| Fr.sp.I2 | CP000820.1: [3485703 . . . 3488125] |
| Fr.sp.I4 | CP000820.1: 1651830 . . . 1653736 |
| Fr.sp.I5 | CP000820.1: 4042148 . . . 4044207 |
| G.v.I1 | BA000045.2: [168850 . . . 171364] |
| Gb.k.I2 | BA000043.1: 1374694 . . . 1376580 |
| Gb.sp.I1 | CP001638.1: [1801642 . . . 1803526] |
| Gb.sp.I2 | CP001638.1: 537253 . . . 539140 |
| Gb.sp.I3 | CP001794.1: [3211355 . . . 3213238] |
| Ge.s.I1 | AE017180.2: 1028655 . . . 1030562 |
| Ge.sp.I1 | CP001390.1: 1082503 . . . 1084403 |
| Ge.ur.I1 | CP000698.1: 1525569 . . . 1527641 |
| Ge.ur.I2 | CP000698.1: 242469 . . . 244398 |
| H.s.I1 | CP000947.1: [614920 . . . 618334] |
| Ha.ch.I1 | CP000155.1: 4094997 . . . 4098178 |
| Ha.ch.I2 | NC_007645.1: 98723 . . . 100647 |
| Hl.mo.I1 | CP000930.2: 1358641 . . . 1360524 |
| Hm.ar.I1 | CU207211.1: 1002053 . . . 1004950 |
| Hp.au.I1 | CP000875.1: 6105650 . . . 6108021 |
| Hp.au.I2 | CP000875.1: 2316638 . . . 2319059 |
| Kl.pn.I1 | DQ153218.1: [2031 . . . 3956] |
| Kl.pn.I2 | EF382672.1: [189794 . . . 192188] |
| Kl.pn.I3 | FJ384365.1: [1787 . . . 3733] |
| Kl.pn.I4 | AJ971342.1: [758 . . . 2678] |
| Kl.pn.I5 | DQ449578.1: [12052 . . . 13960] |
| Kl.pn.I6 | FJ876827.1: [8373 . . . 10644] |
| Ko.ol.I1 | CP001634.1: 1320274 . . . 1322269 |
| L.l.I1 | U50902.4: 7222 . . . 9713 |
| La.re.I1 | AY911856.1: 603 . . . 2512 |
| La.sa.I1 | CP000233.1: 58703 . . . 60602 |
| Le.pn.I1 | AE017354.1: [1176062 . . . 1178351] |
| Le.pn.I2 | CP001828.1: 1305351 . . . 1307556 |
| Le.pn.I3 | CP000675.2: [2799175 . . . 2801059] |
| Ly.sc.I1 | CP000817.1: [3907890 . . . 3910487] |
| Ma.mr.I2 | CP000471.1: [2463983 . . . 2465973] |
| Ma.mr.I3 | CP000471.1: 785727 . . . 787568 |
| Mic.sp.I1 | AF339846.1: 29388 . . . 31287 |
| Mo.th.I1 | CP000232.1: 2324936 . . . 2328581 |
| My.va.I1 | CP000511.1: 2360134 . . . 2362120 |
| N.sp.I1 | BA000019.2: [6207287 . . . 6209592] |
| N.sp.I2 | BA000020.2: 259212 . . . 261420 |
| N.sp.I3 | BA000020.2: 258243 . . . 262762 |
| N.sp.I4 | AP003604.1: 45422 . . . 47908 |
| Na.th.I1 | CP001034.1: [2741438 . . . 2743538] |
| Na.th.I2 | CP001034.1: [2315203 . . . 2317117] |
| Ni.ha.I1 | CP000320.1: [75444 . . . 77354] |
| Ns.e.I1 | AL954747.1: 2285095 . . . 2287101 |
| Nv.a.I1 | AF079317.1: 43084 . . . 45661 |
| Nv.a.I2 | AF079317.1: 53812 . . . 56360 |
| O.i.I1 | BA000028.3: [2785523 . . . 2787411] |
| O.i.I2 | BA000028.3: [2079836 . . . 2081783] |
| Oc.an.I1 | CP000758.1: 1790728 . . . 1792653 |
| OYPI1 | AP006628.2: [388234 . . . 390749] |
| P.a.I1 | AF323437.1: 1 . . . 1924 |
| P.ae.I1 | AY029772.1: [3515 . . . 5441] |
| P.ae.I2 | CP000438.1: [640489 . . . 643335] |
| P.ae.I3 | EF611303.1: 15783 . . . 17632 |
| P.p.I1 | AF101076.1: 1 . . . 1920 |
| P.p.I2 | Y18999.2: 752 . . . 2957 |
| P.p.I3 | AE015451.2: 741473 . . . 743392 |
| P.p.I4 | CP000949.1: [400713 . . . 403556] |
| P.p.I5 | DQ988162.1: [2099 . . . 4689] |
| P.s.I1 | AE016853.1: [2381076 . . . 2382906] |
| P.st.I1 | CP000304.1: 1961716 . . . 1964635 |
| P.st.I2 | CP000304.1: 755737 . . . 757918 |
| Pa.de.I1 | CP000491.1: 19065 . . . 20924 |
| Pbu.ph.I1 | CP001043.1: 506909 . . . 509808 |
| Pbu.ph.I2 | CP001046.1: [235287 . . . 237481] |
| Pbu.ph.I3 | CP001046.1: [170095 . . . 172008] |
| Pe.th.I1 | AP009389.1: [2583061 . . . 2585155] |
| Pe.th.I2 | AP009389.1: 2519125 . . . 2521096 |
| Peb.ca.I1 | CP000142.2: [2649551 . . . 2651540] |
| Peb.ca.I2 | CP000142.2: 1608371 . . . 1610304 |
| Pey.ph.I1 | CP001110.1: [398581 . . . 400415] |
| Ph.p.I2 | CR378677.1: 243610 . . . 245714 |
| Pht.l.I2 | BX571862.1: 232816 . . . 234697 |
| Pol.sp.I1 | CP000316.1: [4896821 . . . 4899617] |
| Pol.sp.I2 | CP000316.1: [5188267 . . . 5191068] |
| Pol.sp.I3 | CP000316.1: [4879088 . . . 4881888] |
| Pol.sp.I4 | CP000316.1: [970429 . . . 973227] |
| Pr.ae.I1 | CP001108.1: 85758 . . . 87667 |
| Pr.ae.I2 | CP001108.1: [682112 . . . 684268] |
| Pr.ae.I3 | CP001108.1: [2285675 . . . 2287507] |
| Pr.ae.I4 | CP001108.1: 688308 . . . 690474 |
| Pr.ae.I5 | CP001108.1: [670818 . . . 672686] |
| Pr.vi.I1 | CP000607.1: 169617 . . . 171449 |
| Ps.tu.I1 | AAOH01000003.1: 353461 . . . 355380 |
| Pt.mo.I1 | CP000879.1: [1358317 . . . 1360205] |
| Pv.r.I1 | AY887109.1: [1698 . . . 3623] |
| R.pi.I1 | CP001068.1: 3298333 . . . 3301217 |
| R.so.I1 | CU694389.1: [49404 . . . 51259] |
| Re.sp.I1 | NZ_AAOE01000010.1: 129694 . . . 133129 |
| Rh.op.I1 | AP011115.1: [3108320 . . . 3110812] |
| Rh.sp.I1 | CP000432.1: [23005 . . . 25058] |
| Ro.in.I1 | FP929050.1: 3738659 . . . 3740507 |
| Ro.in.I2 | FP929049.1: 12968 . . . 14828 |
| Ru.cm.I1 | FP929052.1: 1525395 . . . 1528189 |
| Ru.to.I1 | FP929055.1: 1244183 . . . 1246940 |
| Ru.to.I2 | FP929055.1: 2325690 . . . 2327580 |
| S.ag.I1 | AJ292930.1: 182 . . . 2038 |
| S.ag.I2 | AE014217.1: 10188 . . . 12210 |
| S.eq.I1 | FM204884.1: [1986162 . . . 1988470] |
| S.eq.I2 | FM204884.1: [1951740 . . . 1954234] |
| S.eq.I3 | FM204884.1: [1968250 . . . 1970644] |
| S.mi.I1 | FN568063.1: [1124531 . . . 1126388] |
| S.pn.I1 | AF030367.1: 833 . . . 2754 |
| S.pn.I2 | FM211187.1: [1246152 . . . 1248657] |
| S.py.I1 | CP000262.1: [1657366 . . . 1659841] |
| Sa.en.I1 | AM932669.1: [2406 . . . 4331] |
| Sb.mo.I1 | CP001779.1: 734656 . . . 737020 |
| Sb.mo.I2 | CP001779.1: [752270 . . . 754744] |
| Se.ma.I1 | BX664015.1: [172056 . . . 173964] |
| Se.ma.I2 | AF453998.2: [1526 . . . 3496] |
| Se.ma.I3 | AY884051.1: [2933 . . . 4858] |
| Sg.ce.I1 | AM746676.1: [9205316 . . . 9207294] |
| Sh.ba.I1 | CP000563.1: [2699774 . . . 2701938] |
| Sh.ba.I2 | CP000563.1: 2137684 . . . 2139633 |
| Sh.ba.I3 | CP000891.1: [1164604 . . . 1166759] |
| Sh.fr.I1 | CP000447.1: 4148493 . . . 4150650 |
| Sh.pi.I1 | CP000472.1: 4966032 . . . 4968192 |
| Sh.se.I1 | CP000821.1: 3108662 . . . 3110815 |
| Sh.sp.I1 | CP000446.1: [2526748 . . . 2528903] |
| Sh.sp.I2 | CP000444.1: 645848 . . . 649292 |
| Shg.dy.I1 | CP000035.1: [29397 . . . 31222] |
| Shg.f.I1 | CP001383.1: [1091555 . . . 1093825] |
| Sm.av.I1 | BA000030.4: [264494 . . . 266721] |
| So.us.I2 | CP000473.1: 3231872 . . . 3233814 |
| So.us.I3 | CP000473.1: [9594438 . . . 9596378] |
| So.us.I4 | CP000473.1: 2550504 . . . 2552417 |
| Sp.wi.I1 | CP000701.1: [78388 . . . 80930] |
| Sr.md.I1 | CP000740.1: [1030234 . . . 1032118] |
| Sr.me.I1 | Y11597.2: 1 . . . 1518 |
| Sr.me.I2 | AE006469.1: [1065612 . . . 1067822] |
| Sr.me.I5 | EF066650.1: 146809 . . . 148801 |
| Sy.wo.I1 | CP000448.1: 170134 . . . 172134 |
| Sy.wo.I2 | CP000448.1: [2254240 . . . 2256141] |
| Syb.fu.I1 | CP000478.1: 3922427 . . . 3924309 |
| Syb.th.I1 | AP006840.1: 1010793 . . . 1012672 |
| T.e.I2 | CP000393.1: 5587083 . . . 5589603 |
| T.e.I3 | CP000393.1: [680421 . . . 682946] |
| T.e.I4 | CP000393.1: [675663 . . . 678216] |
| T.e.I5 | CP000393.1: 6034823 . . . 6037198 |
| T.e.I6 | CP000393.1: [7378688 . . . 7381311] |
| T.e.I7 | CP000393.1: [7258498 . . . 7263621] |
| T.e.I8 | CP000393.1: [7261005 . . . 7263468] |
| Ta.it.I1 | CP001936.1: [2442736 . . . 2444647] |
| Ta.ps.I1 | CP000924.1: 45196 . . . 47107 |
| Ta.sp.I1 | CP000923.1: 774653 . . . 776564 |
| Ta.sp.I2 | CP000923.1: 1286631 . . . 1288551 |
| Tc.po.I1 | CP002028.1: 264953 . . . 267042 |
| Th.e.I1 | BA000039.2: 27344 . . . 30566 |
| Th.e.I3 | BA000039.2: [91363 . . . 93748] |
| Th.e.I7 | BA000039.2: 624954 . . . 627338 |
| Tm.sp.I1 | FP475956.1: [3005251 . . . 3007161] |
| UB.I1 | AY691909.1: [2430 . . . 4342] |
| UMB.I1 | AY075117.1: 120 . . . 2136 |
| Vi.an.I1 | NZ_AAOJ01000008.1: [2856 . . . 5011] |
| Vi.ch.I1 | EU116440.1: [1769 . . . 3745] |
| Vi.ha.I1 | CP000789.1: 2600607 . . . 2602762 |
| Vi.ha.I2 | CP000790.1: 1339722 . . . 1341882 |
| Vi.ha.I3 | CP000789.1: [2438671 . . . 2440560] |
| Vi.vu.I1 | GQ292873.1: 3620 . . . 5549 |
| Wo.sp.I1 | CP001391.1: 130825 . . . 133124 |
| Wo.sp.I3 | AE017196.1: 950978 . . . 953271 |
| Wo.sp.I6 | AM999887.1: [177114 . . . 178961] |
| Wo.sp.I7 | AM999887.1: [284826 . . . 286812] |
| X.f.I1 | AE003849.1: 1691283 . . . 1693684 |
| Zu.pr.I1 | CP001650.1: [4279634 . . . 4281497] |
| Zu.pr.I2 | CP001650.1: 3589332 . . . 3591217 |

TABLE 17
Archaea Introns

| Name | GenBank |
|---|---|
| M.a.I1 | AE011073.1: 4279 . . . 6431 |
| M.a.I5 | AE011130.1: [2949 . . . 4823] |
| M.b.I1 | CP000099.1: 2122631 . . . 2124795 |
| M.m.I1 | AE013515.1: 3337 . . . 5483 |
| Mc.b.I1 | CP000300.1: 800706 . . . 803724 |
| Me.hu.I1 | NC_007796.1: 3055567 . . . 3057700 |
| Me.hu.I2 | NC_007796.1: [1521394 . . . 1523527] |
| UA.I1 | AY714843.1: 21625 . . . 23730 |
| UA.I10 | FP565147.1: [502990 . . . 504827] |
| UA.I2 | AY714849.1: [14494 . . . 16717] |
| UA.I3 | AY714856.1: 10054 . . . 11891 |
| UA.I4 | AY714820.1: 20258 . . . 22206 |
| UA.I6 | FP565147.1: 2174430 . . . 2176370 |
| UA.I7 | FP565147.1: [1619711 . . . 1621720] |
| UA.I8 | FP565147.1: 2238167 . . . 2240107 |
| UA.I9 | FP565147.1: [2809849 . . . 2811954] |

TABLE 18
ORF-less Introns

| Name | GenBank |
|---|---|
| B.c.I13 | CP001187.1: [155442 . . . 156188] |
| B.c.I7b | CP000040.1: 3829 . . . 4655 |
| B.th.I2 | DQ025752.1: [16908 . . . 17856] |
| Bu.xe.I3 | CP000270.1: [790064 . . . 790889] |
| C.pe.I2 | DQ787115.1: 1 . . . 834 |
| M.a.I6 | AE010299.1: [4030024 . . . 4030645] |
| OYPI2 | AP006628.2: [544682 . . . 545416] |
| Th.e.I11 | BA000039.2: 1683646 . . . 1684491 |
| Th.e.I2 | BA000039.2: 27972 . . . 28810 |
| Th.e.I4 | BA000039.2: 219750 . . . 220594 |
| Th.e.I5 | BA000039.2: [245778 . . . 246623] |
| Th.e.I6 | BA000039.2: 444131 . . . 445814 |
| Th.e.I9 | BA000039.2: 1159085 . . . 1159922 |

TABLE 19
Twintron (Outer intron)

| Name | GenBank |
|---|---|
| Ba.t.I3 | AE015928.1: [3254698 . . . 3258524] |
| D.h.I1 | CP001336.1: 1633064 . . . 1638161 |
| N.sp.I3 | BA000020.2: 258243 . . . 262762 |
| T.e.I7 | CP000393.1: [7258498 . . . 7263621] |
| Th.e.I1 | BA000039.2: 27344 . . . 30566 |
| Th.e.I6 | BA000039.2: 44413 |

TABLE 20
Twintron (Inner intron)

| Name | GenBank |
|---|---|
| Ba.t.I4 | AE015928.1: [3254752 . . . 3256655] |
| D.h.I2 | CP001336.1: 1634688 . . . 1637195 |
| N.sp.I2 | BA000020.2: 259212 . . . 261420 |
| T.e.I8 | CP000393.1: [7261005 . . . 7263468] |
| Th.e.I2 | BA000039.2: 27972 . . . 28810 |

TABLE 21
Mitochondrial introns

| Species | Genbank |
|---|---|
| Arabidopsis thaliana | X98300 |
| Glycine max | U09988 |
| Oenothera berteriana | M63034 |
| Vicia faba | M30176 |
| Zea mays | U09987 |
| Marchantia polymorpha | M68929 |
| Marchantia polymorpha | M68929 |
| Marchantia polymorpha | M68929 |
| Marchantia polymorpha | M68929 |
| Marchantia polymorpha | M68929 |
| Marchantia polymorpha | M68929 |
| Marchantia polymorpha | M68929 |
| Marchantia polymorpha | M68929 |
| Chara vulgaris | AY267353 |
| Mesostigma viride | NC_008240 |
| Porphyra purpurea | AF114794 |
| Porphyra purpurea | AF114794 |
| Pavlova lutheri | AF045691 |
| Pylaiella littoralis | Z48620 |
| Pylaiella littoralis | Z48620 |
| Pylaiella littoralis | Z72500 |
| Pylaiella littoralis | Z72500 |
| Pylaiella littoralis | Z72500 |
| Thalassiosira pseudoana | NC_007405 |
| Rhodomonas salina | NC_002572 |
| Rhodomonas salina | NC_002572 |
| Allomyces macrogynus | U41288 |
| Rhizphydium sp. 136 | NC_003053 |
| Neurospora crassa | X14669 |
| Podospora anserina | X55026 |
| Podospora anserina | X55026 |
| Podospora anserina | X55026 |
| Podospora comata | Z69899 |
| Podospora curvicolla | Z69898 |
| Venturia inaequalis | AF004559 |
| Candida parapsilosis | NC_005253 |
| Candida stellata | NC_005972 |
| Kluyveromyces lactis | X57546 |
| Saccharomyces cerevisiae | V00694 |
| Saccharomyces cerevisiae | V00694 |
| Schizosaccaromyces pombe EF2 | X54421 |
| Schizosaccaromyces pombe | AJ251292 |
| Schizosaccaromyces pombe | AJ251293 |
| Schizosaccharomyces octosporus | NC_004312 |
| Amoebidium parasiticum | AF538043 |

TABLE 22
Chloroplast introns

| Species | Genbank |
|---|---|
| Nicotiana tabacum | NC_001879 |
| Marchantia polymorpha | X04465 |
| Scenedesmus obliquus | P19593 |
| Oocystacea sp. | S05341 |
| Bryopsis maxima | X55877 |
| Pyrenomonas salina | X81356 |
| Euglena gracilis | X70810 |
| Euglena gracilis | X70810 |
| Euglena deces | Z99833 |
| Euglena myxocylindracea | Z99835 |
| Euglena viridis | Z99836 |
| Lepocinclis buetschlii | Z99834 |

TABLE 23
Bacterial introns Fragments

| Name | Genbank | |
|---|---|---|
| A.sp.F1 | U13767 | (3012-4881) |
| B.c.F1 | NC_004722 | (2621273-2621348) |
| B.h.F1 | AP001513 | (82798-82923) |
| B.h.F2 | AP001509 | (236458-236855) |
| B.h.F3 | AP001518 | (279207-279280) |
| B.j.F1 | AF322013 | (11219-12768) |
| B.j.F2 | AF322013 | (61716-62201) |
| B.j.F3 | AF322013 | (64129-65049) |
| B.j.F4 | AF322012 | (109786-110508) |
| B.j.F5 | AF322012 | (~112711-113404) |
| B.the.F1 | AE016936 | (56021-57802) |
| B.t.F1 | AF547282 | (134-220) |
| B.c.F2 | NC_005957 | (2284679-2284770) |
| C.g.F1 | AP005279 | (216855-216940) |
| C.t.F1 | AE015940 | (57392-57706) |
| E.f.F1 | AF242872 | (5457-~6118) |
| E.c.F1 | X60106 | (205-727) |
| E.c.F2 | D37919 | (1-1122) |
| E.c.F3 | D37918 | (154-1590) |
| E.c.F4 | AF044501 | (1631-1823) |
| E.c.F5 | AE005360 | (2258-2450) |
| E.c.F6 | AP002557 | (72163-72355) |
| E.c.F7 | AF044503 | (8964-9122) |
| E.c.F8 | AE016771 | (291635-291731) |
| E.c.F9 | Y16016 | (83-1186) |
| Ma.sp.F2 | NZ_AAAN01000054 | (6215-6748) |
| Ma.sp.F3 | NZ_AAAN01000014 | (13-1609) |
| Ma.sp.F4 | NZ_AAAN01000039 | (3099-3260) |
| Ma.sp.F5 | NZ_AAAN01000139 | (22445-23847) |
| Ma.sp.F6 | NZ_AAAN01000041 | (1963-3050) |
| M.lo.F1 | AP003008 | (75689-76468) |
| M.b.F1 | NZ_AAAR01001642 | (2313-2979) |
| M.le.F1 | AL583918 | (25817-26960) |
| M.le.F2 | AL583918 | (~200511-~201270) |
| M.t.F1 | AE006920 | (~407-1042) |
| P.al.F1 | AF323438 | (1952-2634) |
| P.l.F1 | BX571862BX571862 | (231958-235010) |
| P.p.F3 | AF134348 | (~720-1494) |
| P.p.F4 | AF006691 | (~19530-20220) |
| P.p.F5 | AE016790 | (208013-209185) |
| P.p.F6 | AF134348 | (825-1494) |
| P.p.F7 | AJ245436 | (17048-17349) |
| P.sp.F1 | X98999 | (4224-4418) |
| P.st.F1 | AF039534 | (682-936) |
| R.e.F1 | AF176227 | (9216-10069) |
| R.e.F2 | U80928 | (223116-224119) |
| R.e.F3 | U80928 | (101332-101767) |
| R.sp.F1 | AE000069 | (5461-6042) |
| S.a.F1 | AP005021 | (264494-266317) |
| S.f.F1 | D26468 | (3211-3612) |
| S.f.F2 | AF348706 | (29835-31623) |
| S.f.F3 | AF348706 | (151605-153082) |
| S.me.F1 | AL603645 | (28607-30146) |
| S.o.F1 | AE015645 | (2404-2930) |
| S.p.F1 | AE007346 | (102-289) |
| S.p.F2 | AE007409 (11094-) to AE007410 (-89) | |
| S.p.F3 | AE007372 | (5515-5697) |
| S.p.F4 | AE007369 | (7460-7914) |
| S.p.F5 | AE008434 | (1474-1963) |
| S.p.F6 | AE008536 | (21-327) |
| S.p.F7 | AE008467 | (9532-9877) |
| S.p.F8 | AE007478 | (241-316) |
| S.p.F9 | AE008471 | (9510-9837) |
| S.c.F1 | AL049661 | (11519-11956) |
| Th.e.F1 | AP005374 | (215166-215259) |
| Th.e.F2 | AP005374 | (216672-217080) |
| Tr.e.F1 | NZ_AAAU01000017 | (18413-20506) |
| Tr.2.F2 | NZ_AAAU01000002 | (9475-10805) |
| Tr.e.F3 | NZ_AAAU01000059 | (16240-17497) |
| Tr.e.F4 | NZ_AAAU01000021 | (53554-54961) |
| Tr.e.F5 | NZ_AAAU01000021 | (55017-56144) |
| Tr.e.F6 | NZ_AAAU01000021 | (56303-58137) |
| Tr.e.F7 | NZ_AAAU01000021 | (58441-59419) |
| Tr.e.F11 | NZ_AAAU01000039 | (19166-20116) |
| Y.e.F1 | AF336309 | (40186-40488) |
| Y.p.F1 | AF074611 | (54417-55543) |

TABLE 24
Archaebacteria Intron fragments

| Name | Genbank |
|---|---|
| M.a.I1-F1 | AE011130(6248-7525) |
| M.a.I1-F2 | AE010979(6611-7239) |
| M.a.I2-F1 | AE011130(7526-9438) |
| M.a.I3-F1 | AE011185(5745-7226) |
| M.a.I6-F1 | AE010902(10030-10335) |
| M.a.F1 | AE010964(6933-8146) |
| M.a.F2 | AE010848(4041-4124) |
| M.a.F3 | AE011134(6345-7286) |
| M.a.F4 | AE010979(3887-6610) |
| M.b.F1 | NZ_AAAR01001642 (2313-2979) |
| M.b.F2 | NZ_AAAR01001590 (749-2058) |
| M.b.F3 | NZ_AAAR01001948 (294-620) |

### 8. EXAMPLES

For a more complete understanding and application of the present invention, the present invention will be described in detail below with reference to the examples and the drawings, and the examples are only intended to illustrate the present invention and are not intended to limit the scope of the present invention. The scope of the present invention is specifically defined by the appended claims.

### 8.1. Example 1. Screening of Group II Introns

This example relates to a method for confirming the in vitro self-splicing capability of natural group II introns.

First, the DNA sequence was directly synthesized according to the natural sequence of a group II intron (Genewiz, Suzhou), and the synthesized DNA sequence comprised, in addition to the group II intron sequence itself, the naturally occurring flanking exon E1 and E2 sequences, especially all or part of the intron binding region in the exon immediately adjacent to the group II intron. The DNA sequence was cloned into the modified expression vector psiCHECK-2 (Promega, C8021) comprising the coding sequence of Rluc (Renilla Luciferase), a T7 promoter and a T7 terminator by molecular biological method. Specifically, psiCHECK-2 was digested with a single endonuclease, XhoI (New England Biolabs (NEB)), and the synthesized DNA sequence was then cloned into an enzymatically digested vector using the DNA seamless assembly method (ABclonal Technology, Wuhan), located 3′ downstream of Rluc, to obtain the corresponding construct. The bakcbone of this expression vector was psiCHECK-2 comprising a T7 promoter and a T7 terminator.

PCR amplification was performed on the above vector using universal primers for T7 promoter and T7 terminator to obtain template DNAs for transcription. The PCR reaction conditions were: 95° C. for 30 s, 60° C. for 20 s, and 72° C. for 60 s, for 23 to 25 cycles. The template DNAs obtained by PCR amplification were extracted with phenol-chloroform at a volume ratio of 1:1, and then precipitated with 2.5 times by volume of absolute ethanol for purification.

The purified template DNAs were transcribed in vitro by T7 RNA polymerase (NEB or Promega), and the transcription reaction was performed according to the conditions recommended by the manufacturer's instructions. The transcripts were digested with DNase I at 37° C. for 30 min to degrade the PCR templates. The transcripts were then purified by column purification to obtain high-purity RNAs.

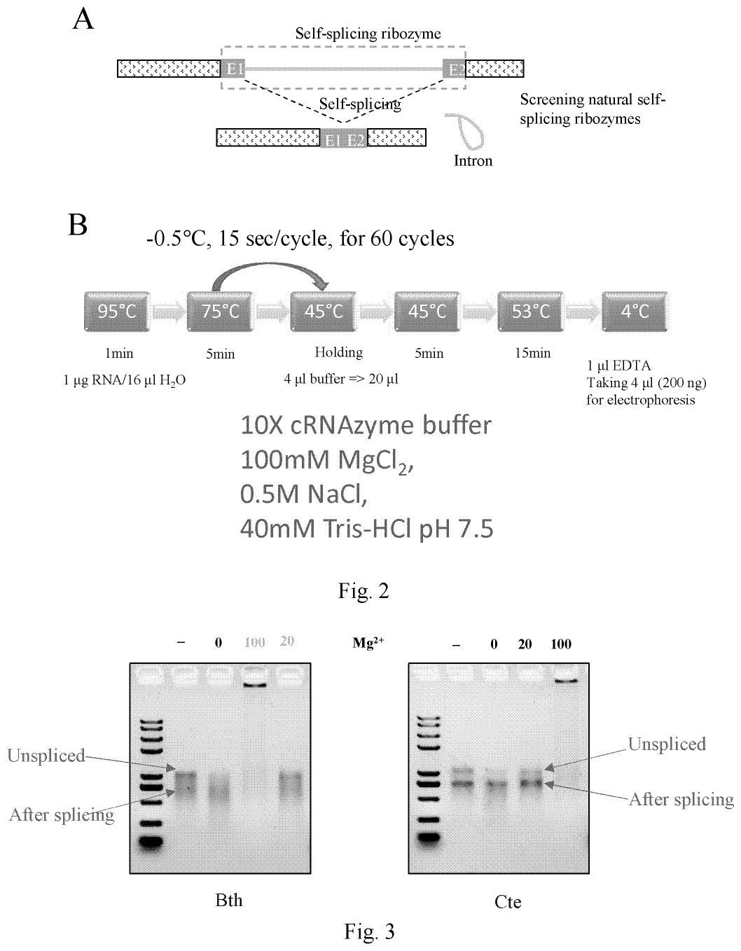

The column-purified transcript RNAs were added to a self-splicing buffer (10, 20, 50 or 100 mM MgCl2, 50 mM NaCl, 40 mM Tris-HCl, pH=7.5) for self-splicing reaction. The reaction conditions were: 95° C. for 1 min, 75° C. to 45° C. (−0.5° C., 15 sec/cycle, for 60 cycles in total), holding at 45° C. with a buffer added, 45° C. for 5 min, and 53° C. for 15 to 30 min (see FIG. 2B). After the self-splicing reaction occurred in vitro, 200 ng of the product was used for electrophoresis analysis on an agarose gel at a concentration of 1.5% to detect the self-splicing efficiency of group II introns.

If self-splicing successfully occurred, two RNAs of different sizes would be produced. The unspliced RNA would be larger in size and at the top in the gel electrophoretogram; and the spliced RNA would be smaller in size and at the bottom in the gel electrophoretogram. For example, FIG. 3 shows the electrophoresis results of two group II introns identified by the above-mentioned method that may perform self-splicing, namely the group II intron Bth from Bacillus thuringiensis and the group II intron Cte from Clostridium tetani (FIG. 3). Arrows in FIG. 3 show unspliced and spliced RNAs separated by electrophoresis, respectively. The group II intron and its flanking exon sequence (such as SEQ ID NO: 1 or SEQ ID NO: 2, comprising the group II introns of Bth and Cte and 6 nucleotides of their flanking E1 and 6 nucleotides of flanking E2, respectively) confirmed by the method of this example was used as a precursor for the preparation of the self-splicing ribozyme construct cRNAzyme, also referred to as a cRNAzyme precursor.

### 8.2. Example 2. Preparation of Expression Constructs Comprising Group II Introns

The self-splicing ribozyme cRNAzyme construct was further prepared on the basis of the group II intron cRNAzyme precursor with the self-splicing property obtained by screening. As mentioned above, the general principles for designing a cRNAzyme construct are that the total length of the intron sequence and the E1 and E2 sequences is as small as possible, and the circularization rate is as high as possible.

This example will set forth in detail the process of designing and preparing a cRNAzyme construct using the Cte cRNAzyme precursor screened in Example 1.

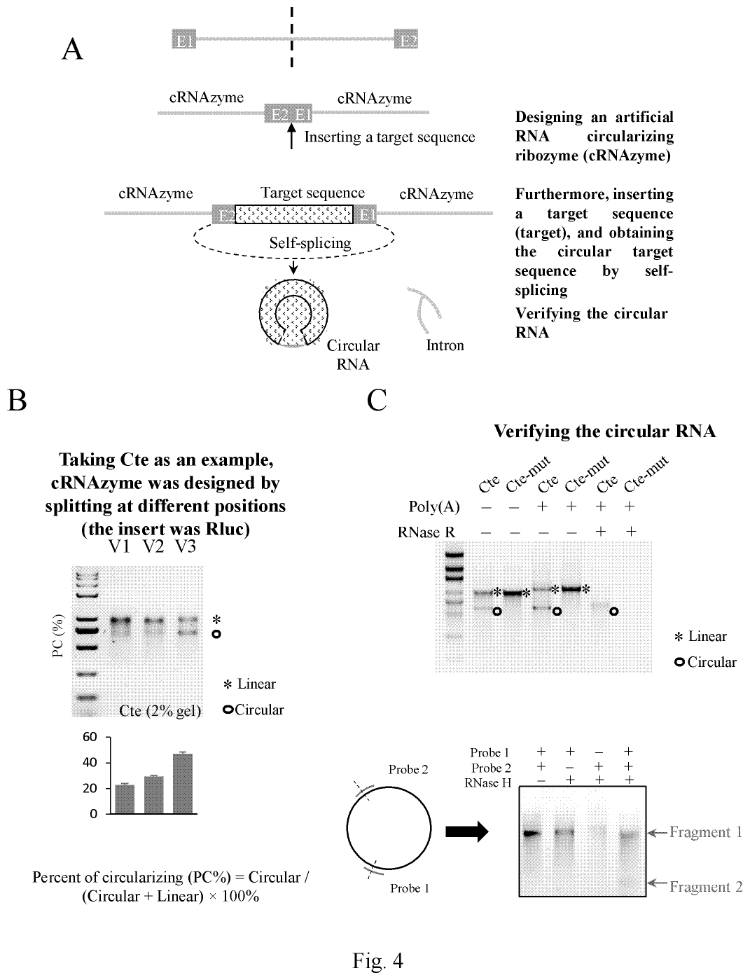

On the basis of the Cte cRNAzyme precursor sequence (SEQ ID NO: 2, a total of 1,028 nucleotides in length, comprising the Cte intron itself and the exon sequences of 6 nucleotides at both ends, i.e., both E1 and E2 being 6 nucleotides in length), the intron-encoded protein (IEP) sequence of 310 nucleotides in domain 4 (nucleotide positions 625 to 934 of SEQ ID NO: 2) was deleted, and the sequence that could fold correctly and maintain self-splicing activity was retained, comprising a few parts of exons at both ends (6 nt each, i.e., IBS1 and IBS3), thereby obtaining the E1-CteΔIEP-E2 sequence. The E1-CteΔIEP-E2 sequence was then split at a position inside the intron. The positions of the two fragments after segmentation were swapped, a first fragment consisting of E1 and a 5′ intron fragment was constructed to the 3′ end of the insert Rluc, and a second fragment consisting of a 3′ intron fragment and E2 was constructed to the 5′ end of the insert Rluc. On this basis, AATACCTTACTTAATAGTAACAATAGAAAATC (SEQ ID NO: 14) was inserted at the 5′ end of the newly formed fragment, and AAGCTAGATCATATTACTATTAAGTAAGGTATT (SEQ ID NO: 15) was inserted at the 3′ end, thereby obtaining a cRNAzyme_Cte construct. The two inserted sequences of SEQ ID NO: 14 and SEQ ID NO: 15 served as “homology arms” to make the 5′ and 3′ splice sites close to each other and improve the splicing efficiency. When segmenting the intron, three different segmentation positions were tried, which were located in loop regions in domain 1 (between positions 369 and 370), domain 3 (between positions 560 and 561), and domain 4 (between positions 825 and 826), respectively. Three cRNAzymes were thus formed, named cRNAzyme_Cte V1, cRNAzyme_Cte V2, and cRNAzyme_Cte V3, respectively. In the presence of cations, a self-splicing circularization reaction was performed in vitro to test the circularization activity of the obtained cRNAzyme. It can be seen from FIG. 4b that the splicing effects were different after segmentation at different positions, among which the third segmentation method (V3) has the best splicing effect, and was used in subsequent experiments. The sequence of this construct was shown in SEQ ID NO: 16.

Based on the fragment size, the band marked as a circle in the gel electrophoretogram of FIG. 4b was considered to represent circular RNAs. The sequence was verified by RT-PCR and Sanger sequencing, and it was determined that the band was a sequence spanning the E1E2 junction, which proved that E1 and E2 had been linked together. However, it has not been confirmed that it was a circular RNA, as the band may also be from a back-spliced transcript, or other sequences. To confirm the successful formation of a circular RNA, the following three methods were used to verify its structure (FIG. 4c) based on the characteristic of the head-to-tail covalent closure of the circular RNA.

### Method I

The splice site in the Cte was mutated to lose its circularization capability, as a linear RNA control of the same length but unable to achieve circularization. Specifically, the splice site (nucleotides 1 to 26) in SEQ ID NO: 2 was mutated to change C at position 3 to A, T at position 5 to G, G at position 17 to T, C at position 18 to T, A at position 21 to C, and T at position 26 to G. It would be understood by those skilled in the art that this mutation was intended to disrupt the circularization capability, and other mutations in different numbers, positions, and types may also be made to achieve similar goal. The mutated Cte was referred to as Cte-mut (SEQ ID NO: 3). A polyA tail was then added to this mutated linear RNA using poly(A) polymerase. Since a circular RNA was closed in a head-to-tail manner and had no 3′ end, it can not be tailed, while a linear RNA may be added with hundreds of adenylates through this reaction, and the changes in RNA size before and after tailing may be resolved by agarose gel electrophoresis.

The specific steps were as follows:
(1) The purified circular RNAs or control linear RNAs were tailed by poly(A) tailing enzyme (NEB), and the reaction conditions were at 37° C. for 30 min;
(2) RNAs were subjected to column purification.

As shown in the upper panel of FIG. 4c, lanes 3 and 4 were products with polyA tails added, while lanes 1 and 2 were products without polyA tails added. In the case of no circularization, the bands of cRNAzyme precursor RNA based on Cte and Cte-mut all shifted upward after polyA addition reaction, indicating that the molecules became larger and polyA was successfully added. In contrast, the bands presumed to be circular RNAs were substantially at the same position and of the same size with and without the polyA addition reaction.

### Method II

Digestion was performed with RNase R. RNase R was a 3′-5′ exoribonuclease that may degrade linear RNA molecules; and circular RNAs with closed loop structures can not be degraded. The digestion of RNAs may be identified by agarose gel electrophoresis.

The specific steps were as follows:
(1) The purified circular RNAs or control linear RNAs were tailed by poly(A) tailing enzyme (NEB), and the reaction conditions were at 37° C. for 30 min;
(2) RNase R (Lucigen) was added to digest the linear RNAs, and the reaction conditions were at 37° C. for 30 min; and
(3) RNAs were subjected to column purification.

As shown in the upper panel of FIG. 4C, lanes 5 and 6 were the results after RNase R treatment, while lanes 1 to 4 were the results without RNase R treatment. It can be seen that the larger linear RNA bands disappeared after RNase R treatment (lanes 5 and 6), while the band presumed to be a circular RNA in lane 5 still existed.

### Method III

Digestion was performed with RNase H. RNase H is an endoribonuclease that may specifically hydrolyze the RNA in hybrid DNA-RNA strands. Since linear RNAs and circular RNAs had different structures, they may be cleaved into fragments of different lengths by RNase H after being bound to the same DNA probe. By agarose gel electrophoresis, the lengths of the RNA fragments produced by cleavage may be resolved, so as to infer the original structure of RNAs. Specifically, for circular RNAs, two DNA probes were used to bind to RNAs and then the product was cleaved, resulting in two bands. In contrast, if there was no circularization, but still linearity, the same method should result in three bands.
(1) Through the annealing reaction, a DNA probe was bound to the RNA. The reaction was run at 95° C. for 2 min, and then slowly and gradually cooled to 25° C. The probe sequences were shown in SEQ ID NOs: 7 and 8.
(2) The DNA/RNA double strands were digested with RNase H (NEB), and the reaction conditions were at 37° C. for 30 min; and
(3) RNAs were subjected to column purification. As shown in the lower panel of FIG. 4C, two bands were obtained for the product of the present application.

The results obtained by the above three methods all confirmed the successful formation of circular RNAs and verified the circularization activity of the constructed cRNAzyme_Cte.

### 8.3. Example 3. Improvement of Self-Splicing Efficiency by Optimizing the Reaction System and Engineering the Construct

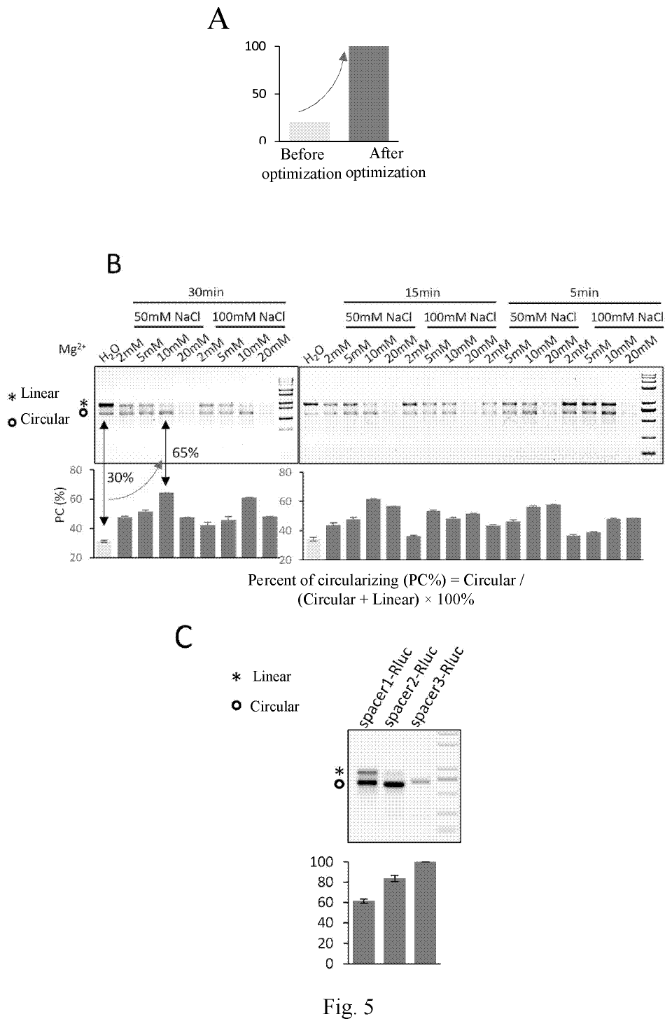

In order to increase the final circular RNA yield, it was first necessary to improve the percent of circularization (FIG. 5a). The inventors optimized the percent of circularization of the expression construct from two aspects.

### Optimization of Reaction Conditions

The inventors tried combinations of various ion concentrations (50 mM and 100 mM NaCl; 2 mM, 5 mM, 10 mM, and 20 mM Mg2+) and various reaction times (5 min, 15 min, and 30 min) in the reaction system to determine the optimal reaction system.

It was found that the reactions in the reaction system of 20 mM Mg2+ and 50 mM NaCl for 15 and 30 minutes can allow the percent of circularization increased from the existing 30% to not less than 60% (FIG. 5B).

### Sequence Optimization

The sequence was further engineered according to the RNA secondary structure. Specifically, after insertion of different target sequences, some sequences can not be efficiently spliced for structural reasons. In this case, the splicing efficiency was improved by incorporating some spacer sequences in the target sequence to increase the flexibility of the structure, for example, the spacer sequence may be an AT-rich sequence.

On the basis of Example 2, three different spacer sequences were inserted in front of Rluc in cRNAzyme_Cte by means of molecular cloning, namely the spacer sequence 1 of SEQ ID No: 4, the spacer sequence 2 of SEQ ID NO: 5, and the spacer sequence 3 of SEQ ID NO: 6, resulting in three further optimized constructs. The three spacer sequence-bearing precursor RNAs were circularized in vitro in the optimal self-splicing reaction system identified in Example 2 (10 mM Mg, 50 mM NaCl, reaction time of 30 minutes). It can be seen from the results of FIG. 5C that the percent of circularization after adding the three spacer sequences were about 60%, 80%, and 98%, respectively (FIG. 5c).

### 8.4. Example 4. Preparation of “Scarless” Circular RNAs without Scar Sequences

The circular RNA obtained using the aforementioned method will still comprise a few non-target sequences, i.e., sequences from exons E1 and E2. To remove these sequences, the construct may be further engineered.

When preparing the cRNAzyme construct, both ends of the target sequence were E2 and E1 with shorter lengths, which were derived from the intron binding sequences (IBS) of the flanking exon regions of the group II intron, respectively, and were generally between 0 to 20 nucleotides in length. In forming a circular RNA, it was undesirable to comprise sequences other than the target sequences, such as exon sequences E1 and E2. If E1 and E2 were removed directly, the self-splicing circularization process would be affected due to the lack of IBS sequence that would interact with the EBS or δ sequence in the intron. The inventors of the present invention have creatively conceived of directly regarding a part of the target sequence as an “IBS” sequence, and modifying the EBS or δ in the intron to allow the EBS to interact with the region in the target sequence that is regarded as “IBS”. Such a method can get rid of the dependence on exon sequences E1 and E2 while ensuring that the cRNAzyme construct has the self-splicing function, and remove the exon sequences from the construct and the final circularization product.

Still taking the Cte ribozyme as an example, the design idea of the cRNAzyme construct in this example was illustrated with different target sequences (GFP, Gluc and 2A peptide).

Based on the sequences of 6 nucleotides at both ends of each target sequence, the EBS1 and upstream sequence of EBS1 (including δ) in the group II intron were respectively replaced with sequences at least partially complementarily paired with the two 6-nucleotide sequences. Specifically, upstream sequence of EBS1 (including δ) was allowed to be complementarily paired with 6 nucleotides at the 5′ end of the target sequence in a linear state (such as the state prior to self-splicing circularization of the cRNAzyme construct), and EBS1 was allowed to be complementarily paired with 6 nucleotides at the 3′ end of the target sequence in a linear state. The specific modified sequences were shown in the right panel of FIG. 6B updated, showing the modified EBS1 and upstream sequence of EBS1 (including δ) designed for three different target sequences. Notably, the engineered EBS1 and upstream sequence of EBS1 (including δ) did not have to be perfectly complementarily paired with the target sequence fragments serving as “IBS1” and “IBS3”. A certain percentage of mismatches, or a slightly less robust pairing of such as A and G, G and U may be allowed. In general, the EBS1 and upstream sequence of EBS1 (including δ) used to substitute the corresponding sequences in group II introns were complementary paired with the corresponding region in the target sequence on at least 60% of the nucleotide positions, or at least 60% identical to the complementary paired sequence of the corresponding region in the target sequence.

It can be seen from the electrophoresis results that circular RNAs were efficiently generated by this engineering method when different target sequences (GFP, Gluc and 2A peptide) were used (FIG. 6B, left). It was confirmed by Sanger sequencing results that such engineering might completely eliminate the exogenous scar sequence (FIG. 6B, right). As shown in FIG. 6B, after the cRNAzyme construct with the EBS and upstream sequence of EBS1 (including δ) modified as above was circularized, both ends of the target sequence (the two stretches of shaded 6-nucleotide sequences, serving as “IBS1” and “IBS3”, respectively) were directly connected in a head-to-tail manner, and the sequence was no longer separated by E1 and E2, which is more beneficial for subsequent applications of the generated circular RNAs. The constructs engineered in this way were referred to as “scarless” constructs, and the circular RNAs after circularization were referred to as “scarless” RNAs.

### 8.5. Example 5. Circularization Results of Target Sequences of Different Lengths

Based on the methods in Examples 2 and 3, the inventors further tested target fragments of different lengths. These target fragments were Gluc of 555 nucleotides, the nucleotide sequence of which was as shown in SEQ ID NO: 9; Rluc1 of 936 nucleotides, the nucleotide sequence of which was as shown in SEQ ID NO: 10; and Rluc2 of 1,160 nucleotides, the nucleotide sequence of which was as shown in SEQ ID NO: 11, respectively. No spacer sequence was added to the Glue and Rluc1 constructs, and the Rluc2 construct consisted of a Cat1 IRES sequence and Rluc.

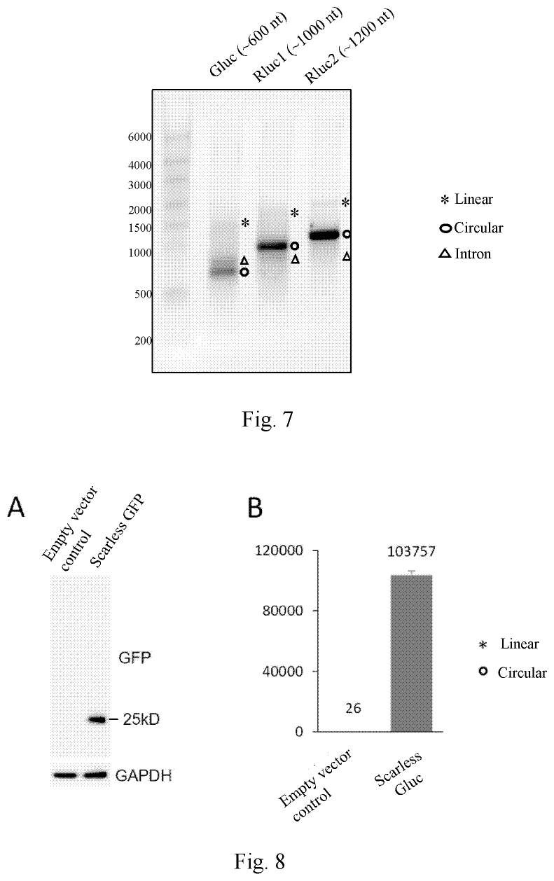

It was found that all tested fragments might perform self-splicing efficiently, resulting in circular RNA products (FIG. 7).

### 8.6. Example 6. Expression of the Gene of Interest Using the Construct of the Present Invention

On this basis, the expression of circular RNA products of different target sequences after transfection of cells with them was further tested.

To minimize immunodegradation caused by linear RNAs, the RNA products were treated in the following three steps prior to transfection.
(1) RNase R treatment was performed to digest linear RNAs. The reaction conditions were at 37° C. for 30 min.
(2) HPLC purification was performed to remove small linear RNAs. HPLC conditions: gel exclusion chromatographic column: Waters Xbridge® BEH450A, column temperature: 40° C., flow rate: 1 min/ml, and elution conditions: 0 to 30 min, 100% buffer A (prepared with 10 mM Tris, 0.5 mM EDTA, and DEPC-treated water). CIP treatment was performed to remove phosphate groups at both ends of the linear RNAs. The reaction conditions were to add quick CIP (NEB), and react at 37° C. for 30 min.

The target RNAs were transfected using lipo RNAmax (Invitrogen), under the transfection conditions according to the supplier's instructions, for 24 hours.

Using the construction methods in Examples 2 and 3 (using spacer sequence 2), and carrying out the modifications described in Example 4 to achieve scarless circularization, different target sequences were tested. To facilitate detection of protein expression, the constructs comprising a fluorescent protein coding sequence were constructed, comprising IRES-GFP (SEQ ID NO: 12) and IRES-Gluc (SEQ ID NO: 13). The addition of IRES might initiate cap-independent non-canonical translation, enabling the translation of coding sequences in circular RNAs into proteins.

For different target sequences, different methods were used to detect protein expression.

In the case that the translation product was GFP, the fluorescence was observed under a microscope, the cells were lysed with RIPA lysis solution (Beyotime), and then the protein expression was detected by Western blotting. It was confirmed that the expression of GFP was obtained by the method of the present invention (FIG. 8A).

In the case that the translation product was luciferase, the cells were first lysed with Passive lysis solution (Promega), and then the protein expression was detected by a microplate reader using a luciferase detection kit (Promega). It was confirmed that the expression of Gluc protein was obtained by the method of the present invention (FIG. 8B)

### 8.7. Example 7. Design and Production of Circular RNAs In Vitro

To efficiently produce circular RNAs, we take advantage of the self-catalyzed splicing reaction by group II introns, which are mobile genetic elements found mainly in bacterial and organellar genomes (Lambowitz, A. M., and Zimmerly, S. (2011). Group II introns: mobile ribozymes that invade DNA. Cold Spring Harb Perspect Biol 3, a003616). All group II introns have six structural domains (D1 to D6), of which the domain 1 (D1) is the largest domain and contains several short exon binding sites (EBS) to determine the splicing specificity (FIG. 12A). The domains 2 and 3 play key roles to assemble the active intron structure and stimulate splicing reaction, whereas the D4 is a stem-loop structure with the long loop containing the ORF of maturase. The highly conserved D5 is the heart of active site for self-splicing (Rybak-Wolf et al. (2015). Circular RNAs in the Mammalian Brain Are Highly Abundant, Conserved, and Dynamically Expressed. Mol Cell 58, 870-885), whereas the D6 contains a bulged adenosine as branching site (FIG. 12A). The in vitro self-splicing of group II intron requires only the correct folding of intronic RNA structure and Mg2+(Peebles, C. L. et al. (1986). A self-splicing RNA excises an intron lariat. Cell 44, 213-223), however the in vivo splicing requires assistance of the maturase (Pyle, A. M. (2016). Group II Intron Self-Splicing. Annu Rev Biophys 45, 183-205).

Based on this domain configuration, we have split the group II self-splicing intron from the surface layer protein of Clostridium tetani (McNeil, B. A., Simon, D. M., and Zimmerly, S. (2014). Alternative splicing of a group II intron in a surface layer protein gene in Clostridium tetani. Nucleic Acids Res 42, 1959-1969) at the D4, generating a split-intron system that contains a customized exon flanked by the upstream D5-D6 and the downstream D1-D2-D3 of the intron. A part of the D4 stem was separated and placed into each end of the resulting RNA, thus forming a complementary structure to help the folding of active intron (FIG. 12A, green lines). Two short 6-nt sequences, IBS3 (intron binding site 3) and IBS1, were include at each junction of the intron and customized exon to provide long-range interactions with the EBS3 and EBS1 of the intron. Upon the in vitro run-off transcription, the resulting RNA precursor could be self-spliced by the group II intron to produce a circular RNA of the customized exon and a branched intron RNA. By including an IRES sequence at the upstream of a gene of interest (GOI), the resulting circular RNA can function as an mRNA to direct protein synthesis through cap-independent translation (FIG. 12A). We named this system as the circular coding RNAs (CirCode), which could serve as a general platform to produce any given circRNA for protein translation.

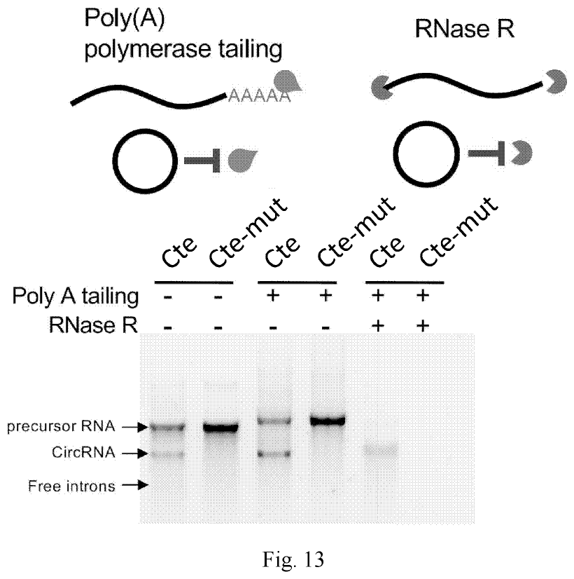

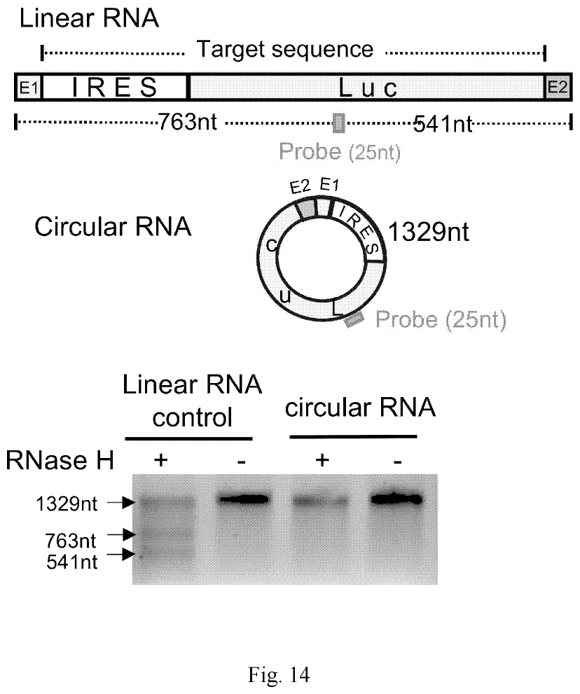

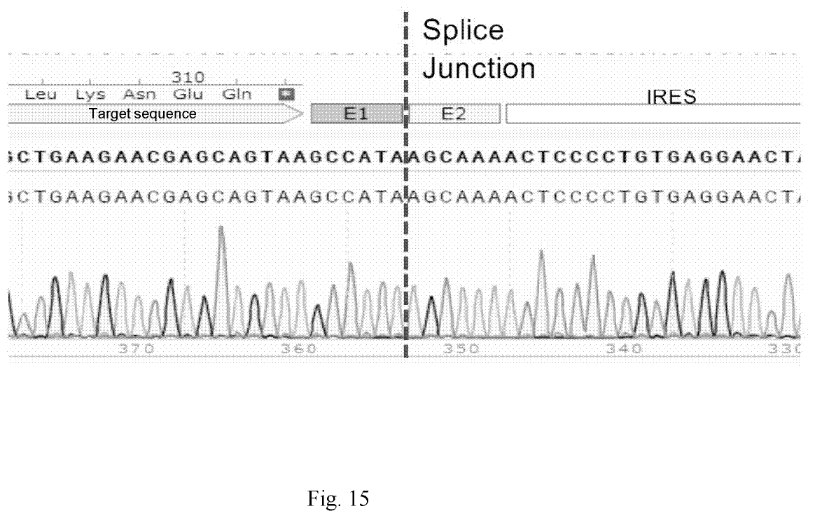

To validate this design, we included an IRESs and the ORF of Renilla luciferase gene into the customized exon, and translate this D5-D5-exon-D1. We found that the resulting RNA precursor can indeed be self-spliced in vitro to produce extra band corresponding to circRNAs, whereas the mutation of the splice junction (at the IBS1) failed to produce the circRNAs (FIG. 1i). We further used two different methods to confirm that the additional band below the precursor RNA is indeed a circRNA, as it cannot be extended by tailing reaction by poly-A polymerase and is resistant to the digestion by RNse R treatment (FIG. 13). In addition, we purified the circRNAs from the gel, and confirmed the circular identify using RNase H digestion (FIG. 14) and directly sequencing of the junction (FIG. 15). Finally, we optimized the reaction conditions using different concentrations of MgCl2 and NaCl in the circularization buffer following the IVT step (see method), and found that using 10-20 mM Mg2+ with 50-100 mM NaCl is the optimal reaction condition for the RNA circularization (FIG. 5B). We also observed certain degree of RNA circularization (˜30%) even when the circularization buffer does not contain any Mg2+, presumably because the RNA circularization happened co-transcriptionally in the IVT buffer containing 24 mM MgCl2.

Platform Optimization of circRNA Production and Translation

The previous self-splicing systems using group I introns for circRNA production also introduced an extraneous sequence from T4 bacteriophage or Anabaena into the final products. This “scar” sequence is usually around 80-180 nt long, which limit the design flexibility of target circRNAs and may introduce some unwanted effect during drug development. Our initial design used two short sequences (IBS1 and IBS3) for the intron-exon recognition, which leaves a shorter scar of 12-nt. To reduce the potential interference by the scar sequence, we further modified the design by changing the exon binding sites the D1 domain, making the EBS1 and EBS3 to respectively form base-pairs with the 3′ and 5′ end of the circular exon (FIG. 16, left). This new design enabled the generation of “scarless circRNAs” without extraneous sequences. The only sequence requirement is the “AG” in the 5′ end of circular exons, which make the design of circular RNAs fully programmable. We validated this design by generating two different circRNAs encoding the EGFP and Rluc-P2A (FIG. 16, right), and observed the efficient circularization of scarless circRNA in different ion conditions. The scarless self-splicing of circRNAs were further confirmed by the sequencing of final circRNA products (FIG. 16, bottom of the right). In summary, we have engineered the CirCode system that can efficiently produce scarless circRNAs of any sequences, providing a general platform for using circRNA as different therapeutic tools.

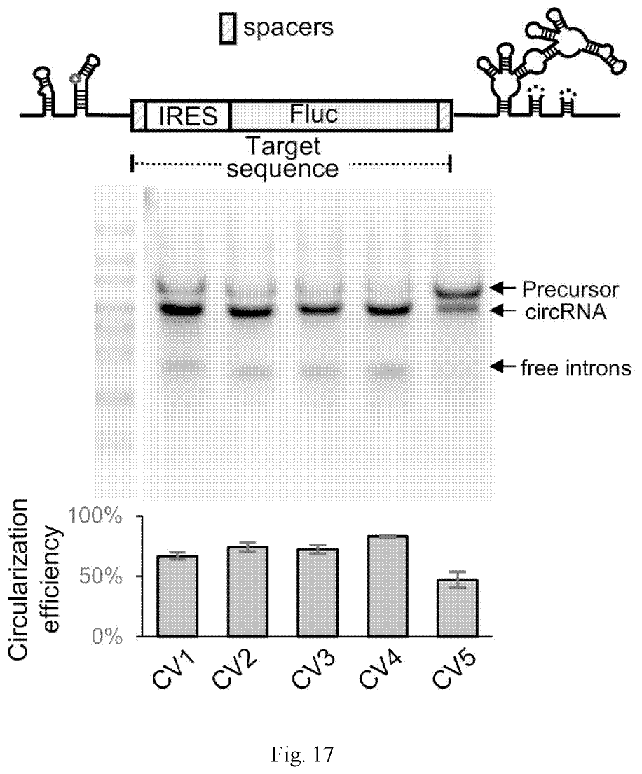

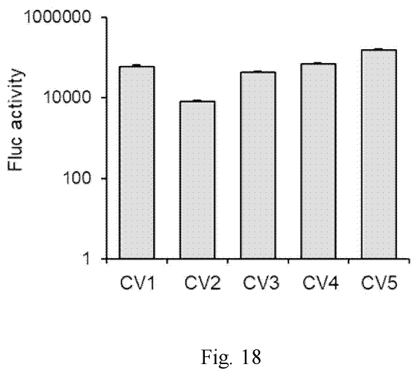

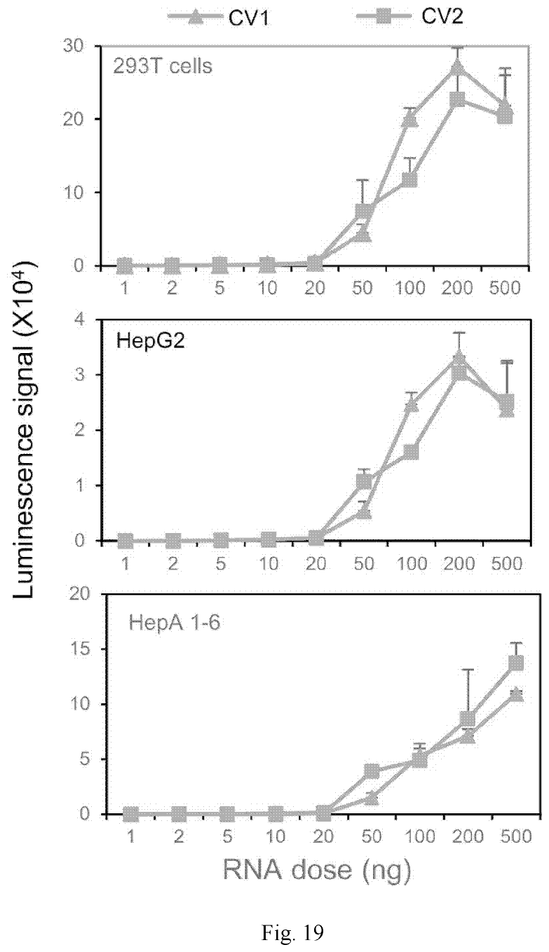

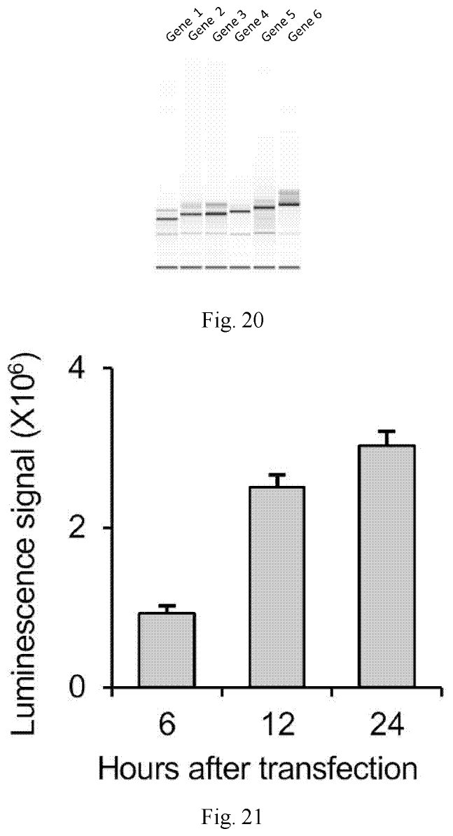

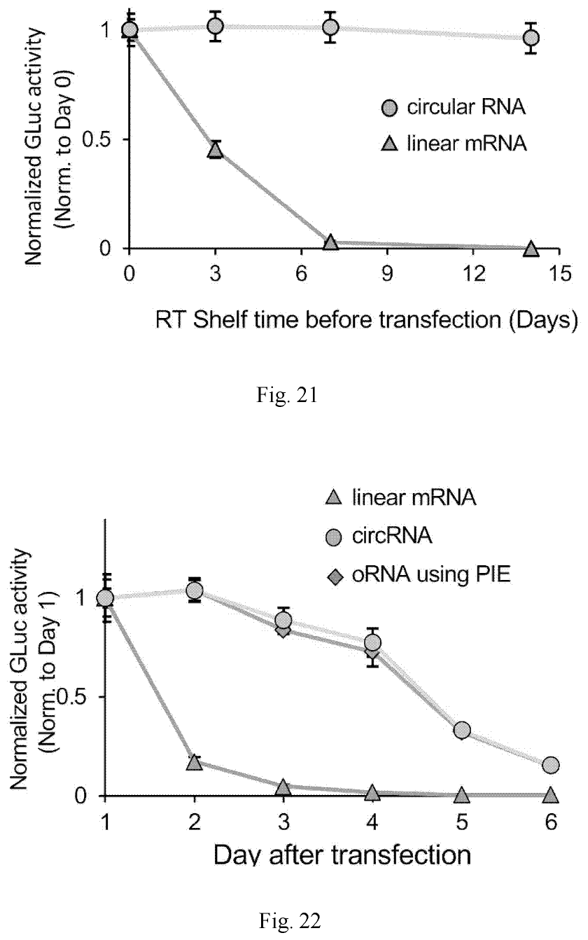

Previous study using PIE method suggested that addition of short spacer regions before the IRES may assist the correct folding of the IRESs and/or the active structure of introns (Wesselhoeft, R. A. et al., 2018, supra), and thus we introduced several versions of spacer sequences at each end of the circular exon to optimize their circularization efficiency (FIG. 17). We found that different spacers indeed affected the RNA circularization efficiency, which ranged from 47% (for SP5) to 83% (for SP4). In addition, we transfected two circRNAs with different spacers into three cell lines at different doses, and observed dose dependent increase of protein production as judged by luciferase activity assay (FIG. 18). Interestingly, the effect of the spacer sequence on translation efficiency appeared to be pretty modest and dependent on the circRNA dose and cell lines. We further examined the time course of protein production upon circRNA transfection, and found that the active protein can be detected in six hours and reach the expression peak of expression at 24 hours after transfection (FIG. 19).

### 8.8. Example 8. circRNAs Mediate Prolonged Protein Production

A major advantage of circular mRNAs is their superb stability because of lacking the free ends, therefore the circRNA should have good shelf life for protein expression compare to linear counterparts. To directly test this, we synthesized both linear and circular mRNA encoding the Gaussia luciferase (Gluc), and stored them parallelly in pure water at room temperature for different days before transfecting them into 293 T cells. We found that the activity of the circRNA to direct protein translation is essentially unchanged during the two weeks, whereas the linear mRNA lost about half of its activity by the day three of storage (FIG. 3A), suggesting that circRNAs have can be stably stored in room temperature. In addition, we found that once transfected into cells, the protein production from linear mRNA decreased rapidly with a >80% at the day 2, whereas the translation from circRNAs last 5-7 days (FIG. 3B). The prolonged protein translation is also consistent with our previous results using back-spliced circRNA reporter (Wang, Y., and Wang, Z. (2015). Efficient backsplicing produces translatable circular mRNAs. RNA 21, 172-179). It is also worth noting that, in this experiment, the protein production from circRNAs may also be suppressed at the end because we did not change the culture medium during the entire week of the experiment (FIG. 16).

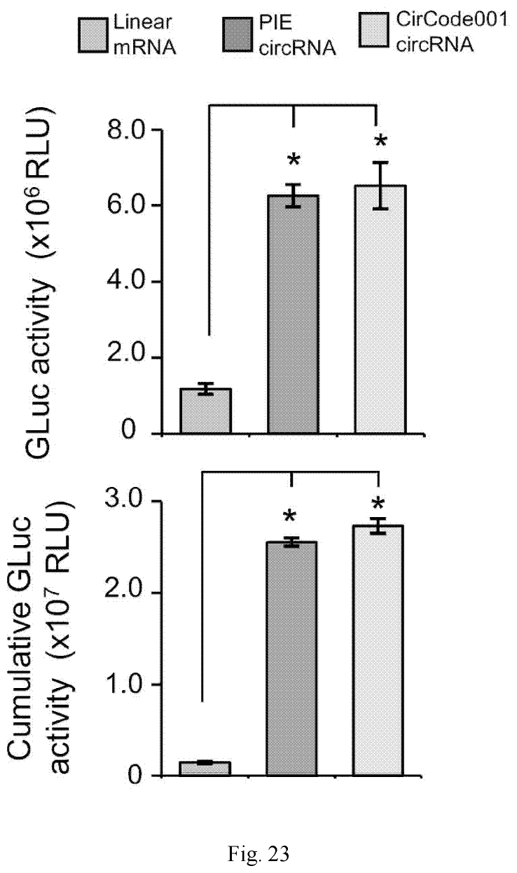

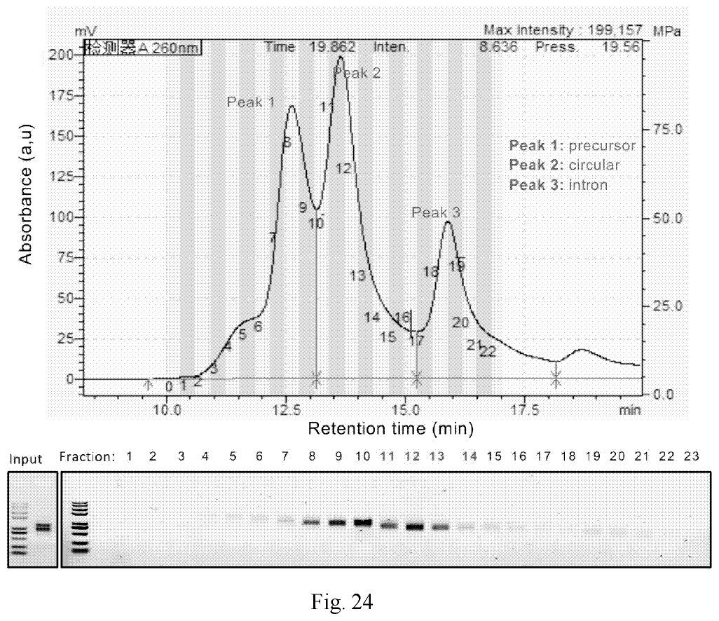

We further compared the protein production from the linear mRNAs and the circRNAs produced using PIE protocol or the new CirCode systems. The capped and unmodified linear mRNAs were generated using IVT with the same coding sequences of Gluc, and transfected into 293 T cells in parallel with the two types of circRNAs containing the CVB3 IRES and Gluc ORF. We found a more robust expression of proteins from both circRNAs compared to the linear mRNA (FIG. 23, top), supporting the previous reports using PIE circRNAs (Wesselhoeft, R. A. et al., 2018, supra). An even larger increase of protein production was observed in circRNAs when we measured the accumulated luciferase activity over the span of 6 days (FIG. 23, bottom), presumably because of the superior stability of circRNAs. In addition, the circRNAs generated from two different protocols showed similar ability in direct protein translation (FIG. 24).

### 8.9. Example 9. Purified circRNAs can Direct Robust Translation of Target Proteins

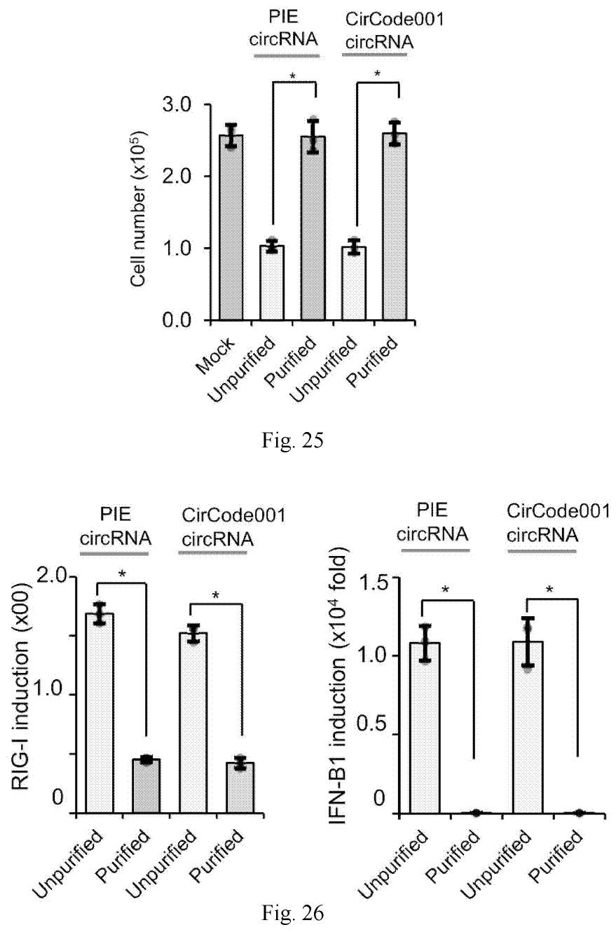

The mRNA purity was found to be a key factor for the protein production and induction of innate immunity, as the removal of dsRNA by HPLC can eliminate immune activation and improves translation of linear nucleoside-modified mRNA (Kariko, K. et al., (2011). Generating the optimal mRNA for therapy: HPLC purification eliminates immune activation and improves translation of nucleoside-modified, protein-encoding mRNA. Nucleic Acids Res 39, e142). However, there are some debates on the immunogenicity of circRNAs. While an early report suggested that in vitro synthesized circRNAs are more prone to induce cellular immune response than the linear RNA (Chen, Y. G. et al., (2017). Sensing Self and Foreign Circular RNAs by Intron Identity. Mol Cell 67, 228-238 e225), it was later reported that purification of circRNAs from byproducts of IVT and circularization reactions, including dsRNA, linear RNA fragments and triphosphate-RNAs, can eliminate the cellular toxicity and immunogenicity of the circRNAs (Wesselhoeft, R. A. et al., (2019). RNA Circularization Diminishes Immunogenicity and Can Extend Translation Duration In Vivo. Mol Cell 74, 508-520 e504; Breuer, J. et al., (2022). What goes around comes around: artificial circular RNAs bypass cellular antiviral responses. Mol Ther Nucleic Acids). A recent study also suggested that the sequence identify and structure are the main determinant of cellular immunity of circRNAs, as the circRNAs produced by different methods showed different immunogenicity (Liu, C. X. et al., (2022). RNA circles with minimized immunogenicity as potent PKR inhibitors. Mol Cell 82, 420-434 e426). To examine if the circRNAs produced using CirCode platform can induce innate immune response and cell toxicity, we purified the circRNAs with gel purification or HPLC (FIG. 3D), and measured if the circRNAs can induce cellular immune response upon transfection of circRNAs. We found that transfection of the unpurified circRNAs cause significant amount of cell death, whereas the purified circRNAs did not show detectable cell toxicity compared to mock transfection (FIG. 25). In addition, compared to the unpurified circRNAs that stimulated innate immune response by inducing RIG-I and IFN-β1 (interferon-01), the purified circRNAs significantly reduced the immunogenicity (FIG. 26), supporting the previous observation that the immunogenicity of circRNAs were mainly caused by the by-products from IVT reaction (Wesselhoeft, R. A. et al., 2019, supra).

### 8.10. Example 10. LNP Encapsulated circRNAs Direct Robust Protein Production in Mouse

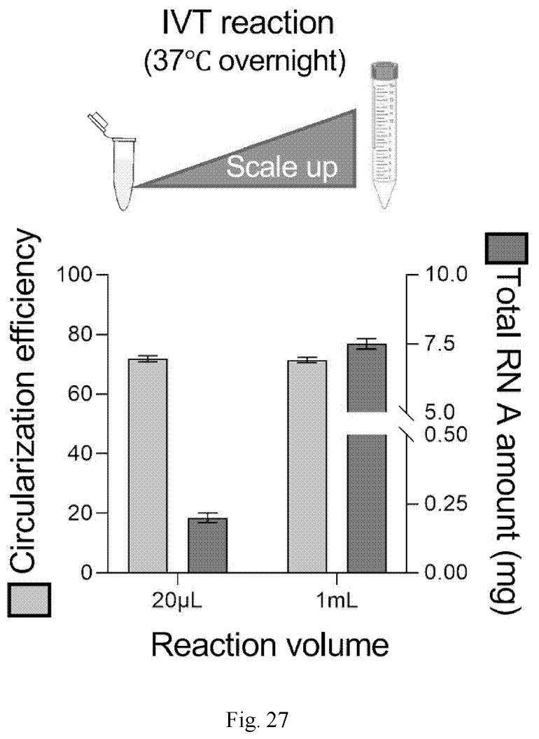

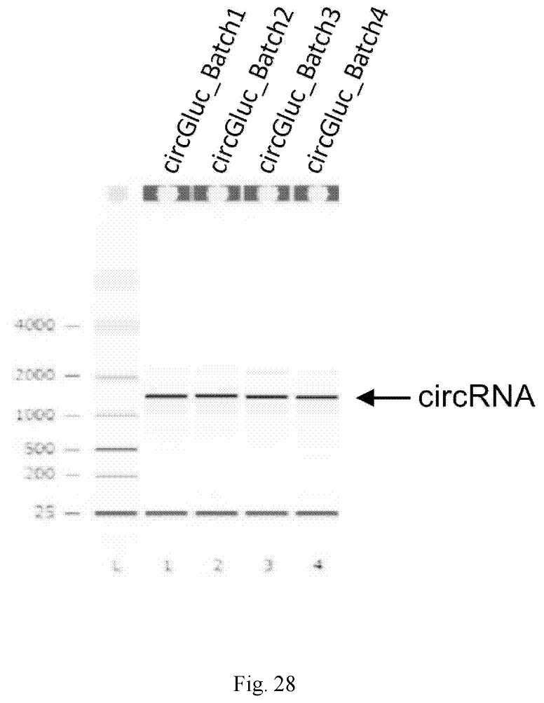

An important question for the therapeutic application of circRNAs is whether the production of circRNAs can be scaled up reliably and how the reproducibility between different batch of production. Because the CirCode platform uses self-splicing intron for RNA circularization without the involvement of RNA ligase or additional co-activators, the scale-up procedure is relatively simple. To test the scalability of this system, we expanded the IVT and circularization reaction system for 50 fold (from 20 μl into 1 ml), and found that the high circularization efficiency (˜70%) stayed essentially unchanged while the total amount of RNA products reached 7.5 mg in a single reaction (FIG. 27). In addition, we found that the IVT/circularization and the HPLC purifications of the circRNAs were highly reproducible between different batches (FIG. 28), laying the ground for the in vivo application of the circRNAs.

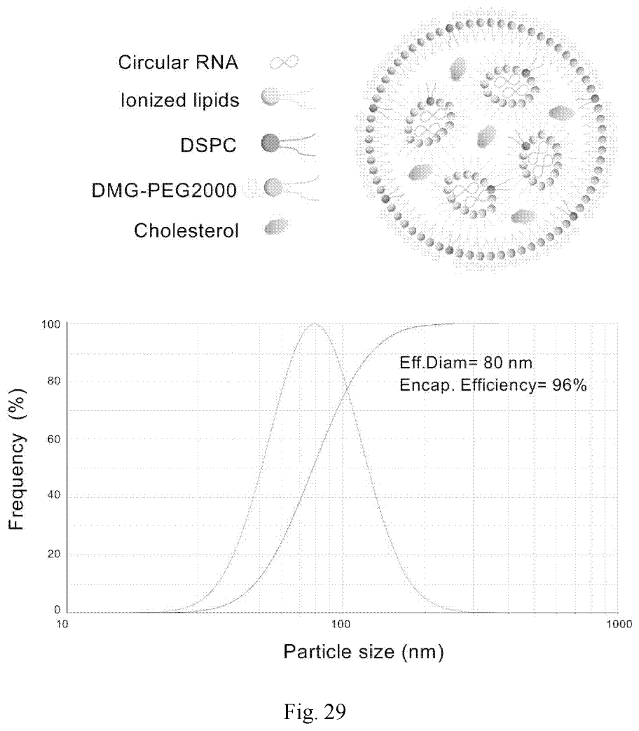

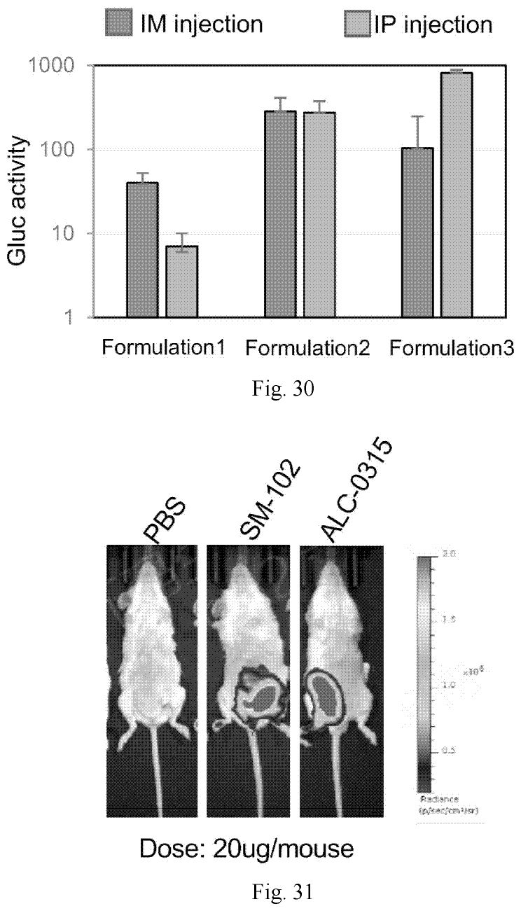

We further generated circRNAs encapsulated with lipid nanoparticles (LNP) to for their in vivo delivery (FIG. 29). The circRNAs in the aqueous solution were packed by ionizable cationic lipids, which form a nanoparticle with other lipid components such as DMG-PEG2000 and Cholesterol (see methods), achieving a ˜95% encapsulate efficiency with effective diameter at ˜80 nm. We have tested three different ionizable cationic lipids in our formulation to encapsulate circRNAs that encode Gluc, and the resulting LNPs were injected into BALC/c mice through intramuscular (IM) or intraperitoneal (IP) injection (n=3 for each experimental group). Three formulations using different ionizable cationic lipids (MC3, SM-102 and ALC-0315) were tested in this experiment. The expressions of luciferase were assayed either using the luciferase luminescence assay with serum (FIG. 30) or bioluminescence imaging of the animals (FIG. 31). We found a robust expression of luciferase from two different formulations of LNP-circRNAs, suggesting that the circRNAs produced through our procedure can reliably induce in vivo protein expression.

### 8.11. Example 11. Generation of a Potential circRNA Vaccine for SARS-coV-2

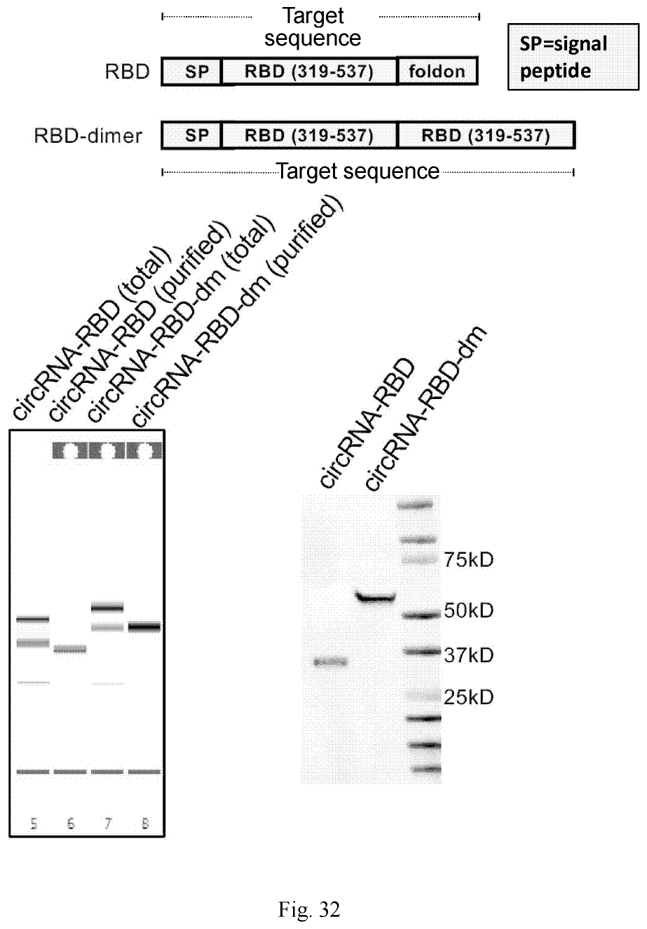

We further tested the application of circRNAs in mRNA therapy by engineering the circRNAs encoding the receptor binding domain (RBD) from the S protein of SARS-cov-2, which can potentially used to produce mRNA vaccine. Based on previous reports, two different antigen designs were constructed into the circRNAs (FIG. 32 top) (Dai, L., and Gao, G. F. (2021). Viral targets for vaccines against COVID-19. Nat Rev Immunol 21, 73-82). The first one used a single RBD fused with the foldon from T4 fibritin that enables trimerization of RBD (Meier, S., et al., (2004). Foldon, the natural trimerization domain of T4 fibritin, dissociates into a monomeric A-state form containing a stable beta-hairpin: atomic details of trimer dissociation and local beta-hairpin stability from residual dipolar couplings. J Mol Biol 344, 1051-1069). The other using a RBD dimmer that was shown to induce strong antibody production in pilot study (Dai, L. et al., (2020). A Universal Design of Betacoronavirus Vaccines against COVID-19, MERS, and SARS. Cell 182, 722-733 e711; Dai, L. et al., 2021, supra). We have designed the coding sequences of both proteins with a modified IRES, and constructed the circRNA vectors. The production and purification of the resulting circRNAs were validated (FIG. 32, bottom left), and translation of the circRNA-encoded proteins was further validated by transfecting into 293 cells and detected by western blot antigen production (FIG. 32, bottom right).

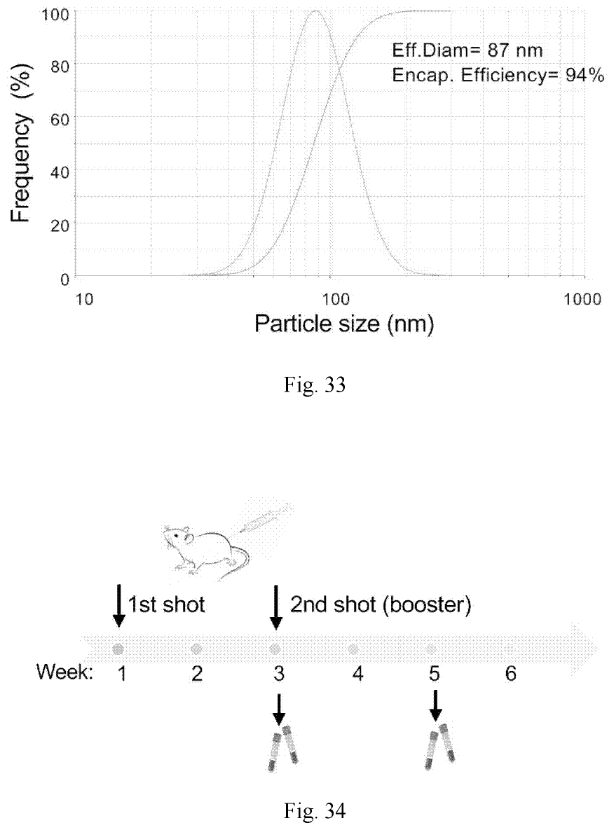

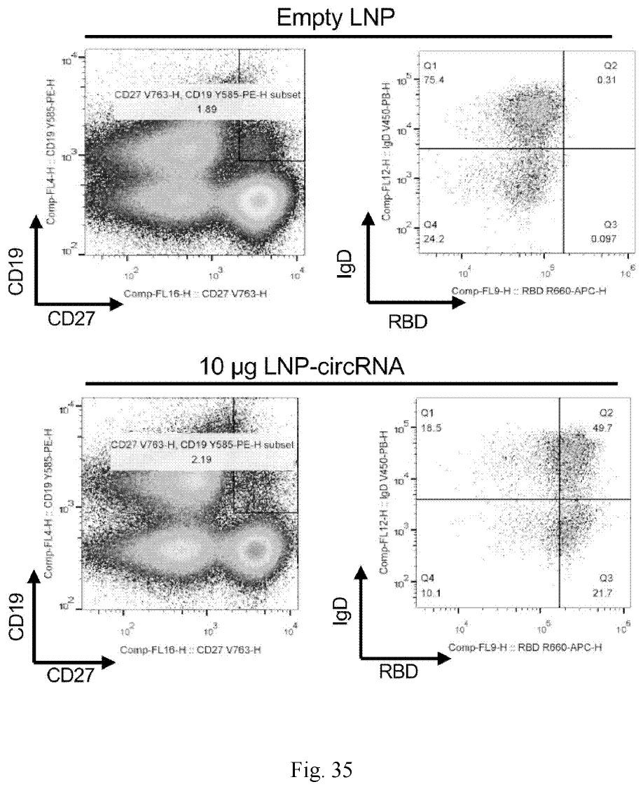

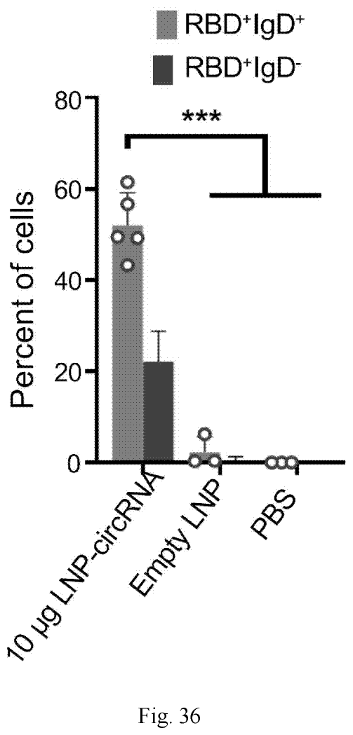

We used two different formulations to produce the LNP-circRNA particles, and achieved high encapsulation efficiency (>90%) with typical nanoparticle size at 90-100 nm (FIG. 33 showing the representative result using SM-102 formulation). The LNP-circRNAs were inoculated into the BALB/c mice with two separated IM injections, and the blood samples were collected at 2 weeks after each inoculation for further analysis (FIG. 34). We tested several formulations and circRNA designs, however the results from the LNP-circRNA-RBP using SM-102 formulation were shown for a better comparison with published results. Using flowcytometry, we found that a large fraction of the memory B-cells (i.e., CD19+CD27+ B-lymphocytes) had strong expression of the RBD antibody at two weeks after the second inoculation (FIG. 35, 36), indicative of a long-lasting immune response induced by antigen. In particular, >70% of CD19+CD27+ B-cells expressed RBD-specific antibody (i.e., RBD+), and ˜50% of the CD19+CD27+ B-cells were also IgD+RBD+(FIG. 36), suggesting a strong antibody response. As a control, the injection of LNP alone do not induce activation of RBD antibody, suggesting a specific response upon inoculation.

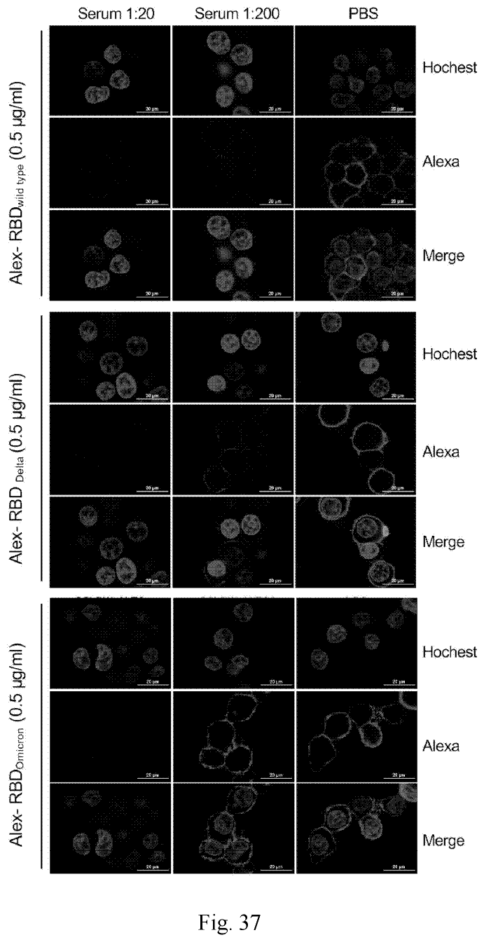

To test the activity of the RBD antibody in mouse serum, we next performed an antibody blocking assay to examine if the mouse serum can block the binding of fluorescence-labeled RDB to the 293 T cells stably expressing ACE2 (FIG. 37). We found that the serum can effectively block the binding of all RBD variants to the cell surface at a modest dilution (1:20). Given that we used a pretty high concentration of RBD (0.5 μg/ml), this protection is very impressive. Even in a very high dilution ratio (1:200), the serum still completely blocked the binding of wild type RBD and significantly reduced the binding of the RBD of the Delta variant. However, the serum did not block the binding of omicron RBD, which is consistent with the finding that the current mRNA vaccine based on the wild type S protein are weak in protecting against the omicron variant.

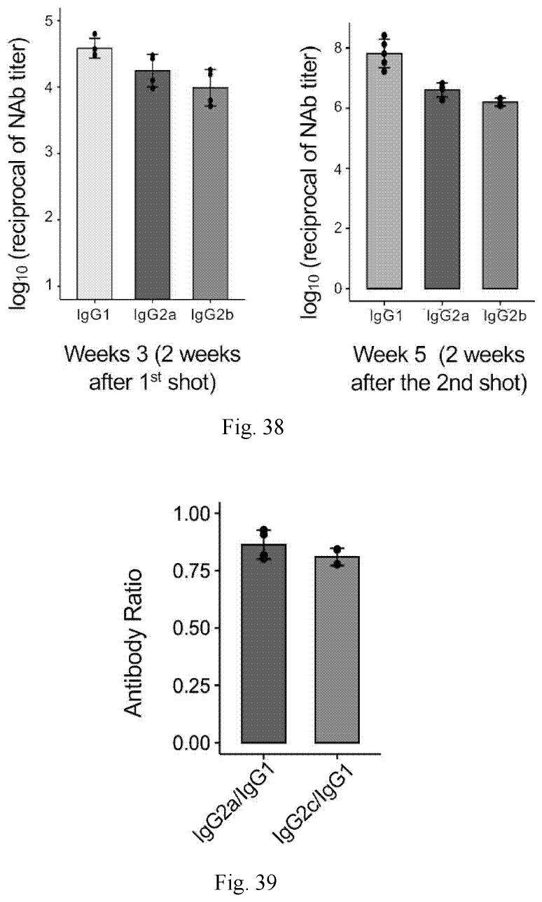

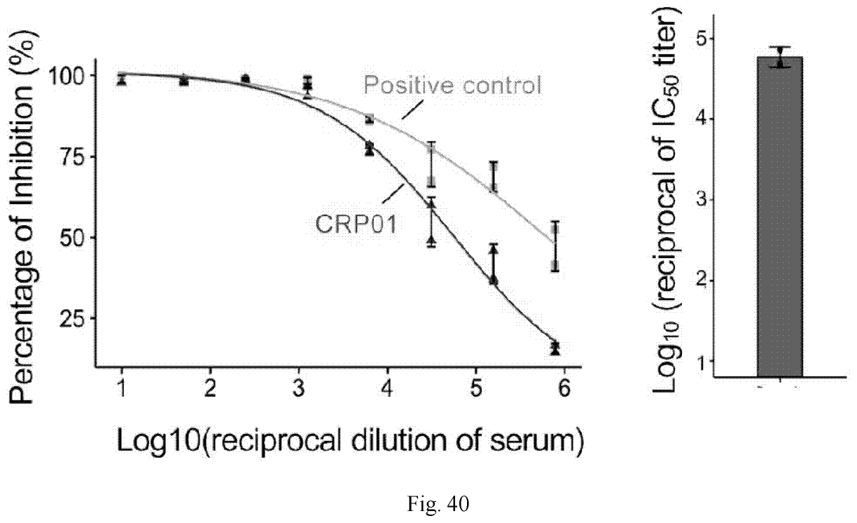

We further measured the titers of the neutralization antibody against RDB after each inoculation, and found a robust production of IgG production against RBD (FIG. 38-39). The antibody titer was close to 105 at two weeks after the first shot and reached near 108 after the second shot, which is at least 10 times higher than the reported mouse results using the LNP encapsulated linear mRNAs with similar formulation or using another circRNA design. In addition, the ratio of IgG2/IgG1 is around 0.75, which is similar to previously reported using linear mRNA vaccine. Finally we measured the antibody titer using the protection assay for SARS-CoV-2 pseudovirus, and found that a strong protection with a titer close to 105 (FIG. 40). Collectively, our preliminary data in mouse model suggested that the CirCode platform had a strong performance in design and generation of circRNA vaccines against SARS-CoV-2, which can potentially be expanded to the vaccine development for other viral pathogens.

### Plasmid Construction

The fragments of the group II intron in Clostridium tetani (CTE) and IRES sequences were chemical synthesized from GENEWIZ, and different protein coding fragments were amplified by PCR. These fragments were cloned into the NheI and XbaI digested backbone containing T7 RNA polymerase promoter and terminator by Gibson assembly.

### RNA Synthesis and Circularization

RNAs were in vitro transcribed from the XbaI digested linearized plasmid DNA template using T7 RiboMAX™ Large-Scale RNA Production System (Promega, P1320) in the presence of unmodified NTPs. After DNase I treatment, the RNA products were column purified with RNA Clean and Concentrator Kit (ZYMO research, R1013) to remove excess NTP and other salt in IVT buffer, as well as the possible small RNA fragments generated during IVT. In some experiments, the purified RNA was further circularized in a new circularization buffer. The RNA was first heated to 75° C. for 5 min and quickly cooled down to 45° C., after which a buffer including indicated magnesium and sodium was added to a final concentration: 50 mM Tris-HCl at pH 7.5, 50/100 mM NaCl, 0-40 mM MgCl2, and was then heated at 53° C. for indicated time for circularization. The best optimized reaction condition including concentration of magnesium and sodium, and incubation time at 53° C., was selected for further experiments.

### Example 14. CircRNA Identification

For the poly A tailing and RNase R treatment, the total RNAs from IVT were purified with by RNA clean-up columns, and then treated with E. coli Poly A Polymerase (NEB, M0276S) following the manufactory instruction. This step will add a poly-A tail to the free end of unspliced RNA precursor. After Poly A-tailing, the purified RNAs were digested by RNase R exoribonuclease (Lucigen, RNR07520) following the manufacturer's instructions, and enriched circRNAs were purified by column.

For the RNase H nicking assay, we incubated the 24 nt ssDNA probe with at 1:20 ratio, the RNase R enriched circRNAs were heated at 65° C. for 5 min. The RNase H buffer was added into the DNA-RNA mixture immediately. Then the mixture slowly cool to room temperature. After annealing, RNase H (Thermo Scientific, EN0201) was added to the mixture for 20 min at 37° C. The sequence of the ssDNA probe is 5′-TGGTGCTCGTAGGAGTAGTGAAAG-3′.

### RNA Gel Electrophoresis and Purification

RNA was run on a low melting point agarose gel (sigma Aldrich, A4018) at 120V using ice-cold DEPC-treated MOPS butter. After electrophoresis, the circRNA lane was cut and purified with Zymoclean Gel RNA Recovery Kit (ZYMO research, R1011). Before transfection, column purified circRNA was treat with phosphatase (NEB, M0525S) to remove potential 5′ phosphate which could produce immunogenicity.

Measurement of the Translation Products from circRNAs

Cells were seeded into 24-well plate at one day before transfection. The purified circRNAs are transfected into cells using Lipofectamine Messenger Max (Invitrogen, LMRNA001) according to the manufacturer's manual. After transfection, cells were cultured at 37° C. for 24 h. The cell lysis and supernatant were collected for luminescence assay using Dual-Luciferase® Reporter Assay System (Promega, E1910)

### RT-PCR of Circular RNA

Column purified RNA after IVT was electrophoresed with agarose gel. Bands corresponding to the circRNA were excised and extracted using a Zymoclean Gel RNA Extraction Kit (Zymogen, R1011). Purified RNA was reverse transcribed into cDNA using a PrimeScript RT Reagent Kit with random primers (TAKARA, RR037B), followed by PCR with primers that can amplify transcripts across the splice junction. The PCR products were Sanger sequenced to confirm the backsplicing of the circular RNA.

### HPLC Purification of Circular RNA

To obtain the high quality circRNA, spin column purified DNase I-treated RNA from IVT was resolved with high performance liquid chromatography. For semi-preparation with SHIMADZ LC-20A (Kyoto, Japan), 40 g RNA was loaded onto a 4.6×300 mm size exclusion column (Waters XBridge, BEH450A, 450A pore diameter, 3.5 m particle size) and eluted with mobile phase containing 10 mM Tris, 1 mM EDTA, 75 mM PB, pH7.4 at 25° C. with flow rate 0.5 ml/min. For full-preparation with Sepure SDL-30 (Suzhou, China), 5 mg RNA was loaded each run onto a 30×300 mm SEC column (Sepax, SRT SEC-1000A, 1000A pore diameter, 5.0 m particle size, Suzhou, China) with mobile phase containing 10 mM Tris, 1 mM EDTA, 75 mM PB, pH7.4 at 25° C. with flow rate 10 ml/min. Fractions were collected as indicated and testified with agarose gel electrophoresis.

Analysis of Circular RNA with Capillary Electrophoresis

Circular RNA purified from HPLC were further analyzed with capillary electrophoresis with Agilent 2100 Bioanalyzer in the RNA mode. Samples were diluted to appropriate concentration and analyzed according to the manufactory instructions.

### LNP Production Process

The circular RNA was encapsulated in a lipid nanoparticle via the NanoAssemblr Ignite system as previously described (Corbett, K. S. et al., (2020). SARS-CoV-2 mRNA vaccine design enabled by prototype pathogen preparedness. Nature 586, 567-571; Polack, F. P. et al., (2020). Safety and Efficacy of the BNT162b2 mRNA Covid-19 Vaccine. N Engl J Med 383, 2603-2615). In brief, an aqueous solution of circRNA at pH 4.0 is rapidly mixed with a lipid mixture dissolved in ethanol, which contain different ionizable cationic lipid, distearoylphosphatidylcholine (DSPC), DMG-PEG2000, and cholesterol. The ratios for the lipid mixture are MC3:DSPC:Cholesterol:PEG-2000=50:10:38.5:1.5 for formulation 1, SM-102:DSPC:Cholesterol:PEG-2000=50:10:38.5:1.5 for formulation 2, and ALC-0315:DSPC:Cholesterol:ALC-0159=46.3:9.4:42.7:1.6 for formulation 3. The resulting LNP mixture was then dialyzed against PBS and stored at −80° C. at a concentration of 0.5 μg/μl for further application.

Administration of LNP-circRNAs into Mice

Female BALB/C mice aged 8 weeks were purchased from Shanghai Model Organisms Center. 20 μg of CircRNA-LNPs in PBS were administrated into mice intramuscularly with 3/10 insulin syringes (BD biosciences). The serum was collected 24 hours after the administration of LNP, and 50 μl serum were used for Luciferase activity assay in vitro. Bioluminescence imaging was performed with an IVIS Spectrum (Roper Scientific). 24 hours after Gluc-LNP injection, 2 mg/kg of Coelenterazine (MedChemExpress, MCE) were administrated to mice intraperitoneally. Mice were then anesthetized after receiving the substrate in a chamber with 2.5% isoflurane (RWD Life Science Co.) and placed on the imaging platform while being maintained on 2% isoflurane via a nose cone. Mice were imaged 5 minutes post substrate injection with 30 seconds exposure time to ensure the signal were effectively and sufficiently acquired.

For immunogenicity studies, 8-week-old female BALB/c mice (Shanghai Model Organisms Center, Inc) were used. 10 μg of CircRNA-RBD-LNP were diluted in 50 μl 1×PBS and intramuscularly administrated into the mice same hind leg for both prime and boost shots. Mice in the control groups received PBS and empty LNPs. The blood samples were collected 2 weeks after prime and boost shots. Mice spleen were also harvested at the end point (2 weeks after boost) for immunostaining and flow cytometry.

1. A polynucleotide construct with self-splicing activity, comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

2. A polynucleotide construct with self-splicing activity, comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

3. A polynucleotide construct with self-splicing activity, comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

4. A polynucleotide construct with self-splicing activity, comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron into two fragments, and the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

5. The polynucleotide construct of any one of claims 1-4, wherein the polynucleotide construct has self-splicing activity in vitro.

6. The polynucleotide construct of any one of claims 1-5, wherein the E1 and/or the E2 is 0 to 20 nucleotides in length, preferably 0 to 10 nucleotides in length, such as 0, 1, 2, 3, 4, 5, 6, 7, 8, 9 or 10 nucleotides in length.

7. The polynucleotide construct of any one of claims 1-6, wherein the 5′ intron fragment and the 3′ intron fragment are obtained by segmenting a group II intron at an unpaired region into two fragments.

8. The polynucleotide construct of any one of claims 1-6, wherein the 5′ intron fragment and the 3′ intron fragment are obtained by segmenting a group II intron at a loop region of a stem-loop structure of domain 1.

9. The polynucleotide construct of any one of claims 1-6, wherein the 5′ intron fragment and the 3′ intron fragment are obtained by segmenting a group II intron at a loop region of a stem-loop structure of domain 2.

10. The polynucleotide construct of any one of claims 1-6, wherein the 5′ intron fragment and the 3′ intron fragment are obtained by segmenting a group II intron at a loop region of a stem-loop structure of domain 3.

11. The polynucleotide construct of any one of claims 1-6, wherein the 5′ intron fragment and the 3′ intron fragment are obtained by segmenting a group II intron at a loop region of a stem-loop structure of domain 4.

12. The polynucleotide construct of any one of claims 1-6, wherein the 5′ intron fragment and the 3′ intron fragment are obtained by segmenting a group II intron at a loop region of a stem-loop structure of domain 5.

13. The polynucleotide construct of any one of claims 1-6, wherein the 5′ intron fragment and the 3′ intron fragment are obtained by segmenting a group II intron at a loop region of a stem-loop structure of domain 6.

14. The polynucleotide construct of any one of claims 1-6, wherein the 5′ intron fragment and the 3′ intron fragment are obtained by segmenting a group II intron at a linear region between domain 1 and domain 2.

15. The polynucleotide construct of any one of claims 1-6, wherein the 5′ intron fragment and the 3′ intron fragment are obtained by segmenting a group II intron at a linear region between domain 2 and domain 3.

16. The polynucleotide construct of any one of claims 1-6, wherein the 5′ intron fragment and the 3′ intron fragment are obtained by segmenting a group II intron at a linear region between domain 3 and domain 4.

17. The polynucleotide construct of any one of claims 1-6, wherein the 5′ intron fragment and the 3′ intron fragment are obtained by segmenting a group II intron at a linear region between domain 4 and domain 5.

18. The polynucleotide construct of any one of claims 1-6, wherein the 5′ intron fragment and the 3′ intron fragment are obtained by segmenting a group II intron at a linear region between domain 5 and domain 6.

19. The polynucleotide construct of any one of claims 1-18, wherein the group II intron comprises a modification of one or more nucleotides relative to its wild-type form, and the modification is selected from one or more of a deletion, a substitution, and an addition.

20. The polynucleotide construct of claim 19, wherein the modification comprises a modification of one or more EBS sequences of the group II intron, wherein the EBS sequences are complementarily paired with one or more regions of a corresponding length in a target sequence on at least 60% of the nucleotide positions respectively.

21. The polynucleotide construct of claim 19, wherein the modification is a modification of the two EBS sequences of the group II intron, such as EBS1 and EBS3, wherein the EBS sequences are complementarily paired with two regions of a corresponding length in a target sequence on at least 60% of the nucleotide positions respectively; preferably, the two regions are located at both ends of the target sequence, respectively.

22. The polynucleotide construct of claim 19, wherein the modification is a modification of the two EBS sequences of the group II intron, such as EBS1′ and EBS3′, wherein the EBS sequences are complementarily paired with two regions of a corresponding length in a target sequence on at least 60% of the nucleotide positions respectively; preferably, the two regions are located at both ends of the target sequence, respectively.

23. The polynucleotide construct of claim 19, wherein the modification is a modification of EBS1 and/or δ sequence of the group II intron, or a modification of EBS1′ and/or δ″ sequence, wherein the EBS1 and/or δ sequence is complementarily paired with a region of a corresponding length in a target sequence on at least 60% of the nucleotide, optionally the modification is a modification of EBS1 and/or δ sequence and its upstream sequence, wherein the EBS1 and/or δ sequence and its upstream is complementarily paired with a region of a corresponding length in a target sequence on at least 60% of the nucleotide.

24. The polynucleotide construct of claim 19, wherein the modification comprises a deletion of part or all of domain 4, such as a deletion of an intron-encoded protein (IEP) sequence in domain 4, preferably a deletion of all of domain 4.

25. The polynucleotide construct of claim 19, wherein the modification comprises a deletion of an open reading frame (ORF).

26. The polynucleotide construct of any one of claims 1-25, wherein the polynucleotide construct is capable of forming a near-scarless circular RNA of the target sequence.

27. The polynucleotide construct of claim 26, wherein the near-scarless circular RNA has a scar region equal to or less than 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides in length.

28. The polynucleotide construct of any one of claims 1-25, wherein the polynucleotide construct is capable of forming a scarless circular RNA of the target sequence.

29. The polynucleotide construct of any one of claims 1-28, wherein E1 and E2 are each 0 nucleotide in length.

30. The polynucleotide construct of any one of claims 1-28, wherein the E1 is 0 nucleotide in length.

31. The polynucleotide construct of any one of claims 1-28, wherein the E2 is 0 nucleotide in length.

32. The polynucleotide construct of any one of claims 1-31, wherein the group II intron is a group II intron derived from a microorganism.

33. The polynucleotide construct of any one of claims 1-32, wherein the noncoding sequence is selected from the group consisting of: a spacer sequence of SEQ ID NOs: 4-6, a polyA sequence, a poly-A-C sequence, a poly-C sequence, a poly-U sequence, an IRES, a ribosome binding site, an aptamer sequence, an RNA scaffold, a riboswitch, a ribozyme other than a self-splicing ribozyme, an antisense oligonucleotide (ASO), a scaffold, a small RNA binding site, a translational regulatory sequence, and a protein binding site.

34. The polynucleotide construct of any one of claims 1-33, wherein the group II intron comprises a nucleic acid sequence selected from the group consisting of:
(a) SEQ ID NO: 33;
(b) SEQ ID NO: 34;
(c) SEQ ID NO: 35;
(d) SEQ ID NO: 36;
(e) SEQ ID NO: 37;
(f) SEQ ID NO: 38;
(g) SEQ ID NO: 39;
(h) SEQ ID NO: 40; and
(i) SEQ ID NO: 41.

35. The polynucleotide construct of any one of claims 1-34, wherein the polynucleotide construct is an RNA polynucleotide construct.

36. The polynucleotide construct of claim 35, wherein the 3′ intron fragment comprises a nucleic acid sequence selected from the group consisting of:
(a) a nucleic acid sequence 95% identical to SEQ ID NO: 42;
(b) a nucleic acid sequence 98% identical to SEQ ID NO: 42;
(c) a nucleic acid sequence 99% identical to SEQ ID NO: 42;
(d) SEQ ID NO: 42;
(e) a nucleic acid sequence 95% identical to SEQ ID NO: 43;
(f) a nucleic acid sequence 98% identical to SEQ ID NO: 43;
(g) a nucleic acid sequence 99% identical to SEQ ID NO: 43;
(h) SEQ ID NO: 43;
(i) a nucleic acid sequence 95% identical to SEQ ID NO: 44;
(j) a nucleic acid sequence 98% identical to SEQ ID NO: 44;
(k) a nucleic acid sequence 99% identical to SEQ ID NO: 44;
(l) SEQ ID NO: 44;
(m) a nucleic acid sequence 95% identical to SEQ ID NO: 45;
(n) a nucleic acid sequence 98% identical to SEQ ID NO: 45;
(o) a nucleic acid sequence 99% identical to SEQ ID NO: 45;
(p) SEQ ID NO: 45;
(q) a nucleic acid sequence 95% identical to SEQ ID NO: 46;
(r) a nucleic acid sequence 98% identical to SEQ ID NO: 46;
(s) a nucleic acid sequence 99% identical to SEQ ID NO: 46;
(t) SEQ ID NO: 46;
(u) a nucleic acid sequence 95% identical to SEQ ID NO: 47;
(v) a nucleic acid sequence 98% identical to SEQ ID NO: 47;
(w) a nucleic acid sequence 99% identical to SEQ ID NO: 47;
(x) SEQ ID NO: 47;
(y) a nucleic acid sequence 95% identical to SEQ ID NO: 48;
(z) a nucleic acid sequence 98% identical to SEQ ID NO: 48;
(aa) a nucleic acid sequence 99% identical to SEQ ID NO: 48;
(bb) a nucleic acid sequence SEQ ID NO: 48;
(cc) a nucleic acid sequence 95% identical to SEQ ID NO: 49;
(dd) a nucleic acid sequence 98% identical to SEQ ID NO: 49;
(ee) a nucleic acid sequence 99% identical to SEQ ID NO: 49;
(ff) SEQ ID NO: 49;
(gg) a nucleic acid sequence 95% identical to SEQ ID NO: 50;
(hh) a nucleic acid sequence 98% identical to SEQ ID NO: 50;
(ii) a nucleic acid sequence 99% identical to SEQ ID NO: 50;
(jj) SEQ ID NO: 50;
(kk) a nucleic acid sequence 95% identical to SEQ ID NO: 51;
(ll) a nucleic acid sequence 98% identical to SEQ ID NO: 51;
(mm) a nucleic acid sequence 99% identical to SEQ ID NO: 51;
(nn) SEQ ID NO: 51;
(oo) a nucleic acid sequence 95% identical to SEQ ID NO: 52;
(pp) a nucleic acid sequence 98% identical to SEQ ID NO: 52;
(qq) a nucleic acid sequence 99% identical to SEQ ID NO: 52; and
(rr) SEQ ID NO: 52.

37. The polynucleotide construct of claim 35 or 36, wherein the E2 comprises a nucleic acid sequence selected from the group consisting of:
(a) SEQ ID NO: 53;
(b) SEQ ID NO: 54;
(c) SEQ ID NO: 55;
(d) SEQ ID NO: 56.
(e) SEQ ID NO: 57;
(f) SEQ ID NO: 58;
(g) SEQ ID NO: 59;
(h) SEQ ID NO: 60.
(i) SEQ ID NO: 61;
(j) SEQ ID NO: 62; and
(k) SEQ ID NO: 63.

38. The polynucleotide construct of any one of claims 35-37, wherein the E1 comprises a nucleic acid sequence selected from the group consisting of:
(a) SEQ ID NO: 64;
(b) SEQ ID NO: 65;
(c) SEQ ID NO: 66;
(d) SEQ ID NO: 67.
(e) SEQ ID NO: 68;
(f) SEQ ID NO: 69;
(g) SEQ ID NO: 70;
(h) SEQ ID NO: 71.
(i) SEQ ID NO: 72;
(j) SEQ ID NO: 73; and
(k) SEQ ID NO: 74.

39. The polynucleotide construct of any one of claims 35-38, wherein the 5′ intron fragment comprises a nucleic acid sequence selected from the group consisting of:
(a) a nucleic acid sequence 95% identical to SEQ ID NO: 75;
(b) a nucleic acid sequence 98% identical to SEQ ID NO: 75;
(c) a nucleic acid sequence 99% identical to SEQ ID NO: 75;
(d) SEQ ID NO: 75;
(e) a nucleic acid sequence 95% identical to SEQ ID NO: 76;
(f) a nucleic acid sequence 98% identical to SEQ ID NO: 76;
(g) a nucleic acid sequence 99% identical to SEQ ID NO: 76;
(h) SEQ ID NO: 76;
(i) a nucleic acid sequence 95% identical to SEQ ID NO: 77;
(j) a nucleic acid sequence 98% identical to SEQ ID NO: 77;
(k) a nucleic acid sequence 99% identical to SEQ ID NO: 77;
(l) SEQ ID NO: 77;
(m) a nucleic acid sequence 95% identical to SEQ ID NO: 78;
(n) a nucleic acid sequence 98% identical to SEQ ID NO: 78;
(o) a nucleic acid sequence 99% identical to SEQ ID NO: 78;
(p) SEQ ID NO: 78;
(q) a nucleic acid sequence 95% identical to SEQ ID NO: 79;
(r) a nucleic acid sequence 98% identical to SEQ ID NO: 79;
(s) a nucleic acid sequence 99% identical to SEQ ID NO: 79;
(t) SEQ ID NO: 79;
(u) a nucleic acid sequence 95% identical to SEQ ID NO: 80;
(v) a nucleic acid sequence 98% identical to SEQ ID NO: 80;
(w) a nucleic acid sequence 99% identical to SEQ ID NO: 80;
(x) SEQ ID NO: 80;
(y) a nucleic acid sequence 95% identical to SEQ ID NO: 81;
(z) a nucleic acid sequence 98% identical to SEQ ID NO: 81;
(aa) a nucleic acid sequence 99% identical to SEQ ID NO: 81;
(bb) SEQ ID NO: 81;
(cc) a nucleic acid sequence 95% identical to SEQ ID NO: 82;
(dd) a nucleic acid sequence 98% identical to SEQ ID NO: 82;
(ee) a nucleic acid sequence 99% identical to SEQ ID NO: 82;
(ff) SEQ ID NO: 82;
(gg) a nucleic acid sequence 95% identical to SEQ ID NO: 83;
(hh) a nucleic acid sequence 98% identical to SEQ ID NO: 83;
(ii) a nucleic acid sequence 99% identical to SEQ ID NO: 83;
(jj) SEQ ID NO: 83;
(kk) a nucleic acid sequence 95% identical to SEQ ID NO: 84;
(ll) a nucleic acid sequence 98% identical to SEQ ID NO: 84;
(mm) a nucleic acid sequence 99% identical to SEQ ID NO: 84;
(nn) SEQ ID NO: 84;
(oo) a nucleic acid sequence 95% identical to SEQ ID NO: 85;
(pp) a nucleic acid sequence 98% identical to SEQ ID NO: 85;
(qq) a nucleic acid sequence 99% identical to SEQ ID NO: 85;
(rr) SEQ ID NO: 85;
(ss) a nucleic acid sequence 95% identical to SEQ ID NO: 86;
(tt) a nucleic acid sequence 98% identical to SEQ ID NO: 86;
(uu) a nucleic acid sequence 99% identical to SEQ ID NO: 86;
(vv) SEQ ID NO: 86;
(ww) a nucleic acid sequence 95% identical to SEQ ID NO: 87;
(xx) a nucleic acid sequence 98% identical to SEQ ID NO: 87;
(yy) a nucleic acid sequence 99% identical to SEQ ID NO: 87;
(zz) SEQ ID NO: 87;
(aaa) a nucleic acid sequence 95% identical to SEQ ID NO: 88;
(bbb) a nucleic acid sequence 98% identical to SEQ ID NO: 88;
(ccc) a nucleic acid sequence 99% identical to SEQ ID NO: 88; and
(ddd) SEQ ID NO: 88.

40. The polynucleotide construct of any one of claims 3-40, wherein the 5′ homology arm comprises the nucleic acid sequence of SEQ ID NO: 105.

41. The polynucleotide construct of any one of claims 3-40, wherein the 3′ homology arm comprises the nucleic acid sequence of SEQ ID NO: 106.

42. The polynucleotide construct of any one of claims 3-41, wherein the 5′ homology arm or 3′ homology arm is 15 to 60 nucleotides in length.

43. The polynucleotide construct of any one of claims 3-42, wherein the 5′ homology arm or 3′ homology arm sequence has up to 10% base mismatches.

44. The polynucleotide construct of any one of claims 1-43, wherein the target sequence comprises a 5′ arm sequence selected from the group consisting of:
(a) SEQ ID NO: 89;
(b) SEQ ID NO: 90;
(c) SEQ ID NO: 91;
(d) SEQ ID NO: 92;
(e) SEQ ID NO: 93;
(f) SEQ ID NO: 94;
(g) SEQ ID NO: 95; and
(h) SEQ ID NO: 96.

45. The polynucleotide construct of any one of claims 1-44, wherein the target sequence comprises a 3′ arm sequence selected from the group consisting of:
(a) SEQ ID NO: 97;
(b) SEQ ID NO: 98;
(c) SEQ ID NO: 99;
(d) SEQ ID NO: 100;
(e) SEQ ID NO: 101;
(f) SEQ ID NO: 102;
(g) SEQ ID NO: 103; and
(h) SEQ ID NO: 104.

46. The polynucleotide construct of any one of claims 1-45, wherein the target sequence comprises Formula I:
TI-(L)ₙ-Z1 (I)
wherein:
TI is an engineered translation initiation element comprising an internal ribosome entry site (IRES)-like polynucleotide sequence or a natural IRES sequence,
Z1 is an expression sequence encoding a therapeutic product;
L is a linker sequence;
A1 and B1 are a pair of sequences capable of circularization of the RNA polynucleotide; and
n is an integer selected from 0 to 2.

47. The polynucleotide construct of claim 46, wherein Z1 comprises a nucleic acid sequence selected from the group consisting of:
(a) a nucleic acid sequence 95% identical to SEQ ID NO: 107;
(b) a nucleic acid sequence 98% identical to SEQ ID NO: 107;
(c) a nucleic acid sequence 99% identical to SEQ ID NO: 107;
(d) SEQ ID NO: 107;
(e) a nucleic acid sequence 95% identical to SEQ ID NO: 108;
(f) a nucleic acid sequence 98% identical to SEQ ID NO: 108;
(g) a nucleic acid sequence 99% identical to SEQ ID NO: 108;
(h) SEQ ID NO: 108;
(i) a nucleic acid sequence 95% identical to SEQ ID NO: 109;
(j) a nucleic acid sequence 98% identical to SEQ ID NO: 109;
(k) a nucleic acid sequence 99% identical to SEQ ID NO: 109;
(l) SEQ ID NO: 109;
(m) a nucleic acid sequence 95% identical to SEQ ID NO: 110;
(n) a nucleic acid sequence 98% identical to SEQ ID NO: 110;
(o) a nucleic acid sequence 99% identical to SEQ ID NO: 110;
(p) SEQ ID NO: 110;
(q) a nucleic acid sequence 95% identical to SEQ ID NO: 111;
(r) a nucleic acid sequence 98% identical to SEQ ID NO: 111;
(s) a nucleic acid sequence 99% identical to SEQ ID NO: 111;
(t) SEQ ID NO: 111;
(u) a nucleic acid sequence 95% identical to SEQ ID NO: 112;
(v) a nucleic acid sequence 98% identical to SEQ ID NO: 112;
(w) a nucleic acid sequence 99% identical to SEQ ID NO: 112; and
(x) SEQ ID NO: 112.

48. The polynucleotide construct of claim 46, wherein Z1 comprises a nucleic acid sequence encoding the amino acid sequence selected from the group consisting of:
(a) SEQ ID NO: 113;
(b) SEQ ID NO: 114;
(c) SEQ ID NO: 115;
(d) SEQ ID NO: 116;
(e) SEQ ID NO: 117; and
(f) SEQ ID NO: 118.

49. The polynucleotide construct of any one of claims 1-48, comprising a modified RNA nucleotide and/or modified nucleoside.

50. The polynucleotide construct of any one of claims 1-49, comprising 10% to 100% modified RNA nucleotide and/or modified nucleoside.

51. The polynucleotide construct of any one of claims 49-50, wherein at least one of the modified RNA nucleotide and/or modified nucleoside is m5C (5-methylcytidine).

52. The polynucleotide construct of any one of claims 49-50, wherein at least one of the modified RNA nucleotide and/or modified nucleoside is m5U (5-methyluridine).

53. The polynucleotide construct of any one of claims 49-50, wherein at least one of the modified RNA nucleotide and/or modified nucleoside is m6A (N6-methyladenosine).

54. The polynucleotide construct of any one of claims 49-50, wherein at least one of the modified RNA nucleotide and/or modified nucleoside is Y (pseudouridine).

55. The polynucleotide construct of any one of claims 49-50, wherein at least one of the modified RNA nucleotide and/or modified nucleoside is m1A (1-methyladenosine).

56. The polynucleotide construct of any one of claims 49-55, wherein at least one of the modified RNA nucleotide and/or modified nucleoside is introduced at transcription (IVT).

57. The polynucleotide construct of any one of claims 49-50, wherein the modified nucleoside is selected from the group consisting of: m5C (5-methylcytidine), m5U (5-methyluridine), m6A (N6-methyladenosine), s2U (2-thiouridine), Y (pseudouridine), Um (2′-O-methyluridine), m1A (1-methyladenosine), m2A (2-methyladenosine), Am (2′-O-methyladenosine), ms2 m6A (2-methylthio-N6-methyladenosine), i6A (N6-isopentenyladenosine), ms2i6A (2-methylthio-N6 isopentenyladenosine), io6A (N6-(cis-hydroxyisopentenyl)adenosine), ms2io6A (2-methylthio-N6-(cis-hydroxyisopentenyl)adenosine), g6A (N6-glycinylcarbamoyladenosine), t6A (N6-threonylcarbamoyladeno sine), ms2t6A (2-methylthio-N6-threonyl carbamoyladenosine), m6t6A (N6-methyl-N6-threonylcarbamoyladenosine), hn6A (N6-hydroxynorvalylcarbamoyladenosine), ms2hn6A (2-methylthio-N6-hydroxynorvalyl carbamoyladenosine), Ar(p) (2′-O-ribosyladenosine (phosphate)), I (inosine), m1I (1-methylinosine), mlhn (1,2′-O-dimethylinosine), m3C (3-methylcytidine), Cm (2′-O-methylcytidine), s2C (2-thiocytidine), ac4C (N4-acetylcytidine), (5-formylcytidine), m5Cm (5,2′-O-dimethylcytidine), ac4Cm (N4-acetyl-2′-O-methylcytidine), k2C (lysidine), m!G (1-methylguanosine), m2G (N2-methylguanosine), m7G (7-methylguanosine), Gm (2′-O-methylguanosine), m2 2G (N2,N2-dimethylguanosine), m2Gm (N2,2′-O-dimethylguanosine), m2 aGm (N2,N2,2′-O-trimethylguanosine), Gr(p) (2′-O-ribosylguanosine(phosphate)), yW (wybutosine), oayW (peroxywybutosine), OHyW (hydroxy wybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQo (7-cyano-7-deazaguanosine), preQi (7-aminomethyl-7-deazaguanosine), G+(archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxy acetic acid), mcmo5U (uridine 5-oxy acetic acid methyl ester), chm5U (5-(carboxyhydroxymethyl)uridine)), mchm5U (5-(carboxyhydroxymethyl)uridine methyl ester), mcm5U (5-methoxycarbonylmethyluridine), mcm5Um (5-methoxycarbonylmethyl-2′-O-methyluridine), mcm5s2U (5-methoxycarbonylmethyl-2-thiouridine), nm5S2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyluridine), ncm5Um (5-carbamoylmethyl-2′-O-methyluridine), cmnm⁵U (5-carboxymethylaminomethyluridine), cmnm⁵Um (5-carboxymethylaminomethyl-2′-O-methyluridine), cmnm⁵s2U (5-carboxymethylaminomethyl-2-thiouridine), m6 2A (N6,N6-dimethyladenosine), Im (2′-O-methylinosine), m4C (N4-methylcytidine), m4Cm (N4,2′-O-dimethylcytidine), hm5C (5-hydraxymethylcytidine), m3U (3-methyluridine), cm5U (5-carboxymethyluridine), m6Am (N6,2′-O-dimethyladenosine), m6 2Am (N6,N6,0-2′-trimethyladenosine), m2,7G (N2,7-dimethylguanosine), m2,2,7G (N2,N2,7-trimethylguanosine), m3Um (3,2′-O-dimethyluridine), m5D (5-methyldihydrouridine), f5Cm (5-formyl-2′-O-methylcytidine), m′Gm (1,2′-O-dimethylguanosine), m′Am (1,2′-O-dimethyladenosine), rm 5U (5-taurinomethyluridine), rm5s2U (5-taurinomethyl-2-thiouridine)), imG-14 (4-demethylwyosine), imG2 (isowyosine), or ac6A (N6-acetyladenosine), pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5-methylcytosine, pseudouridine, and 1-methylpseudouridine.

58. A circular RNA produced by the polynucleotide construct of any of claims 1-57.

59. The circular RNA of claim 58, not comprising any other sequences that do not belong to the target sequence, such as not comprising all or part of an E2 sequence and an E1 sequence.

60. A method of making a circular RNA, said method comprising: preparing a vector comprising the following operably linked elements from 5′ to 3′:
(a) a 3′ intron fragment;
(b) an exon fragment 2 (E2);
(c) a target sequence;
(d) an exon fragment 1 (E1); and
(e) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

61. A method of making circular RNA, said method comprising: preparing a vector comprising the following operably linked elements from 5′ to 3′:
(a) a 3′ intron fragment;
(b) an exon fragment 2 (E2);
(c) a linker sequence;
(d) a target sequence;
(e) a linker sequence;
(f) an exon fragment 1 (E1); and
(g) a 5′ intron fragment,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

62. A method of making circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a target sequence;
(e) an exon fragment 1 (E1);
(f) a 5′ intron fragment; and
(g) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

63. A method of making circular RNA, said method comprising: preparing a vector comprising the following operably linked elements from 5′ to 3′:
(a) a 5′ homology arm;
(b) a 3′ intron fragment;
(c) an exon fragment 2 (E2);
(d) a linker sequence;
(e) a target sequence;
(f) a linker sequence;
(g) an exon fragment 1 (E1);
(h) a 5′ intron fragment; and
(i) a 3′ homology arm,
wherein:
the 5′ intron fragment and the 3′ intron fragment are each a fragment of a group II intron, wherein the 5′ intron fragment is located on the 5′ side of the 3′ intron fragment in the group II intron,
the E1 is a 5′ adjacent exon fragment of the group II intron, which is ≥0 nucleotides in length,
the E2 is a 3′ adjacent exon fragment of the group II intron, which is ≥0 nucleotides in length, and
the target sequence is absent, or is a protein coding sequence, a noncoding sequence, or a combination thereof.

64. A method for expressing a protein in a cell, comprising (a) transfecting the cell with the circular RNA of any one of claims 58-63, or (b) subjecting the polynucleotide construct of any of claims 1-57 to a self-splicing circularization reaction to form a circular RNA, and transfecting the cell with the circular RNA; wherein, preferably the cell is a eukaryotic cell.

65. A method for expressing a protein in a cell, comprising (a) transfecting the cell with the circular RNA of any one of claims 58-63, or (b) subjecting the construct of any of claims 1-57 to a self-splicing circularization reaction to form a circular RNA, and transfecting the cell with the circular RNA; wherein, preferably the cell is a hepatocyte, epithelial cell, hematopoietic cell, epithelial cell, endothelial cell, lung cell, bone cell, stem cell, mesenchymal cell, neural cell (e.g., meninge, astrocyte, motor neuron, cell of the dorsal root ganglia and anterior horn motor neuron), photoreceptor cell (e.g., rod and cone), retinal pigmented epithelial cell, secretory cell, cardiac cell, adipocyte, vascular smooth muscle cell, cardiomyocyte, skeletal muscle cell, beta cell, pituitary cell, synovial lining cell, ovarian cell, testicular cell, fibroblast, B cell, T cell, dendritic cell, macrophage, reticulocyte, leukocyte, granulocyte, tumor cell, NK cell, liver starlet cell, HEK293, HEK293T, HeLa, MCF7, PC3, A549, NCI-H727, HCT-116, MCF10A, HPReC, FHC, immortalized cell lines, primary cell, yeast cell, Saccharomyces cerevisiae, Pichia pastoris, bacteria cell, Escherichia coli, insect cell, Spodoptera frugiperda sf9, Mimic Sf9, sf21, or Drosophila S2.

66. A method for generating a sequence with self-splicing activity using a group II intron, the method comprising the steps of:
(a) defining the sequence of the group II intron; optionally examining the in vitro self-splicing activity of the group II intron using a splicing assay (linear splicing);
(b) splitting the group II intron into two fragments,
(c) reversing the order of the two intron fragments, and
(d) confirming the in vitro circularization of RNA using a splicing assay.
